SEQUENCE LISTING (128 sequences)

SEQ ID NO: 1-32 - skipped (32 entries)

SEQ ID NO: 33 - DNA, 381 bp, synthetic construct
  source 1..381: mol_type = other DNA
gaggtgcagttggtggagtctgggggaggcttggtacagcctggggggtccctgagactctcctgtgcagcctctggattcacttttagcaactatgccatgaactgggtccgccaggctccagggaaggggctggagtgggtctcaactattagcggaagtggtggtgccacaaggtatgcagactccgtgaagggccgattcaccatatccagagacaattccaggaacacggtgtatctgcaaatgaacagcctgagagtcgaggacacggccgttttttactgtacgaaagatcggctcattatggctacggttcggggaccctattactacggtatggacgtctggggccaagggaccacggtcaccgtctcctca

SEQ ID NO: 34 - protein, 127 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSS

SEQ ID NO: 35 - DNA, 24 bp, synthetic construct
  source 1..24: mol_type = other DNA
ggattcacttttagcaactatgcc

SEQ ID NO: 36 - protein, 8 aa, synthetic construct
GFTFSNYA

SEQ ID NO: 37 - DNA, 24 bp, synthetic construct
  source 1..24: mol_type = other DNA
attagcggaagtggtggtgccaca

SEQ ID NO: 38 - protein, 8 aa, synthetic construct
ISGSGGAT

SEQ ID NO: 39 - DNA, 60 bp, synthetic construct
  source 1..60: mol_type = other DNA
acgaaagatcggctcattatggctacggttcggggaccctattactacggtatggacgtc

SEQ ID NO: 40 - protein, 20 aa, synthetic construct
TKDRLIMATVRGPYYYGMDV

SEQ ID NO: 41 - DNA, 15 bp, synthetic construct
  source 1..15: mol_type = other DNA
aactatgccatgaac

SEQ ID NO: 42 - protein, 5 aa, synthetic construct
NYAMN

SEQ ID NO: 43 - DNA, 51 bp, synthetic construct
  source 1..51: mol_type = other DNA
actattagcggaagtggtggtgccacaaggtatgcagactccgtgaagggc

SEQ ID NO: 44 - protein, 17 aa, synthetic construct
TISGSGGATRYADSVKG

SEQ ID NO: 45 - DNA, 54 bp, synthetic construct
  source 1..54: mol_type = other DNA
gatcggctcattatggctacggttcggggaccctattactacggtatggacgtc

SEQ ID NO: 46 - protein, 18 aa, synthetic construct
DRLIMATVRGPYYYGMDV

SEQ ID NO: 47 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaacctcctgatctatgctgcatccagtttgcaaagtggggtcccatcaaggttcagtggcagtggatctgagacagatttcactctcaccatcagcagtctgcaacctgaagattttgcaacttactactgtcaacagagtcacagtgtctcattcactttcggccctgggaccaaagtggatatcaaa

SEQ ID NO: 48 - protein, 107 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIK

SEQ ID NO: 49 - DNA, 18 bp, synthetic construct
  source 1..18: mol_type = other DNA
cagagcattagcagctat

SEQ ID NO: 50 - protein, 6 aa, synthetic construct
QSISSY

SEQ ID NO: 51-52 - skipped (2 entries)

SEQ ID NO: 53 - DNA, 27 bp, synthetic construct
  source 1..27: mol_type = other DNA
caacagagtcacagtgtctcattcact

SEQ ID NO: 54 - protein, 9 aa, synthetic construct
QQSHSVSFT

SEQ ID NO: 55 - DNA, 33 bp, synthetic construct
  source 1..33: mol_type = other DNA
cgggcaagtcagagcattagcagctatttaaat

SEQ ID NO: 56 - protein, 11 aa, synthetic construct
RASQSISSYLN

SEQ ID NO: 57 - DNA, 21 bp, synthetic construct
  source 1..21: mol_type = other DNA
gctgcatccagtttgcaaagt

SEQ ID NO: 58 - protein, 7 aa, synthetic construct
AASSLQS

SEQ ID NO: 59 - DNA, 27 bp, synthetic construct
  source 1..27: mol_type = other DNA
caacagagtcacagtgtctcattcact

SEQ ID NO: 60 - protein, 9 aa, synthetic construct
QQSHSVSFT

SEQ ID NO: 61 - DNA, 1362 bp, synthetic construct
  source 1..1362: mol_type = other DNA
gaagtgcaactggtggagtccggaggaggcctggtgcagcctggaggaagcctgaggctgagctgtgccgccagcggcttcaccttcagcaactacgccatgaactgggtgaggcaggcccctggcaagggactggagtgggtctccaccatcagcggctccggaggcgctacacggtacgccgatagcgtgaagggccggtttaccatttcccgggacaactcccggaacaccgtgtacctccagatgaacagcctgagggtggaggataccgccgtgttctactgcaccaaggacaggctgattatggccaccgtgaggggaccttactactatggcatggatgtgtggggccagggcacaaccgtcaccgtgtcctccgcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 62 - protein, 454 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - DNA, 642 bp, synthetic construct
  source 1..642: mol_type = other DNA
gacatccagatgacccagtccccttcctccctgagcgctagcgtgggagatagggtgaccatcacctgcagggcctcccaaagcatctcctcctacctgaactggtaccagcagaaacccggcaaggcccccaacctgctgatctacgctgcctcctccctccagtccggcgtgcctagcaggtttagcggctccggaagcgagaccgacttcaccctgaccatctcctccctgcagcccgaggacttcgccacctactactgccagcaatcccacagcgtgtccttcaccttcggccccggcaccaaggtggacatcaagaggaccgtggccgccccctccgtgttcatctttcccccctccgatgaacagctgaagagcggcaccgctagcgtggtgtgcctgctgaacaacttctaccccagggaggccaaggtgcagtggaaggtggacaatgccctgcagtccggcaacagccaggagagcgtgaccgagcaggactccaaggacagcacctacagcctgtcctccaccctgaccctgtccaaggccgactacgagaagcacaaagtgtacgcctgcgaagtgacccatcagggcctgagctcccccgtgaccaagtcctttaacaggggcgagtgc

SEQ ID NO: 64 - protein, 214 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65-127 - skipped (63 entries)

SEQ ID NO: 128 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0059289 A1
(43) Pub. Date: Feb. 20, 2025

(54) DOSING AND TREATMENT OF IMMUNE-MEDIATED DISEASES AND BIOMARKERS ASSOCIATED WITH IMMUNE-MEDIATED DISEASES

(71) Applicant: KYMAB LIMITED, Cambridge (GB)

(72) Inventors: Charlotte BERNIGAUD, Paris (FR); Sonya DAVEY, Cambridge, MA (US); Fabrice HURBIN, Paris (FR); John O'MALLEY, Cambridge, MA (US); Natalie RYNKIEWICZ, Paris (FR); Johannes-Christoph SCHNEIDER, Frankfurt am Main (DE); Marisa STEBEGG-WAGNER, Paris (FR); Gilles TIRABOSCHI, Paris (FR); Karl YEN, Paris (FR)

(21) Appl. No.: 18/748,458

(22) Filed: Jun. 20, 2024

Related U.S. Application Data
(60) Provisional application No. 63/521,985, filed on Jun. 20, 2023.
(60) Provisional application No. 63/522,039, filed on Jun. 20, 2023.
(60) Provisional application No. 63/522,098, filed on Jun. 20, 2023.
(60) Provisional application No. 63/522,784, filed on Jun. 23, 2023.
(60) Provisional application No. 63/522,822, filed on Jun. 23, 2023.
(60) Provisional application No. 63/523,284, filed on Jun. 26, 2023.
(60) Provisional application No. 63/541,102, filed on Sep. 28, 2023.
(60) Provisional application No. 63/541,111, filed on Sep. 28, 2023.
(60) Provisional application No. 63/541,137, filed on Sep. 28, 2023.
(60) Provisional application No. 63/588,829, filed on Oct. 9, 2023.
(60) Provisional application No. 63/588,839, filed on Oct. 9, 2023.
(60) Provisional application No. 63/588,847, filed on Oct. 9, 2023.
(60) Provisional application No. 63/605,649, filed on Dec. 4, 2023.
(60) Provisional application No. 63/619,620, filed on Jan. 10, 2024.
(60) Provisional application No. 63/551,294, filed on Feb. 8, 2024.
(60) Provisional application No. 63/561,582, filed on Mar. 5, 2024.
(60) Provisional application No. 63/644,272, filed on May 8, 2024.

(51) Int. Cl. C07K 16/28; A61K 39/00; A61P 17/00

(52) U.S. Cl. CPC C07K 16/2875; A61P 17/00; A61K 2039/505; A61K 2039/545

(57) ABSTRACT

The present disclosure provides methods of treating an immune-mediated disease in a subject in need thereof. The present disclosure provides methods of treating atopic dermatitis in a subject in need thereof. The present disclosure provides methods of treating moderate-to-severe atopic dermatitis in a subject in need thereof. The present disclosure provides a method of treating atopic dermatitis (AD) in a subject in need thereof, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control.

### RELATED APPLICATIONS

This application claims the benefit of U.S. Provisional Patent Application Ser. Nos. 63/521,985, filed Jun. 20, 2023, 63/522,039, filed Jun. 20, 2023, 63/522,098, filed Jun. 20, 2023, 63/522,784, filed Jun. 23, 2023, 63/522,822, filed Jun. 23, 2023, 63/523,284, filed Jun. 26, 2023, 63/541,102, filed Sep. 28, 2023, 63/541,111, filed Sep. 28, 2023, 63/541,137, filed Sep. 28, 2023, 63/588,829, filed Oct. 9, 2023, 63/588,839, filed Oct. 9, 2023, 63/588,847, filed Oct. 9, 2023, 63/605,649, filed Dec. 4, 2023, 63/619,620, filed Jan. 10, 2024, 63/551,294, filed Feb. 8, 2024, 63/561,582, filed Mar. 5, 2024, and 63/644,282, filed May 8, 2024, each of which are incorporated by reference herein in their entirety.

### SEQUENCE LISTING

The instant application contains a Sequence Listing which has been submitted electronically in XML format and is hereby incorporated by reference in its entirety. Said XML file, created on Sep. 25, 2024, is named 755511_SA9-652_ST26.xml and is 51,370 bytes in size.

### BACKGROUND

OX40 ligand (OX40L) is a TNF family member and is a 34 kDa type II transmembrane protein. The crystallized complex of human OX40 and OX40L is a trimeric configuration of one OX40L (trimer) and three OX40 monomers. The human extracellular domain is 42% homologous to mouse OX40L.

OX40L is not constitutively expressed but can be induced on professional APCs such as B-cells, dendritic cells (DCs) and macrophages. Other cell types such as Langerhans cells, endothelial cells, smooth muscle cells, mast cells and natural killer (NK) cells can be induced to express OX40L. T-cells can also express OX40L. The OX40L receptor, OX40, is expressed on activated T cells (CD4⁺ and CD8⁺ T cells, Th2, Th1 and Th17 cells) and CD4⁺Foxp3⁺ cells, even in the absence of activation.

### SUMMARY

The present disclosure is based on the discovery of a method for treating immune mediated diseases such as atopic dermatitis in a subject comprising administering to the subject an anti-OX40 ligand (OX40L) antibody or antigen binding fragment thereof.

The present disclosure is also based on the discovery of a method for treating immune mediated diseases such as atopic dermatitis in an adolescent subject comprising administering to the subject an anti-OX40 ligand (OX40L) antibody or antigen binding fragment thereof.

In one aspect, a method for treating an immune mediated disease in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject receives directly doses every 12 weeks (Q12W), without prior dose administration every 4 weeks (Q4W). By “the subject receives directly doses every 12 weeks (Q12W), without prior dose administration every 4 weeks (Q4W)” herein, we include the meaning that at the start of treatment the subject receives a first dose and thereafter receives subsequent doses every 12 weeks (e.g. first dose at 0 weeks, second dose at 12 weeks, third dose at 24 weeks, and so on). Thus, the subject may receive doses every 12 weeks, from the start of the treatment (i.e. directly doses every 12 weeks from the start of treatment).

In one aspect, a method for treating an immune mediated disease in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54, wherein the subject receives directly doses every 12 weeks (Q12W), without prior dose administration every 4 weeks (Q4W). The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO: 48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No: 64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist. In another aspect, a method for treating atopic dermatitis in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject receives directly doses every 12 weeks (Q12W), without prior dose administration every 4 weeks (Q4W).

In another aspect, a method for treating atopic dermatitis in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54, wherein the subject receives directly doses every 12 weeks (Q12W), without prior dose administration every 4 weeks (Q4W). The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the antibody or antigen binding fragment thereof is amlitelimab or a variant thereof. In certain exemplary embodiments, the antibody is amlitelimab. In certain exemplary embodiments, the antibody or antigen binding fragment thereof is administered by prefilled syringe, pen delivery device or autoinjector.

In certain exemplary embodiments, the subject receives an initial dose of about 500 mg of the antibody or antigen binding fragment thereof followed by one or more secondary doses of about 250 mg of the antibody or antigen binding fragment thereof.

In certain exemplary embodiments, the subject receives an initial dose of about 250 mg of the antibody or antigen binding fragment thereof followed by one or more secondary doses of about 250 mg of the antibody or antigen binding fragment thereof.

In certain exemplary embodiments, the subject receives an initial dose of about 125 mg of the antibody or antigen binding fragment thereof followed by one or more secondary doses of about 125 mg of the antibody or antigen binding fragment thereof.

In certain exemplary embodiments, the subject receives an initial dose of about 62.5 mg of the antibody or antigen binding fragment thereof followed by one or more secondary doses of about 62.5 mg of the antibody or antigen binding fragment thereof.

Whenever the terms “about 500 mg”, “about 250 mg”, “about 125 mg” and “about 62.5 mg” are used herein we include the meaning of “500 mg”, “250 mg”. “125 mg” and “62.5 mg” respectively.

In certain exemplary embodiments, each secondary dose is administered every 12 weeks (Q12W). In certain exemplary embodiments, each secondary dose is administered every 12 weeks (Q12W), or up to when the patient has achieved vIGA O/1, or has clear or substantially clear skin, or has achieved EAS175 or has achieved EASI90. For example, the subject may receive an initial dose of about 500 mg of the antibody or antigen binding fragment thereof followed by one or more secondary doses of about 250 mg of the antibody or antigen binding fragment thereof, wherein the secondary dose is administered every 12 weeks (Q12W).

In certain exemplary embodiments, the subject receives directly doses every 12 weeks, from the start of the treatment. In certain exemplary embodiments, the subject receives directly doses every 12 weeks, without prior dose administration every 4 weeks.

In certain alternative exemplary embodiments, each secondary dose is administered every 4 weeks (Q4W) during 24 weeks and then every 12 weeks (Q12W).

In certain alternative exemplary embodiments, each secondary dose is administered every 4 weeks (Q4W) during 24 weeks, or up to when the patient has achieved vIGA O/1, or has clear or substantially clear skin, or has achieved EASI-75 or has achieved EASI-90.

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or when those therapies are not advisable. By “not adequately controlled with systemic therapies” herein, it will be appreciated that we include the meaning of systemic therapies that are intended to treat the disorder (e.g. atopic dermatitis) other than an anti-OX40L antibody or antigen binding fragment thereof as described herein.

In certain exemplary embodiments, Eczema Area Surface Index (EASI) score is reduced in the subject. In certain exemplary embodiments, wherein the EASI score is selected from the group consisting of EASI-75, EASI-90 and EASI-100.

In certain exemplary embodiments, Investigator Global Assessment (IGA) score is reduced in the subject.

In certain exemplary embodiments, Peak Pruritis Numeric Rating Scale (PP-NRS) score is reduced in the subject.

In certain exemplary embodiments, Skin Pain Numeric Rating Scale (SP-NRS) score is reduced in the subject.

In certain exemplary embodiments, Sleep Disturbance Numeric Rating Scale (SD-NRS) score is reduced in the subject.

In certain exemplary embodiments, Dermatology Quality of Life Index (DLQI) score is reduced in the subject. By “reduced in the subject” in the context of the EASI, IGA, PP-NRS, SP-NRS, SD-NRS and DLQI scores herein, we include the meaning that following treatment of the subject by administering to the subject an anti-OX40 ligand (OX40L) antibody or antigen binding fragment thereof, the score is reduced relative to the score from the subject prior to treatment.

In certain exemplary embodiments. Hospital Anxiety and Depression Scale (HADS) score is improved in the subject. By “improved in the subject”, we include the meaning that following treatment of the subject by administering to the subject an anti-OX40 ligand (OX40L) antibody or antigen binding fragment thereof, the score is improved relative to the score from the subject prior to treatment.

In certain exemplary embodiments, the subject is administered a formulation comprising a 125 mg/mL amlitelimab solution supplied as a prefilled syringe that delivers 250 mg of amlitelimab in a 2 mL injection.

In another aspect, a method of treating an immune mediated disease in an adolescent subject in need thereof is provided, comprising administering to the adolescent subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60.

In another aspect, a method of treating an immune mediated disease in an adolescent subject in need thereof is provided, comprising administering to the adolescent subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the antibody or antigen binding fragment thereof is amlitelimab or a variant thereof. In certain exemplary embodiments, the antibody is amlitelimab.

In certain exemplary embodiments, the antibody or antigen binding fragment thereof is administered by prefilled syringe, pen delivery device or autoinjector.

In certain exemplary embodiments, the adolescent subject has a body weight ranging from about 25 kg to about 40 kg.

In certain exemplary embodiments, the adolescent subject receives an initial loading dose of about 250 mg of the antibody or antigen binding fragment thereof followed by one or more secondary doses of about 125 mg of the antibody or antigen binding fragment thereof.

In certain exemplary embodiments, each secondary dose is administered every 4 weeks (Q4W) during 24 weeks or up to when the patient when a patient has achieved vIGA O/1, or has clear or almost clear skin, or has achieved EAS175 or has achieved EASI90.

In certain exemplary embodiments, secondary doses are administered every 4 weeks after the initial dose (Q4W) during 24 weeks, and then wherein each subsequent secondary doses is administered every 12 weeks (Q12W).

In certain exemplary embodiments, each dose is administered Q12W. In certain exemplary embodiments, the subject receives doses Q12W from the start of treatment. In certain exemplary embodiments, the subject receives directly doses Q12W, without prior dose administration Q4W. For example, the adolescent subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W).

In certain exemplary embodiments, the subject is aged 12 years or older (e.g. the subject is aged 12-17 years old).

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or when those therapies are not advisable.

In certain exemplary embodiments, Eczema Area Surface Index (EASI) score is reduced in the subject. In certain exemplary embodiments, wherein the EASI score is selected from the group consisting of EASI-75, EASI-90 and EASI-100.

In certain exemplary embodiments, Investigator Global Assessment (IGA) score is reduced in the subject.

In certain exemplary embodiments, Peak Pruritis Numeric Rating Scale (PP-NRS) score is reduced in the subject.

In certain exemplary embodiments, Skin Pain Numeric Rating Scale (SP-NRS) score is reduced in the subject.

In certain exemplary embodiments, Sleep Disturbance Numeric Rating Scale (SD-NRS) score is reduced in the subject.

In certain exemplary embodiments, Dermatology Quality of Life Index (DLQI) score is reduced in the subject.

In certain exemplary embodiments, Hospital Anxiety and Depression Scale (HADS) score is improved in the subject.

In certain exemplary embodiments, the subject weighs equal to or greater than 25 kg and less than 40 kg, and wherein the subject is administered a formulation comprising a 62.5 mg/mL amlitelimab solution supplied as a prefilled syringe that delivers 125 mg of amlitelimab in a 2 mL injection. In certain exemplary embodiments, the subject weighs equal to or greater than 25 kg and less than 40 kg, and wherein the subject is administered a formulation comprising a 125 mg/mL amlitelimab solution supplied as a prefilled syringe that delivers 125 mg of amlitelimab in a 1 mL injection.

In another aspect, a method of treating atopic dermatitis in an adolescent subject in need thereof is provided, comprising administering to the adolescent subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60.

In another aspect, a method of treating atopic dermatitis in an adolescent subject in need thereof is provided, comprising administering to the adolescent subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the antibody or antigen binding fragment thereof is amlitelimab or a variant thereof. In certain exemplary embodiments, the antibody is amlitelimab.

In certain exemplary embodiments, the antibody or antigen binding fragment thereof is administered by prefilled syringe, pen delivery device or autoinjector.

In certain exemplary embodiments, the adolescent subject has a body weight ranging from about 25 kg to about 40 kg.

In certain exemplary embodiments, the adolescent subject receives an initial loading dose of about 250 mg of the antibody or antigen binding fragment thereof followed by one or more secondary doses of about 125 mg of the antibody or antigen binding fragment thereof.

In certain exemplary embodiments, each secondary dose is administered every 4 weeks (Q4W) during 24 weeks or up to when the patient when a patient has achieved vIGA O/1, or has clear or almost clear skin, or has achieved EAS175 or has achieved EASI90

In certain exemplary embodiments, secondary doses are administered every 4 weeks after the initial dose (Q4W) during 24 weeks, and then wherein each subsequent secondary doses is administered every 12 weeks (Q12W).

In certain exemplary embodiments, each dose is administered Q12W. In certain exemplary embodiments, the subject receives doses Q12W from the start of treatment. In certain exemplary embodiments, the subject receives directly doses Q12W, without prior dose administration Q4W. For example, the adolescent subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W).

In certain exemplary embodiments, the subject is aged 12 years or older (e.g. the subject is aged 12-17 years old).

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or when those therapies are not advisable.

In certain exemplary embodiments, Eczema Area Surface Index (EASI) score is reduced in the subject. In certain exemplary embodiments, wherein the EASI score is selected from the group consisting of EASI-75, EASI-90 and EASI-100.

In certain exemplary embodiments, Investigator Global Assessment (IGA) score is reduced in the subject.

In certain exemplary embodiments, Peak Pruritis Numeric Rating Scale (PP-NRS) score is reduced in the subject.

In certain exemplary embodiments, Skin Pain Numeric Rating Scale (SP-NRS) score is reduced in the subject.

In certain exemplary embodiments, Sleep Disturbance Numeric Rating Scale (SD-NRS) score is reduced in the subject.

In certain exemplary embodiments, Dermatology Quality of Life Index (DLQI) score is reduced in the subject.

In certain exemplary embodiments, Hospital Anxiety and Depression Scale (HADS) score is improved in the subject.

In certain exemplary embodiments, the subject weighs equal to or greater than 25 kg and less than 40 kg, and wherein the subject is administered a formulation comprising a 62.5 mg/mL amlitelimab solution supplied as a prefilled syringe that delivers 125 mg of amlitelimab in a 2 mL injection.

In certain exemplary embodiments, the subject weighs equal to or greater than 25 kg and less than 40 kg, and wherein the subject is administered a formulation comprising a 125 mg/mL amlitelimab solution supplied as a prefilled syringe that delivers 125 mg of amlitelimab in a 1 mL injection.

In another aspect, a method of treating an immune mediated disease in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject has a body weight ranging from about 25 kg to about 40 kg.

In another aspect, a method of treating an immune mediated disease in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54, wherein the subject has a body weight ranging from about 25 kg to about 40 kg. The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the antibody or antigen binding fragment thereof is amlitelimab or a variant thereof. In certain exemplary embodiments, the antibody is amlitelimab.

In certain exemplary embodiments, the antibody or antigen binding fragment thereof is administered by prefilled syringe, pen delivery device or autoinjector.

In certain exemplary embodiments, the adolescent subject receives an initial loading dose of about 250 mg of the antibody or antigen binding fragment thereof followed by one or more secondary doses of about 125 mg of the antibody or antigen binding fragment thereof.

In certain exemplary embodiments, each secondary dose is administered every 4 weeks (Q4W) during 24 weeks or up to when the patient when a patient has achieved vIGA O/1, or has clear or almost clear skin, or has achieved EAS175 or has achieved EASI90

In certain exemplary embodiments, secondary doses are administered every 4 weeks after the initial dose (Q4W) during 24 weeks, and then wherein each subsequent secondary doses is administered every 12 weeks (Q12W).

In certain exemplary embodiments, each dose is administered Q12W. In certain exemplary embodiments, the subject receives doses Q12W from the start of treatment. In certain exemplary embodiments, the subject receives directly doses Q12W, without prior dose administration Q4W. For example, the adolescent subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W).

In certain exemplary embodiments, the subject is aged 12 years or older (e.g. the subject is aged 12-17 years old).

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or when those therapies are not advisable.

In certain exemplary embodiments, Eczema Area Surface Index (EASI) score is reduced in the subject. In certain exemplary embodiments, wherein the EASI score is selected from the group consisting of EASI-75, EASI-90 and EASI-100.

In certain exemplary embodiments, Investigator Global Assessment (IGA) score is reduced in the subject.

In certain exemplary embodiments, Peak Pruritis Numeric Rating Scale (PP-NRS) score is reduced in the subject.

In certain exemplary embodiments, Skin Pain Numeric Rating Scale (SP-NRS) score is reduced in the subject.

In certain exemplary embodiments, Sleep Disturbance Numeric Rating Scale (SD-NRS) score is reduced in the subject.

In certain exemplary embodiments, Dermatology Quality of Life Index (DLQI) score is reduced in the subject.

In certain exemplary embodiments, Hospital Anxiety and Depression Scale (HADS) score is improved in the subject.

In certain exemplary embodiments, the subject weighs equal to or greater than 25 kg and less than 40 kg, and wherein the subject is administered a formulation comprising a 62.5 mg/mL amlitelimab solution supplied as a prefilled syringe that delivers 125 mg of amlitelimab in a 2 mL injection.

In certain exemplary embodiments, the subject weighs equal to or greater than 25 kg and less than 40 kg, and wherein the subject is administered a formulation comprising a 125 mg/mL amlitelimab solution supplied as a prefilled syringe that delivers 125 mg of amlitelimab in a 1 mL injection.

In another aspect, a method of treating atopic dermatitis in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject has a body weight ranging from about 25 kg to about 40 kg.

In another aspect, a method of treating atopic dermatitis in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54, wherein the subject has a body weight ranging from about 25 kg to about 40 kg. The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the antibody or antigen binding fragment thereof is amlitelimab or a variant thereof. In certain exemplary embodiments, the antibody is amlitelimab.

In certain exemplary embodiments, the antibody or antigen binding fragment thereof is administered by prefilled syringe, pen delivery device or autoinjector.

In certain exemplary embodiments, the adolescent subject receives an initial loading dose of about 250 mg of the antibody or antigen binding fragment thereof followed by one or more secondary doses of about 125 mg of the antibody or antigen binding fragment thereof.

In certain exemplary embodiments, each secondary dose is administered every 4 weeks (Q4W) during 24 weeks or up to when the patient when a patient has achieved vIGA O/1, or has clear or almost clear skin, or has achieved EAS175 or has achieved EASI90

In certain exemplary embodiments, secondary doses are administered every 4 weeks after the initial dose (Q4W) during 24 weeks, and then wherein each subsequent secondary doses is administered every 12 weeks (Q12W).

In certain exemplary embodiments, each dose is administered Q12W. In certain exemplary embodiments, the subject receives doses Q12W from the start of treatment. In certain exemplary embodiments, the subject receives directly doses Q12W, without prior dose administration Q4W. For example, the adolescent subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W).

In certain exemplary embodiments, the subject is aged 12 years or older (e.g. the subject is aged 12-17 years old).

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or when those therapies are not advisable.

In certain exemplary embodiments, Eczema Area Surface Index (EASI) score is reduced in the subject. In certain exemplary embodiments, wherein the EASI score is selected from the group consisting of EASI-75, EASI-90 and EASI-100.

In certain exemplary embodiments, Investigator Global Assessment (IGA) score is reduced in the subject.

In certain exemplary embodiments, Peak Pruritis Numeric Rating Scale (PP-NRS) score is reduced in the subject.

In certain exemplary embodiments, Skin Pain Numeric Rating Scale (SP-NRS) score is reduced in the subject.

In certain exemplary embodiments, Sleep Disturbance Numeric Rating Scale (SD-NRS) score is reduced in the subject.

In certain exemplary embodiments, Dermatology Quality of Life Index (DLQI) score is reduced in the subject.

In certain exemplary embodiments, Hospital Anxiety and Depression Scale (HADS) score is improved in the subject.

In certain exemplary embodiments, the subject weighs equal to or greater than 25 kg and less than 40 kg, and wherein the subject is administered a formulation comprising a 62.5 mg/mL amlitelimab solution supplied as a prefilled syringe that delivers 125 mg of amlitelimab in a 2 mL injection.

In certain exemplary embodiments, the subject weighs equal to or greater than 25 kg and less than 40 kg, and wherein the subject is administered a formulation comprising a 125 mg/mL amlitelimab solution supplied as a prefilled syringe that delivers 125 mg of amlitelimab in a 1 mL injection.

The present disclosure is also based on the discovery of biomarkers associated with atopic dermatitis.

The present disclosure relates to the use of at least one, or all of the biomarkers selected from the group consisting of TARC, IL-5, eosinophil count, and LDH, in treating immune-mediated diseases such as atopic dermatitis (AD) in a subject in need thereof.

The present disclosure relates to the use of at least one, or all of the biomarkers selected from the group consisting of IL-5, TARC, eotaxin-3, Total IgE, IL-13, IL-17A, IL-22, and eosinophil count in treating immune-mediated diseases such as asthma in a subject in need thereof.

The present disclosure relates to the use of at least one, or all of the biomarkers selected from the group consisting of IL-17A, IL-13 and IL-31 in treating immune-mediated diseases such as interstitial lung disease-systemic sclerosis (ILD-SSc) in a subject in need thereof.

The present disclosure relates to the use of at least one, or all of the biomarkers selected from the group consisting of IL-5, IL-22 and TARC in treating immune-mediated diseases such as systemic sclerosis (SSc) in a subject in need thereof.

The present disclosure relates to the use of at least one, or all of the biomarkers selected from the group consisting of IL-17A, IL-13, IL-31 and periostin in treating immune-mediated diseases such as SSc in a subject in need thereof.

The present disclosure relates to the use of the biomarker IL-17A in treating immune-mediated diseases such as hidradenitis suppurativa (HS) in a subject in need thereof.

The present disclosure relates to the use of at least one, or all of the biomarkers selected from the group consisting of IL-13, IL-22, IL-17A and IL-31 in treating immune-mediated diseases such as alopecia areata (AA) in a subject in need thereof.

The present disclosure relates to the use of at least one, or all of the biomarkers selected from the group consisting of IL-17A, IL-22, IL-5 and IL-13 in treating immune-mediated diseases such as celiac disease in a subject in need thereof.

The present disclosure relates to the use of at least one, or all of the biomarkers selected from the group consisting of IL-17A, IL-22 and IL-13 in treating immune-mediated diseases such as celiac disease in a subject in need thereof.

By “use of biomarkers” in the context of treating immune-mediated diseases or atopic dermatitis, we include the meaning of using the biomarkers to select subjects for treatment, and/or to monitor the efficacy of treatment.

In another aspect, a method of treating an immune mediated disease in a subject in need thereof is provided, comprising administering to the subject an anti-OX40 ligand (OX40L) antibody or antigen binding fragment thereof in an amount effective to decrease a level of at least one biomarker selected from the group consisting of TARC, IL-5, eosinophil count and LDH, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, the subject is administered an anti-OX40L antibody or antigen binding fragment thereof in an amount effective to decrease a level of all biomarkers selected from the group consisting of TARC, IL-5, eosinophil count and LDH.

In another aspect, a method of treating an immune mediated disease in a subject in need thereof is provided, comprising administering to the subject an anti-OX40 ligand (OX40L) antibody or antigen binding fragment thereof in an amount effective to decrease a level of at least one biomarker selected from the group consisting of TARC, IL-5, eosinophil count and LDH, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, the subject is administered an anti-OX40L antibody or antigen binding fragment thereof in an amount effective to decrease a level of all biomarkers selected from the group consisting of TARC. IL-5, eosinophil count and LDH.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In one embodiment, the method results in a decrease of the level of the at least one biomarker in the subject, for example relative to the level of the at least one biomarker prior to administering the anti-OX40 ligand (OX40L) antibody or antigen binding fragment thereof.

In another aspect, a method of treating an immune mediated disease in a subject in need thereof is provided, comprising selecting a subject having an immune-mediate disease and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control. By an “elevated level of a biomarker relative to a control” as used herein, we include the meaning of a level of a biomarker being increased relative to the level of that biomarker from a subject that does not have the immune mediated disease.

In another aspect, a method of treating an immune mediated disease in a subject in need thereof is provided, comprising selecting a subject having an immune-mediate disease and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In another aspect, a method of treating atopic dermatitis (AD) in a subject in need thereof is provided, comprising administering to the subject an anti-OX40 ligand (OX40L) antibody or antigen binding fragment thereof in an amount effective to decrease a level of at least one biomarker selected from the group consisting of TARC. IL-5, eosinophil count and LDH, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, the subject is administered an anti-OX40L antibody or antigen binding fragment thereof in an amount effective to decrease a level of all biomarkers selected from the group consisting of TARC. IL-5, eosinophil count and LDH.

In another aspect, a method of treating atopic dermatitis (AD) in a subject in need thereof is provided, comprising administering to the subject an anti-OX40 ligand (OX40L) antibody or antigen binding fragment thereof in an amount effective to decrease a level of at least one biomarker selected from the group consisting of TARC. IL-5, eosinophil count and LDH, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 528, and an LCDR3 of SEQ ID NO: 54. In an embodiment, the subject is administered an anti-OX40L antibody or antigen binding fragment thereof in an amount effective to decrease a level of all biomarkers selected from the group consisting of TARC. IL-5, eosinophil count and LDH.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In one embodiment, the method results in a decrease of the level of the at least one biomarker in the subject, for example relative to the level of the at least one biomarker prior to administering the anti-OX40 ligand (OX40L) antibody or antigen binding fragment thereof.

In another aspect, a method of treating atopic dermatitis (AD) in a subject in need thereof is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control. By an “elevated level of a biomarker relative to a control” as used herein, we include the meaning of a level of a biomarker being increased relative to the level of that biomarker from a subject that does not have atopic dermatitis.

In another aspect, a method of treating atopic dermatitis (AD) in a subject in need thereof is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In another aspect, a method of treating an immune-mediated disease in a subject in need thereof is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60; and reducing the level of at least one biomarker selecting from TARC. IL-5, eosinophil counts. LDH, or combinations thereof. In an embodiment, the level of all biomarkers selected from TARC. IL-5, eosinophil counts and LDH are reduced.

In another aspect, a method of treating an immune-mediated disease in a subject in need thereof is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54; and reducing the level of at least one biomarker selecting from TARC. IL-5, eosinophil counts. LDH, or combinations thereof. In an embodiment, the level of all biomarkers selected from TARC. IL-5, eosinophil counts and LDH are reduced.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In another aspect, a method of treating an immune mediated disease in a subject in need thereof is provided, comprising selecting a subject having an immune mediated disease and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof to reduce the level of at least one biomarker selecting from TARC. IL-5, eosinophil counts. LDH, or combinations thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, a subject is selected having an elevated level of at least one biomarker selected from the group consisting of thymus and activation-regulated chemokine (TARC), interleukin-5 (IL-5), eosinophil count, and lactate dehydrogenase (LDH) relative to a control, and the subject is administered an anti-OX40L antibody or antigen binding fragment thereof to reduce the level of at least one biomarker selecting from TARC. IL-5, eosinophil counts, and LDH. In a further embodiment, a subject is selected having an elevated level of at least one biomarker selected from the group consisting of thymus and activation-regulated chemokine (TARC), interleukin-5 (IL-5), eosinophil count, and lactate dehydrogenase (LDH) relative to a control, and the subject is administered an anti-OX40L antibody or antigen binding fragment thereof to reduce the level of all biomarkers selecting from TARC. IL-5, eosinophil counts, and LDH.

In another aspect, a method of treating an immune mediated disease in a subject in need thereof is provided, comprising selecting a subject having an immune mediated disease and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof to reduce the level of at least one biomarker selecting from TARC. IL-5, eosinophil counts. LDH, or combinations thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, a subject is selected having an elevated level of at least one biomarker selected from the group consisting of thymus and activation-regulated chemokine (TARC), interleukin-5 (IL-5), eosinophil count, and lactate dehydrogenase (LDH) relative to a control, and the subject is administered an anti-OX40L antibody or antigen binding fragment thereof to reduce the level of at least one biomarker selecting from TARC. IL-5, eosinophil counts, and LDH. In a further embodiment, a subject is selected having an elevated level of at least one biomarker selected from the group consisting of thymus and activation-regulated chemokine (TARC), interleukin-5 (IL-5), eosinophil count, and lactate dehydrogenase (LDH) relative to a control, and the subject is administered an anti-OX40L antibody or antigen binding fragment thereof to reduce the level of all biomarkers selecting from TARC. IL-5, eosinophil counts, and LDH.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In another aspect, a method of treating atopic dermatitis (AD) in a subject in need thereof is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60; and reducing the level of at least one biomarker selecting from TARC. IL-5, eosinophil counts. LDH, or combinations thereof. In an embodiment, the level of all biomarkers selected from TARC, IL-5, eosinophil counts, and LDH are reduced.

In another aspect, a method of treating atopic dermatitis (AD) in a subject in need thereof is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54; and reducing the level of at least one biomarker selecting from TARC. IL-5, eosinophil counts. LDH, or combinations thereof. In an embodiment, the level of all biomarkers selected from TARC, IL-5, eosinophil counts, and LDH are reduced.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In another aspect, a method of treating atopic dermatitis (AD) in a subject in need thereof is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

In another aspect, a method of treating atopic dermatitis (AD) in a subject in need thereof is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In yet another aspect, a method of reducing a biomarker in a subject having atopic dermatitis (AD) is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60; wherein the biomarker is selected from TARC. IL-5, eosinophil counts, and LDH. In an embodiment, the level of all biomarkers selected from TARC. IL-5, eosinophil counts, and LDH is reduced.

In yet another aspect, a method of reducing a biomarker in a subject having atopic dermatitis (AD) is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54; wherein the biomarker is selected from TARC. IL-5, eosinophil counts, and LDH. In an embodiment, the level of all biomarkers selected from TARC. IL-5, eosinophil counts, and LDH is reduced.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In yet another aspect, a method of reducing a biomarker in a subject having atopic dermatitis (AD) is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

In yet another aspect, a method of reducing a biomarker in a subject having atopic dermatitis (AD) is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, a subject is selected having an elevated level of all biomarkers selected from the group consisting of thymus and activation-regulated chemokine a control.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In yet another aspect, a method of reducing a biomarker in a subject having an immune mediated disease is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60; wherein the biomarker is selected from TARC. IL-5, eosinophil counts, and LDH. In an embodiment, the level of all biomarkers selected from TARC. IL-5, eosinophil counts, and LDH is reduced. In yet another aspect, a method of reducing a biomarker in a subject having an immune mediated disease is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54; wherein the biomarker is selected from TARC. IL-5, eosinophil counts, and LDH. In an embodiment, the level of all biomarkers selected from TARC, IL-5, eosinophil counts, and LDH is reduced.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In yet another aspect, a method of reducing a biomarker in a subject having an immune mediated disease is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, a subject is selected having an elevated level of all biomarkers selected from the group consisting of thymus and activation-regulated chemokine a control.

In yet another aspect, a method of reducing a biomarker in a subject having an immune mediated disease is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In various embodiments disclosed herein, the antibody or antigen binding fragment thereof is amlitelimab or a variant thereof. In certain embodiments, the antibody is amlitelimab.

In various embodiments disclosed herein, the method results in a decrease in the level of at least one biomarker selecting from TARC, IL-5, eosinophil counts, LDH, or combinations thereof, relative to the baseline level of the corresponding biomarker in the subject before administering the effective amount of an anti-OX40 ligand (OX40L) antibody or antigen binding fragment thereof.

In various embodiments disclosed herein, the effective amount of the anti-OX40 ligand (OX40L) antibody or antigen binding fragment thereof ranges from 50 mg to 500 mg.

In various embodiments disclosed herein, the effective amount of the anti-OX40 ligand (OX40L) antibody or antigen binding fragment thereof is about 62.5 mg, 125 mg, 250 mg, or 500 mg.

In various embodiments disclosed herein, the subject receives an initial dose of about 500 mg of the antibody or antigen binding fragment thereof followed by one or more secondary doses of about 250 mg of the antibody or antigen binding fragment thereof.

In various embodiments disclosed herein, the subject receives an initial dose of about 250 mg of the antibody or antigen binding fragment thereof followed by one or more secondary doses of about 250 mg of the antibody or antigen binding fragment thereof.

In various embodiments disclosed herein, the subject receives an initial dose of about 250 mg of the antibody or antigen binding fragment thereof followed by one or more secondary doses of about 125 mg of the antibody or antigen binding fragment thereof.

In various embodiments disclosed herein, the subject receives an initial dose of about 125 mg of the antibody or antigen binding fragment thereof followed by one or more secondary doses of about 125 mg of the antibody or antigen binding fragment thereof.

In various embodiments disclosed herein the subject receives an initial dose of about 62.5 mg of the antibody or antigen binding fragment thereof followed by one or more secondary doses of about 62.5 mg of the antibody or antigen binding fragment thereof.

In various embodiments disclosed herein, each secondary dose may be administered every 4 weeks (Q4W) during 24 weeks.

In various embodiments disclosed herein, a first secondary dose is administered every 4 weeks after the initial dose (Q4W) up to week 24, and wherein each subsequent secondary dose is administered every 12 weeks (Q12W).

In various embodiments disclosed herein, including of those aspects that refers to particular biomarkers, each dose is administered Q12W. In certain exemplary embodiments, the subject receives doses Q12W from the start of treatment. In certain exemplary embodiments, the subject receives directly doses Q12W, without prior dose administration Q4W. For example, an adult subject may receive an initial dose of about 500 mg of the antibody or antigen binding fragment thereof followed by one or more secondary doses of about 250 mg of the antibody or antigen binding fragment thereof, wherein the secondary dose is administered every 12 weeks (Q12W). In another example an adolescent subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W).

In various embodiments disclosed herein, the antibody or antigen binding fragment thereof is administered by prefilled syringe, pen delivery device or autoinjector.

In various embodiments disclosed herein, the subject is treated with the antibody or antigen binding fragment thereof for a duration of about 4 to about 24 weeks.

In various embodiments disclosed herein, the subject is treated with the antibody or antigen binding fragment thereof for a duration of about 4 weeks, 16 weeks, 24 weeks or 52 weeks.

In various embodiments disclosed herein, the atopic dermatitis is moderate-to-severe atopic dermatitis.

In various embodiments disclosed herein, the moderate-to-severe atopic dermatitis is not adequately controlled with topical prescription therapies or with systemic therapies or when those therapies are not advisable.

In another aspect, a method of treating atopic dermatitis (AD) in a subject in need thereof is provided, comprising selecting a subject with an elevated biomarker level, wherein the biomarker is selected from the group consisting of thymus and activation-regulated chemokine (TARC), interleukin-5 (IL-5), eosinophil count and lactate dehydrogenase (LDH), and administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

In another aspect, a method of treating atopic dermatitis (AD) in a subject in need thereof is provided, comprising selecting a subject with an elevated biomarker level, wherein the biomarker is selected from the group consisting of thymus and activation-regulated chemokine (TARC), interleukin-5 (IL-5), eosinophil count and lactate dehydrogenase (LDH), and administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54. In an embodiment, a subject is selected having an elevated level of all biomarkers selected from the group consisting of thymus and activation-regulated chemokine (TARC), interleukin-5 (IL-5), eosinophil count, and lactate dehydrogenase (LDH) relative to a control.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the method results in a decrease of the level of at least one biomarker in the subject relative to a control, for example relative to the level of the at least one biomarker prior to administering the anti-OX40 ligand (OX40L) antibody or antigen binding fragment thereof.

In certain exemplary embodiments, the antibody or antigen binding fragment thereof is amlitelimab or a variant thereof. In certain exemplary embodiments, the antibody is amlitelimab.

In certain exemplary embodiments, the effective amount of the anti-OX40 ligand (OX40L) antibody or antigen binding fragment thereof ranges from 50 mg to 500 mg. In certain exemplary embodiments, the effective amount of the anti-OX40 ligand (OX40L) antibody or antigen binding fragment thereof is about 62.5 mg, 125 mg, 250 mg, or 500 mg.

In certain exemplary embodiments, the subject receives an initial dose of about 250 mg of the antibody or antigen binding fragment thereof followed by one or more secondary doses of about 125 mg of the antibody or antigen binding fragment thereof.

In certain exemplary embodiments, the subject receives an initial dose of about 500 mg of the antibody or antigen binding fragment thereof followed by one or more secondary doses of about 250 mg of the antibody or antigen binding fragment thereof.

In certain exemplary embodiments, the subject receives an initial dose of about 250 mg of the antibody or antigen binding fragment thereof followed by one or more secondary doses of about 250 mg of the antibody or antigen binding fragment thereof.

In certain exemplary embodiments, the subject receives an initial dose of about 125 mg of the antibody or antigen binding fragment thereof followed by one or more secondary doses of about 125 mg of the antibody or antigen binding fragment thereof.

In certain exemplary embodiments, the subject receives an initial dose of about 62.5 mg of the antibody or antigen binding fragment thereof followed by one or more secondary doses of about 62.5 mg of the antibody or antigen binding fragment thereof.

In certain exemplary embodiments, each secondary dose is administered every 4 weeks (Q4W).

In certain exemplary embodiments, each dose is administered Q12W. In certain exemplary embodiments, the subject receives doses Q12W from the start of treatment. In certain exemplary embodiments, the subject receives directly doses Q12W, without prior dose administration Q4W. For example, an adult subject may receive an initial dose of about 500 mg of the antibody or antigen binding fragment thereof followed by one or more secondary doses of about 250 mg of the antibody or antigen binding fragment thereof, wherein the secondary dose is administered every 12 weeks (Q12W). In another example an adolescent subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W).

In certain exemplary embodiments, the antibody or antigen binding fragment thereof is administered by prefilled syringe, pen delivery device or autoinjector.

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or wherein topical prescription therapies or with systemic therapies are not advisable.

In certain exemplary embodiments, EASI score is reduced in the subject. In certain exemplary embodiments, wherein the EASI score is selected from the group consisting of EASI-75, EASI-90 and EASI-100.

In certain exemplary embodiments, IGA score is reduced in the subject.

In certain exemplary embodiments, PP-NRS score is reduced in the subject.

In certain exemplary embodiments, SP-NRS score is reduced in the subject.

In certain exemplary embodiments, SD-NRS score is reduced in the subject.

In certain exemplary embodiments, DLQI score is reduced in the subject.

In certain exemplary embodiments, HADS score is improved in the subject.

In certain exemplary embodiments, a level of at least one biomarker selected from the group consisting of TARC, IL-5, eosinophil count, and LDH is reduced in the subject.

In another aspect, a method of reducing a biomarker in a subject having atopic dermatitis (AD) is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the biomarker is selected from TARC, IL-5, eosinophil count and LDH. In an embodiment, the level of all biomarkers selected from TARC, IL-5, eosinophil count and LDH is reduced.

In another aspect, a method of reducing a biomarker in a subject having atopic dermatitis (AD) is provided, comprising administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54, wherein the biomarker is selected from TARC, IL-5, eosinophil count and LDH. In an embodiment, the level of all biomarkers selected from TARC, IL-5, eosinophil count and LDH is reduced.

The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the method results in a decrease of the level of at least one biomarker in the subject relative to a control, for example relative to the level of the at least one biomarker prior to administering the anti-OX40 ligand (OX40L) antibody or antigen binding fragment thereof.

In certain exemplary embodiments, the antibody or antigen binding fragment thereof is amlitelimab or a variant thereof. In certain exemplary embodiments, the antibody is amlitelimab.

In certain exemplary embodiments, the method results in a decrease in the level of at least one biomarker selecting from TARC, IL-5, eosinophil count and LDH, or combinations thereof, relative to the baseline level of the corresponding biomarker in the subject before administering the effective amount of the anti-OX40L antibody or antigen binding fragment thereof.

In certain exemplary embodiments, the effective amount of the anti-OX40L antibody or antigen binding fragment thereof ranges from 50 mg to 500 mg. In certain exemplary embodiments, the effective amount of the anti-OX40L antibody or antigen binding fragment thereof is about 62.5 mg, 125 mg, 250 mg, or 500 mg.

In certain exemplary embodiments, the subject receives an initial dose of about 250 mg of the antibody or antigen binding fragment thereof followed by one or more secondary doses of about 125 mg of the antibody or antigen binding fragment thereof.

In certain exemplary embodiments, the subject receives an initial dose of about 500 mg of the antibody or antigen binding fragment thereof followed by one or more secondary doses of about 250 mg of the antibody or antigen binding fragment thereof.

In certain exemplary embodiments, the subject receives an initial dose of about 250 mg of the antibody or antigen binding fragment thereof followed by one or more secondary doses of about 250 mg of the antibody or antigen binding fragment thereof.

In certain exemplary embodiments, the subject receives an initial dose of about 125 mg of the antibody or antigen binding fragment thereof followed by one or more secondary doses of about 125 mg of the antibody or antigen binding fragment thereof.

In certain exemplary embodiments, the subject receives an initial dose of about 62.5 mg of the antibody or antigen binding fragment thereof followed by one or more secondary doses of about 62.5 mg of the antibody or antigen binding fragment thereof.

In certain exemplary embodiments, each secondary dose is administered every Q4W.

In certain exemplary embodiments, the antibody or antigen binding fragment thereof is administered by prefilled syringe, pen delivery device or autoinjector.

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or wherein topical prescription therapies or with systemic therapies are not advisable.

In certain exemplary embodiments, EASI score is reduced in the subject. In certain exemplary embodiments, wherein the EASI score is selected from the group consisting of EASI-75, EASI-90 and EASI-100.

In certain exemplary embodiments, IGA score is reduced in the subject.

In certain exemplary embodiments. PP-NRS score is reduced in the subject.

In certain exemplary embodiments. SP-NRS score is reduced in the subject.

In certain exemplary embodiments. SD-NRS score is reduced in the subject.

In certain exemplary embodiments. DLQI score is reduced in the subject.

In certain exemplary embodiments. HADS score is improved in the subject.

In another aspect, a method of treating an immune-mediated disease in a subject in need thereof is provided, comprising: administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, and wherein the immune-mediated disease is selected from the group consisting of atopic dermatitis (AD), asthma, interstitial lung disease-systemic sclerosis (ILD-SSc), systemic sclerosis (SSc), hidradenitis suppurativa (HS), alopecia areata (AA) and celiac disease.

In another aspect, a method of treating an immune-mediated disease in a subject in need thereof is provided, comprising: administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54, and wherein the immune-mediated disease is selected from the group consisting of atopic dermatitis (AD), asthma, interstitial lung disease-systemic sclerosis (ILD-SSc), systemic sclerosis (SSc), hidradenitis suppurativa (HS), alopecia areata (AA) and celiac disease. The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, the immune-mediated disease is AD. In certain exemplary embodiments, the immune-mediated disease is asthma. In certain exemplary embodiments, the immune-mediated disease is ILD-SSc. In certain exemplary embodiments, the immune-mediated disease is SSc. In certain exemplary embodiments, the immune-mediated disease is HS. In certain exemplary embodiments, the immune-mediated disease is AA. In certain exemplary embodiments, the immune-mediated disease is celiac disease. In certain exemplary embodiments, systemic therapies are not advisable.

In certain exemplary embodiments, the method results in a decrease of the level of at least one biomarker in the subject relative to a control, for example relative to the level of the at least one biomarker prior to administering the anti-OX40 ligand (OX40L) antibody or antigen binding fragment thereof.

In certain exemplary embodiments, the antibody or antigen binding fragment thereof is amlitelimab or a variant thereof. In certain exemplary embodiments, the antibody is amlitelimab.

In certain exemplary embodiments, the method results in a decrease in the level of at least one biomarker selecting from TARC, IL-5, eosinophil count and LDH, or any combination thereof, relative to the baseline level of the corresponding biomarker in the subject before administering the effective amount of an anti-OX40L antibody or antigen binding fragment thereof.

In certain exemplary embodiments, the effective amount of the anti-OX40L antibody or antigen binding fragment thereof ranges from 50 mg to 500 mg. In certain exemplary embodiments, the effective amount of the anti-OX40L antibody or antigen binding fragment thereof is about 62.5 mg, 125 mg, 250 mg, or 500 mg. In certain exemplary embodiments, the effective amount of the anti-OX40L antibody or antigen binding fragment thereof is 62.5 mg, 125 mg, 250 mg, or 500 mg.

In certain exemplary embodiments, the subject receives an initial dose of about 250 mg of the antibody or antigen binding fragment thereof followed by one or more secondary doses of about 125 mg of the antibody or antigen binding fragment thereof. In certain exemplary embodiments, the subject receives an initial dose of 250 mg of the antibody or antigen binding fragment thereof followed by one or more secondary doses of 125 mg of the antibody or antigen binding fragment thereof.

In certain exemplary embodiments, the subject receives an initial dose of about 500 mg of the antibody or antigen binding fragment thereof followed by one or more secondary doses of about 250 mg of the antibody or antigen binding fragment thereof. In certain exemplary embodiments, the subject receives an initial dose of 500 mg of the antibody or antigen binding fragment thereof followed by one or more secondary doses of 250 mg of the antibody or antigen binding fragment thereof.

In certain exemplary embodiments, the subject receives an initial dose of about 250 mg of the antibody or antigen binding fragment thereof followed by one or more secondary doses of about 250 mg of the antibody or antigen binding fragment thereof. In certain exemplary embodiments, the subject receives an initial dose of 250 mg of the antibody or antigen binding fragment thereof followed by one or more secondary doses of 250 mg of the antibody or antigen binding fragment thereof.

In certain exemplary embodiments, the subject receives an initial dose of about 125 mg of the antibody or antigen binding fragment thereof followed by one or more secondary doses of about 125 mg of the antibody or antigen binding fragment thereof. In certain exemplary embodiments, the subject receives an initial dose of 125 mg of the antibody or antigen binding fragment thereof followed by one or more secondary doses of 125 mg of the antibody or antigen binding fragment thereof.

In certain exemplary embodiments, the subject receives an initial dose of about 62.5 mg of the antibody or antigen binding fragment thereof followed by one or more secondary doses of about 62.5 mg of the antibody or antigen binding fragment thereof. In certain exemplary embodiments, the subject receives an initial dose of 62.5 mg of the antibody or antigen binding fragment thereof followed by one or more secondary doses of 62.5 mg of the antibody or antigen binding fragment thereof.

In certain exemplary embodiments, each secondary dose is administered Q4W.

In certain exemplary embodiments, the antibody or antigen binding fragment thereof is administered by prefilled syringe, pen delivery device or autoinjector.

In certain exemplary embodiments, the atopic dermatitis is moderate-to-severe atopic dermatitis. In certain exemplary embodiments, the moderate-to-severe atopic dermatitis is not adequately controlled with topical prescription therapies or with systemic therapies or wherein topical prescription therapies or with systemic therapies are not advisable.

In certain exemplary embodiments, EASI score is reduced in the subject. In certain exemplary embodiments, wherein the EASI score is selected from the group consisting of EASI-75, EASI-90) and EASI-100.

In certain exemplary embodiments, IGA score is reduced in the subject.

In certain exemplary embodiments, PP-NRS score is reduced in the subject.

In certain exemplary embodiments, SP-NRS score is reduced in the subject.

In certain exemplary embodiments, SD-NRS score is reduced in the subject.

In certain exemplary embodiments, DLQI score is reduced in the subject.

In certain exemplary embodiments, HADS score is improved in the subject.

In certain exemplary embodiments, a level of at least one biomarker selected from the group consisting of TARC, IL-5, eosinophil count, and LDH is reduced in the subject.

In another aspect, a method of treating atopic dermatitis in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein efficacy is maintained at week 28 off of treatment or at week 32 after final administration of the antibody or antigen binding fragment thereof to the subject. By “efficacy is maintained at week 28 off of treatment or at week 32 after final administration of the antibody or antigen binding fragment thereof” herein, we include the meaning of efficacy being maintained 28 or 32 weeks after the final injection of the antibody of antigen binding fragment thereof to the subject.

In another aspect, a method of treating atopic dermatitis in a subject in need thereof is provided, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 36, an HCDR2 of SEQ ID NO: 38, and an HCDR3 of SEQ ID NO: 40, and a light chain complementarity determining region (LCDR) 1 of SEQ ID NO: 50, an LCDR2 of SEQ ID NO: 52, and an LCDR3 of SEQ ID NO: 54, wherein efficacy is maintained at week 28 off of treatment or at week 32 after final administration of the antibody or antigen binding fragment thereof to the subject. The OX40L antibody or antigen binding fragment thereof may comprise a variable heavy (VH) domain comprising an amino acid sequence of SEQ ID NO: 34, or a VH domain amino acid sequence that is at least 80% (e.g. at least 85%) identical to SEQ ID NO:34 and/or a variable light (VL) domain comprising an amino acid sequence of SEQ ID NO: 48, or a VL domain amino acid sequence that is at least 80% (e.g. at least 85%, 90% or 95%) identical to SEQ ID NO:48. Thus, the OX40L antibody or antigen binding fragment thereof may comprise a VH domain comprising an amino acid sequence of SEQ ID NO: 34 and a VL domain comprising an amino acid sequence of SEQ ID NO: 48. Similarly, the antibody, either defined by reference to the CDRs above or the VH and/or VL domains above, may comprise a heavy chain and a light chain, the heavy chain amino acid sequence consisting of the sequence of SEQ ID No:62 and the light chain amino acid sequence consisting of the sequence of SEQ ID No:64. It will be appreciated that the antibody, as defined by the CDRs above, the VH and/or VL domains above, or the heavy chain and/or light chain sequences above, may also comprises an IgG4 constant region, optionally wherein the constant region is IgG4-PE (SEQ ID NO: 128). The OX40L antibody or antigen binding fragment thereof may be an OX40L antagonist.

In certain exemplary embodiments, each dose is administered Q12W. In certain exemplary embodiments, the subject receives doses Q12W from the start of treatment. In certain exemplary embodiments, the subject receives directly doses Q12W, without prior dose administration Q4W. For example, an adult subject may receive an initial dose of about 500 mg of the antibody or antigen binding fragment thereof followed by one or more secondary doses of about 250 mg of the antibody or antigen binding fragment thereof, wherein the secondary dose is administered every 12 weeks (Q12W). In another example an adolescent subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W).

In certain exemplary embodiments, the subject is a vIGA 0/1 responder and vIGA O/1 response efficacy is maintained in the subject, or wherein the subject is an AESI 75 responder and EASI 75 response efficacy is maintained in the subject. In certain exemplary embodiments, the subject is a vIGA 0/1 responder and vIGA O/1 response efficacy is maintained in the subject. In certain exemplary embodiments, the subject is an AESI 75 responder and EASI 75 efficacy is maintained in the subject.

In certain exemplary embodiments, the subject does not experience a treatment-emergent adverse event (TEAE).

In another aspect, a method of treating atopic dermatitis in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating atopic dermatitis in an adolescent subject in need thereof is provided, comprising administering to the adolescent subject amlitelimab, wherein the adolescent subject receives an initial dose of about 250 mg of amlitelimab followed by one or more secondary doses of about 125 mg of amlitelimab, and wherein the subject receives doses Q12W from the start of treatment.

In another aspect, a method of treating atopic dermatitis in a subject in need thereof is provided, comprising administering to the subject amlitelimab, wherein the subject has a body weight ranging from about 25 kg to about 40 kg, wherein the subject receives an initial dose of about 250 mg of amlitelimab followed by one or more secondary doses of about 125 mg of amlitelimab, and wherein the subject receives doses Q12W from the start of treatment.

In another aspect, a method of treating atopic dermatitis (AD) in a subject in need thereof is provided, comprising administering to the subject amlitelimab, wherein the subject receives directly doses every 12 weeks (Q12W), without prior dose administration every 4 weeks (Q4W), wherein the subject receives an initial dose of about 500 mg of amlitelimab followed by one or more secondary doses of about 250 mg of amlitelimab, and wherein the method results in a decrease of the level of at least one biomarker in the subject relative to a control, for example relative to the level of the at least one biomarker prior to administering amlitelimab.

In another aspect, a method of treating atopic dermatitis (AD) in a subject in need thereof is provided, comprising administering to the subject amlitelimab, wherein the subject receives directly doses every 12 weeks (Q12W), without prior dose administration every 4 weeks (Q4W), wherein the subject receives an initial dose of about 500 mg of amlitelimab followed by one or more secondary doses of about 250 mg of amlitelimab, and reducing the level of at least one biomarker selecting from thymus and activation-regulated chemokine (TARC), interleukin-5 (IL-5), eosinophil count and lactate dehydrogenase (LDH), or combinations thereof.

In another aspect, a method of reducing a biomarker in a subject having atopic dermatitis (AD) is provided, comprising administering to the subject amlitelimab, wherein the subject receives directly doses every 12 weeks (Q12W), without prior dose administration every 4 weeks (Q4W), wherein the subject receives an initial dose of about 500 mg of amlitelimab followed by one or more secondary doses of about 250 mg of amlitelimab, and wherein the biomarker is selected from thymus and activation-regulated chemokine (TARC), interleukin-5 (IL-5), eosinophil count and lactate dehydrogenase (LDH).

In another aspect, a method of treating atopic dermatitis (AD) in a subject in need thereof is provided, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating atopic dermatitis (AD) in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg amlitelimab.

In another aspect, a method of treating atopic dermatitis (AD) in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating asthma in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating interstitial lung disease-systemic sclerosis in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating systemic sclerosis in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating hidradenitis suppurativa in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating alopecia areata in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating celiac disease in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

In another aspect, a method of treating atopic dermatitis in a subject in need thereof is provided, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab, and wherein efficacy is maintained at week 28 off of treatment or at week 32 after final administration of amlitelimab.

### BRIEF DESCRIPTION OF THE DRAWINGS

The accompanying drawings, which are incorporated herein and constitute part of this specification, illustrate exemplary embodiments of the disclosure, and, together with the general description given above and the detailed description given below, serve to explain the features of the disclosure.

FIG. 1 depicts main inclusion and exclusion criteria of the STREAM-AD study.

FIG. 2 depicts the primary analysis specifics of the STREAM-AD study.

FIG. 3 depicts baseline demographics for STREAM-AD study.

FIG. 4 depicts baseline disease characteristics for STREAM-AD study.

FIG. 5 depicts a primary endpoint at week 16: EASI, % change from baseline (LS mean).

FIG. 6 depicts a key secondary endpoint: EASI-75 over time, % patients.

FIG. 7 depicts a key secondary endpoint: IGA 0/1 over time, % patients.

FIG. 8 depicts a key secondary endpoint: PP-NRS ≥4 over time, % patients.

FIG. 9 is a comparison of key efficacy results at week 16 and week 24.

FIG. 10 is a PopPK/EASI model extending to Q12W after Q4W induction phase with loading dose. A comparable effect is observed with the three doses simulated. There is maintenance of the effect for more than 24 weeks after the last dose with a longer maintenance with the highest dose (less impact in case of missing doses).

FIG. 11 is a PopPK/EASI model for direct Q12W. Only minor differences are observed between the two dosing regimens (125/62.5 mg Q12W and 500/250 mg Q2W). Maintenance of the effect was for more than 24 weeks after the last dose, with a longer maintenance with the highest dose (less impact in case of missing doses).

FIG. 12 depicts a summary of Ph 2b endpoints.

FIG. 13 depicts the Ph 2b STREAM-AD trial design showing Part 1 (week 0 to week 24) and Part 2 (week 24 to week 52).

FIG. 14 depicts amlitelimab Cmax (left) and AUC4W (right) by dose arm in atopic dermatitis participants after first dose (week 1) and last dose (week 20) following Q4W administration (10th, 25th, 50th, 75th and 90th percentiles, and individual values).

FIG. 15 depicts amlitelimab steady state exposure (AUC4W) in atopic dermatitis participants by baseline body weight category (left) and in moderate versus severe atopic dermatitis (right) for each dose arm (10th, 25th, 50th, 75th and 90th percentiles, and individual values).

FIG. 16 depicts the predicted AUC12W and Cmax in AD participants from KY1005-CT05/DRI17366 (STREAM-AD) study after 250 mg Q12W (with a 500 mg loading dose) compared to 62.5 mg Q4W (without loading dose) and 250 mg Q4W (with a 500 mg loading dose) (n=301) for 24 weeks—0-12 week interval (above) and 12-24 week interval (below) (10th, 25th, 50th, 75th and 90th percentiles and individual values).

FIG. 17 schematically depicts a representation of the final PopPK model.

FIG. 18 schematically represents the final Population PK and PopPK/EASI models.

FIG. 19 depicts predicted percent change from baseline in EASI score in responder and non-responder participants (at week 24) with 250 mg Q12W (with a 500 mg loading dose) for 24 weeks (2000 participants simulated: 1200 responders and 800 non-responders). Non-responders, top data line; responders, bottom data line; responders+non-responders, middle data line.

FIG. 20 depicts simulation of two doses (125 loading dose+62.5 mg maintenance dose and 500 mg loading dose+250 mg maintenance dose) with a Q12W dosing interval. Shown are the median and the 10th and 90th percentile of the simulations of a cohort with 200 virtual patients. 125/62.5 mg, top data line; 500/250 mg, bottom data line.

FIG. 21 schematically depicts a legend for the pathway figures.

FIG. 22 schematically depicts a pharmacokinetics model for amlitelimab.

FIG. 23 schematically depicts a pharmacokinetics model for an anti-IL-13 antibody (antibody Y) and an anti-IL-4 receptor antibody (antibody X). Drugs from both PK models are handled within the model as total, free and bound (bound to drug target) drug in units of concentration.

FIG. 24 schematically depicts a combinatorial model of drug-target binding. DC Mature Lymph=mature dendritic cells in the lymph compartment, these cells express OX40L.

FIG. 25 schematically depicts biomarker production.

FIG. 26 schematically depicts cell migration and priming of T-helper cells.

FIG. 27 schematically depicts the combinatorial model of drug-target binding in skin.

FIG. 28 schematically depicts activation and life cycle of Th2 cells in the skin compartment. DC=dendritic cells, Treg=regulatory T-cells.

FIG. 29 schematically depicts activation and life cycle of Th22 cells in the skin compartment. DC=dendritic cells, Treg=regulatory T-cells.

FIG. 30 schematically depicts activation and life cycle of Treg cells in the skin compartment. Treg ChKs=chemokines attracting Tregs; DC=dendritic cells, Treg=regulatory T-cells.

FIG. 31 schematically depicts activation and life cycle of mast cells in the skin compartment.

FIG. 32 schematically depicts maturation and life cycle of mast cells in the skin compartment.

FIG. 33 schematically depicts the life cycle of keratinocytes.

FIG. 34 schematically depicts production and elimination of mediators in skin.

FIG. 35 schematically depicts processes that generate and control pruritus. KC=keratinocyte, MC=mast cell.

FIG. 36 schematically depicts a sequence of calculations for and the control of barrier function and keratinocyte quality. CC=corneocyte, KC=keratinocyte, MC=mast cell.

FIG. 37 schematically depicts a sequence of calculations for clinical outcome scores. DC=dendritic cell, MC=mast cell.

FIG. 38 depicts amlitelimab exposure after Q4W loading dosing by body weight band showing AUC_{4w} after loading dose (top) and Cₘₐₓ after loading dose (bottom).

FIG. 39 depicts amlitelimab exposure after Q4W loading dosing by body weight band showing steady state AUC_{4w} after loading dose (top) and steady state Cₘₐₓ after loading dose (bottom).

FIG. 40 depicts amlitelimab exposure after Q12W loading dosing by body weight band showing AUC_{12w} after loading dose (top) and Cₘₐₓ after loading dose (bottom).

FIG. 41 depicts amlitelimab exposure after Q12W loading dosing by body weight band showing steady state AUC_{12w} after loading dose (top) and steady state Cₘₐₓ after loading dose (bottom).

FIG. 42A-FIG. 42B depict treatment effect assessment bases on biomarker TARC.

FIG. 43A-FIG. 43B depict treatment effect assessment bases on biomarker LDH.

FIG. 44A-FIG. 44B depict treatment effect assessment bases on biomarker eosinophil counts.

FIG. 45A-FIG. 45B depict treatment effect assessment bases on biomarker IL-5.

FIG. 46A-FIG. 46B depict a treatment effect assessment model of TARC.

FIG. 47 depicts a treatment effect assessment model of LDH.

FIG. 48 depicts a treatment effect assessment model of blood eosinophils.

FIG. 49 graphically depicts that amlitelimab modulates all measured biomarkers with highest observed effect in Th2/Th22 biomarkers at week 24. Median % change from baseline was measured at week 24 for IgE, iL-13, IL-17A, IL-22, TARC, LDH and eosinophils.

FIG. 50 graphically depicts a treatment effect assessment model: Th2-associated biomarker IL-13.

FIG. 51 graphically depicts a treatment effect assessment model: Th2-associated biomarker IL-31.

FIG. 52 graphically depicts a treatment effect assessment model: Th2-associated biomarker Eotaxin-3.

FIG. 53 graphically depicts a treatment effect assessment model: IL-17A.

FIG. 54 graphically depicts a treatment effect assessment model: IL-22.

FIG. 55 graphically depicts a treatment effect assessment model: IgE.

FIG. 56 graphically depicts a treatment effect assessment model: Th2-associated biomarker IL-13.

FIG. 57 graphically depicts a treatment effect assessment model: Th2-associated biomarker IL-31.

FIG. 58 graphically depicts a treatment effect assessment model: Th2-associated biomarker IL-5.

FIG. 59 graphically depicts a treatment effect assessment model: Th2-associated biomarker eotaxin-3.

FIG. 60 graphically depicts a treatment effect assessment model: Th17/22-associated biomarkers: IL-17A.

FIG. 61 graphically depicts a treatment effect assessment model: Th17/22-associated biomarkers: IL-22.

FIG. 62 graphically depicts a treatment atopic dermatitis disease related biomarkers IgE.

FIG. 63 graphically depicts that amlitelimab modulates all measured biomarkers with highest observed effect in Th2/Th22 biomarkers.

FIG. 64 is a table showing that amlitelimab modulates biomarkers TARC, IgE, eosinophils, and eotaxin-3.

FIG. 65 graphically depicts a treatment effect assessment model: IL-22.

FIG. 66 graphically depicts a treatment effect assessment model: IL-17A.

FIG. 67A-FIG. 67B graphically depict a treatment effect assessment model: IL-13.

FIG. 68 depicts a table showing the distribution of responders from Part 1 that entered Part 2 of the STREAM-AD trial.

FIG. 69 is a table depicting the baseline demographics and disease characteristics at week 0 for Part 1 (overall) vs. Part 2 (responder) populations.

FIG. 70 is a table depicting the baseline disease characteristics at week 24 re-randomization.

FIG. 71 is a table depicting maintenance of IGA 0/1 response in week 24 IGA 0/1 responder participants at week 52 (28 weeks off-treatment). *Non-responder imputation (NRI) analysis: All participants that were IGA 0/1 responders at re-randomization visit were included. Patients who discontinued treatment due to lack of efficacy or received rescue medications or prohibited medications/procedures impacting efficacy on or after the re-randomization date or first dose date and before the corresponding timepoint in Part 2 were considered as non-responders. Patients who received rescue medications or prohibited medications/procedures impacting efficacy while in Part I were not considered as non-responders. Patients with missing data were considered non-responders. **Treatment policy analysis: All participants that were IGA 0/1 responders at re-randomization visit were included. Patients with missing data were considered non-responders.

FIG. 72 is a table depicting maintenance of EASI-75 response in week 24 EASI-75 responder participants at week 52 (28 weeks off-treatment). *Non-responder imputation (NRI) analysis: All participants that were EASI-75 responders at re-randomization visit were included. Patients who discontinued treatment due to lack of efficacy or received rescue medications or prohibited medications/procedures impacting efficacy on or after the re-randomization date or first dose date and before the corresponding timepoint in Part 2 were considered as non-responders. Patients who received rescue medications or prohibited medications/procedures impacting efficacy while in Part 1 were not considered as non-responders. Patients with missing data were considered non-responders. **Treatment policy analysis: All participants that were EASI-75 responders at re-randomization visit were included. Patients with missing data were considered non-responders.

FIG. 73 is a table depicting maintenance of IGA 0/1 and/or EASI-75 response in week 24 IGA 0/1 and/or EASI-75 responder participants at week 52 (28 weeks off-treatment). *Non-responder imputation (NRI) analysis: All participants that were IGA 0/1 and/or EASI-75 responders at re-randomization visit were included. Patients who discontinued treatment due to lack of efficacy or received rescue medications or prohibited medications/procedures impacting efficacy on or after the re-randomization date or first dose date and before the corresponding timepoint in Part 2 were considered as non-responders. Patients who received rescue medications or prohibited medications/procedures impacting efficacy while in Part I were not considered as non-responders. Patients with missing data were considered non-responders. **Treatment policy analysis: All participants that were IGA 0/1 and/or EASI-75 responders at re-randomization visit were included. Patients with missing data were considered non-responders.

FIG. 74 graphically depicts the durability of the EASI-75 response after treatment withdrawal. Percentages are based on the number of patients reaching EASI-75 at re-randomization (week 24), calculated as time from re-randomization to the first event date or censoring date for patients who had no events. Censoring date was week 68+1 day (EOS), calculated as the number of patients with an event divided by total patient years for the event. The time to loss is censored at the time of rescue medications and/or selected prohibited medications/procedures impacting efficacy use, or time of study discontinuation/completion, whichever was earlier. Participants that received rescue in Part 1 were able to enter Part 2 in STREAM-AD.

FIG. 75 is a table depicting that Amlitelimab was generally well-tolerated and demonstrated an acceptable safety profile across pooled dose arms for Part 2 safety population (week 0 to week 52). *For participants who entered Part 2, data are shown for Part 1+Part 2. Treatment with amlitelimab was given through week 52 (the last dose was given at week 48) with SFU through week 68. Data cut-off was when the last participant reached week 52. However, data presented include some participants up to 68 weeks. **Includes TEAEs that occurred while on amlitelimab in Part 1 as well as while on placebo in Part 2. ³Includes patients with “nasopharyngitis” and “pharyngitis.”

FIG. 76 is a table summarizing anti-drug antibody (ADA) data from week 0 to week 68. All treatment-emergent ADA were identified as treatment-induced ADA. *Treatment with amlitelimab was given through week 52 (the last dose was given at week 48) with SFU through week 68. Data cut-off was when the last participant reached week 52. However, data presented included some participants up to 68 weeks.

FIG. 77 graphically depicts that a durable clinical response (IGA 0/1 and EASI-75) was maintained with treatment (pooled dose arms). LLOQ; lowest level of quantification. ^{a}Data from the multiple ascending dose study in healthy subjects showed no effect on KLH immunization for amlitelimab concentrations≤4 μg/mL. ^{b}NRI: Patients who discontinued treatment due to lack of efficacy/received rescue/prohibited medications or procedures impacting efficacy on or after the re-randomization date/first dose date and before the corresponding time point in Part 2 were considered non-responders. Patients who received rescue/prohibited medications or procedures impacting efficacy while in Part I were not considered non-responders. Patients with missing data were considered non-responders. ^{c}All data (treatment policy): Patients with missing data were considered non-responders.

FIG. 78 graphically depicts that a durable clinical response was maintained following drug withdrawal despite serum amlitelimab reaching negligible levels^{a}. LLOQ; lowest level of quantification. ^{a}Data from the multiple ascending dose study in healthy subjects showed no effect on KLH immunization for amlitelimab concentrations≤4 μg/mL. ^{b}NRI: Patients who discontinued treatment due to lack of efficacy/received rescue/prohibited medications or procedures impacting efficacy on or after the re-randomization date/first dose date and before the corresponding time point in Part 2 were considered non-responders. Patients who received rescue/prohibited medications or procedures impacting efficacy while in Part I were not considered non-responders. Patients with missing data were considered non-responders. ^{c}All data (treatment policy): Patients with missing data were considered non-responders.

FIG. 79 graphically depicts that TARC levels (Th2) remained suppressed after amlitelimab was cleared from the serum. Fold-change from baseline (±CI) in biomarkers from mixed model repeated measures for respective visits up to week 52 for EASI-75 and/or IGA 0/1 responders who entered Part 2 (all data with a baseline and at least one evaluable biomarker assessment between week 24 and 52). A single model was used to estimate means for both continuing amlitelimab and withdrawn from amlitelimab groups.

FIG. 80 graphically depicts that eosinophil levels (Th2) remained suppressed after amlitelimab was cleared from the serum. Fold-change from baseline (±CI) in biomarkers from mixed model repeated measures for respective visits up to week 52 for EASI-75 and/or IGA 0/1 responders who entered Part 2 (all data with a baseline and at least one evaluable biomarker assessment between week 24 and 52). A single model was used to estimate means for both continuing amlitelimab and withdrawn from amlitelimab groups.

FIG. 81 graphically depicts that IL-22 levels (Th22) remained suppressed after amlitelimab was cleared from the serum. Fold-change from baseline (±CI) in biomarkers from mixed model repeated measures for respective visits up to week 52 for EASI-75 and/or IGA 0/1 responders who entered Part 2 (all data with a baseline and at least one evaluable biomarker assessment between week 24 and 52). A single model was used to estimate means for both continuing amlitelimab and withdrawn from amlitelimab groups.

FIG. 82A-FIG. 82H graphically depict fold-changes from baseline (±CI) in AD-related biomarkers during part 1 for all patients and part 2 (right side) for clinical responders. (FIG. 82A) TARC; (FIG. 82B) IgE; (FIG. 82C) IL-13; (FIG. 82D) IL-31; (FIG. 82E) IL-22; (FIG. 82F) IL-17A; (FIG. 82G) LDH; (FIG. 82H) blood eosinophils. Clinical responders were all patients with a baseline and at least one post-baseline biomarker result (part 1) and at least one biomarker result in part 2 between week 24 and week 52. Concentrations below the lower limit of quantification (LLOQ) were imputed to ½ LLOQ. Placebo (n: part 12=75, part 2=15), filled grey circle; 250 mg (+LD) amlitelimab (n: part 1=75, part 2=13) filled blue circle; 250 mg amlitelimab (n: part 1=74, part 2=12) filled red triangle; 125 mg amlitelimab (n: part 1=75, part 2=12), filled green diamond; 62.5 mg amlitelimab (n: part 1=78, part 2=7), filled yellow square; 250 mg (+LD) withdraw (n=34) dashed open blue circle; 250 mg withdraw (n=28), dashed open orange triangle; 125 mg withdraw (n=32), dashed open green diamond; 62.5 mg withdraw (n=34), dashed open yellow square.

### DETAILED DESCRIPTION

Embodiments of the present disclosure provide methods of treating immune mediated diseases or atopic dermatitis wherein the subject receives directly doses every 12 weeks (Q12W), without prior dose administration every 4 weeks (Q4W), methods of treating immune mediated diseases or atopic dermatitis in adolescent subjects, and methods of treating immune mediated diseases or atopic dermatitis that refer to particular biomarkers.

### Definitions

Listed below are definitions of various terms used to describe the embodiments disclosed herein. These definitions apply to the terms as they are used throughout this specification and claims, unless otherwise limited in specific instances, either individually or as part of a larger group.

Unless otherwise defined, all scientific and technical terms used herein shall have the meanings that are commonly understood by those of ordinary skill in the art. Further, unless otherwise required by context, singular terms shall include pluralities and plural terms shall include the singular. Generally, nomenclature utilized in connection with, and techniques of, cell and tissue culture, molecular biology, and protein and oligo- or polynucleotide chemistry and hybridization described herein are those well-known and commonly used in the art.

As used in this specification and the appended claims, the singular forms “a.” “an” and “the” include plural referents unless the content clearly dictates otherwise. Thus, for example, reference to “a molecule” optionally includes a combination of two or more such molecules, and the like. The use of “or” herein is the inclusive or.

As used herein, the term “about” or “approximately” will be understood by persons of ordinary skill in the art and will vary to some extent on the context in which it is used. When referring to a measurable value such as an amount, a temporal duration, and the like, the term “about” or “approximately” is meant to encompass variations of ±20%, ±15%, or ±10%, including ±5%, ±1%, and ±0.1% from the specified value, as such variations are appropriate to perform the disclosed methods.

As used in this specification and claim(s), the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “consisting essentially of” refers to those elements required for a given embodiment. The term permits the presence of elements that do not materially affect the basic and novel or functional characteristic(s) of that embodiment.

The abbreviation, “e.g.” is derived from the Latin exempli gratia, and is used herein to indicate a non-limiting example. Thus, the abbreviation “e.g.” is synonymous with the term “for example.”

As used herein, the term “amino acid” includes alanine (Ala or A); arginine (Arg or R); asparagine (Asn or N); aspartic acid (Asp or D); cysteine (Cys or C); glutamine (Gin or Q); glutamic acid (Glu or E); glycine (Gly or G); histidine (His or H); isoleucine (lie or I); leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, ornithine, norvaline, homoserine, and other amino acid residue analogues such as those described in Ellman et al. Meth. Enzymol. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q. C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

As used herein, the term “conservative amino acid substitutions” refers to amino acid substitutions result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide. (i.e. the ability of the peptide to penetrate the blood brain barrier (BBB)). Conservative substitution tables providing functionally similar amino acids are well known in the art. For example, the following six groups each contain amino acids that are conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can select the amino acid which will substitute an existing amino acid based on the location of the existing amino acid, i.e. its exposure to solvents (i.e. if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al. J. Mol Biol, 1999, 217, 721-739 and Taylor et al., J. Theor. Biol. 119 (1986): 205-218 and S. French and B. Robson, J. Mol. Evol., 19 (1983) 171. Accordingly, one can select conservative amino acid substitutions suitable for amino acids on the exterior of a protein or peptide (i.e. amino acids exposed to a solvent), for example, but not limited to, the following substitutions can be used: substitution of Y with F. T with S or K. P with A, E with D or Q. N with D or G. R with K. G with N or A. T with S or K. D with N or E, I with L or V. F with Y. S with T or A, R with K, G with N or A, K with R. A with S. K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F. T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

As used herein, the term “administering,” refers to dispensing, delivering, or applying an active compound, i.e., an antibody or antigen-binding fragment thereof, according to the present disclosure, in a pharmaceutical formulation to a subject by any suitable route for delivery of the active compound to the subject. Examples of routes of administration include, but are not limited to, subcutaneous, intravenous, e.g., intravenous injection and intravenous infusion, e.g., via central venous access, intramuscular, oral, nasal, and pulmonary administration.

As used herein, the term “antibody” generally refers to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CLI). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the terms “antagonistic antibody” or “antagonist antibody” are used herein equivalently and include an antibody that is capable of inhibiting and/or neutralizing the biological signalling activity of OX40, for example by blocking binding or substantially reducing binding of OX40 to OX40 ligand (OX40L) and thus inhibiting or reducing the signalling pathway triggered by OX40 and/or inhibiting or reducing an OX40-mediated cell response like lymphocyte proliferation, cytokine expression, or lymphocyte survival.

Unless specifically indicated otherwise, the term “antibody,” as used herein, shall be understood to encompass complete antibody molecules as well as antigen-binding fragments thereof. The term “antigen-binding portion” or “antigen-binding fragment” of an antibody (or simply “antibody portion” or “antibody fragment”), as used herein, refers to one or more fragments of an antibody that retain the ability to specifically bind to a target antigen such as human OX40L or an epitope thereof.

As used herein, the term “antibody fragment” refers to a polypeptide that includes at least one immunoglobulin variable domain or immunoglobulin variable do-main sequence and which specifically binds a given antigen. An antibody fragment can comprise an antibody or a polypeptide comprising an antigen-binding domain of an antibody. In some embodiments, an antibody fragment can comprise a monoclonal antibody or a polypeptide comprising an antigen-binding domain of a monoclonal anti-body. For example, an antibody can include a heavy (H) chain variable region (abbreviated herein as VH), and an OX40L (L) chain variable region (abbreviated herein as VL). In another example, an antibody includes two heavy (H) chain variable regions and two OX40L (L) chain variable regions. The term “antibody fragment” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol., 1996; 26 (3): 629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include minibodies, humanized antibodies, chimeric antibodies, and the like.

As used herein, “antibody variable domain” refers to the portions of the OX40L and heavy chains of antibody molecules that include amino acid sequences of complementarity determining regions (CDRs; i.e., CDR1, CDR2, and CDR3), and framework regions (FRs). VH refers to the variable domain of the heavy chain. VL refers to the variable domain of the Light chain. According to the methods used in this disclosure, the amino acid positions assigned to CDRs and FRs may be defined according to Kabat (Sequences of Proteins of Immunological Interest (National Institutes of Health, Bethesda, Md., 1987 and 1991)) or according to IMGT nomenclature.

As used herein, the term “antibody binding site” refers to a polypeptide or do-main that comprises one or more CDRs of an antibody and is capable of binding an antigen. For example, the polypeptide comprises a CDR3 (e.g., HCDR3). For example the polypeptide comprises CDRs 1 and 2 (e.g., HCDR1 and 2) or CDRs 1-3 of a variable domain of an antibody (e.g., HCDRs1-3). In one example, the antibody binding site is provided by a single variable domain (e.g., a VH or VL domain). In another example, the binding site comprises a VH/VL pair or two or more of such pairs.

As used herein, “OX40L antagonistic antibody” or “OX40L antagonist antibody” refers to an antibody or antigen-binding fragment thereof that is capable of inhibiting and/or neutralizing the biological signaling activity of OX40L, for example by blocking binding or substantially reducing binding of OX40 to OX40L.

As used herein, a “buffer” refers to a chemical agent that is able to absorb a certain quantity of acid or base without undergoing a strong variation in pH.

As used herein, the term “cell” is meant to refer to a cell that is in vitro, ex vivo, or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “dose” refers to a specified amount or quantity of a medication taken or recommended to be taken at a particular time. As used herein, it is typically expressed in mg of the antibody or fragment thereof. It may alternatively be expressed in terms of mg/kg, accounting for patient bodyweight. A “daily dose” refers to the total dosage amount administered to an individual in a single 24-hour day.

As used herein, the term “dosage” refers to the administering of a specific amount, number, and frequency of doses over a specified period of time. Dosage implies duration. A “dosage regimen” is a treatment plan for administering a drug over a period of time.

As used herein, the terms “improve,” “improving” or “improvement” or grammatical variations thereof used in relation to behaviors refer to the ability to achieve a measurable increase in performance in relation to tasks used to test these behaviors in a subject, including humans or non-human animals.

As used herein, “injection” refers to a means of administration and encompasses for example IV and subcutaneous injections. An IV injection may be referred to as an infusion. It is also used herein to refer to an instance of administration wherein that administration is by injection, for example in the phrase “one or more induction phase injections”. Each injection will involve administration of a dose of antibody or fragment thereof.

As used herein, “injection device” refers to a device that is designed for carrying out injections, an injection including the steps of temporarily fluidically coupling the injection device to a person's tissue, typically the subcutaneous tissue. An injection further includes administering an amount of aqueous drug into the tissue and decoupling or removing the injection device from the tissue. In some embodiments, an injection device can be an intravenous device or IV device, which is a type of injection device used when the target tissue is the blood within the circulatory system, e.g., the blood in a vein. A common, but non-limiting example of an injection device is a needle and syringe.

As used herein, “instructions” refers to a display of written, printed or graphic matter on the immediate container of an article, for example the written material displayed on a vial containing a pharmaceutically active agent, or details on the formulation and use of a product of interest included in a kit containing a formulation of interest. Instructions set forth the method of the treatment as contemplated to be administered or performed.

As used herein, the terms “isolated antibody” or “purified antibody” refers to an antibody that by virtue of its origin or source of derivation has one to four of the following: (1) is not associated with naturally associated components that accompany it in its native state, (2) is free or substantially free of other proteins from the same species. (3) is expressed by a cell from a different species, or (4) does not occur in nature. An isolated antibody is substantially free of other antibodies having different antigenic specificities.

As used herein, the term “formulation” as it relates to an antibody is meant to describe the antibody in combination with a pharmaceutically acceptable excipient comprising at least one buffer, at least one stabilizer, at least one surfactant, at least one chelating agent, and wherein the pH is as defined. As used herein, the term “formulation” may be used interchangeable with the term “composition.”

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, a “humanized” antibody refers to forms of non-human (e.g. murine) antibodies that are chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. In exemplary embodiments, humanized antibodies are human immunoglobulins (recipient antibody) in which residues from a CDR of the recipient are replaced by residues from a

CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

As used herein, the terms “level” and “levels” can be used interchangeably with the terms “concentration” and “concentrations.”

As use herein, the term “patient” or “subject” or “animal” or “host” refers to mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, NY, 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6 aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green and Wuts, PROTECTING GROUPS IN ORGANIC SYNTHESIS (John Wiley and Sons, 1991). The functional groups of a peptide can also be derivatized (e.g., alkylated) using art-known methods.

As used herein, the term “pharmaceutical formulation” or “drug formulation” refers to a preparation which is in such form as to permit the biological activity of the active ingredients to be effective. “Pharmaceutical formulation” and the term “drug formulation” refer to a mixture or a structure in which different chemical substances, including the active drug, are combined to form a final medicinal product, such as a sterile product, a solution, a powder, an emulsion, a capsule, a tablet, a granule, a topical preparation, a non-conventional product such as semi-solid or sustained-release preparations, liquid, etc. Pharmaceutical formulation is prepared according to a specific procedure, a “formula.” The drug formed varies by the route of administration. As used herein, the term “formulation” as it relates to an antibody is meant to describe the antibody in combination with a pharmaceutically acceptable excipient comprising at least one buffer, at least one stabilizer, at least one surfactant, at least one chelating agent, and wherein the pH is as defined.

As used herein, the term “pharmaceutical formulation” is interchangeable with the term “pharmaceutical composition,” which further refers to the active agent in combination with a pharmaceutically acceptable carrier e.g., a carrier commonly used in the pharmaceutical industry. The phrase “pharmaceutically acceptable” is employed herein to refer to those compounds, materials, compositions or formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipients” (vehicles, additives) are those, which can safely be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the aqueous formulations herein. Exemplary diluents include, but are not limited to, sterile water and bacteriostatic water for injection (BWFI).

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, the term “pharmaceutically acceptable carrier” also includes any and all coatings, antibacterial and antifungal agents, and absorption delaying agents, and the like that are compatible with the activity of the compound useful within the present disclosure, and are physiologically acceptable to the subject. In certain situations, supplementary active compounds may also be incorporated into a pharmaceutical formulation. The “pharmaceutically acceptable carrier” may further include a pharmaceutically acceptable salt of the compound disclosed herein. Other additional ingredients that may be included in a pharmaceutical formulation are known in the art and described, for example, in Remington's Pharmaceutical Sciences (Genaro, Ed., Mack Publishing Co., 1985, Easton, PA), which is incorporated herein by reference.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two: generally, non-aqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the terms “portion,” “fragment,” “variant,” “derivative” and “analog,” when referring to a polypeptide of the present disclosure include any polypeptide that retains at least some biological activity referred to herein (e.g., antigen binding).

As used herein, the term “immune mediated disease” includes the meaning of a “hOX40L-mediated disease” or a “hOX40L-mediated condition”, which are used interchangeably and include a reference to any disease or condition that is completely or partially caused by or is the result of hOX40L. In certain embodiments, hOX40L is aberrantly (e.g., highly) expressed on the surface of a cell. In some embodiments, hOX40L may be aberrantly upregulated on a particular cell type. In other embodiments, normal, aberrant or excessive cell signaling is caused by binding of hOX40L to a hOX40L ligand. In certain embodiments, the hOX40L ligand is OX40, for example, that is expressed on the surface of a cell. The immune mediated disease may be selected from an autoimmune disease or condition, a systemic inflammatory disease or condition, or transplant rejection.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms ‘treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not contemplated. The method can in certain aspects include cure as well.

As used herein, the term “mg/kg” refers to the dose of a substance administered to an individual in milligrams per kilogram of body weight of the individual.

As used herein, “packaging” refers to how the components are organized and/or restrained into a unit fit for distribution and/or use. Packaging can include, e.g., boxes, bags, syringes, ampoules, vials, tubes, clamshell packaging, barriers and/or containers to maintain sterility, labeling, etc.

As used herein, the term “recombinant antibody” is intended to include all antibodies that are prepared, expressed, created or isolated by recombinant means, for example antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, such recombinant human antibodies can be subjected to in vitro mutagenesis.

As used herein, “compounding” or “sterile compound” involves preparing medication in an environment free from bacteria, viruses, or any other potentially infectious microorganisms. Sterile compounding is used for preparations that will be administered either through an IV, injection, or directly into the eyes.

As used herein, the phrases “systemic administration,” “administered systemically,” “peripheral administration” and “administered peripherally” as used herein mean the administration of a compound, drug or other material other than directly into a target tissue (e.g., the nervous system), such that it enters the animal's system and, thus, is subject to metabolism and other like processes, for example, subcutaneous administration.

As used herein, the term “sequence identity,” “percent identity,” “percent homology,” or, for example, comprising a “sequence 80% identical to,” refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, at least 40%, at least 50%, 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 42 (HCDR1). In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 44 (HCDR2). In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 46 (HCDR3). In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 56 (LCDR1). In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 58 (LCDR2). In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 60 (LCDR3).

In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 34 (VH). In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 48 (VL).

In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 62 (HC). In certain embodiments, the length of a reference sequence aligned for comparison purposes is at least 70%, 75%, 80%, 85%, 90%, 91%, 92%, 93%, 94%, 95%, 96%, 97%, 98%, 99%, or 100% identical to SEQ ID NO: 64 (LC).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2. 3, 4, 5, or 6. In yet another exemplary embodiment, the percent identity between two nucleotide sequences is determined using the GAP program in the GCG software package, using an NWSgapdna. CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4:11-17) which has been incorporated into the ALIGN program (version 2.0), using a PAM120 weight residue table, a gap length penalty of 12 and a gap penalty of 4.

For instance, the peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215:403-10). BLAST nucleotide searches can be performed with the NBLAST program, score=100, wordlength=12 to obtain nucleotide sequences homologous to nucleic acid molecules of the disclosure. BLAST protein searches can be performed with the XBLAST program, score=50, wordlength=3 to obtain amino acid sequences homologous to protein molecules of the disclosure. To obtain gapped alignments for comparison purposes. Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

Definitions of common terms in cell biology and molecular biology can be found in “The Merck Manual of Diagnosis and Therapy,” 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0)-632-02182-9); Benjamin Lewin, Genes X, published by Jones & Bartlett Publishing, 2009 (ISBN-10:0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, standard procedures were used, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss: 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various embodiments of the disclosure.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the formulations, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure of the detailed description. All such modifications are intended to be included within the scope of the appended claims.

### Atopic Dermatitis-Associated Biomarkers

Examples of AD-associated biomarkers include, but are not limited to, one or any combination of IgE, thymus activation regulated chemokine (TARC), lactate dehydrogenase (LDH), interleukin-5 (IL-5), IL-13, IL-17A, IL-22, IL-31, eotaxin-3, and eosinophil count. In certain embodiments, one or more AD-associated biomarkers may be detected from a biological sample derived from a subject (e.g. blood or serum).

In certain embodiments, the subject experiences an improvement in AD as measured by a biomarker relative to the biomarker measurement at baseline (e.g. the level of the biomarker prior to treatment with the OX40L antibody or antigen binding fragment thereof). For example, the biomarker may be one or more or all of IgE levels, TARC levels, LDH levels, IL-5 levels, IL-13 levels, IL-17A levels, IL-22 levels, IL-31 levels, eotaxin-3 levels, and eosinophil count levels. By levels we include the meaning of biomarker expression (e.g. RNA and protein levels of the biomarkers). In certain exemplary embodiments, TARC level is decreased relative to baseline. In certain exemplary embodiments, LDH level is decreased relative to baseline. In certain exemplary embodiments, total IgE level is decreased relative to baseline. In certain exemplary embodiments, IL-5 level is decreased relative to baseline. In certain exemplary embodiments, TARC level is decreased relative to baseline. In certain exemplary embodiments, IL-13 level is decreased relative to baseline. In certain exemplary embodiments, IL-17A level is decreased relative to baseline. In certain exemplary embodiments, IL-22 level is decreased relative to baseline. In certain exemplary embodiments, IL-31 level is decreased relative to baseline. In certain exemplary embodiments, eotaxin-3 level is decreased relative to baseline. In certain exemplary embodiments, eosinophil level is decreased relative to baseline. In certain embodiments, an improvement in AD is indicated by a reduction at week 4, week 12, week 24, etc., following treatment relative to baseline AD. In other words, an improvement in AD may be indicated by a reduction in any one or more or all of IgE levels, TARC levels, LDH levels, IL-5 levels, IL-13 levels, IL-17A levels, IL-22 levels, IL-31 levels, eotaxin-3 levels, and eosinophil count levels at week 4, week 12, week 24, etc., following treatment relative to the respective levels prior to administration of the OX40L antibody or antigen binding fragment thereof.

Improvement of an AD-associated parameter can be expressed as a percentage. For example, a score can be improved by 30% or more, by 40% or more, by 50% or more, by 60% or more, by 70% or more, or by 80% or more, or by 90% or more or by 100% relative to baseline.

Biomarker expression or levels, as discussed above, can be assayed by detection of protein or RNA in serum. In some embodiments, RNA samples are used to determine RNA levels (non-genetic analysis), e.g., RNA levels of biomarkers; and in other embodiments, RNA samples are used for transcriptome sequencing (e.g., genetic analysis).

To determine whether an AD-associated parameter has “improved,” the parameter is quantified at baseline and at a time point after administration of the antibody or antigen binding fragment thereof, or pharmaceutical composition of the present invention. For example, an AD-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 52, or longer, after the initial treatment with a pharmaceutical composition described herein. In some embodiments, the parameter is measured daily (e.g., once, or twice per day), weekly, biweekly, or monthly. In other embodiments, the parameter is measured daily, and the mean value determined over the course of a month is compared to baseline. By AD-associated parameter in this context we include the meaning of all of the biomarkers discussed above, and the clinical scores of AD discussed below.

The difference between the value of the parameter at a particular time point following initiation of treatment and the value of the parameter at baseline is used to establish whether there has been an “improvement” in the AD-associated parameter (e.g., an increase or decrease depending on the specific parameter being measured).

### Various Testing Types

### Eczema Area Surface Index (EASI)

An EASI score measures the extent (area) and severity of AD. The calculation for each region is severity score×area score×multiplier. A score of 0 indicates clear or no eczema, 0.1 to 1.0 indicates almost clear, 1.1 to 7 indicates mild disease, 7.1 to 21 indicates moderate disease, 21.1 to 50 indicates severe disease, and greater than 51 indicates very severe disease.

For extent, four body regions are scored: 1) Head and neck (the face occupies 33% (17% each side), the neck 33% (17% front and back and the scalp 33%); 2) Trunk (including genital area, (front is 55% and back is 45%)); 3) Upper limbs (each arm occupies 50% of the upper limbs region (front or back of one arm is 25%)); and 4) Lower limbs ((including buttocks), each leg is 45% (front or back of one leg is 22.5%) and buttocks are 10%)). An area score is calculated for each of the four regions of the body. The area score is the percentage of skin affected by eczema for each body region. An area score of 0=no active eczema in the region. An area score of 1=1-9% of skin affected in the region. An area score of 2=10-29% of skin affected in the region. An area score of 3=30-49% of skin affected in the region. An area score of 4=50-69% of skin affected in the region. An area score of 5=70-89% of skin affected in the region. An area score of 6=90-100% of skin affected in the region (the entire region is affected by eczema.

For severity, each of the four regions of the body is scored. The severity score is the sum of the intensity scores for four signs. The four signs are: Redness (erythema, inflammation):

Thickness (induration, papulation, swelling—acute eczema); Scratching (excoriation); and Lichenification (lined skin, furrowing, prurigo nodules—chronic eczema). The average intensity of each sign in each body region is assessed as: none/absent (0); mild/just perceptible (1); moderate/obvious (2); and severe (3). Half scores are allowed.

EASI-75 indicates a ≥75% improvement in EASI score from baseline. EASI-90 indicates a ≥90% improvement in EASI score from baseline. EASI-100 indicates a 100% improvement in EASI score from baseline.

### Investigator Global Assessment (IGA)

IGA is a four-point scoring system of the overall severity of AD skin lesions. A score of 0)=clear, no inflammatory signs of AD. A score of 1=almost clear, with just perceptible erythema, and just perceptible papulation/infiltration. A score of 2=mild disease, with mild erythema and mild papulation/infiltration. A score of 3=moderate disease, with moderate erythema and moderate papulation/infiltration. A score of 4=severe disease, with severe erythema and severe papulation/infiltration.

### Peak Pruritis Numeric Rating Scale (PP-NRS)

PP-NRS is a PRO designed to measure peak pruritus, or ‘worst’ itch, over the previous 24 h based on the following question: “On a scale of 0 to 10, with 0 being “no itch” and 10 being “worst itch imaginable.”, how would you rate your itch at the worst moment during the previous 24 hours?′.

### Skin Pain Numeric Rating Scale (SP-NRS)

SP-NRS is a PRO that measures “worst skin pain” in the past 24 hours on an 11-point scale (0=no pain, and 10=worst pain imaginable).

### Sleep Disturbance Numeric Rating Scale (SD-NRS)

SD-NRS is a PRO that measures sleep by asking a subject to rate their sleep loss during the previous night on a scale of 0 (“no sleep loss”) to 10 (“I did not sleep at all”).

### Dermatology Quality of Life Index (DLQI)

The DLQI is a patient-reported outcome (PRO) developed to measure dermatology-specific HRQoL in a subject. The instrument comprises 10 items assessing the impact of skin disease on a participant's health-related quality of life (HRQoL) over the previous week. The items cover symptoms, leisure activities, work/school or holiday time, personal relationships including intimate, the side effects of treatment, and emotional reactions to having a skin disease. It is a validated questionnaire used in clinical practice and clinical trials. The response scale is a 4-point Likert scale (0=“not at all” and 3=“very much”) for 9 items. The remaining 1 item about work/studying asks whether work/study has been prevented and then (if “no”) to what degree the skin condition has been a problem at work/study; the item is rated on a 3-point Likert scale (“not at all” to “a lot”). Overall scoring ranges from 0 to 30, with a high score indicative of a poor HRQoL.

### Hospital Anxiety and Depression Scale (HADS)

HADS is a fourteen-item scale with seven items each for anxiety and depression subscales. Scoring for each item ranges from zero to three. A subscale score >8 denotes anxiety or depression. A subscale score of 8-10 is mild, a subscale score of 11-14 is moderate, and a subscale score of 15-21 is severe. A score of 7 or less is considered a non-case.

### Scoring of Atopic Dermatitis (SCORAD)

SCORAD is a clinical tool used to assess the extent and severity of AD using area score, intensity score and subjective symptom score. The SCORAD score range is between 0 and 103 points and defines three classes of AD severity (i.e., mild if SCORAD <25, moderate if 25≤SCORAD≤50 and severe if SCORAD>50). The total SCORAD score is A/5+7B/2+C.

To determine area, the sites affected by eczema are shaded on a drawing of a body. The rule of 9 is used to calculate the affected area (A) as a percentage of the whole body: Head and neck 9%; Upper limbs 9% each; Lower limbs 18% each; Anterior trunk 18%; Back 18%; and Genitals 1%. The score for each area is added up. The total area is “A,” having a maximum score of 100%.

To determine intensity, a representative area of eczema is selected. In this area, the intensity of each of the following signs is assessed as none (0), mild (1), moderate (2) or severe (3): Redness; Swelling; Oozing/crusting; Scratch marks; Skin thickening (lichenification); and Dryness (this is assessed in an area where there is no inflammation). The intensity scores are added together to give “B,” having a maximum score of 18.

To determine subjective symptoms (i.e., itch and sleeplessness), each are scored by the patient or relative using a visual analogue scale where 0 is no itch (or no sleeplessness) and 10 is the worst imaginable itch (or sleeplessness). These scores are added to give “C,” having a maximum score of 20.

### Administration Regimens

According to certain embodiments, multiple doses of an anti-OX40L antibody or antigen binding fragment thereof may be administered to a subject over a defined time course. Such methods comprise sequentially administering to a subject multiple doses of an anti-OX40L antibody or antigen binding fragment thereof. As used herein, “sequentially administering” means that each dose of an anti-OX40L antibody or antigen binding fragment thereof is administered to the subject at a different point in time, e.g., on different days separated by a predetermined interval (e.g., hours, days, weeks, or months). Methods that comprise sequentially administering to the patient a single initial dose of an anti-OX40L antibody or antigen binding fragment thereof, followed by one or more secondary doses of the anti-OX40L antibody or antigen binding fragment thereof, and optionally followed by one or more tertiary doses of the anti-OX40L antibody or antigen binding fragment thereof, are provided.

Methods comprising administering to a subject a pharmaceutical composition comprising an anti-OX40L antibody or antigen binding fragment thereof at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (q10w), once every eleven weeks (q11w), once every twelve weeks (q12w), or less frequently so long as a therapeutic response is achieved, are provided.

In certain embodiments involving the administration of a pharmaceutical composition comprising an anti-OX40L antibody or antigen binding fragment thereof, once a week dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every two weeks dosing (every two weeks is used interchangeably with every other week, bi-weekly or q2w) of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every three weeks dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every four weeks dosing (monthly dosing) of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every five weeks dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every six weeks dosing of an amount of about 62.5 mg, about 125 mg, about 250) mg, about 300 mg, about 400 mg, about 500 mg or about 600 mg can be employed. In other embodiments involving the administration of a pharmaceutical composition comprising an anti-OX40L antibody or antigen binding fragment thereof, once every eight weeks dosing of an amount of about 62.5 mg, about 125 mg, about 250) mg, about 300 mg, about 400 mg, about 500 mg or about 600 mg can be employed. In other embodiments involving the administration of a pharmaceutical composition comprising an anti-OX40L antibody or antigen binding fragment thereof, once every twelve weeks dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, a subject receives doses once every twelve weeks from the start of treatment in an amount of about 62.5 mg, about 125 mg, about 250) mg, about 300 mg, about 400 mg, about 500 mg or about 600 mg. In certain exemplary embodiments, the route of administration is subcutaneous.

In certain embodiments involving the administration of a pharmaceutical composition comprising an anti-OX40L antibody or antigen binding fragment thereof, once a week dosing of an amount of 62.5 mg, 125 mg, 250 mg, 300 mg, 400 mg, 500 mg or 600 mg can be employed. In other embodiments involving the administration of a pharmaceutical composition comprising an anti-OX40L antibody or antigen binding fragment thereof, once every two weeks dosing (every two weeks is used interchangeably with every other week, bi-weekly or q2w) of an amount of 62.5 mg, 125 mg, 250 mg, 300 mg, 400 mg, 500 mg or 600 mg can be employed. In other embodiments involving the administration of a pharmaceutical composition comprising an anti-OX40L antibody or antigen binding fragment thereof, once every three weeks dosing of an amount of 62.5 mg, 125 mg, 250 mg, 300 mg, 400 mg, 500 mg or 600 mg can be employed. In other embodiments involving the administration of a pharmaceutical composition comprising an anti-OX40L antibody or antigen binding fragment thereof, once every four weeks dosing (monthly dosing) of an amount of 62.5 mg, 125 mg, 250 mg, 300 mg, 400 mg, 500 mg or 600 mg can be employed. In other embodiments involving the administration of a pharmaceutical composition comprising an anti-OX40L antibody or antigen binding fragment thereof, once every five weeks dosing of an amount of 62.5 mg, 125 mg, 250 mg, 300 mg, 400 mg, 500 mg or 600 mg can be employed. In other embodiments involving the administration of a pharmaceutical composition comprising an anti-OX40L antibody or antigen binding fragment thereof, once every six weeks dosing of an amount of 62.5 mg. 125 mg, 250 mg, 300 mg, 400 mg, 500 mg or 600 mg can be employed. In other embodiments involving the administration of a pharmaceutical composition comprising an anti-OX40L antibody or antigen binding fragment thereof, once every eight weeks dosing of an amount of 62.5 mg, 125 mg, 250 mg, 300 mg, 400 mg, 500 mg or 600 mg can be employed. In other embodiments involving the administration of a pharmaceutical composition comprising an anti-OX40L antibody or antigen binding fragment thereof, once every twelve weeks dosing of an amount of 62.5 mg, 125 mg, 250 mg, 300 mg, 400 mg, 500 mg or 600 mg can be employed. In other embodiments involving the administration of a pharmaceutical composition comprising an anti-OX40L antibody or antigen binding fragment thereof, a subject receives doses once every twelve weeks from the start of treatment in an amount of 62.5 mg, 125 mg, 250 mg, 300 mg, 400 mg, 500 mg or 600 mg. In certain exemplary embodiments, the route of administration is subcutaneous.

In certain embodiments involving the administration of a pharmaceutical composition comprising an anti-OX40L antibody or antigen binding fragment thereof, the subject may receive an initial dose of about 500 mg of the antibody or antigen binding fragment thereof followed by one or more secondary doses of about 250 mg of the antibody or antigen binding fragment thereof, wherein the secondary dose is administered every 12 weeks (Q12W), the subject is an adult subject, and the administration is subcutaneous.

In certain embodiments involving the administration of a pharmaceutical composition comprising an anti-OX40L antibody or antigen binding fragment thereof, the subject may receive an initial dose of about 250 mg of the antibody or antigen binding fragment thereof followed by one or more secondary doses of about 125 mg of the antibody or antigen binding fragment thereof, wherein the secondary dose is administered every 12 weeks (Q12W), the subject is an adolescent subject, and the administration is subcutaneous.

The term “week” or “weeks” refers to a period of (n×7 days)+3 days, e.g., (n×7 days)±2 days, (n×7 days)±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of an anti-OX40L antibody or antigen binding fragment thereof. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of an anti-OX40L antibody or antigen binding fragment thereof, or may differ from one another in terms of frequency of administration. In certain embodiments, however, the amount of an anti-OX40L antibody or antigen binding fragment thereof contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis. In one embodiment, the secondary dose may be lower than the loading dose. For example, one or more initial doses or loading doses of 62.5 mg, 125 mg, 250 mg or 500 mg of an anti-OX40L antibody or antigen binding fragment thereof may be administered followed by secondary doses of about 62.5 mg, 125 mg, 250 mg or 500 mg. In one embodiment, the secondary dose may be equal to the initial dose/loading dose. For example, one or more initial doses/loading doses of about 62.5 mg, 125 mg, 250 mg or 500 mg of an anti-OX40L antibody or antigen binding fragment thereof may be administered followed by secondary doses of about 62.5 mg, 125 mg, 250 mg or 500 mg, respectively.

In certain embodiments, the initial dose is about 50 mg to about 600 mg of the anti-OX40L antibody or antigen binding fragment thereof. In certain embodiments, the initial dose is about 62.5 mg of the anti-OX40L antibody or antigen binding fragment thereof. In certain embodiments, the initial dose is about 125 mg of the anti-OX40L antibody or antigen binding fragment thereof. In certain embodiments, the initial dose is about 250 mg of the anti-OX40L antibody or antigen binding fragment thereof. In another embodiment, the initial dose is 500 mg of the anti-OX40L antibody or antigen binding fragment thereof.

In certain embodiments, the secondary dose(s) are about 50 mg to about 600 mg of the anti-OX40L antibody or antigen binding fragment thereof. In one embodiment, the secondary dose is about 62.5 mg of the anti-OX40L antibody or antigen binding fragment thereof. In one embodiment, the secondary dose is about 125 mg of the anti-OX40L antibody or antigen binding fragment thereof. In one embodiment, the secondary dose is about 125 mg of the anti-OX40L antibody or antigen binding fragment thereof. In one embodiment, the secondary dose is about 250 mg of the anti-OX40L antibody or antigen binding fragment thereof.

In certain embodiments, an initial loading dose is about 500 mg (two injections of 250 mg), followed by a dose of about 250 mg (one injection of 250 mg) administered Q12W.

In certain embodiments, an initial loading dose is about 500 mg (two injections of 250 mg), followed by a dose of about 250 mg (one injection of 250 mg) administered Q4W, and for patients with clear or almost clear skin at 24 weeks of treatment, followed by a dose of about 250 mg administered Q12W.

### Treatment Populations

The methods provided herein include administering to a subject in need thereof a therapeutic composition comprising an anti-OX40L antibody or antigen binding fragment thereof. The expression “a subject in need thereof” means a human or non-human animal that exhibits one or more symptoms or indicia of AD, or who has been diagnosed with AD. For example, “a subject in need thereof” may include, e.g., subjects who, prior to treatment, exhibit (or have exhibited) one or more AD-associated parameters, such as, e.g., impaired EASI, impaired IGA, impaired PP-NRS, impaired SP-NRS, impaired SD-NRS, impaired DLQI, and/or impaired HADS. In various embodiments, the methods may be used to treat mild, moderate-to-severe (e.g., uncontrolled moderate-to-severe), and severe AD in patients in need thereof.

In some embodiments, a “subject in need thereof is selected from the group consisting of: a subject age 18 years old or older, a subject 12 years or older, a subject age 12 to 17 years old (12 to <18 years old), a subject age 6 to 11 years old (6 to <12 years old), and a subject age 2 to 5 years old (2 to <6 years old). In some embodiments, a “subject in need thereof” is selected from the group consisting of: an adult, an adolescent, and a child. In some embodiments, a “subject in need thereof” is selected from the group consisting of: an adult age 18 years of age or older, an adolescent age 12 to 17 years old (12 to <18 years old), a child age 6 to 11 years old (6 to <12 years old), and a child age 2 to 5 years old (2 to <6 years old). The subject can be less than 2 years of age, e.g., 12 to 23 months, or 6 to 11 months. In some embodiments, a “subject in need thereof” has a body weight of between about 25 kg and about 40 kg.

### Dosage

The amount of anti-OX40 ligand (OX40L) antibody or antigen binding fragment thereof administered to a subject according to the methods described herein is, generally, a therapeutically effective amount. As used herein, the phrase “therapeutically effective amount” means an amount of anti-OX40L antibody or antigen binding fragment thereof that results in an improvement in one or more AD-associated parameters (as defined elsewhere herein). A “therapeutically effective amount” also includes an amount of anti-OX40L antibody or antigen binding fragment thereof that inhibits, prevents, lessens, or delays the progression of AD in a subject.

The dose may be from 20 mg to 1000 mg. The dose may be from 20 mg to 600 mg. The dose may be up to 550 mg, up to 500 mg, up to 450 mg, up to 400 mg, up to 350 mg, up to 300 mg, up to 250 mg, up to 200 mg, up to 150 mg, up to 120 mg, up to 100 mg, or up to 50 mg. The dose may be up to 500 mg, up to 250 mg, or up to 150 mg. The dose may be at least 50 mg, at least 100 mg, at least 120 mg, at least 150 mg, at least 200 mg, at least 250 mg, at least 300 mg, at least 350 mg, at least 400 mg, at least 450 mg, at least 500 mg or at least 550 mg. The dose may be at least 50 mg, at least 120 mg or at least 150 mg. The dose may be selected from the group consisting of from 25 mg to 500 mg; from 50 mg to 450 mg; from 100 mg to 350 mg; from 120 mg to 300 mg; from 150 mg to 250 mg; and from 200 mg to 250 mg. The dose may be selected from the group consisting of from 60 mg to 500 mg; from 100 mg to 300 mg or from 125 mg to 150 mg.

The dose may be 62.5 mg, 125 mg, 150 mg, 250 mg or 500 mg. The dose may be 125 mg or 150 mg. The dose may be 125 mg. The dose may be 150 mg. The dose may be 62.5 mg. The dose may be 250 mg. The dose may be 500 mg.

The dose may be of up to 0.6 mg/kg, up to 0.7 mg/kg, up to 0.8 mg/kg, up to 0.9 mg/kg, up to 1 mg/kg, up to 1.1 mg/kg, up to 1.2 mg/kg, up to 1.3 mg/kg, up to 1.4 mg/kg, up to 1.5 mg/kg, up to 1.6 mg/kg, up to 1.7 mg/kg, up to 1.8 mg/kg, up to 1.9 mg/kg, up to 2 mg/kg, up to 2.1 mg/kg, up to 2.2 mg/kg, up to 2.3 mg/kg, up to 2.4 mg/kg, up to 2.5 mg/kg, up to 2.6 mg/kg, up to 2.7 mg/kg, up to 2.8 mg/kg, up to 2.9 mg/kg, up to 3 mg/kg, up to 4 mg/kg, up to 5 mg/kg, up to 6 mg/kg, up to 7 mg/kg, up to 8 mg/kg, up to 9 mg/kg, up to 10 mg/kg, up to 11 mg/kg or up to 12 mg/kg. The dose may be of up to 6 mg/kg or up to 3 mg/kg.

The dose may be of at least 0.45 mg/kg, at least 0.5 mg/kg, at least 0.6 mg/kg, at least 0.7 mg/kg, at least 0.8 mg/kg, at least 0.9 mg/kg, at least 1 mg/kg, at least 1.1 mg/kg, at least 1.2 mg/kg, at least 1.3 mg/kg, at least 1.4 mg/kg, at least 1.5 mg/kg, at least 1.6 mg/kg, at least 1.7 mg/kg, at least 1.8 mg/kg, at least 1.9 mg/kg, at least 2 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, or at least 12 mg/kg. The dose may be of at least 0.45 mg/kg. The dose may be of at least 0.7 mg/kg or at least 1.4 mg/kg.

The dose may be selected from the group consisting of from 0.1 mg/kg to 12 mg/kg; from 0.4 mg/kg to 11 mg/kg; from 0.7 mg/kg to 10 mg/kg; from 1 mg/kg to 9 mg/kg; from 1.3 mg/kg to 8 mg/kg; from 1.6 mg/kg to 7 mg/kg; from 1.9 mg/kg to 6 mg/kg; from 2.2 mg/kg to 5 mg/kg; from 2.5 mg/kg to 4 mg/kg; from 2.6 mg/kg to 3.8 mg/kg; from 2.7 mg/kg to 3.6 mg/kg; from 2.6 mg/kg to 3.4 mg/kg; from 2.7 mg/mg to 3.3 mg/kg; from 2.8 mg/kg to 3.2 mg/kg; and from 2.9 mg/kg to 3.1 mg/kg. The dose may be selected from the group consisting of from 0.6 mg/kg to 11 mg/kg; from 0.7 mg/kg to 10 mg/kg; from 0.8 mg/kg to 9 mg/kg; from 0.9 mg/kg to 8 mg/kg; from 1 mg/kg to 7 mg/kg; from 1.1 mg/kg to 6 mg/kg; from 1.2 mg/kg to 5 mg/kg; from 1.3 mg/kg to 4 mg/kg; from 1.4 mg/kg to 3 mg/kg; from 1.5 mg/kg to 2.9 mg/kg; from 1.6 mg/kg to 2.8 mg/kg; from 1.7 mg/mg to 2.7 mg/kg; from 1.8 mg/kg to 2.6 mg/kg; from 1.9 mg/kg to 2.5 mg/kg; from 2 mg/kg to 2.4 mg/kg; and from 2.1 mg/kg to 2.3 mg/kg.

The dose may be from 0.7 mg/kg to 6 mg/kg. The dose may be from 1.4 mg/kg to 3 mg/kg.

In certain embodiments, 62.5 mg of an anti-OX40L antibody or antigen binding fragment thereof is administered. In certain embodiments, 125 mg of an anti-OX40L antibody or antigen binding fragment thereof is administered. In certain embodiments, 250 mg of an anti-OX40L antibody or antigen binding fragment thereof is administered. In certain embodiments, 500 mg of an anti-OX40L antibody or antigen binding fragment thereof is administered.

In certain embodiments, a subject weighs equal to or greater than 25 kg and less than 40 kg, and the subject is administered a formulation comprising a 62.5 mg/ml amlitelimab solution supplied as a prefilled syringe that delivers 125 mg of amlitelimab in a 2 mL injection.

In certain exemplary embodiments, the subject weighs equal to or greater than 25 kg and less than 40 kg, and wherein the subject is administered a formulation comprising a 125 mg/mL amlitelimab solution supplied as a prefilled syringe that delivers 125 mg of amlitelimab in a 1 mL injection.

### Administration Regimens

According to certain embodiments, multiple doses of an anti-OX40L antibody or antigen binding fragment thereof may be administered to a subject over a defined time course. Such methods comprise sequentially administering to a subject multiple doses of an anti-OX40L antibody or antigen binding fragment thereof. As used herein, “sequentially administering” means that each dose of an anti-OX40L antibody or antigen binding fragment thereof is administered to the subject at a different point in time, e.g., on different days separated by a predetermined interval (e.g., hours, days, weeks, or months). Methods that comprise sequentially administering to the patient a single initial dose of an anti-OX40L antibody or antigen binding fragment thereof, followed by one or more secondary doses of the anti-OX40L antibody or antigen binding fragment thereof, and optionally followed by one or more tertiary doses of the anti-OX40L antibody or antigen binding fragment thereof, are provided.

Methods comprising administering to a subject a pharmaceutical composition comprising an anti-OX40L antibody or antigen binding fragment thereof at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (q10w), once every eleven weeks (q11w), once every twelve weeks (q12w), or less frequently so long as a therapeutic response is achieved, are provided.

In certain embodiments involving the administration of a pharmaceutical composition comprising an anti-OX40L antibody or antigen binding fragment thereof, once a week dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every two weeks dosing (every two weeks is used interchangeably with every other week, bi-weekly or q2w) of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every three weeks dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every four weeks dosing (monthly dosing) of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every five weeks dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every six weeks dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every eight weeks dosing of an amount of about 62.5 mg, about 125 mg, about 250 mg, about 300 mg, about 400 mg, about 500 mg or about 600 mg can be employed. In other embodiments involving the administration of a pharmaceutical composition comprising an anti-OX40L antibody or antigen binding fragment thereof, once every twelve weeks dosing of an amount of about 62.5 mg, about 125 mg, about 250 mg, about 300 mg, about 400 mg, about 500 mg or about 600 mg can be employed. In certain exemplary embodiments, the route of administration is subcutaneous.

The term “week” or “weeks” refers to a period of (n×7 days)+3 days, e.g., (n×7 days)±2 days, (n×7 days)±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of an anti-OX40L antibody or antigen binding fragment thereof. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of an anti-OX40L antibody or antigen binding fragment thereof, or may differ from one another in terms of frequency of administration. In certain embodiments, however, the amount of an anti-OX40L antibody or antigen binding fragment thereof contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses. For example, one or more initial doses or loading doses of 62.5 mg, 125 mg, 250 mg or 500 mg of an anti-OX40L antibody or antigen binding fragment thereof may be administered followed by secondary doses of about 62.5 mg, 125 mg, 250 mg or 500 mg. In one embodiment, the secondary dose may be equal to the initial dose/loading dose. For example, one or more initial doses/loading doses of about 62.5 mg, 125 mg, 250) mg or 500 mg of an anti-OX40L antibody or antigen binding fragment thereof may be administered followed by secondary doses of about 62.5 mg, 125 mg, 250 mg or 500 mg, respectively.

In certain embodiments, the initial dose is about 50 mg to about 600 mg of the anti-OX40L antibody or antigen binding fragment thereof. In certain embodiments, the initial dose is about 62.5 mg of the anti-OX40L antibody or antigen binding fragment thereof. In certain embodiments, the initial dose is about 125 mg of the anti-OX40L antibody or antigen binding fragment thereof. In certain embodiments, the initial dose is about 250 mg of the anti-OX40L antibody or antigen binding fragment thereof. In another embodiment, the initial dose is 500 mg of the anti-OX40L antibody or antigen binding fragment thereof.

In certain embodiments, the secondary dose(s) are about 50 mg to about 600 mg of the anti-OX40L antibody or antigen binding fragment thereof. In one embodiment, the secondary dose is about 62.5 mg of the anti-OX40L antibody or antigen binding fragment thereof. In one embodiment, the secondary dose is about 125 mg of the anti-OX40L antibody or antigen binding fragment thereof. In one embodiment, the secondary dose is about 125 mg of the anti-OX40L antibody or antigen binding fragment thereof. In one embodiment, the secondary dose is about 250 mg of the anti-OX40L antibody or antigen binding fragment thereof.

In certain embodiments, an initial loading dose is about 500 mg (two injections of 250 mg), followed by a dose of about 250 mg (one injection of 250 mg) administered Q12W.

In certain embodiments, an initial loading dose is about 500 mg (two injections of 250 mg), followed by a dose of about 250 mg (one injection of 250 mg) administered Q4W, and for patients with clear or almost clear skin at 24 weeks of treatment, followed by a dose of about 250 mg administered Q12W.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. Any part of this disclosure may be read in combination with any other part of the disclosure, unless otherwise apparent from the context.

In the following examples, amlitelimab (also known as SAR 445229 and KY1005) was used exemplarily as the antibody specifically binding hOX40L. Amlitelimab is a fully human IgG4 Kappa monoclonal antibody having a “IgG4-PE” constant region with Leu235Glu and Ser228Pro Fc mutations. Amlitelimab comprises a VH domain comprising an amino acid sequence set forth in SEQ ID NO: 34 and a VL domain comprising an amino acid sequence set forth in SEQ ID NO: 48. Amlitelimab comprises a heavy chain (HC) comprising an amino acid sequence set forth in SEQ ID NO: 62 and a light chain (LC) comprising an amino acid sequence set forth in SEQ ID NO: 64.

### EXAMPLES

The following examples further illustrate aspects of the present disclosure. However, it is in no way a limitation of the teachings of the present disclosure as set forth. It should be understood that this Example is given by way of illustration only. From the above discussion and this Example, one of ordinary skill in the art can ascertain the essential characteristics of embodiments of the present disclosure. Without departing from the spirit and scope thereof, one skilled in the art can make various changes and modifications of the disclosure to adapt it to various usages and conditions. All publications, including patents and non-patent literature, referred to in this specification are expressly incorporated by reference herein.

### Example I

### Positive Phase 2b Data of Novel Investigational Anti-OX40-Ligand Amlitelimab Show Statistically Significant Improvements in Signs and Symptoms of Moderate-to-Severe Atopic Dermatitis

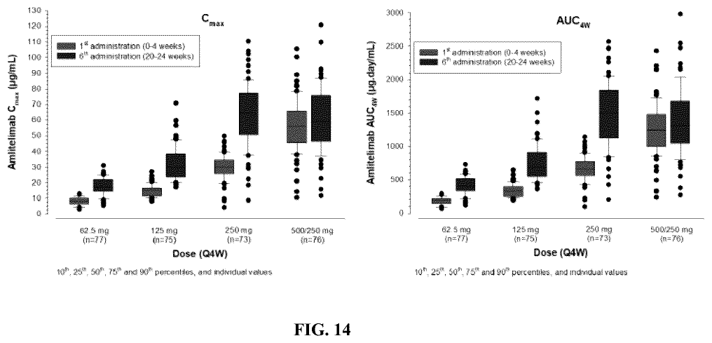

The study design is outlined in FIG. 14. After washing out from prior AD treatments and undergoing a screening period of up to 28 days, participants were randomized in a 1:1:1:1:1 ratio to receive either a SC dose of amlitelimab 500 mg loading dose (given as 2×2 mL SC administration) followed by 250 mg SC Q4W; amlitelimab 250 mg SC Q4W; amlitelimab 125 mg SC Q4W; amlitelimab 62.5 mg SC Q4W; or placebo Q4W from baseline up to Week 24 (Day 169).

The key inclusion criteria were: Adults (218 years but <75 years of age) with AD, for 1 year or longer at baseline; eczema area severity index (EASI) of 12 or higher at the screening visit and 16 or higher at baseline; IGA of 3 or 4 at baseline; AD involvement of 10% or more of body surface area (BSA) at baseline; Weekly average of daily worst/maximum pruritus NRS of 24 at baseline; Documented history, within 6 months prior to baseline, of either inadequate response to topical treatments or inadvisability of topical treatments.

Percentage change from baseline in EASI score was assessed as the primary endpoint at week 16 (day 113). The primary analysis (Part 1) occurred once all participants had completed or had the opportunity to complete week 24 (day 169). The last dose in Part 1 was administered at week 20 (day 141). Safety was assessed in all randomly assigned participants exposed to study drug.

The key secondary efficacy endpoints were: Percentage change from baseline in EASI at week 24 (day 169); Percentage of participants with at least a 75% reduction from baseline in EASI (EASI-75) at week 16 (day 113) and week 24 (day 169); Percentage of participants with a response of IGA 0 (clear) or 1 (almost clear) and a reduction from baseline of ≥2 points at week 16 (Day 113) and week 24 (Day 169); Proportion of participants with improvement (reduction) of weekly average of pruritus NRS ≥4 with a baseline pruritis NRS of ≥4 from baseline to week 16 (day 113) and week 24 (day 169).

Regarding the ongoing post-week 24 study period, Part 2 (i.e., withdrawal/maintenance part), participants who achieved EASI—75 or who attain IGA 0 (clear) or 1 (almost clear) were re-randomized 3:1 to either enter a maintenance withdrawal phase or continue on their pre-Week 24 dose/interval at week 24 (day 169). Those participants who were re-randomized at week 24 (day 169) and who subsequently lost clinical response, defined during that study period as the first instance of <EASI—50 and where rescue therapy is no longer permitted on or after the week 28 (day 197) study visit, were offered the opportunity to receive treatment with amlitelimab in the long-term extension KY1005-CT06/LTS17367 (RIVER-AD) study. At Week 52 (Day 365), participants who completed the second study period without loss of clinical response had an additional 112 days' safety follow-up to week 68 (day 477) and were then discharged from the study. The last dose was administered no later than week 48 (day 337). If during safety follow-up or after the end of study this cohort of participants experienced worsening of their AD, and in the investigator's opinion re-treatment with amlitelimab is considered appropriate, they may be considered for enrolment in the long-term extension KY1005-CT06/LTS17367 (RIVER-AD) study (participant to meet inclusion/exclusion criteria).

The study enrolled a total of 390 participants. The 390 participants were randomized to SC study intervention arms as follows: n=77, n=78, n=77, n=79, and n=79 in the amlitelimab arms 250 mg Q4W with 500 mg loading dose, 250 mg Q4W, 125 mg Q4W, 62.5 mg Q4W or placebo Q4W arm respectively. Of these randomized participants, 388 (99.5%) received at least one dose of the study intervention (1 participant in each of the amlitelimab 62.5 mg Q4W and placebo Q4W arms was randomized and not treated). There were no participants who were exposed to study intervention but not randomized. A total of 323 (83.2%) participants completed the 24-week double-blind treatment period, 65 (16.8%) of participants discontinued treatment and 51 (13.1%) discontinued the study prior to week 24 visit.

Treatment discontinuation rates were lower in the amlitelimab arms 250 mg Q4W with 500 mg loading dose, 250 mg Q4W, 125 mg Q4W or 62.5 mg Q4W (9 [11.7%], 16 [20.5%], 8 [10.4%], 11 [14.1%] participants, respectively) than in the placebo group (21 [26.9%] participants). The most frequently reported reasons for treatment discontinuation prior to week 24 were: Withdrawal by participant (with 2 [2.6%], 9 [11.5%], 6 [7.8%], 2 [2.6%] participants in amlitelimab arms 250 mg Q4W with 500 mg loading dose, 250 mg Q4W; 125 mg Q4W and 62.5 mg Q4W respectively, and 8 [10.3%] participants in the placebo group); adverse effects (AEs) (with 3 [3.9%], 5 [6.4%], 1 [1.3%], 6 [6.7%]) participants in amlitelimab arms respectively, and 4 [5.1%] participants in the placebo group; Lack of efficacy (with 0) [0.0%], 1 [1.3%], 1 [1.3%], 2 [2.6%] participants in amlitelimab arms respectively, and 5 [6.4%] participants in the placebo group).

Study discontinuation rates prior to Week 24 visit were lower in the amlitelimab arms 250 mg Q4W with 500 mg loading dose, 250 mg SC Q4W, 125 mg Q4W, 62.5 mg Q4W (8 [10.4%], 12 [15.4%], 8 [10.4%], 7 [8.9%] participants respectively) than in the placebo group (16 [20.3%] participants). The most frequently reported reason for discontinuation prior to week 24 was withdrawal by participant (with 5 [6.5%], 11 [14.1%], 8 [10.4%], 6 [7.6%] participants in amlitelimab arms 250 mg Q4W with 500 mg loading dose, 250 mg Q4W, 125 mg Q4W or 62.5 mg Q4W respectively, and 14 [17.7%] participants in the placebo group). (Table 1)

TABLE 1
Disposition of participants (week 24 period)

| Amlitelimab | Amlitelimab | | | | |
|---|---|---|---|---|---|
| 250 mg | 250 mg | Amlitelimab | Amlitelimab | | |
| (500 mg LD) | (no LD) | 125 mg | 62.5 mg | Placebo | Total |
| (N = 77) | (N = 78) | (N = 77) | (N = 79) | (N = 79) | (N = 589) |
| n (%) | n (%) | n (%) | n (%) | n (%) | n (%) |

Total number of participants

| Screened | | | | | | | | | | | 589 | |
|---|---|---|---|---|---|---|---|---|---|---|---|---|
| Screen failures^{a} | | | | | | | | | | | 199 | (33.8) |
| Randomized^{b} | 77 | (100.0) | 78 | (100.0) | 77 | (100.0) | 79 | (100.0) | 79 | (100.0) | 390 | (100.0) |
| Randomized and treated^{b} | 77 | (100.0) | 78 | (100.0) | 77 | (100.0) | 78 | (98.7) | 78 | (98.7) | 388 | (99.5) |
| Randomized and not | 0 | | 0 | | 0 | | 1 | (1.3) | 1 | (1.3) | 2 | (0.5) |
| treated^{b} | | | | | | | | | | | | |
| Participants provide consent | 25 | (32.5) | 16 | (20.5) | 20 | (26.0) | 15 | (19.0) | 19 | (24.1) | 95 | (24.4) |
| for the skin biopsy sub- | | | | | | | | | | | | |
| study^{b} | | | | | | | | | | | | |
| Completed Part 1 | 68 | (88.3) | 62 | (79.5) | 69 | (89.6) | 67 | (85.9) | 57 | (73.1) | 323 | (83.2) |
| treatment^{c} | | | | | | | | | | | | |
| Discontinued treatment | 9 | (11.7) | 16 | (20.5) | 8 | (10.4) | 11 | (14.1) | 21 | (26.9) | 65 | (16.8) |
| during Part 1^{c} | | | | | | | | | | | | |

Primary reason for discontinuation from treatment during Part 1^{c}

| Adverse event | 3 | (3.9) | 5 | (6.4) | 1 | (1.3) | 6 | (7.7) | 4 | (5.1) | 19 | (4.9) |
|---|---|---|---|---|---|---|---|---|---|---|---|---|
| Lack of efficacy | 0 | | 1 | (1.3) | 1 | (1.3) | 2 | (2.6) | 5 | (6.4) | 9 | (2.3) |
| Physician decision | 1 | (1.3) | 0 | | 0 | | 0 | | 0 | | 1 | (0.3) |
| Protocol deviation | 2 | (2.6) | 0 | | 0 | | 1 | (1.3) | 0 | | 3 | (0.8) |
| Withdrawal by participant | 2 | (2.6) | 9 | (11.5) | 6 | (7.8) | 2 | (2.6) | 8 | (10.3) | 27 | (7.0) |
| Other | 1 | (1.3) | 1 | (1.3) | 0 | | 0 | | 4 | (5.1) | 6 | (1.5) |
| Completed Week 24^{b} | 68 | (88.3) | 65 | (83.3) | 69 | (89.6) | 71 | (89.9) | 60 | (75.9) | 333 | (85.4) |
| Discontinued from study | 8 | (10.4) | 12 | (15.4) | 8 | (10.4) | 7 | (8.9) | 16 | (20.3) | 51 | (13.1) |
| prior to Week 24 (last IMP | | | | | | | | | | | | |
| administration in Part 1)^{c} | | | | | | | | | | | | |

Primary reason for discontinuation from study prior to Week 24^{b}

| Adverse event | 1 | (1.3) | 1 | (1.3) | 0 | | 0 | | 1 | (1.3) | 3 | (0.8) |
|---|---|---|---|---|---|---|---|---|---|---|---|---|
| Withdrawal by participant | 5 | (6.5) | 11 | (14.1) | 8 | (10.4) | 6 | (7.6) | 14 | (17.7) | 44 | (11.3) |

| | | | | | | | | | | |
|---|---|---|---|---|---|---|---|---|---|---|
| Other | 2 | (2.6) | 0 | 0 | 1 | (1.3) | 1 | (1.3) | 4 | (1.0) |

LD: loading dose
Part 1 is the period from baseline to Week 24 before the rerandomization (primary analysis).
^{a}Percentages are based on the number of screened participants.
^{b}Percentages are based on the number of randomized participants.
^{c}Percentages are based on the number of randomized and treated participants.
Part 1 treatment includes treatment from Day 1 to Week 20.
Completed Part 1 treatment summary includes participants who have completed the treatment for Part 1. Discontinued treatment during Part 1 includes participants who discontinued treatment based on the End of Treatment CRF form and did not reach Week: 20 treatment.
Completed Week 24 summary includes participants who have reached Week 24 regardless of whether they completed the Part 1 treatment or not. Discontinued from study prior to Week 24 includes participants who discontinued from the study based on the End of Study CRF form and did not reach Week 24 visit.

The number (%) of participants with at least one critical or major protocol deviation was similar in the amlitelimab arms (250 mg Q4W with 500 mg loading dose, 250 mg Q4W, 125 mg Q4W, and 62.5 mg Q4W with 15 [19.5%], 16 [20.5%], 11 [14.3%], 20 [25.3%] participants, respectively and the placebo arm (16 [20.3%] participants) (Table 2). The most frequently reported critical or major protocol deviations (at least 3 participants in any treatment arm) were related to: Prohibited medications including non-permitted rescue therapy within the amlitelimab arms 250 mg Q4W with 500 mg loading dose, 250 mg Q4W, 125 mg Q4W and 62.5 mg Q4W; 2 [2.6%], 9 [11.5%], 6 [7.8%], 4 [5.1%] participants, respectively and in the placebo arm: 8 [10.1%] participants; Study treatment administration/dispense within the amlitelimab arms 250 mg Q4W with 500 mg loading dose, 250 mg Q4W, 125 mg Q4W and 62.5 mg Q4W; 1 [1.3%], 3 [3.8%], 3 [3.9%], 3 [3.8%] participants, respectively and in the placebo arm: 2 [2.5%] participants; Visit scheduling due to out of window visits beyond 2 weeks for Week 16 and/or Week 24 visits within in the amlitelimab arms 250 mg Q4W with 500 mg loading dose, 250 mg Q4W, 125 mg Q4W and 62.5 mg Q4W; 3 [3.9%], none, 1 [1.3%], 4 [5.1%] participants, respectively and in the placebo arm: 1 [1.3%] participant; Exclusion criteria mostly due to the use of prohibited medications prior to baseline visit within the amlitelimab arms 250 mg Q4W with 500 mg loading dose, 250 mg Q4W, 125 mg Q4W and 62.5 mg Q4W: 4 [5.2%], none, none, 4 [5.1%] participants, respectively and in the placebo arm: 1 [1.3%] participant.

TABLE 2
Critical and major protocol deviations (week 24 period)

| | Amlitelimab | | Amlitelimab | | | | | | | | | |
|---|---|---|---|---|---|---|---|---|---|---|---|---|
| | 250 mg | | 250 mg | | Amlitelimab | | Amiitelimab | | | | | |
| | (500 mg LD) | | (no LD) | | 125 mg | | 62.5 mg | | Placebo | | Total | |
| | (N = 77) | | (N = 78) | | (N = 77) | | (N = 79) | | (N = 79) | | (N = 390) | |
| | n (%) | | n (%) | | n (%) | | n (%) | | n (%) | | n (%) | |
| Number of participants | 15 | | 16 | | 11 | | 20 | | 17 | | 79 | |
| with at least one | | | | | | | | | | | | |
| significant deviation | | | | | | | | | | | | |
| Protocol deviation | 15 | (19.5) | 16 | (20.5) | 11 | (14.3) | 20 | (25.3) | 16 | (20.3) | 78 | (20.0) |
| Concomitant medication | 2 | (2.6) | 9 | (11.5) | 6 | (7.8) | 4 | (5.1) | 8 | (10.1) | 29 | (7.4) |
| Study treatment | 1 | (1.3) | 3 | (3.8) | 3 | (3.9) | 3 | (3.8) | 2 | (2.5) | 12 | (3.1) |
| admin/dispense | | | | | | | | | | | | |
| Exclusion criteria | 4 | (5.2) | 0 | | 0 | | 4 | (5.1) | 1 | (1.3) | 9 | (2.3) |
| Inclusion criteria | 2 | (2.6) | 2 | (2.6) | 1 | (1.3) | 2 | (2.5) | 2 | (2.5) | 9 | (2.3) |
| Visit scheduling | 3 | (3.9) | 0 | | 1 | (1.3) | 4 | (5.1) | 1 | (1.3) | 9 | (2.3) |
| Study procedures/ | 2 | (2.6) | 1 | (1.3) | 1 | (1.3) | 2 | (2.5) | 2 | (2.5) | 8 | (2.1) |
| assessments | | | | | | | | | | | | |
| Study treatment | 0 | | 1 | (1.3) | 0 | | 1 | (1.3) | 2 | (2.5) | 4 | (1.0) |
| randomization | | | | | | | | | | | | |

| | | | | | | | | | |
|---|---|---|---|---|---|---|---|---|---|
| Other protocol deviation | 0 | 0 | 1 | (1.3) | 1 | (1.3) | 0 | 2 | (0.5) |

| | | | | | | | | |
|---|---|---|---|---|---|---|---|---|
| Informed consent | 1 | (1.3) | 0 | 0 | 0 | 0 | 1 | (0.3) |

| | | | | | | | | | |
|---|---|---|---|---|---|---|---|---|---|
| ICH/GCP deviation | 0 | 1 | (1.3) | 0 | 0 | 1 | (1.3) | 2 | (0.5) |
| Inv safety reporting (CRF) | 0 | 1 | (1.3) | 0 | 0 | 1 | (1.3) | 2 | (0.5) |

CRF: case report form;
ICH/GCP: International Council for Harmonization/Good Clinical Practice;
LD: loading dose
Percentages are based on the number of participants randomized.
Only Significant deviations are displayed. Participants can have more than one deviation recorded; each participant is counted once for each protocol deviation category.

Deviations for inclusion criteria were mostly related to lack of documentation of prior use of topical treatments within the amlitelimab arms 250 mg Q4W with 500 mg loading dose, 250 mg Q4W, 125 mg Q4W and 62.5 mg Q4W: 2 [2.6%], 2 [2.6%], 1 [1.3%], 2 [2.5%] participants, respectively and in the placebo arm: 2 [2.5%] participants.

For two participants “ICH/GCP deviations” due to delay in reporting SAEs within 24 hours in the eCRF were reported. Two “other deviations” were reported for two participants who changed their emollients without additives to emollients with additives during the study. One deviation for “informed consent form (ICF)” was reported for a participant who had a skin biopsy performed prior to sub-study ICF signature.

These identified major protocol deviations were not considered to have undermined the integrity of the results or conclusions of the study.

The number of participants included in each analysis population is provided in Table 3.

TABLE 3
Analysis populations up to week 24

| | Amlitelimab | | | | | | | | | | |
|---|---|---|---|---|---|---|---|---|---|---|---|
| | 250 mg | Amlitelimab | | | | | | | | | |
| | (500 mg | 250 mg | | Amlitelimab | | Amlitelimab | | | | | |
| | LD) | (no LD) | | 125 mg | | 62.5 mg | | Placebo | | Total | |
| | (N = 77) | (N = 78) | | (N = 77) | | (N = 79) | | (N = 79) | | (N = 390) | |
| | n (%) | n (%) | | n (%) | | n (%) | | n (%) | | n (%) | |
| All enrolled^{a} | 77 (100.0) | 78 | (100.0) | 77 | (100.0) | 79 | (100.0) | 79 | (100.0) | 390 | (100.0) |
| All randomized^{a} | 77 (100.0) | 78 | (100.0) | 77 | (100.0) | 79 | (100.0) | 79 | (100.0) | 390 | (100.0) |
| Full Analysis Set for | 77 (100.0) | 78 | (100.0) | 77 | (100.0) | 79 | (100.0) | 79 | (100.0) | 390 | (100.0) |
| Part 1^{a} | | | | | | | | | | | |
| Safety Analysis Set | 77 (100.0) | 78 | (100.0) | 77 | (100.0) | 78 | (98.7) | 78 | (98.7) | 388 | (99.5) |
| for Part 1^{b} | | | | | | | | | | | |
| Pharmacokinetic Set | 77 (100.0) | 76 | (97.4) | 77 | (100.0) | 78 | (98.7) | 0 | | 306 | (79.0) |
| for Part 1^{b} | | | | | | | | | | | |
| ADA Set for Part 1^{b} | 77 (100.0) | 78 | (100.0) | 76 | (98.7) | 78 | (98.7) | 16 | (20.3) | 325 | (83.3) |

ADA: antidrug antibody;
LD: loading dose
The number x at the end of the analysis set name should be understood as ‘for Pad x’.
Percentages are based on the number of participants in the enrolled analysis set.
^{a}Planned arm is used as numerator.
^{b}Actual arm is used as numerator.
Source Data: ADSL

### All Enrolled Set

The all enrolled analysis set included all participants who signed an ICF with no screen failures. Analysis was based on study intervention allocated at randomization.

### Full Analysis Set for Part 1

The full analysis set (FAS1) for part 1 included all randomized participants up to week 24. Efficacy analysis was based on study intervention allocated at randomization. The randomization was stratified by disease severity (moderate/severe) and region (Asia-Pacific, EMEA, North America) at baseline. Disease severity at baseline was defined as moderate (baseline EASI≤21) and severe (baseline EASI >21).

### Safety Analysis Set for Part 1

The safety set (SAF1) for part 1 consisted of all participants who took at least one dose of study intervention, including placebo up to week 24. Any analysis based on the SAF1 was based on the study intervention actually received, regardless of assigned study intervention according to the planned randomization.

### Pharmacokinetic Set for Part 1

The PK set consisted of all randomized participants in the SAF1 with at least one post-baseline PK sample (with adequate documentation of dosing and sampling dates and times) up to week 24. Any analysis based on the PKI was based on the study intervention actually received, regardless of assigned study intervention according to the planned randomization. Participants who received only placebo were not included.

### Antidrug Antibody Set for Part 1

The ADA set consisted of all randomized participants treated with amlitelimab in the SAF1 with at least one post-Baseline ADA result (positive, negative or inconclusive) up to week 24. Any analysis based on the ADA1 was based on the study intervention actually received, regardless of assigned study intervention according to the planned randomization. Participants who received only placebo were not included.

### Statistical Methods

Efficacy analyses were conducted using full analysis set (FAS) which includes all randomized participants based on treatment allocated at randomization. For all efficacy variables, the analysis was comparisons of each of the amlitelimab treatment groups versus the placebo group.

The primary efficacy endpoint was the percentage change in EASI from baseline to Week 16 (Day 113). Mean differences were estimated between each active arm and placebo from analysis of covariance (ANCOVA “\f Abbreviation \t ‘analysis of covariance’”) with treatment, randomization strata (region, disease severity) as fixed effects, and baseline value as a covariate, as well as 95% CI.

### Primary Analysis Approach

Different types of intercurrent events were defined and data handling approach for the assessment after intercurrent event were defined as following. For treatment discontinuation due to reason other than lack of efficacy prior to week 16, treatment policy strategy was applied, that is, all efficacy assessments collected after treatment discontinuation were used in the analysis. For treatment discontinuation due to lack of efficacy, and/or use of rescue medications, or use of prohibited concomitant medications or procedures impacting efficacy prior to week 16, composite variable strategy was applied, that is, the primary endpoint was assessed based on assessments as the worst post-baseline value on or before the time of study treatment discontinuation due to lack of efficacy, or rescue medication/prohibited medication/procedure impacting efficacy usage, whichever earlier (WOCF). Multiple imputation (MI) approach was used to impute missing endpoint value using all participants excluding participants who had taken prohibited medication/procedure impacting efficacy and/or rescue medication prior to week 16 and excluding participants who discontinued due to lack of efficacy prior to week 16.

### Treatment Policy Approach

For treatment discontinuation regardless of whether it is due to lack of efficacy, and/or use of rescue medications, or use of prohibited concomitant medication or procedure impacting efficacy prior to week 16, treatment policy strategy was applied, that is, all efficacy assessments collected after treatment discontinuation, or after use of rescue medications/prohibited concomitant medication or procedure impacting efficacy were used in the analysis. MI using all participants was used to impute missing endpoint value.

Continuous secondary efficacy endpoints were analyzed using a similar approach as the primary efficacy endpoint.

For categorical secondary endpoints, Cochran-Mantel-Haenszel method stratified by randomization strata (region, disease severity) was used. For the primary analysis approach in the handling of intercurrent events, participants who discontinued treatment due to lack of efficacy, and/or use of rescue medications, or use of prohibited concomitant medication or impacting efficacy on or prior to week 16 were considered as non-responders. For the treatment policy approach, all data were analyzed regardless of treatment discontinuation, regardless of use of rescue medications, or use of prohibited concomitant medication or procedure impacting efficacy prior to week 16.

For the primary endpoint, the overall type I error rate was controlled using a hierarchical testing procedure across the four dose regimens against placebo. The hierarchical testing procedure is a sequential test with a pre-specified order (i.e., from the highest dose to the lowest dose).

### Summary Statements on Safety Data/Measurements Analyzed During the 24-Week Treatment Period

For the TEAEs, all AEs that started on or after the first IMP date up to (Week 24 visit (re-randomized IMP date) are included in the TEAE summary tables for Part 1.

For lab by visit: all assessments including early termination or safety follow-up visits are mapped to analysis visits and are included in the safety summary tables for Part 1 while unscheduled assessments are excluded. Some Week 24 assessments are mapped to Week 28 analysis visit and are presented.

For vital signs, physical exam, ECG by visit: all assessments including unscheduled, early termination or safety follow-up visits are mapped to analysis visits and are included in the safety summary tables for Part 1. Some Week 24 assessments are mapped to Week 28 analysis visit and are presented.

For PCSA tables: the abnormality (PCSA) at unscheduled, early termination or safety follow-up visits including those done at Week 24 that are mapped to Week 28 analysis visits are considered in PCSA summary tables for Part 1.

### Demographic Characteristics

The baseline demographics were generally balanced across the treatment arms. Overall, the mean (SD) age was 37.8 (14.36) years (range: 18 to 72 years) and 20 [5.1%] participants were age 65 years or older. The median BMI was 25.4 kg/m2, with 84 (21.5%) participants having a BMI >30 kg/m².

### Disease and Other Characteristics at Baseline

Overall, baseline disease characteristics were generally balanced across the intervention arms with respect to the extent of disease, intensity of signs, severity of symptoms, duration of AD, and other baseline characteristics, and were representative of the global moderate-to-severe AD population.

The mean (SD) duration of AD from first diagnosis in the FAS was 22.27 (16.5) years.

Baseline disease characteristics were mean (SD) EASI 28.9 (10.65), 279 [71.5%] participants with EASI >21 and 111 [28.5%] participants with EASI ≤21:280 [71.8%] participants with IGA 3 (moderate) and 110 [28.2%] participants with IGA 4 (severe); mean (SD) baseline weekly average pruritus NRS 7.32 (1.3); mean (SD) affected BSA involvement 46.21 (19%) and mean (SD) SCORAD 67.17 (12.1).

Other baseline characteristics related to the participants' QoL and symptoms associated with AD were similar between intervention groups. The participants had an overall mean (SD) POEM score at baseline of 20.22 (5.5), a mean (SD) DLQI of 15.43 (6.8), a mean (SD) HADS of 13.22 (8.4) and a mean (SD) Atopic Dermatitis Control Tool (ADCT) of 15.98 (4.5).

### AD Concomitant Medications

All participants were on a stable dose of topical emollients/moisturizers at baseline and continued applying these throughout the study.

### Exposure

The median duration of IMP exposure in the safety population was similar between the intervention arms (23.9 weeks in each of the amlitelimab groups and 23.9 weeks in the placebo group) (Table 4). A total of 325/388 [83.8%] of participants were exposed to IMP for a duration of time between ≥20 weeks and <24 weeks, and 145/388 [37.4%] participants were exposed to IMP for ≥24 weeks.

TABLE 4
Study Treatment Exposure up to Week 24 - Safety Analysis Set

| Amlitelimab | Amlitelimab | | Amlitelimab | | |
|---|---|---|---|---|---|
| 250 mg | 250 mg | Amlitelimab | 62.5 mg | | |
| (500 mg LD) | (no LD) | 125 mg | KY1005 | Placebo | Total |
| (N = 77) | (N = 78) | (N = 77) | (N = 78) | (N = 78) | (N = 388) |

Duration of exposure (Weeks)^{a}

| n | 77 | | 78 | | 77 | | 78 | | 78 | | 388 | |
|---|---|---|---|---|---|---|---|---|---|---|---|---|
| Mean (SD) | 22.5 | (5.09) | 21.2 | (6.19) | 22.5 | (4.71) | 22.4 | (4.53) | 20.5 | (6.52) | 21.8 | (5.50) |

| Median | 23.9 | 23.9 | 23.9 | 23.9 | 23.9 | 23.9 |
|---|---|---|---|---|---|---|
| Min, Max | 4, 28 | 4, 26 | 4, 26 | 4, 27 | 4, 27 | 4, 28 |

Duration of exposure cumulative n (%)

| | | | | | | | | | | | | | |
|---|---|---|---|---|---|---|---|---|---|---|---|---|---|
| ≥4 | weeks | 75 | (97.4) | 73 | (93.6) | 75 | (97.4) | 76 | (97.4) | 74 | (94.9) | 373 | (96.1) |
| ≥8 | weeks | 73 | (94.8) | 69 | (88.5) | 74 | (96.1) | 74 | (94.9) | 71 | (91.0) | 361 | (93.0) |
| ≥12 | weeks | 70 | (90.9) | 69 | (88.5) | 71 | (92.2) | 74 | (94.9) | 64 | (82.1) | 348 | (89.7) |
| ≥20 | weeks | 68 | (88.3) | 62 | (79.5) | 69 | (89.6) | 69 | (88.5) | 57 | (73.1) | 325 | (83.8) |
| ≥24 | weeks | 31 | (40.3) | 22 | (28.2) | 33 | (42.9) | 28 | (35.9) | 31 | (39.7) | 145 | (37.4) |

LD: loading dose; SD: standard deviation.
Percentages are based on the number of subjects who take at least one dose of study treatment, Including Placebo.
^{a}Duration of exposure (weeks) = (Last dose date in Part 1 - First dose date in Part 1 + 1)/7 + 4 weeks.

A higher number of participants in the amlitelimab arms 250 mg Q4W with 500 mg loading dose, 250 mg Q4W, 125 mg Q4W and 62.5 mg Q4W (67 [87.0%], 61 [78.2%], 68 [88.3%] and 65 [83.3%] participants, respectively) received a total of 7 injections over the 24-week period, compared to placebo (53 [67.9%] participants) (Table 5).

TABLE 5
Summary of Injections Administered up to Week 24 - Safety Analysis Set

| | Amlitelimab | Amlitelimab | | | | |
|---|---|---|---|---|---|---|
| | 250 mg | 250 mg | Amlitelimab | Amlitelimab | | |
| | (500 mg LD) | (no LD) | 125 mg | 62.5 mg | Placebo | Total |
| | (N = 77) | (N = 78) | (N = 77) | (N = 78) | (N = 78) | (N = 388) |
| | n (%) | n (%) | n (%) | n (%) | n (%) | n (%) |
| Number of injections | 154 | 158 | 154 | 156 | 156 | 778 |
| administered et | | | | | | |
| baseline | | | | | | |

Number of injections administered up to week 24

| 1 | 0 | | 0 | | 0 | | 0 | | 0 | | 0 | |
|---|---|---|---|---|---|---|---|---|---|---|---|---|
| 2 | 2 | (2.6) | 5 | (6.4) | 2 | (2.6) | 2 | (2.6) | 4 | (5.1) | 15 | (3.9) |
| 3 | 5 | (6.5) | 4 | (5.1) | 3 | (3.9) | 2 | (2.6) | 6 | (7.7) | 20 | (5.2) |
| 4 | 0 | | 2 | (2.6) | 1 | (1.3) | 1 | (1.3) | 5 | (6.4) | 9 | (2.3) |
| 5 | 2 | (2.6) | 4 | (5.1) | 2 | (2.6) | 4 | (5.1) | 5 | (6.4) | 17 | (4.4) |
| 6 | 1 | (1.3) | 2 | (2.6) | 1 | (1.3) | 4 | (5.1) | 5 | (6.4) | 13 | (3.4) |
| 7 | 67 | (87.0) | 61 | (78.2) | 68 | (68.3) | 65 | (83.3) | 53 | (67.9) | 314 | (80.9) |

LD: loading dose
Percentages are based on the number of subjects who take at least one dose of study treatment, including Placebo.
Two injections are expected per subject at baseline, followed by one injection Q4W up to Week 24.

Compliance with Study Intervention

A higher mean (SD) compliance with administration of IMP was observed for participants in the amlitelimab arms 250 mg Q4W with 500 mg loading dose, 250 mg Q4W, 125 mg Q4W and 62.5 mg Q4W: 93.5 (18.18), 89.6 (22.04), 94.4 (16.74), 94.0 (16.04) compared to placebo: 86.4 (22.78).

There was high compliance to study intervention across treatment groups. Amlitelimab arm participants generally had overall higher rates of compliance compared to placebo. A higher rate of compliance ≥100% with administration of IMP was observed for participants in the amlitelimab arms 250 mg Q4W with 500 mg loading dose, 250 mg Q4W, 125 mg Q4W and 62.5 mg Q4W: 67 (87%), 61 (78.2%), 68 (88.3%), 65 (83.3%) compared to placebo: 53 (67.9%) (Table 6).

TABLE 6
Summary of Treatment Compliance up to Week 24 - Safety Analysis Set

| Amlitelimab | Amlitelimab | | | |
|---|---|---|---|---|
| 250 mg | 250 mg | Amlitelimab | Amlitelimab | |
| (500 mg LD) | (no LD) | 125 mg | 62.5 mg | Placebo |
| (N = 77) | (N = 78) | (N = 77) | (N = 78) | (N = 78) |

Compliance

| N | 77 | | 78 | | 77 | | 78 | | 78 | |
|---|---|---|---|---|---|---|---|---|---|---|
| Mean (SD) | 93.5 | (18.16) | 89.6 | (22.04) | 94.4 | (16.74) | 94.0 | (16.04) | 86.4 | (22.78) |

| Median | 100.0 | 100.0 | 100.0 | 100.0 | 100.0 |
|---|---|---|---|---|---|
| Min, max | 29, 100 | 29, 100 | 29, 100 | 29, 100 | 29, 100 |

Compliance N (%)

| | | | | | | | | | | |
|---|---|---|---|---|---|---|---|---|---|---|
| <80% | 9 | (11.7) | 15 | (19.2) | 8 | (10.4) | 9 | (11.5) | 20 | (25.6) |
| 80%-<100% | 1 | (1.3) | 2 | (2.6) | 1 | (1.3) | 4 | (5.1) | 5 | (6.4) |
| ≥100% | 67 | (87.0) | 61 | (78.2) | 68 | (88.3) | 65 | (83.3) | 53 | (67.9) |

Compliant (overall)
n (%)^{a}

| | | | | | | | | | | |
|---|---|---|---|---|---|---|---|---|---|---|
| Yes | 68 | (88.3) | 63 | (80.8) | 69 | (89.6) | 69 | (88.5) | 58 | (74.4) |
| No | 9 | (11.7) | 15 | (19.2) | 8 | (10.4) | 9 | (11.5) | 20 | (25.6) |

LD: loading dose;
SD: standard deviation
^{a}A participant is considered compliant if overall study drug compliance is greater than or equal to 80%.
Compliance is based on the number of injections received divided by 7 planned injections for Part 1.

### Efficacy Summary

The study assessed the effect of amlitelimab on AD lesions, itch, health-related QoL, anxiety and depression of participants with AD whose disease was inadequately controlled on topical medications or when those therapies were not advisable. The primary endpoint was the change in EASI from baseline at week 16.

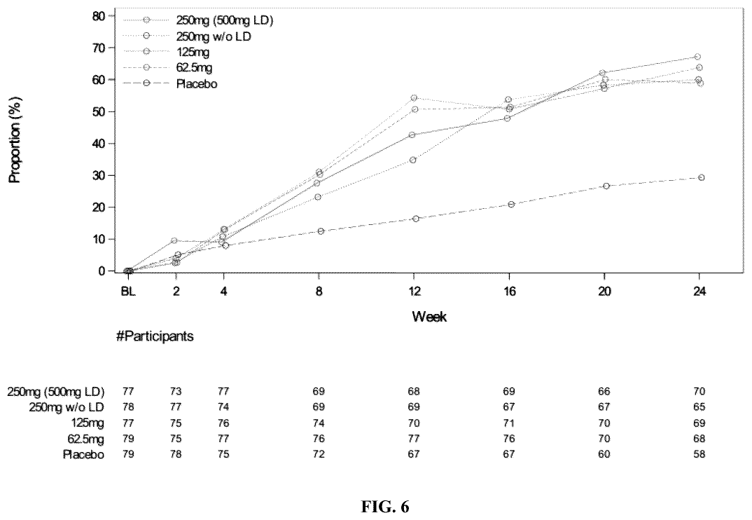

Statistically significant improvements in the primary endpoint (% change in EASI from baseline to week 16) were observed, where all dose arms showed statistically significant mean % reductions in EASI score versus placebo at week 16 (Table 7) and nominally significant reductions at week 24 (Table 9). The 250 mg Q4W arm with 500 mg loading dose showed the greatest numerical difference for both week 16 and week 24 compared to placebo, and compared to other arms (Table 7 and Table 9). The 250 mg Q4W arm with 500 mg loading dose arm showed greater early separation from placebo (as early as week 2, seen similarly with the 62.5 mg Q4W dose), and this separation progressively increased throughout the 24-week intervention period with the greatest increase observed with the 250 mg Q4W with loading dose arm compared to other active arms (FIG. 5). No dose-response proportionality was observed.

TABLE 7
Primary endpoint, main analysis: percentage change in EASI from
baseline at week 16 (estimand 1) - full analysis set for part 1.

| Part 1 Treatment | | | | |
|---|---|---|---|---|
| Amlitelimab | Amlitelimab | | | |
| 250 mg | 250 mg | Amlitelimab | Amlitelimab | |
| (500 mg LD) | (no LD) | 125 mg | 62.5 mg | Placebo |
| (N = 77) | (N = 78) | (N = 77) | (N = 79) | (N = 79) |

Baseline

| N | 77 | | 78 | | 77 | | 79 | | 79 | |
|---|---|---|---|---|---|---|---|---|---|---|
| Mean (SD) | 30.35 | (11.658) | 28.67 | (10.531) | 30.26 | (12.431) | 28.70 | (10.093) | 26.44 | (7.851) |

| Median | 27.95 | 25.83 | 27.50 | 27.20 | 25.30 |
|---|---|---|---|---|---|
| Q1, Q3 | 20.80, 36.30 | 20.80, 36.40 | 20.75, 36.40 | 19.45, 34.40 | 20.00, 31.30 |
| Min, Max | 16.1, 69.4 | 14.6, 68.0 | 16.0, 69.6 | 16.1, 57.2 | 11.1, 55.3 |

Week 16

| N | 70 | | 69 | | 73 | | 76 | | 69 | |
|---|---|---|---|---|---|---|---|---|---|---|
| Mean (SD) | 11.35 | (10.536) | 12.66 | (13.536) | 14.33 | (14.178) | 11.18 | (10.981) | 19.36 | (12.727) |

| Median | 7.55 | 7.20 | 10.00 | 7.25 | 16.80 |
|---|---|---|---|---|---|
| Q1, Q3 | 3.60, 16.80 | 3.60, 17.40 | 3.10, 21.80 | 3.65, 14.10 | 10.50, 26.75 |
| Min, Max | 0.0, 56.0 | 0.0, 64.5 | 0.0, 70.5 | 0.0, 55.0 | 0.0, 51.8 |

Percent Change from Baseline

| N | 70 | | 69 | | 73 | | 78 | | 69 | |
|---|---|---|---|---|---|---|---|---|---|---|
| Mean (SD) | −62.35 | (32.322) | −56.98 | (37.444) | −52.50 | (40.820) | −61.51 | (31.663) | −28.25 | (41.173) |
| Median | −71.84 | | −71.89 | | −69.84 | | −71.60 | | −33.67 | |
| Q1, Q3 | −87.61, −44.50 | | −88.36, −27.80 | | −84.85, −16.50 | | −87.79, −42.22 | | −62.50, 0.00 | |
| Min, Max | −100.0, 49.0 | | −100.0, 62.3 | | −100.0, 62.1 | | −100.0, 18.2 | | −100.0, 88.0 | |
| LS Adjusted Mean | −61.5 | (4.68) | −56.8 | (4.59) | −51.6 | (4.59) | −59.6 | (4.53) | −29.4 | (4.76) |
| (SE)^{a} | | | | | | | | | | |

LS Mean Difference versus Placebo

| (SE)^{a} | −32.1 | (6.01) | −27.3 | (5.98) | −22.2 | (6.01) | −30.2 | (5.95) |
|---|---|---|---|---|---|---|---|---|
| 95% CI^{a} | −43.9, −20.3 | | −39.1, −15.6 | | −34.0, −10.4 | | −41.9, −18.5 | |
| P-value versus | <0.0001 | | <0.0001 | | 0.0002 | | <0.0001 | |
| Placebo^{a} | | | | | | | | |

EASI: Eczama Ansa and Severity Index;
LD: leading dose;
LS: least square,
SD: standard deviation;
SE: standard Error
^{a}Each of the imputed complete data is analyzed by fitting an ANCOVA model with treatment group, region, and disease severity (EASI ≤ 21 versus EASI > 21) as fixed effects and bassline value as covariate.
Baseline is the last assessment prior to the first dose or in case of participants not treated, randomization date (Day 1 pre-dose).
Data collected after early treatment discontinuation due to reasons other than lack of efficacy prior to Week 16 are included. Any data on or after the earliest of rescue medication start date or prohibited medications impacting efficacy or treatment discontinuation due to lack of efficacy were set to missing and imputed by WOCF. Any other Unobserved values or other missing data are imputed by MI.
Multiple Imputation model adjusted with covariates treatment arms, region, disease severity (EASI ≤ 21 versus EASI > 21) at baseline and baseline value of the response endpoint with 40 replications of observed values is used to impute missing value.
No particular missing data handling strategy is implemented for treatment discontinuation due to reasons other than lack of efficacy prior to Week 16.
Descriptive statistics at Week 16 include participants after WOCF at Week 16, and participants whose values are imputed by MI at Week 16 were excluded from the descriptive analysis.

Improvements were also demonstrated in key secondary endpoints.

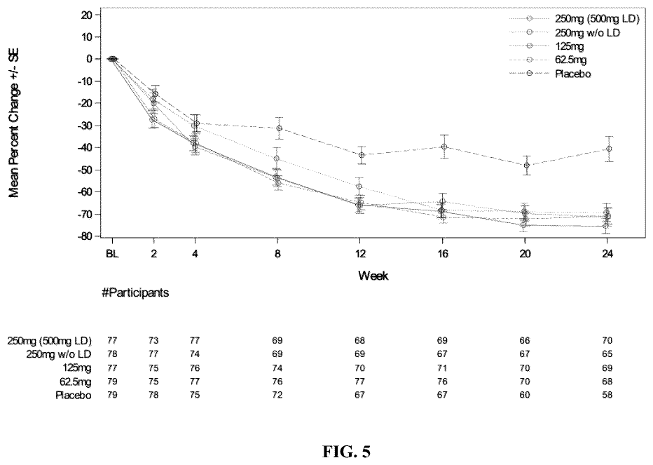

All dose arms showed clinically meaningful and nominal statistically significant greater proportions of participants reaching EASI-75 (% participants with at least a 75% reduction from baseline in EASI) compared to placebo at week 16 (Table 11) and at week 24 (Table 13) compared to placebo. The 250 mg Q4W with 500 mg loading dose showed the greatest numerical difference for week 24 compared to placebo and the other amlitelimab arms (Table 13). The 250 mg Q4W arm with 500 mg loading dose arm showed greater early separation at week 2 from placebo. From week 4 onward, this separation from placebo between all amlitelimab dosing arms progressively increased throughout the remainder of the 24-week intervention period with the greatest increase observed with the 250 mg Q4W arm with 500 mg loading dose at week 24 (FIG. 4). No dose-response proportionality was observed.

IGA 0/1 (% participants with a response of IGA 0 [clear] or 1 [almost clear] and a reduction from baseline of ≥2 points) met nominal statistical significance at week 16 and at week 24 in all amlitelimab dose arms (except the 250 mg Q4W arm at week 16) (Table 15 and Table 17). Continuous improvements were observed in all amlitelimab dose arms through week 24 (with the exception of the 62.5 mg Q4W arm which demonstrated comparable proportions between week 16 and week 24). The 250 mg Q4W with 500 mg loading dose arm showed the greatest numerical difference for week 24 compared to placebo and other amlitelimab arms (Table 17). The 250 mg Q4W arm with 500 mg loading dose arm showed greater early separation from placebo at week 4 compared to other active arms. This separation from placebo between all amlitelimab dosing arms progressively increased throughout the remainder of the 24-week intervention period with the greatest increase observed with the 250 mg Q4W arm with 500 mg loading dose (FIG. 5). No dose-response proportionality was observed.

The endpoint for % participants with >4-point reduction in baseline pruritus NRS met nominal statistical significance in all amlitelimab dose arms at week 16 and at week 24 (Table 19 and Table 21). Improvements were observed for all amlitelimab dose arms at week 24 in comparison to week 16. The 250 mg amlitelimab Q4W with 500 mg loading dose arm showed the greatest numerical difference for week 16 and week 24 compared to placebo and other amlitelimab arms (Table 19 and Table 21). The 250 mg amlitelimab Q4W with 500 mg loading dose arm showed the greatest early separation from placebo at week 3, compared to other active arms. Over the rest of the 24-week treatment period, there was a generally progressive increase in separation from placebo with the greatest separation seen at week 24 that was similar between all amlitelimab dose arms (FIG. 8). No dose-response proportionality was observed.

Analysis for the primary endpoint using treatment policy strategy were consistent with the primary analysis at week 16 (Table 8). At Week 24, there was a larger overall treatment effect versus placebo using treatment policy as compared to the primary analysis approach for the primary endpoint (% change EASI from baseline) (Table 10), EASI-75 (Table 36), IGA 0/1 and 4-point reduction in pruritus NRS. There was a suggestion of a dose-response dependency observed in the analysis using treatment policy for the IGA 0/1 endpoint.

TABLE 8
Primary endpoint: treatment policy approach: percentage change in EASI
from baseline to week 16 (estimand 2) - full analysis set for part 1.

| | Amlitelimab | Amlitelimab | | | |
|---|---|---|---|---|---|
| | 250 mg (500 | 250 mg | Amlitelimab | Amlitelimab | |
| | mg LD) | (no LD) | 125 mg | 62.5 mg | Placebo |
| Part 1 Treatment | (N = 77) | (N = 78) | (N = 77) | (N = 79) | (N = 79) |

Baseline

| N | 77 | | 78 | | 77 | | 79 | | 79 | |
|---|---|---|---|---|---|---|---|---|---|---|
| Mean (SD) | 30.35 | (11.658) | 28.67 | (10.531) | 30.26 | (12.431) | 28.70 | (10.093) | 26.44 | (7.851) |

| | | | | | |
|---|---|---|---|---|---|
| Median | 27.95 | 25.83 | 27.50 | 27.20 | 25.30 |
| Q1, Q3 | 20.80, 36.30 | 20.80, 36.40 | 20.75, 36.40 | 19.45, 34.40 | 20.00, 31.30 |
| Min, Max | 16.1, 69.4 | 14.6, 68.0 | 16.0, 69.6 | 16.1, 57.2 | 11.1, 55.3 |

Week 16

| N | 69 | | 67 | | 71 | | 76 | | 67 | |
|---|---|---|---|---|---|---|---|---|---|---|
| Mean (SD) | 9.37 | (8.199) | 9.18 | (9.714) | 11.03 | (11.351) | 8.16 | (7.510) | 16.56 | (13.337) |

| | | | | | |
|---|---|---|---|---|---|
| Median | 7.00 | 6.15 | 6.90 | 5.80 | 12.40 |
| Q1, Q3 | 3.40, 12.40 | 2.90, 12.00 | 2.90, 15.00 | 2.95, 10.90 | 6.60, 24.55 |
| Min, Max | 0.0, 35.9 | 0.0, 56.4 | 0.0, 52.7 | 0.0, 34.4 | 0.0, 60.0 |

Percent Change from Baseline

| N | 69 | | 67 | | 71 | | 76 | | 67 | |
|---|---|---|---|---|---|---|---|---|---|---|
| Mean (SD) | −68.78 | (25.252) | −68.24 | (28.134) | −64.18 | (30.766) | −71.45 | (22.925) | −39.59 | (43.492) |
| Median | −72.49 | | −76.92 | | −75.86 | | −78.62 | | −58.68 | |
| Q1, Q3 | −87.88, −55.98 | | −89.38, −50.00 | | −86.29, −46.50 | | −89.76, −58.77 | | −68.74, −5.03 | |
| Min, Max | −100.0, 18.0 | | −100.0, 11.9 | | −100.0, 51.3 | | −100.0, −8.0 | | −100.0, 99.6 | |
| LS Adjusted Mean (SE)^{a} | −68.7 | (4.07) | −66.2 | (4.08) | −64.1 | (4.04) | −70.4 | (3.96) | −38.8 | (4.24) |

LS Mean Difference versus Placebo

| | | | | | | | | |
|---|---|---|---|---|---|---|---|---|
| (SE)^{a} | −30.0 | (5.31) | −27.5 | (5.36) | −25.4 | (5.40) | −31.6 | (5.21) |

| | | | | |
|---|---|---|---|---|
| 95% CI^{a} | −40.4:−19.6 | −38.0:−17.0 | −36.0:−14.8 | −41.8:−21.4 |
| P-value versus Placebo^{a} | <0.0001 | <0.0001 | <0.0001 | <0.0001 |

EASI: Eczema Area and Severity Index;
LD: loading dose;
LS: least square,
SD: standard deviation;
SE: standard Error
^{a}Each of the imputed complete data is analyzed by filling an ANCOVA model with treatment group, region, and disease severity (EASI ≤21 versus EASI >21) as fixed effects and baseline value as covariate.
Baseline is the last assessment prior to the first dose or in case of subjects not treated, randomization date (Day 1 pre-dose).
Data collected after early treatment discontinuation are included.
No imputations are done for post rescue medications/prohibited medications impacting efficacy use or treatment discontinuation prior to Week 16.
Participants whose values are imputed by MI at Week 16 are excluded from the descriptive analysis.

Other secondary efficacy endpoints (EASI-90, EASI-100, BSA, SCORAD, see Table 23, Table 24, Table 25 and Table 26) as well as other secondary endpoints including patient-reported outcomes (POEM, ADCT and DLQI as shown in Table 27, Table 29 and Table 30) showed improvements in participants receiving amlitelimab compared to those receiving placebo with nominal statistical significance in all dose arms at both week 16 and week 24. For the HADS endpoint, nominal statistical significance was not achieved in any dose arms but there was a trend toward statistical significance in the 250 mg amlitelimab Q4W with 500 mg loading dose arm at week 16 (p=0.0997), but this was not consistent at week 24 (p=0.7080) (Table 28).

Primary Endpoint: Percentage Change in EASI from Baseline to Week 16

Mean (SD) EASI scores were similar across treatment and placebo groups at baseline in the 250 mg Q4W with 500 mg loading dose, 250 mg Q4W, 125 mg Q4W, and 62.5 mg Q4W amlitelimab groups (30.35 [11.66], 28.67 [10.53], 30.26 [12.43], 28.70 [10.09], respectively) and 26.44 (7.85) in the placebo group (Table 7).

### Primary Analysis

A statistically significant decrease in the percent change in EASI score from baseline was observed at week 16 in all of the amlitelimab dose regimens as compared to placebo. The 250 mg Q4W arm with 500 mg loading dose showed the greatest numerical difference at week 16 compared to placebo, and compared to other amlitelimab arms (Table 7).

### Analysis Using Treatment Policy Data Handling Approach

Analysis using the treatment policy strategy data handling approach for data after all intercurrent events provides consistent efficacy results as the primary analysis at week 16 (Table 8). A nominally statistically significant decrease in the percent change in EASI score from baseline was observed at week 16 in all of the dose regimens studied in the amlitelimab group as compared to placebo. The 62.5 mg Q4W arm and the 250 mg Q4W arm with 500 mg loading dose showed the greatest numerical differences at Week 16 compared to placebo and the other amlitelimab arms.

### Key Secondary Efficacy Endpoints

Percentage Change from Baseline in EASI at Week 24 Primary Analysis

A nominally statistically significant decrease in the percent change in EASI score from baseline was observed at week 24 in all of the dose regimens studied in the amlitelimab group as compared to placebo. The 250 mg Q4W arm with 500 mg loading dose showed the largest numerical difference at week 24 compared to placebo and the other amlitelimab arms (Table 9).

TABLE 9
Key secondary endpoint, main analysis: percentage change in EASI from
baseline to week 24 (estimand 4a) - full analysis set for part 1

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Baseline

| N | 77 | | 78 | | 77 | | 79 | | 79 | |
|---|---|---|---|---|---|---|---|---|---|---|
| Mean (SD) | 30.35 | (11.658) | 28.67 | (10.531) | 30.26 | (12.431) | 28.70 | (10.093) | 26.44 | (7.851) |

| | | | | | |
|---|---|---|---|---|---|
| Median | 27.95 | 25.83 | 27.50 | 27.20 | 25.30 |
| Q1, Q3 | 20.80, 36.30 | 20.80, 36.40 | 20.75, 36.40 | 19.45, 34.40 | 20.00, 31.30 |
| Min, Max | 16.1, 69.4 | 14.6, 68.0 | 16.0, 69.6 | 16.1, 57.2 | 11.1, 55.3 |

Week 24

| N | 71 | | 68 | | 72 | | 70 | | 67 | |
|---|---|---|---|---|---|---|---|---|---|---|
| Mean (SD) | 8.90 | (10.299) | 12.86 | (14.189) | 13.17 | (15.347) | 12.22 | (12.069) | 19.37 | (13.974) |

| | | | | | |
|---|---|---|---|---|---|
| Median | 3.75 | 9.88 | 6.15 | 8.78 | 17.30 |
| Q1, Q3 | 1.40, 14.60 | 1.60, 18.63 | 1.20, 22.38 | 2.55, 18.20 | 9.20, 27.20 |
| Min, Max | 0.0, 56.0 | 0.0, 64.5 | 0.0, 70.5 | 0.0, 55.0 | 0.0, 51.8 |

Percent Change from Baseline

| N | 71 | | 68 | | 72 | | 70 | | 67 | |
|---|---|---|---|---|---|---|---|---|---|---|
| Mean (SD) | −68.01 | (36.052) | −55.84 | (40.299) | −56.72 | (44.271) | −57.37 | (40.225) | −28.55 | (44.004) |
| Median | −86.41 | | −58.88 | | −76.75 | | −68.35 | | −28.97 | |
| Q1, Q3 | −95.44, −38.17 | | −93.62, −23.85 | | −95.82, −16.43 | | −90.20, −30.94 | | −65.22, 3.39 | |
| Min, Max | −100.0, 49.0 | | −100.0, 62.3 | | −100.0, 62.1 | | −100.0, 114.5 | | −100.0, 88.0 | |
| LS Adjusted Mean | −64.4 | (5.17) | −52.2 | (5.14) | −53.7 | (5.08) | −54.4 | (5.09) | −27.6 | (5.29) |

(SE)^{a}

LS Mean Difference versus Placebo

| | | | | | | | | |
|---|---|---|---|---|---|---|---|---|
| (SE)^{a} | −36.8 | (6.62) | −24.6 | (6.67) | −26.2 | (6.65) | −26.8 | (6.58) |

| | | | | |
|---|---|---|---|---|
| 95% CI^{a} | −49.8, −23.8 | −37.7, −11.6 | −39.2, −13.1 | −39.7, −13.9 |
| P-value versus | <0.0001 | 0.0002 | <0.0001 | <0.0001 |
| Placebo^{a} | | | | |

EASI: Eczema Area and Severity Index;
LD: loading dose;
LS: least square,
SD: standard deviation;
SE: standard Error
^{a}Each of the imputed complete data is analyzed by fitting an ANCOVA model with treatment group, region, and disease severity (EASI ≤21 versus EASI >21) as fixed effects and baseline value as covariate.
Baseline is the last assessment prior to the first dose or in case of subjects not treated, randomization date (Day 1 pre-dose).
Data collected after early treatment discontinuation due to reasons other than lack of efficacy prior to Week 24 are included. Any data after the earliest of rescue medication start date or prohibited medications impacting efficacy or treatment discontinuation due to lack of efficacy were set to missing and imputed by WOCF. No particular missing data handling strategy is implemented for treatment discontinuation due to reasons other than lack of efficacy prior to Week 24.
Descriptive statistics at Week 24 include participants after WOCF at Week 24, and participants whose values are imputed by MI at Week 24 were excluded from the descriptive analysis.

### Analysis Using Treatment Policy Data Handling Approach

Analysis using the treatment policy strategy data handling approach for data after all intercurrent events showed a larger overall treatment effect on % change in EASI from baseline to week 24 versus placebo as compared to the primary analysis approach (Table 10). A nominally statistically significant decrease in the percent change in EASI score from baseline was observed at week 24 in all of the dose regimens studied in the amlitelimab group as compared to placebo. The 250 mg Q4W arm with 500 mg loading dose showed the greatest numerical difference at week 24 compared to placebo and the other amlitelimab arms.

TABLE 10
Key secondary endpoint, treatment policy approach: percentage change in
EASI from baseline to week 24 (estimand 4b) - full analysis for part 1

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Baseline

| N | 77 | | 78 | | 77 | | 79 | | 79 | |
|---|---|---|---|---|---|---|---|---|---|---|
| Mean (SD) | 30.35 | (11.658) | 28.67 | (10.531) | 30.26 | (12.431) | 28.70 | (10.093) | 26.44 | (7.851) |

| | | | | | |
|---|---|---|---|---|---|
| Median | 27.95 | 25.83 | 27.50 | 27.20 | 25.30 |
| Q1, Q3 | 20.80, 36.30 | 20.80, 36.40 | 20.75, 36.40 | 19.45, 34.40 | 20.00, 31.30 |
| Min, Max | 16.1, 69.4 | 14.6, 68.0 | 16.0, 69.6 | 16.1, 57.2 | 11.1, 55.3 |

Week 24

| N | 70 | | 65 | | 69 | | 68 | | 58 | |
|---|---|---|---|---|---|---|---|---|---|---|
| Mean (SD) | 6.77 | (7.452) | 8.84 | (11.267) | 8.55 | (11.119) | 8.07 | (8.348) | 15.92 | (13.477) |

| | | | | | |
|---|---|---|---|---|---|
| Median | 3.25 | 3.80 | 3.70 | 3.90 | 12.15 |
| Q1, Q3 | 1.30, 12.00 | 1.20, 15.60 | 1.20, 10.95 | 1.85, 14.00 | 4.10, 23.95 |
| Min, Max | 0.0, 32.1 | 0.0, 66.0 | 0.0, 53.3 | 0.0, 37.6 | 0.0, 51.6 |

Percent Change from Baseline

| N | 70 | | 65 | | 69 | | 68 | | 58 | |
|---|---|---|---|---|---|---|---|---|---|---|
| Mean (SD) | −75.39 | (28.154) | −69.25 | (33.377) | −71.29 | (34.241) | −70.88 | (30.036) | −40.55 | (43.118) |
| Median | −88.62 | | −86.54 | | −84.72 | | −80.79 | | −41.14 | |
| Q1, Q3 | −95.54, −61.95 | | −95.70, −44.23 | | −96.14, −54.40 | | −93.60, −52.17 | | −81.82, −8.12 | |
| Min, Max | −100.0, −11.4 | | −100.0, 31.0 | | −100.0, 77.3 | | −100.0, 37.3 | | −100.0, 51.0 | |
| LS Adjusted Mean (SE)^{a} | −73.1 | (4.49) | −64.8 | (4.59) | −68.0 | (4.43) | −68.4 | (4.39) | −35.5 | (4.67) |

LS Mean Difference versus Placebo

| | | | | | | | | |
|---|---|---|---|---|---|---|---|---|
| (SE)^{a} | −37.6 | (5.90) | −29.3 | (5.82) | −32.5 | (6.03) | −32.9 | (5.79) |

| | | | | |
|---|---|---|---|---|
| 95% CI^{a} | −49.2:−26.1 | −40.7:−17.9 | −44.3:−20.7 | −44.3:−21.5 |
| P-value versus Placebo^{a} | <0.0001 | <0.0001 | <0.0001 | <0.0001 |

EASI: Eczema Area and Severity Index;
LD: loading dose;
LS: least square,
SD: standard deviation;
SE: standard Error
^{a}Each of the imputed complete data is analyzed by fitting an ANCOVA model with treatment group, region, and disease severity (EASI ≤21 versus EASI >21) as fixed effects and baseline value as covariate.
Baseline is the last assessment prior to the first dose or in case of subjects not treated, randomization date (Day 1 pre-dose).
Data collected after early treatment discontinuation are included. Unobserved values or other missing data are imputed by MI.
Any other unobserved values or other missing data are imputed by MI.
Multiple Imputation model adjusted with covariates treatment arms, region, disease severity (EASI ≤21 versus EASI >21) at baseline and baseline value of the response endpoint with 40 replications of observed values is used to impute missing value.
No imputations are done for post rescue medications/prohibited medications impacting efficacy use or treatment discontinuation prior to Week 24.
Participants whose values are imputed by MI at Week 24 are excluded from the descriptive analysis.

Mean % Change EASI from Baseline Over Time

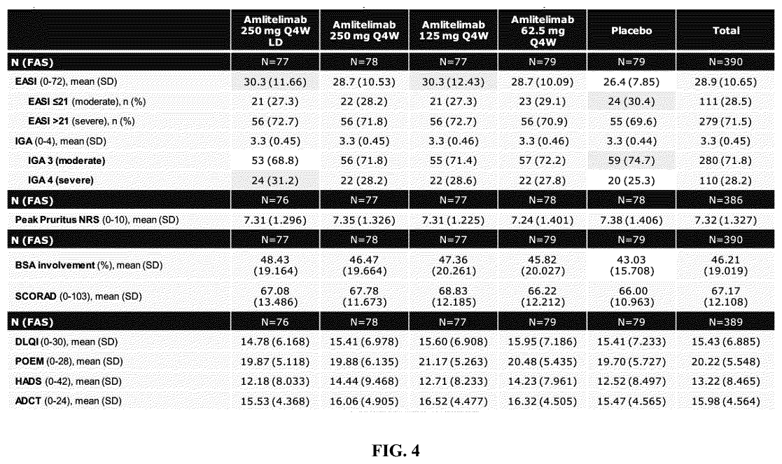

Over the 24-week treatment period using observed data, both the 250 mg Q4W with 500 mg loading dose and 62.5 mg Q4W dose arms showed the greatest early separation from placebo, as early as week 2, compared to the other active arms (FIG. 3). This separation from placebo between all amlitelimab dosing arms progressively increased throughout the remainder of the 24-week intervention period with the greatest increase observed with the 250 mg Q4W with 500 mg loading dose arm.

### Primary Analysis (Week 16)

All dose arms showed clinically meaningful and nominal statistically significant greater proportions of participants reaching EASI-75 compared to placebo at week 16. The 125 mg Q4W arm showed the greatest numerical difference at week 16 compared to placebo and the other amlitelimab arms (Table 11).

TABLE 11
Key secondary endpoint, main analysis: proportion of participants
with EASI-75 at week 16 (estimand 5a) - full analysis for part 1

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

EASI reduction from baseline ≥75% to Week 16

| | | | | | | | | | | |
|---|---|---|---|---|---|---|---|---|---|---|
| Responders | 31 | (40.3%) | 30 | (38.5%) | 33 | (42.9%) | 32 | (40.5%) | 9 | (11.4%) |
| Non-responders | 46 | (59.7%) | 48 | (61.5%) | 44 | (57.1%) | 47 | (59.5%) | 70 | (88.6%) |
| Imputed non-responders | 17 | (22.1%) | 25 | (32.1%) | 21 | (27.3%) | 16 | (20.3%) | 42 | (53.2%) |

Proportion difference versus Placebo

| | | | | | | | | |
|---|---|---|---|---|---|---|---|---|
| (95% CI)^{a} | 0.29 | (0.16, 0.42) | 0.27 | (0.14, 0.40) | 0.31 | (0.18, 0.44) | 0.29 | (0.16, 0.42) |

| | | | | |
|---|---|---|---|---|
| P-value versus Placebo^{b} | <0.0001 | <0.0001 | <0.0001 | <0.0001 |

CI: confidence interval; EASI: Eczema Area and Severity Index; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation due to ressons other than lack of efficacy prior to Week 16 were included.
Participants who discontinued treatment due to lack of efficacy or received rescue medications or prohibited medications impacting efficacy prior to Week 16 were considered as non-responders. Participants with missing data at Week 16 were considered as non-responders.

### Analysis Using Treatment Policy Data Handling Approach (Week 16)

Analysis using the treatment policy strategy data handling approach for data after all intercurrent events provides consistent efficacy results as the primary analysis approach at week 16. All dose arms showed clinically meaningful and nominal statistically significant greater proportions of participants reaching EASI-75 compared to placebo at week 16. The 62.5 mg Q4W arm showed the greatest numerical difference at week 16 compared to placebo and the other amlitelimab arms (Table 12).

TABLE 12
Key secondary endpoint, treatment policy approach: proportion of participants
with EASI-75 at week 16 (estimand 5b) - full analysis set for part 1.

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

EASI reduction from baseline ≥75% to Week 16

| | | | | | | | | | | |
|---|---|---|---|---|---|---|---|---|---|---|
| Responders | 33 | (42.9%) | 36 | (46.2%) | 36 | (46.8%) | 39 | (49.4%) | 14 | (17.7%) |
| Non-responders | 44 | (57.1%) | 42 | (53.8%) | 41 | (53.2%) | 40 | (50.6%) | 65 | (82.3%) |
| Imputed non-responders | 8 | (10.4%) | 11 | (14.1%) | 6 | (7.8%) | 3 | (3.8%) | 12 | (15.2%) |

Proportion difference versus Placebo

| | | | | | | | | |
|---|---|---|---|---|---|---|---|---|
| (95% CI)^{a} | 0.25 | (0.11, 0.39) | 0.28 | (0.15, 0.42) | 0.29 | (0.15, 0.43) | 0.32 | (0.18, 0.46) |

| | | | | |
|---|---|---|---|---|
| P-value versus Placebo^{b} | 0.0008 | 0.0001 | <0.0001 | <0.0001 |

CI: confidence interval; EASI: Eczema Area and Severity Index; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation were included. Participants with missing data at Week 16 were considered as non-responders.

### Primary Analysis (Week 24)

All dose arms showed clinically meaningful and nominal statistically significant greater proportions of participants reaching EASI-75 compared to placebo at week 24. The 250 mg Q4W with 500 mg loading dose arm showed greatest numerical difference at week 24 compared to placebo and the other amlitelimab arms (Table 13).

TABLE 13
Key secondary endpoint, main analysis: proportion of participants with
EASI-75 at week 24 (estimand 5c) - full analysis set for part 1.

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

EASI reduction from baseline 275% to Week 24

| | | | | | | | | | | |
|---|---|---|---|---|---|---|---|---|---|---|
| Responders | 42 | (54.5%) | 30 | (38.5%) | 38 | (49.4%) | 32 | (40.5%) | 14 | (17.7%) |
| Non-responders | 35 | (45.5%) | 48 | (61.5%) | 39 | (50.6%) | 47 | (59.5%) | 65 | (82.3%) |
| Imputed non-responders | 17 | (22.1%) | 28 | (35.9%) | 26 | (33.8%) | 25 | (31.6%) | 46 | (58.2%) |

Proportion difference versus Placebo

| | | | | | | | | |
|---|---|---|---|---|---|---|---|---|
| (95% CI)^{a} | 0.36 | (0.23, 0.50) | 0.21 | (0.07, 0.34) | 0.31 | (0.17, 0.45) | 0.23 | (0.09, 0.36) |

| | | | | |
|---|---|---|---|---|
| P-value versus Placebo^{b} | <0.0001 | 0.0040 | <0.0001 | 0.0016 |

CI: confidence interval; EASI: Eczema Area and Severity Index; EASI-75: ≥75% improvement from baseline EASI; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment diecontinuation due to reasons other than lack of efficacy prior to Week 24 were included.
Participants who discontinued treatment due to lack of efficacy or received rescue medications or prohibited medications impacting efficacy prior to Week 24 were considered as non-responders. Missing data at Week 24 were considered as non-responders.

### Analysis Using Treatment Policy Data Handling Approach (Week 24)

Analysis using the treatment policy strategy data handling approach for data after all intercurrent events at week 24 showed a larger overall treatment effect versus placebo as compared to the primary analysis approach. All dose arms showed clinically meaningful and nominal statistically significant greater proportions of participants reaching EASI-75 compared to placebo at week 24. The 250 mg Q4W with loading dose arm showed the greatest numerical difference at week 24 compared to placebo and the other amlitelimab arms (Table 14).

TABLE 14
Key secondary endpoint, treatment policy approach: proportion of participants
with EASI-75 at week 24 (estimand 5d) - full analysis set for part 1

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

EASI reduction from baseline ≥75% to Week 24

| | | | | | | | | | | |
|---|---|---|---|---|---|---|---|---|---|---|
| Responders | 47 | (61.0%) | 39 | (50.0%) | 44 | (57.1%) | 40 | (50.6%) | 17 | (21.5%) |
| Non-responders | 30 | (39.0%) | 39 | (50.0%) | 33 | (42.9%) | 39 | (49.4%) | 62 | (78.5%) |
| Imputed non-responders | 7 | (9.1%) | 13 | (16.7%) | 8 | (10.4%) | 11 | (13.9%) | 21 | (26.6%) |

Proportion difference versus Placebo]

| | | | | | | | | |
|---|---|---|---|---|---|---|---|---|
| (95% CI)^{a} | 0.39 | (0.25, 0.53) | 0.28 | (0.14, 0.43) | 0.35 | (0.21, 0.49) | 0.29 | (0.15, 0.43) |

| | | | | |
|---|---|---|---|---|
| P-value versus Placebo^{b} | <0.0001 | 0.0002 | <0.0001 | 0.0002 |

CI: confidence interval; EASI: Eczema Area and Severity Index; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation were included. Participants with missing data at Week 24 were considered as non-responders.

### Proportion of Participants Reaching EASI-75 Over Time

Over the 24-week treatment period using observed data, the 250 mg Q4W arm with 500 mg loading dose arm showed the greatest early separation from placebo at week 2. From week 4 onward, this separation from placebo between all amlitelimab dosing arms progressively increased throughout the remainder of the 24-week intervention period with the greatest increase observed with the 250 mg Q4W arm with 500 mg loading dose at week 24 (FIG. 4).

### Primary Analysis (Week 16)

All dose arms with the exception of the 250 mg Q4W arm (p=0.0562) showed clinically meaningful and nominal statistically significant greater proportions of participants reaching IGA 0 (clear) or 1 (almost clear) compared to placebo at week 16. The 62.5 mg Q4W arm showed the greatest numerical difference at week 16 compared to placebo and the other amlitelimab arms (Table 15).

TABLE 15
Key secondary endpoint, main analysis: proportion of participants with
IGA 0/1 at week 16 (estimand 6a) - full analysis set for part 1

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

IGA 0/1 at Week 16

| | | | | | | | | | | |
|---|---|---|---|---|---|---|---|---|---|---|
| Responders | 17 | (22.1%) | 11 | (14.1%) | 15 | (19.5%) | 20 | (25.3%) | 4 | (5.1%) |
| Non-responders | 60 | (77.9%) | 67 | (85.9%) | 62 | (80.5%) | 59 | (74.7%) | 75 | (94.9%) |
| Imputed non-responders | 17 | (22.1%) | 25 | (32.1%) | 21 | (27.3%) | 16 | (20.3%) | 42 | (53.2%) |

Proportion difference versus Placebo

| | | | | | | | | |
|---|---|---|---|---|---|---|---|---|
| (95% CI)^{a} | 0.17 | (0.06, 0.27) | 0.09 | (0.00, 0.18) | 0.14 | (0.04, 0.24) | 0.20 | (0.10, 0.31) |

| | | | | |
|---|---|---|---|---|
| P-value versus Placebo^{b} | 0.0022 | 0.0562 | 0.0054 | 0.0003 |

CI: confidence interval; IGA: Investigator Global Assessment; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation due to reasons other than lack of efficacy prior to Week 16 were included.
Participants who discontinued treatment due to lack of efficacy or received rescue medications or prohibited medications impacting efficacy prior to Week 16 were considered as non-responders. Participants with missing data at Week 16 were considered as non-responders.

### Analysis Using Treatment Policy Data Handling Approach (Week 16)

Analysis using the treatment policy strategy data handling approach for data after all intercurrent events provides consistent efficacy results as the primary analysis approach at week 16. All dose arms showed clinically meaningful and nominal statistically significant greater proportions of participants reaching IGA 0 (clear) or 1 (almost clear) compared to placebo at week 16. The 62.5 mg Q4W arm showed the greatest numerical difference at week 16 compared to placebo and the other amlitelimab arms (Table 16).

TABLE 16
Key secondary endpoint, treatment policy approach: proportion of participants
with IGA 0/1 at week 16 (estimand 6b) - full analysis set for part 1

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

IGA 0/1 at Week 16

| | | | | | | | | | | |
|---|---|---|---|---|---|---|---|---|---|---|
| Responders | 18 | (23.4%) | 15 | (19.2%) | 17 | (22.1%) | 22 | (27.8%) | 6 | (7.6%) |
| Non-responders | 59 | (76.6%) | 63 | (80.8%) | 60 | (77.9%) | 57 | (72.2%) | 73 | (92.4%) |
| Imputed non-responders | 8 | (10.4%) | 11 | (14.1%) | 6 | (7.8%) | 3 | (3.8%) | 12 | (15.2%) |

Proportion difference versus Placebo

| | | | | | | | | |
|---|---|---|---|---|---|---|---|---|
| (95% CI)^{a} | 0.16 | (0.04, 0.27) | 0.11 | (0.01, 0.22) | 0.14 | (0.03, 0.25) | 0.20 | (0.09, 0.32) |

| | | | | |
|---|---|---|---|---|
| P-value versus Placebo^{b} | 0.0078 | 0.0315 | 0.0112 | 0.0008 |

CI: confidence interval; Investigator Global Assessment; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation were included. Participants with missing data at Week 16 were considered as non-responders.

### Primary Analysis (Week 24)

All dose arms showed clinically meaningful and nominal statistically significant greater proportions of participants reaching IGA 0 (clear) or 1 (almost clear) compared to placebo at week 24. Improvements were seen in all amlitelimab arms at week 24 in comparison to week 16 (with the exception of the 62.5 mg Q4W arm which demonstrated comparable proportions between week 16 and week 24). The 250 mg Q4W with 500 mg loading dose arm showed the greatest numerical difference at week 24 compared to placebo and the other amlitelimab arms (Table 17).

TABLE 17
Key secondary endpoint, main analysis: proportion of participants with
IGA 0/1 at week 16 (estimand 6c) - full analysis set for part 1

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

IGA 0/1 at Week 24

| | | | | | | | | | | |
|---|---|---|---|---|---|---|---|---|---|---|
| Responders | 35 | (45.5%) | 26 | (33.3%) | 31 | (40.3%) | 23 | (29.1%) | 9 | (11.4%) |
| Non-responders | 42 | (54.5%) | 52 | (66.7%) | 46 | (59.7%) | 56 | (70.9%) | 70 | (88.6%) |
| Imputed non-responders | 17 | (22.1%) | 28 | (35.9%) | 26 | (33.8%) | 25 | (31.6%) | 46 | (58.2%) |

Proportion difference versus Placebo

| | | | | | | | | |
|---|---|---|---|---|---|---|---|---|
| (95% CI)^{a} | 0.34 | (0.21, 0.47) | 0.22 | (0.10, 0.34) | 0.29 | (0.16, 0.41) | 0.18 | (0.06, 0.30) |

| | | | | |
|---|---|---|---|---|
| P-value versus Placebo^{b} | <0.0001 | 0.0008 | <0.0001 | 0.0046 |

CI: confidence interval; IGA: Investigator Global Assessment; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation due to reasons other than lack of efficacy prior to Week 24 were included.
Participants who discontinued treatment due to lack of efficacy or received rescue medications or prohibited medications impacting efficacy prior to Week 24 were considered as non-responders. Participants with missing data at Week 24 were considered as non-responders.

### Analysis Using Treatment Policy Data Handling Approach (Week 24)

Analysis using the treatment policy strategy data handling approach for data after all intercurrent events at week 24 showed a larger overall treatment effect versus placebo as compared to the primary analysis approach. All dose arms showed clinically meaningful and nominal statistically significant greater proportions of participants reaching IGA 0/1 compared to placebo at week 24. The 250 mg Q4W with 500 mg loading dose arm showed greatest numerical difference at week 24 compared to placebo; and compared to other arms (Table 18).

TABLE 18
Key Secondary endpoint, treatment policy approach: proportion of participants
with IGA 0/1 at week 24 (estimand 6d) - full analysis set for part 1

| | Amlitelimab | Amlitelimab | | Amlitelimab | |
|---|---|---|---|---|---|
| | 250 mg | 250 mg | Amlitelimab | 62.5 mg | |
| | (500 mg LD) | (no LD) | 125 mg | KY1005 | Placebo |
| Part 1 Treatment | (N = 77) | (N = 78) | (N = 77) | (N = 79) | (N = 79) |

IGA 0/1 at Week 24

| | | | | | | | | | | |
|---|---|---|---|---|---|---|---|---|---|---|
| Responders | 38 | (49.4%) | 33 | (42.3%) | 33 | (42.9%) | 29 | (36.7%) | 9 | (11.4%) |
| Non-responders | 39 | (50.6%) | 45 | (57.7%) | 44 | (57.1%) | 50 | (63.3%) | 70 | (88.6%) |
| Imputed non-responders | 7 | (9.1%) | 13 | (16.7%) | 8 | (10.4%) | 11 | (13.9%) | 21 | (26.6%) |

Proportion difference versus Placebo

| | | | | | | | | |
|---|---|---|---|---|---|---|---|---|
| (95% CI)^{a} | 0.38 | (0.25, 0.51) | 0.31 | (0.18, 0.44) | 0.31 | (0.18, 0.44) | 0.25 | (0.13, 0.38) |

| | | | | |
|---|---|---|---|---|
| P-value versus Placebo^{b} | <0.0001 | <0.0001 | <0.0001 | 0.0001 |

CI: confidence interval; IGA: Investigator Global Assessment; LD: loading dose.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation were included. Participants with missing data at Week 24 were considered as non-responders.

Proportion of Participants with a Response of IGA 0 (Clear) or 1 (Almost Clear) and a Reduction from Baseline of ≥2 Points Over Time

Over the 24-week treatment period using observed data, the 250 mg Q4W with 500 mg loading dose arm showed the greatest early separation from placebo at week 4, compared to other active arms. This separation from placebo between all amlitelimab dosing arms progressively increased throughout the remainder of the 24-week intervention period with the greatest increase observed with the 250 mg Q4W arm with 500 mg loading dose (FIG. 5).

Proportion of Participants with Improvement (Reduction) of Weekly Average of Pruritus NRS ≥4 from Baseline to Week 16 and Week 24

### Primary Analysis (Week 16)

All dose arms showed clinically meaningful and nominal statistically significant greater proportions of participants reaching a ≥4-point reduction of weekly average of pruritus NRS compared to placebo at week 16. The 250 mg Q4W arm with 500 mg loading dose showed the greatest numerical difference at week 16 compared to placebo and the other amlitelimab arms (Table 19).

TABLE 19
Key secondary endpoint, main analysis: proportion of participants with improvement of weekly
average pruritis ≥NRS 4 from baseline at week 16 (estimand 7a) - full analysis set for part 1.

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Pruritus NRS improvement from baseline ≥4 points at Week 16

| | | | | | | | | | | |
|---|---|---|---|---|---|---|---|---|---|---|
| Responders | 19 | (24.7%) | 15 | (19.2%) | 16 | (20.8%) | 18 | (22.8%) | 4 | (5.1%) |
| Non-responders | 58 | (75.3%) | 63 | (80.8%) | 61 | (79.2%) | 61 | (77.2%) | 75 | (94.9%) |
| Imputed non-responders | 15 | (19.5%) | 26 | (33.3%) | 23 | (29.9%) | 17 | (21.5%) | 43 | (54.4%) |

Proportion difference versus Placebo

| | | | | | | | | |
|---|---|---|---|---|---|---|---|---|
| (95% CI)^{a} | 0.19 | (0.09, 0.30) | 0.14 | (0.04, 0.24) | 0.16 | (0.05, 0.26) | 0.18 | (0.07, 0.28) |

| | | | | |
|---|---|---|---|---|
| P-value versus Placebo^{b} | 0.0006 | 0.0057 | 0.0038 | 0.0011 |

CI: confidence interval;
LD: loading dose;
NRS: numerical rating scale
^{a}Derived from Mantel-Haenszel estimator, ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation due to reasons other than lack of efficacy prior to Week 16 were included.
Participants who discontinued treatment due to lack of efficacy or received rescue medications or prohibited medications impacting efficacy prior to Week 16 were considered as non-responders. Participants with missing data at Week 16 were considered as non-responders.

### Analysis Using Treatment Policy Data Handling Approach (Week 16)

Analysis using the treatment policy strategy data handling approach for data after all intercurrent events at week 16 showed a slightly larger overall treatment effect versus placebo as compared to the primary analysis approach. All dose arms showed clinically meaningful and nominal statistically significant greater proportions of participants reaching a ≥4-point reduction of weekly average of pruritus NRS compared to placebo at week 16. The 250 mg Q4W with 500 mg loading dose and the 125 mg Q4W dose arms showed the greatest numerical difference at week 16 compared to placebo and the other amlitelimab arms (Table 20).

TABLE 20
Key secondary endpoint, treatment policy approach: proportion of participants with improvement of weekly
average pruritis ≥NRS 4 from baseline at week 16 (estimand 7b) - full analysis set for part 1.

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Pruritis NRS improvement from baseline ≥4 points at Week 16

| | | | | | | | | | | |
|---|---|---|---|---|---|---|---|---|---|---|
| Responders | 21 | (27.3%) | 19 | (24.4%) | 21 | (27.3%) | 20 | (25.3%) | 4 | (5.1%) |
| Non-responders | 56 | (72.7%) | 59 | (75.6%) | 56 | (72.7%) | 59 | (74.7%) | 75 | (94.9%) |
| Imputed non-responders | 6 | (7.8%) | 12 | (15.4%) | 7 | (9.1%) | 5 | (6.3%) | 16 | (20.3%) |

Proportion difference versus Placebo

| | | | | | | | | |
|---|---|---|---|---|---|---|---|---|
| (95% CI)^{a} | 0.22 | (0.11, 0.33) | 0.19 | (0.09, 0.30) | 0.22 | (0.11, 0.33) | 0.20 | (0.10, 0.31) |

| | | | | |
|---|---|---|---|---|
| P-value versus Placebo^{b} | 0.0002 | 0.0006 | 0.0002 | 0.0003 |

CI: confidence interval; LD: loading dose; NRS: numerical rating scale.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤21 versus EASI >21) at baseline.
Note:
Data collected after early treatment discontinuation were included. Participants with missing data at Week 16 were considered as non-responders.

### Primary Analysis (Week 24)

All dose arms showed clinically meaningful and nominal statistically significant greater proportions of participants reaching a ≥4-point reduction of weekly average of peak pruritus NRS compared to placebo at week 24. Improvements were seen in all amlitelimab dose arms at week 24 in comparison to week 16. The 250 mg Q4W with 500 mg loading dose arm showed the greatest numerical difference at week 24 compared to placebo and the other amlitelimab dose arms (Table 21).

TABLE 21
Key secondary endpoint, main analysis: proportion of participants with improvement of weekly
average pruritis ≥NRS 4 from baseline at week 24 (estimand 7c) - full analysis set for part 1.

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Pruritis NRS improvement from baseline ≥4 points at Week 24

| | | | | | |
|---|---|---|---|---|---|
| Responders | 24 (31.2%) | 19 (24.4%) | 22 (28.6%) | 22 (27.8%) | 6 (7.6%) |
| Non-responders | 53 (68.8%) | 59 (75.6%) | 55 (71.4%) | 57 (72.2%) | 73 (92.4%) |
| Imputed non- | 16 (23.4%) | 30 (38.5%) | 28 (36.4%) | 24 (30.4%) | 45 (57.0%) |
| responders | | | | | |

Proportion difference versus Placebo

| | | | | |
|---|---|---|---|---|
| (95% CI)^{a} | 0.23 (0.11, 0.35) | 0.17 (0.06, 0.26) | 0.21 (0.09, 0.32) | 0.20 (0.09, 0.32) |
| P-value versus | 0.0002 | 0.0038 | 0.0006 | 0.0008 |
| Placebo^{b} | | | | |

CI: confidence interval; LD: loading dose; NRS: numerical rating scale.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤ 21 versus EASI > 21) at baseline.
Note:
Data collected after early treatment discontinuation due to reasons other than lack of efficacy prior to Week 24 were included. Participants who discontinued treatment due to lack of efficacy or received rescue medications or prohibited medications impacting efficacy prior to Week 24 were considered as non-responders. Participants with missing data at Week 24 were considered as non-responders.

### Analysis Using Treatment Policy Data Handling Approach (Week 24)

Analysis using the treatment policy strategy data handling approach for data after all intercurrent events at week 24 showed a larger overall treatment effect versus placebo as compared to the primary analysis approach. All dose arms showed clinically meaningful and nominal statistically significant greater proportions of participants a ≥4-point reduction of weekly average of pruritus NRS compared to placebo at week 24. All amlitelimab dose arms demonstrated comparable differences from placebo at week 24. The 250 mg Q4W with 500 mg loading dose, the 125 mg Q4W and the 62.5 mg Q4W dose arms showed greatest numerical difference at week 24 compared to placebo, but with the 250 mg Q4W arm being comparable (Table 22).

TABLE 22
Key secondary endpoint, treatment policy approach: proportion of participants with improvement of weekly
average pruritis ≥NRS 4 from baseline at week 24 (estimand 7d) - full analysis set for part 1.

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Pruritis NRS improvement from baseline ≥4 points at Week 24

| | | | | | |
|---|---|---|---|---|---|
| Responders | 25 (32.5%) | 25 (32.1%) | 25 (32.5%) | 26 (32.9%) | 6 (7.6%) |
| Non-responders | 52 (67.5%) | 53 (67.9%) | 52 (67.5%) | 53 (67.1%) | 73 (92.4%) |
| Imputed non-responders | 9 (11.7%) | 15 (19.2%) | 11 (14.3%) | 10 (12.7%) | 20 (25.3%) |

Proportion difference versus Placebo

| | | | | |
|---|---|---|---|---|
| (95% CI)^{a} | 0.25 (0.12, 0.37) | 0.24 (0.13, 0.36) | 0.25 (0.13, 0.36) | 0.25 (0.14, 0.37) |
| P-value versus Placebo^{b} | 0.0001 | 0.0001 | <0.0001 | <0.0001 |

CI: confidence interval; LD: loading dose; NRS: numerical rating scale.
^{a}Derived from Mantel-Haenszel estimator. ‘NE’ in case the Mantel-Fleiss Criterion is <5.
^{b}P-value based on Cochran-Mantel-Haenszel (CMH) test stratified by region and disease severity (EASI ≤ 21 versus EASI > 21) at baseline.
Note:
Data collected after early treatment discontinuation were included. Participants with missing data at Week 24 were considered as non-responders.

Proportion of Participants with Improvement of Weekly Average of Pruritus NRS ≥4 from Baseline Over Time

Over the 24-week treatment period using observed data, the 250 mg Q4W arm with 500 mg loading dose arm showed the greatest early separation from placebo at week 3, compared to other active arms. Over the rest of the treatment period, there was a generally progressive increase in separation from placebo with the greatest separation seen at week 24 that was similar between all amlitelimab dose arms (FIG. 6).

### Other Secondary Endpoints

Proportion of Participants with at Least a 90% Reduction from Baseline in EASI (EASI-90) at Week 16 and Week 24

### Week 16

All dose arms showed clinically meaningful and nominal statistically significant greater proportions of participants reaching EASI-90 compared to placebo at week 16. The 62.5 mg Q4W arm showed the greatest numerical difference at week 16 compared to placebo and the other amlitelimab arms (Table 23).

### Week 24

All dose arms showed clinically meaningful and nominal statistically significant greater proportions of participants reaching EASI-90 compared to placebo at week 24. The 250 mg Q4W with 500 mg loading dose arm showed the greatest numerical difference at week 24 compared to placebo and the other amlitelimab arms (Table 23).

TABLE 23
Other secondary endpoints, main analysis: EASI-90 - proportion of participants
with at least a 90% reduction from baseline in at week 16 and week 24

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |
| Week 16 | | | | | |
| Responders | 12 (15.6%) | 11 (14.1%) | 13 (16.9%) | 15 (19.0%) | 3 (3.8%) |
| Non-responders | 65 (84.4%) | 67 (85.9%) | 64 (83.1%) | 64 (81.0%) | 76 (96.2%) |
| Imputed non-responders | 17 (22.1%) | 25 (32.1%) | 21 (27.3%) | 16 (20.3%) | 42 (53.2%) |
| Proportion difference | 0.12 | 0.10 | 0.13 | 0.15 | |
| versus Placebo (95% CI)^{a} | (0.03, 0.21) | (0.01, 0.19) | (0.04. 0.22) | (0.06, 0.25) | |
| P-value versus Placebo^{b} | 0.0142 | 0.0250 | 0.0072 | 0.0030 | |
| Week 24 | | | | | |
| Responders | 29 (37.7%) | 21 (26.9%) | 25 (32.5%) | 19 (24.1%) | 9 (11.4%) |
| Non-responders | 48 (62.3%) | 57 (73.1%) | 52 (67.5%) | 60 (75.9%) | 70 (88.6%) |
| Imputed non-responders | 17 (22.1%) | 28 (35.9%) | 26 (33.8%) | 25 (31.6%) | 46 (58.2%) |
| Proportion difference | 0.26 | 0.16 | 0.21 | 0.13 | |
| versus Placebo (95% CI)^{a} | (0.13, 0.38) | (0.04, 0.27) | (0.08, 0.33) | (0.01, 0.24) | |
| P-value versus Placebo^{b} | 0.0001 | 0.0118 | 0.0015 | 0.0326 | |

CI: confidence interval; EASI: Eczema Area Severity Index; LD: loading dose.
^{a}Derived from Mantel-Haenszel estimator. ‘NE’ in case the Mantel-Fleiss Criterion is <5.
^{b}P-value based on Cochran-Mantal-Haenszel (CMH) test stratified by region and disease severity (EASI ≤ 21 versus EASI > 21) at baseline.
Note:
Participants who discontinued treatment due to lack of efficacy or received rescue medications or prohibited medications impacting efficacy before the corresponding timepoint were considered as non-responders. Participants with missing data were considered as non-responders.

Proportion of Participants with at Least a 100% Reduction from Baseline in EASI (EASI-100) at Week 16 and Week 24

### Week 16

There were 3 [3.9%], 1 [1.3%], 2 [2.6%], and 1 [1.3%] participants in amlitelimab 250 mg Q4W with 500 mg loading dose, 250 mg Q4W, 125 mg Q4W, 62.5 mg Q4W arms respectively and 1 [1.3%] participant in the placebo Q4W arm who reached EASI-100 at week 16. The 250 mg Q4W with 500 mg loading dose arm showed the greatest numerical proportion of participants reaching EASI-100 at week 16 compared to placebo and the other amlitelimab arms. No arms were statistically significatively different to placebo (Table 24).

### Week 24

More participants reached EASI-100 at week 24 compared to week 16:6 [7.8%], 5 [6.4%], 7 [9.1%], and 1 [1.3%] in amlitelimab 250 mg Q4W with 500 mg loading dose, 250 mg Q4W, 125 mg Q4W, 62.5 mg Q4W arms respectively and 3 [3.8%] in the placebo Q4W arm. The 125 mg Q4W arm showed the greatest numerical proportion of participants reaching EASI-100 at Week 24 compared to placebo and the other amlitelimab arms. No arms demonstrated significant differences from the placebo group. (Table 24).

TABLE 24
Other secondary endpoints, main analysis: EASI-100 - proportion of participants
with at least a 100% reduction from baseline in at week 16 and week 24

| | Amlitelimab | Amlitelimab | | | |
|---|---|---|---|---|---|
| | 250 mg | 250 mg | Amlitelimab | Amlitelimab | Amlitelimab |
| | (500 mg LD) | (no LD) | 125 mg | 62.5 mg | Placebo |
| Part 1 Treatment | (N = 77) | (N = 78) | (N = 77) | (N = 79) | (N = 79) |
| Week 16 | | | | | |
| Responders | 3 (3.9%) | 1 (1.3%) | 2 (2.6%) | 1 (1.3%) | 1 (1.3%) |
| Non-responders | 74 (96.1%) | 77 (98.7%) | 75 (97.4%) | 78 (98.7%) | 78 (98.7%) |
| Imputed non-responders | 17 (22.1%) | 25 (32.1%) | 21 (27.3%) | 16 (20.3%) | 42 (63.2%) |
| Proportion difference versus | | | | | |
| Placebo (95% CI)^{a} | NE | NE | NE | NE | |
| P-value versus Placebo^{b} | NE | NE | NE | NE | |
| Week 24 | | | | | |
| Responders | 6 (7.8%) | 5 (6.4%) | 7 (9.1%) | 1 (1.3%) | 3 (3.8%) |
| Non-responders | 71 (92.2%) | 73 (93.6%) | 70 (90.9%) | 78 (98.7%) | 76 (96.2%) |
| Imputed non-responders | 17 (22.1%) | 28 (35.9%) | 26 (33.8%) | 25 (31.6%) | 46 (58.2%) |
| Proportion difference versus | NE | NE | NE | NE | |
| Placebo (95% CI)^{a} | | | | | |
| P-value versus Placebo^{b} | NE | NE | NE | NE | |

CI: confidence interval; EASI: Eczema Area Severity Index; LD: loading dose; NE = Not estimable.
^{a}Derived from Mantel-Haenszel estimator. ‘NE’ in case the Mantal-Fleiss Criterion is <5.
^{b}P-value based on Cochran-Mantel-Haenszel (CMH) test stratified by region and disease severity (EASI ≤ 21 versus EASI > 21) at baseline. ‘NE’ in case the Mantel-Fleiss Criterion is <5.
Note:
Participants who discontinued treatment due to lack of efficacy or received rescue medications or prohibited medications impacting efficacy before the corresponding timepoint were considered as non-responders. Participants with missing data ware considered as non-responders.

Percentage Change from Baseline in Affected BSA at Week 16 and Week 24

Mean (SD) affected BSA scores were well-balanced in the treatment and placebo groups at baseline in the 250 mg Q4W with 500 mg loading dose, 250 mg Q4W, 125 mg Q4W, and 62.5 mg Q4W amlitelimab groups (48.43 (19.16), 46.47 (19.66), 47.36 (20.26), 45.82 (20.03), respectively) and 43.03 (15.71) in the placebo group.

### Week 16

A nominally statistically significant decrease in the percent change in affected BSA score from baseline was observed at week 16 in all of the amlitelimab dose regimens as compared to placebo. The 62.5 mg Q4W arm showed the greatest numerical difference at Week 16 compared to placebo and the other amlitelimab arms (Table 25).

### Week 24

A nominally statistically significant decrease in the percent change in affected BSA score from baseline was observed at week 24 in all of the amlitelimab dose regimens as compared to placebo. The 250 mg Q4W arm with 500 mg loading dose showed the greatest numerical difference at Week 24 compared to placebo and the other amlitelimab arms (Table 25).

TABLE 25
Other secondary endpoints, main analysis: affected BSA (%), percentage change
from baseline using WOCF at week 16 and week 24 - full analysis set for part 1

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Baseline

| | | | | | |
|---|---|---|---|---|---|
| N | 77 | 78 | 77 | 79 | 79 |
| Mean (SD) | 48.43 (19.164) | 48.47 (19.664) | 47.36 (20.261) | 45.82 (20.027) | 43.03 (15.708) |
| Median | 46.00 | 42.00 | 43.00 | 45.00 | 43.00 |
| Q1, Q3 | 33.00, 60.00 | 32.00, 58.00 | 31.00, 57.00 | 29.00, 58.00 | 31.00, 53.00 |
| Min, Max | 18.0, 96.0 | 19.0, 95.0 | 19.0, 97.0 | 14.0, 93.0 | 17.0, 96.0 |

Percent Change from Baseline to Week 16

| | | | | | |
|---|---|---|---|---|---|
| N | 70 | 69 | 73 | 76 | 68 |
| Mean (SD) | −54.58 (32.206) | −49.44 (40.861) | −48.35 (43.511) | −56.36 (35.593) | −22.37 (40.483) |
| Median | −64.24 | −65.71 | −57.41 | −71.03 | −20.20 |
| Q1, Q3 | −82.29, −27.54 | −83.33, −2.94 | −83.33, −10.53 | −83.16, −38.20 | −56.39, 0.00 |
| Min, Max | −100.0, 0.0 | −100.0, 63.5 | −100.0, 61.9 | −100.0, 45.8 | −100.0, 76.0 |
| LS Adjusted Mean (SE)^{a} | −54.3 (5.15) | −49.1 (5.07) | −45.8 (4.94) | −56.0 (4.86) | −22.8 (5.19) |

LS Mean Difference versus Placebo

| | | | | |
|---|---|---|---|---|
| (SE)^{a} | −31.5 (6.59) | −26.3 (6.60) | −23.0 (6.52) | −33.2 (6.44) |
| 95% CI^{a} | −44.5:−18.5 | −39.3:−13.3 | −35.8:−10.2 | −45.9:−20.5 |
| P-value versus Placebo^{a} | <0.0001 | <0.0001 | 0.0005 | <0.0001 |

Percent Change from Baseline to Week 24

| | | | | | |
|---|---|---|---|---|---|
| N | 71 | 68 | 70 | 70 | 67 |
| Mean (SD) | −63.02 (36.184) | −49.42 (43.012) | −48.11 (48.748) | −52.84 (44.872) | −23.76 (44.394) |
| Median | −80.60 | −56.55 | −68.04 | −61.71 | −18.29 |
| Q1, Q3 | −93.75, −23.64 | −1.49, −3.87 | −90.48, −5.77 | −88.64, −30.77 | −53.33, 2.70 |
| Min, Max | −100.0, 0.0 | −100.0, 63.5 | −100.0, 61.9 | −100.0, 162.5 | −100.0, 76.0 |
| LS Adjusted Mean (SE)^{a} | −59.4 (5.73) | −46.3 (5.77) | −44.9 (5.64) | −50.1 (5.71) | −21.1 (5.83) |

LS Mean Difference versus Placebo

| | | | | |
|---|---|---|---|---|
| (SE)^{a} | −38.2 (7.43) | −25.2 (7.48) | −23.8 (7.44) | −28.9 (7.43) |
| 95% CI^{a} | −52.8:−23.6 | −39.9:−10.5 | −38.4:−9.1 | −43.6:−14.3 |
| P-value versus Placebo^{a} | <0.0001 | 0.0008 | 0.0015 | 0.0001 |

BSA: Body surface area; CI: confidence interval; LD: loading dose; LS: least square; SD: standard deviation; SE: standard error; WOCF: Worst observation carried forward.
Body surface area (BSA) affected by AD is assessed for each section of the body (the possible highest score for each region is: head and neck [9%], anterior trunk [18%], back [18%], upper limbs (18%], lower limbs [36%], and genitals [1%]) and reported as a percentage of all major body sections combined.
^{a}ANCOVA model with treatment group, region, and disease severity (EASI ≤ 21 versus EASI > 21) as fixed effects and baseline value as covariate.
Baseline is the last assessment prior to the first dose or in case of subjects not treated, randomization date (Day 1 pre-dose).
Note:
Data collected after early treatment discontinuation due to reasons other than lack of efficacy were included. Any data after the earliest of rescue medication start date or prohibited medications impacting efficacy or treatment discontinuation due to lack of efficacy were set to missing and imputed by WOCF.

Percentage Change from Baseline in SCORAD at Week 24

Mean (SD) SCORAD scores were well-balanced in the treatment and placebo groups at baseline in the 250 mg Q4W with 500 mg loading dose, 250 mg Q4W, 125 mg Q4W, and 62.5 mg Q4W amlitelimab groups (67.08 (13.49), 67.78 (11.67), 68.83 (12.19), 66.22 (12.21), respectively) and 66.00 (10.96) in the placebo group.

### Week 16

A nominally statistically significant decrease in the percent change in SCORAD score from baseline was observed at week 16 in all of the amlitelimab dose regimens as compared to placebo. The 250 mg Q4W arm with 500 mg loading dose showed the greatest numerical difference at week 16 compared to placebo and the other amlitelimab arms (Table 26).

### Week 24

A nominally statistically significant decrease in the percent change in affected BSA score from baseline was observed at week 24 in all of the amlitelimab dose regimens as compared to placebo. The 250 mg Q4W arm with 500 mg loading dose showed the greatest numerical difference at week 24 compared to placebo and the other amlitelimab arms (Table 26).

TABLE 26
Other secondary endpoints, main analysis: SCORAD Index, percentage change from
baseline using WOCF at week 16 and week 24 - full analysis set for part 1

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| The Part 1 Treatment | (N = 77) | (N = 78) | (N = 77) | (N = 79) | (N = 79) |

Baseline

| | | | | | |
|---|---|---|---|---|---|
| N | 77 | 78 | 77 | 79 | 79 |
| Mean (SD) | 67.08 (13.486) | 67.78 (11.673) | 68.83 (12.185) | 66.22 (12.212) | 66.00 (10.963) |
| Median | 66.00 | 67.20 | 68.80 | 66.60 | 66.00 |
| Q1, Q3 | 57.00, 76.40 | 59.10, 74.80 | 60.90, 76.20 | 58.20, 75.10 | 57.80, 74.80 |
| Min, Max | 37.3, 98.4 | 43.4, 98.6 | 44.2, 97.2 | 38.5, 94.1 | 43.3, 95.4 |

Percent Change from Baseline to Week 16

| | | | | | |
|---|---|---|---|---|---|
| N | 70 | 69 | 73 | 76 | 68 |
| Mean (SD) | −43.25 (28.555) | −37.18 (28.395) | −37.33 (29.071) | −41.44 (26.134) | −20.87 (26.791) |
| Median | −44.49 | −42.63 | −42.52 | −41.12 | −17.67 |
| Q1, Q3 | −64.24, −19.25 | −60.10, −13.64 | −61.24, −12.83 | −64.53, −21.75 | −41.49, 0.00 |
| Min. Max | −93.0, 0.0 | −85.5, 32.4 | −97.8, 26.3 | −88.5, 15.3 | −87.9, 38.2 |
| LS Adjusted Mean (SE)^{a} | −41.2 (3.60) | −35.4 (3.54) | −35.2 (3.44) | −39.9 (3.40) | −19.9 (3.61) |

LS Mean Difference versus Placebo

| | | | | |
|---|---|---|---|---|
| (SE)^{a} | −21.2 (4.57) | −15.5 (4.58) | −15.3 (4.53) | −20.0 (4.47) |
| 95% CI^{a} | −30.2:−12.3 | −24.5:−6.5 | −24.2:−6.3 | −28.8:−11.2 |
| P-value versus Placebo^{a} | <0.0001 | 0.0008 | 0.0008 | <0.0001 |

Percent Change from Baseline to Week 24

| | | | | | |
|---|---|---|---|---|---|
| N | 71 | 68 | 70 | 70 | 67 |
| Mean (SD) | −51.87 (32.609) | −41.04 (34.212) | −42.58 (35.812) | −41.49 (29.263) | −22.21 (33.233) |
| Median | −58.30 | −43.77 | −50.72 | −38.31 | −11.76 |
| Q1. Q3 | −78.14, −25.74 | −73.21, −7.05 | −69.22, −6.71 | −63.92, −20.11 | −41.44, 0.00 |
| Min, Max | −100.0, 4.5 | −100.0, 32.4 | −100.0, 26.3 | −96.9, 36.7 | −100.0, 38.2 |
| LS Adjusted Mean (SE)a | −46.5 (4.29) | −36.3 (4.32) | −37.6 (4.21) | −37.0 (4.28) | −18.0 (4.35) |

LS Mean Difference versus Placebo

| | | | | |
|---|---|---|---|---|
| (SE)^{a} | −28.5 (5.52) | −18.3 (5.57) | −19.6 (5.55) | −19.0 (5.53) |
| 95% CI^{a} | −39.4:−17.7 | −29.3:−7.4 | −30.5:−8.7 | −29.9:−8.1 |
| P-value versus Placebo^{a} | <0.0001 | 0.0011 | 0.0005 | 0.0007 |

CI: confidence interval; LD: loading dose;: LS: least square; SCORAD: SCORing of atopic dermatitis; SD: standard deviation; SE: standard error; WOCF: Worst observation carried forward.
^{a}Each of the impused complete data is analyzed by fitting an ANCOVA model with treatment group, region, and disease severity (EASI ≤ 21 versus EASI > 21) as fixed effects and bassline value as covariate.
Baseline is the last assessment prior to the first dose or in case of subjects not treated, randomization date (Day 1 pre-dose).
Note:
Data collected after early treatment discontinuation due to reasons other than lack of efficacy were included. Any data earliest of the rescue medication start date or prohibited medications impacting efficacy or treatment discontinuation due to lack of efficacy were set to missing and impuled by WOCF.

### Participant Self-Assessment (POEM, HADS, ADCT, DLQI)

Percentage Change in POEM from Baseline to Week 16 and to Week 24

Mean (SD) POEM scores were well-balanced in the treatment and placebo groups at baseline in the 250 mg Q4W with 500 mg loading dose, 250 mg Q4W, 125 mg Q4W, and 62.5 mg Q4W amlitelimab arms (19.87 (5.12), 19.88 (6.14), 21.17 (5.26), 20.48 (5.44), respectively) and 19.70 (5.73) in the placebo arm.

### Week 16

A nominally statistically significant decrease in the percent change in POEM score from baseline was observed at week 16 in all of the amlitelimab dose regimens as compared to placebo. The 250 mg Q4W arm with 500 mg loading dose showed the greatest numerical difference at week 16 compared to placebo and the other amlitelimab arms (Table 27).

### Week 24

A nominally statistically significant decrease in the percent change in POEM score from baseline was observed at week 24 in all of the amlitelimab dose regimens as compared to placebo. The 250 mg Q4W arm with 500 mg loading dose showed the greatest numerical difference at week 24 compared to placebo and the other amlitelimab arms (Table 27).

TABLE 27
Other secondary endpoints, main analysis: POEM Index, percentage change from
baseline using WOCF at week 16 and week 24 - full analysis set for part 1

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Baseline

| | | | | | |
|---|---|---|---|---|---|
| N | 78 | 78 | 77 | 79 | 79 |
| Mean (SD) | 19.87 (5.118) | 19.88 (6.135) | 21.17 (5.263) | 20.48 (5.435) | 19.70 (5.727) |
| Median | 20.00 | 20.50 | 22.00 | 21.00 | 20.00 |
| Q1, Q3 | 16.00, 24.00 | 16.00, 25.00 | 17.00, 25.00 | 18.00, 25.00 | 15.00, 24.00 |
| Min, Max | 4.0, 28.0 | 0.0, 28.0 | 6.0, 28.0 | 2.0, 28.0 | 6.0, 28.0 |

Percent Change from Baseline to Week 16

| | | | | | |
|---|---|---|---|---|---|
| N | 69 | 68 | 73 | 76 | 68 |
| Mean (SD) | −37.08 (52.391) | −34.71 (34.574) | −32.78 (36.449) | −35.49 (32.862) | −8.12 (45.515) |
| Median | −42.11 | −31.25 | −33.33 | −36.60 | −4.08 |
| Q1, Q3 | −68.42, −15.00 | −66.03, 0.00 | −65.22, 0.00 | −63.30, −11.00 | −34.06, 4.65 |
| Min, Max | −100.0, 300.0 | −100.0, 47.4 | −94.7, 58.3 | −90.5, 53.3 | −95.2, 171.4 |
| LS Adjusted Mean (SE)^{a} | −36.6 (5.35) | −34.3 (5.25) | −30.7 (5.11) | −34.4 (5.01) | −8.5 (5.31) |

LS Mean Difference versus Placebo

| | | | | |
|---|---|---|---|---|
| (SE)^{a} | −28.0 (6.80) | −25.8 (6.82) | −22.2 (6.72) | −25.9 (6.64) |
| 95% CI^{a} | −41.4:−14.7 | −39.2:−12.4 | −35.4:−8.9 | −39.0:−12.8 |
| P-value versus | <0.0001 | 0.0002 | 0.0011 | 0.0001 |
| Placebo^{a} | | | | |

Percent Change from Baseline to Week 24

| | | | | | |
|---|---|---|---|---|---|
| N | 70 | 67 | 70 | 70 | 67 |
| Mean (SD) | −44.69 (61.808) | −33.91 (48.273) | −36.65 (40.947) | −36.81 (34.444) | −6.98 (49.837) |
| Median | −58.95 | −35.00 | −39.21 | −34.17 | −4.00 |
| Q1, Q3 | −80.00, −20.00 | −68.75, 0.00 | −77.78, 0.00 | −88.42, −15.00 | −33.33, 3.70 |
| Min, Max | −100.0, 375.0 | −100.0, 220.0 | −100.0, 58.3 | −96.0, 53.3 | −100.0, 171.4 |
| LS Adjusted Mean (SE)^{a} | −42.7 (6.21) | −32.5 (6.25) | −33.1 (6.09) | −34.5 (6.17) | −5.8 (6.25) |

LS Mean Difference versus Placebo

| | | | | |
|---|---|---|---|---|
| (SE)^{a} | −36.9 (8.00) | −26.8 (8.08) | −27.4 (8.02) | −28.7 (8.00) |
| 95% CI^{a} | −52.7:−21.2 | −42.7:−10.9 | −43.1:−11.6 | −44.4:−13.0 |
| P-value versus Placebo^{a} | <0.0001 | 0.0010 | 0.0007 | 0.0004 |

CI: confidence interval; LD: loading dose; LS: least square: POEM: patient-oriented eczema measure; SD: standard deviation; SE: standard error; WOCF: Worst observation carried forward.
^{a}Each of the imputed complete data is analyzed by fitting an ANCOVA model with treatment group, region, and disease severity (EASI ≤ 21 versus EASI > 21) as fixed effects and baseline value as covarate
Baseline is the last assessment prior to the first dose or in case of subjects not treated, randomization date (Day 1 pre-dose).
Note:
Data collected after early treatment discontinuation due to reasons other than lack of efficacy were included. Any data earliest of the rescue medication start date or prohibited medications impacting efficacy or treatment discontinuation due to lack of efficacy were set to missing and imputed by WOCF.

Percentage Change in HADS from Baseline to Week 16 Ad to Week 24

Mean (SD) HADS scores were well-balanced in the treatment and placebo groups at baseline in the 250 mg Q4W with 500 mg loading dose, 250 mg Q4W, 125 mg Q4W, and 62.5 mg Q4W amlitelimab groups (12.18 (8.03), 14.44 (9.47), 12.71 (8.23), 14.23 (7.96), respectively) and 12.52 (8.497) in the placebo group.

### Week 16

The percent change in HADS score from baseline at week 16 in all of the amlitelimab dose regimens as compared to placebo was not statistically significant although there was a slight trend toward statistical significance in the 250 mg Q4W with 500 mg loading dose arm [p=0.0997]) (Table 28).

### Week 24

The percent change in HADS score from baseline at Week 24 in all of the amlitelimab dose regimens as compared to placebo was not statistically significant (Table 28).

TABLE 28
Other secondary endpoints, main analysis: HADS, percentage change from baseline
using WOCF at week 16 and week 24 - full analysis set for part 1

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Baseline

| | | | | | |
|---|---|---|---|---|---|
| N | 76 | 78 | 77 | 79 | 79 |
| Mean (SD) | 12.18 (8.033) | 14.44 (9.468) | 12.71 (8.233) | 14.23 (7.961) | 12.52 (8.497) |
| Median | 10.00 | 15.00 | 13.00 | 13.00 | 12.00 |
| Q1, Q3 | 5.50, 19.00 | 5.00, 21.00 | 8.00, 19.00 | 8.00, 20.00 | 5.00, 18.00 |
| Min, Max | 0.0, 31.0 | 0.0, 37.0 | 0.0, 36.0 | 1.0, 34.0 | 0.0, 36.0 |

Percent Change from Baseline to Week 16

| | | | | | |
|---|---|---|---|---|---|
| N | 66 | 65 | 69 | 76 | 65 |
| Mean (SD) | −18.12 (56.175) | −11.72 (54.678) | −15.84 (54.001) | −17.47 (94.346) | 2.82 (101.930) |
| Median | −18.33 | 0.00 | −11.76 | −32.29 | 0.00 |
| Q1, Q3 | −57.14, 0.00 | −45.45, 0.00 | −46.67, 0.00 | −56.70, −4.10 | −31.82, 0.00 |
| Min, Max | −100.0, 150.0 | −100.0, 200.0 | −100.0, 240.0 | −100.0, 575.0 | −100.0, 533.3 |
| LS Adjusted Mean (SE)^{a} | −22.4 (10.29) | −12.4 (10.20) | −18.0 (9.85) | −19.2 (9.54) | −0.7 (10.36) |

LS Mean Difference versus Placebo

| | | | | |
|---|---|---|---|---|
| (SE)^{a} | −21.7 (13.15) | −11.7 (13.23) | −17.3 (13.02) | −18.5 (12.72) |
| 95% CI^{a} | −47.6:4.2 | −37.7:14.3 | −42.9:8.3 | −43.5:6.5 |
| P-value versus Placebo [a] | 0.0997 | 0.3769 | 0.1854 | 0.1473 |

Percent Change from Baseline to Week 24

| | | | | | |
|---|---|---|---|---|---|
| N | 69 | 65 | 66 | 70 | 64 |
| Mean (SD) | −13.22 (97.953) | −8.67 (60.563) | −17.75 (52.049) | −12.07 (120.061) | −8.17 (77.872) |
| Median | −36.36 | −16.67 | −5.56 | −28.59 | −3.13 |
| Q1, Q3 | −70.00, 0.00 | −47.37, 0.00 | −52.63, 0.00 | −58.33, 0.00 | −32.58, 0.00 |
| Min, Max | −100.0, 550.0 | −100.0, 200.0 | −100.0, 240.0 | −100.0, 700.0 | −100.0, 500.0 |
| LS Adjusted Mean (SE)^{a} | −11.3 (11.41) | −3.5 (11.65) | −15.2 (11.32) | −7.8 (11.30) | −5.8 (11.75) |

LS Mean Difference versus Placebo

| | | | | |
|---|---|---|---|---|
| (SE)^{a} | −5.5 (14.80) | 2.2 (15.05) | −9.4 (14.98) | −2.1 (14.76) |
| 95% CI^{a} | −34.7:23.6 | −27.4:31.8 | −38.9:20.0 | −31.1:27.0 |
| P-value versus Placebo^{a} | 0.7080 | 0.8824 | 0.5299 | 0.8887 |

CI: confidence interval: HADS: Hospital Anxiety and Depression Scale; LD: loading dose: LS: least square; SD: standard deviation; SE: standard error; WOCF: Worst observation carried forward.
^{a}Each of the imputed complete data is analyzed by fitting an ANCOVA model with treatment group, region, and disease severity (EASI ≤ 21 versus EASI > 21) as fixed effects and baseline value as covariate
Baseline is the last assessment prior to the first dose or in case of subjects not treated, randomization date (Day 1 pre-dose).
HADS is the total of anxiety and depression scores.
Note:
Data collected after early treatment discontinuation due to reasons other than lack of efficacy were included. Any data earliest of the rescue medication start date or prohibited medications impacting efficacy or treatment discontinuation due to lack of efficacy were set to missing and imputed by WOCF.

Percentage Change in ADCT from Baseline to Week 16 and to Week 24

Mean (SD) ADCT scores were balanced in the treatment and placebo groups at baseline in the 250 mg Q4W with 500 mg loading dose, 250 mg Q4W, 125 mg Q4W, and 62.5 mg Q4W amlitelimab groups (15.53 (4.37), 16.06 (4.91), 16.52 (4.48), 16.32 (4.51), respectively) and 15.47 (4.57) in the placebo group.

### Week 16

A nominally statistically significant decrease in the percent change in ADCT score from baseline was observed at week 16 in all of the amlitelimab dose regimens as compared to placebo. The 250 mg Q4W arm with 500 mg loading dose showed the greatest numerical difference at week 16 compared to placebo and the other amlitelimab arms. (Table 29)

### Week 24

A nominally statistically significant decrease in the percent change in ADCT score from baseline was observed at week 24 in all of the amlitelimab dose regimens as compared to placebo. The 250 mg Q4W arm with 500 mg loading dose showed the greatest numerical difference at week 24 compared to placebo and the other amlitelimab arms. (Table 29)

TABLE 29
Other secondary endpoints, main analysis: ADCT, percentage change from baseline
using WOCF at week 16 and week 24 - full analysis set for part 1

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Baseline

| | | | | | |
|---|---|---|---|---|---|
| N | 76 | 78 | 77 | 79 | 79 |
| Mean (SD) | 15.53 (4.368) | 16.06 (4.905) | 16.52 (4.477) | 16.32 (4.505) | 15.47 (4.565) |
| Median | 16.00 | 16.00 | 17.00 | 17.00 | 16.00 |
| Q1, Q3 | 12.00, 19.00 | 12.00, 20.00 | 13.00, 20.00 | 14.00, 19.00 | 12.00, 19.00 |
| Min, Max | 6.0, 24.0 | 6.0, 24.0 | 7.0, 24.0 | 1.0, 24.0 | 5.0, 24.0 |

Percent Change from Baseline to Week 16

| | | | | | |
|---|---|---|---|---|---|
| N | 67 | 66 | 73 | 76 | 68 |
| Mean (SD) | −40.56 (37.340) | −33.42 (34.745) | −36.42 (37.103) | −38.72 (30.762) | −17.04 (32.553) |
| Median | −50.00 | −26.79 | −40.00 | −39.80 | 0.00 |
| Q1, Q3 | −66.67, −13.04 | −70.83, 0.00 | −66.67, 0.00 | −61.54, −7.69 | −39.82, 0.00 |
| Min, Max | −100.0, 77.8 | −95.0, 33.3 | −94.7, 70.0 | −100.0, 9.1 | −100.0, 44.4 |
| LS Adjusted Mean (SE)^{a} | −39.2 (4.63) | −31.9 (4.57) | −34.6 (4.39) | −37.2 (4.31) | −16.4 (4.56) |

LS Mean Difference versus Placebo

| | | | | |
|---|---|---|---|---|
| (SE)^{a} | −22.8 (5.88) | −15.5 (5.91) | −18.3 (5.77) | −20.8 (5.71) |
| 95% CI^{a} | −34.3:−11.2 | −27.1:−3.9 | −29.6:−6.9 | −32.0:−9.6 |
| P-value versus Placebo^{a} | 0.0001 | 0.0092 | 0.0017 | 0.0003 |

Percent Change from Baseline to Week 24

| | | | | | |
|---|---|---|---|---|---|
| N | 65 | 66 | 70 | 68 | 67 |
| Mean (SD) | −45.18 (42.438) | −36.20 (41.363) | −40.45 (40.708) | −39.35 (31.799) | −12.31 (37.504) |
| Median | −55.00 | −34.52 | −46.18 | −35.29 | 0.00 |
| Q1, Q3 | −81.25, 0.00 | −68.42, 0.00 | −75.00, 0.00 | −66.88, −11.81 | −23.53, 0.00 |
| Min, Max | −100.0, 77.8 | −100.0, 133.3 | −100.0, 70.0 | −94.1, 11.8 | −100.0, 111.1 |
| LS Adjusted Mean (SE)^{a} | −41.6 (5.25) | −32.9 (5.19) | −37.1 (5.02) | −36.6 (5.12) | −9.6 (5.15) |

LS Mean Difference versus Placebo

| | | | | |
|---|---|---|---|---|
| (SE)^{a} | −32.0 (6.71) | −23.3 (6.68) | −27.5 (6.59) | −27.0 (6.63) |
| 95% CI^{a} | −45.23:−18.8 | −36.5:−10.2 | −40.5:−14.6 | −40.0:−13.9 |
| P-value versus Placebo^{a} | <0.0001 | 0.0005 | <0.0001 | <0.0001 |

ADCT: Alopic Dermatitis Control Tool: CI: confidence interval; LD: loading dose; LS: least square: SD: standard deviation; SE: standard error: WOCF: Worst observation carried forward.
^{a}ANCOVA model with treatment group, region, and disease severity (EASI ≤ 21 versus EASI > 21) as fixed effects and baseline value as covariate.
Baseline is the last assessment prior to the first dose or in case of subjects not treated, randomization date (Day 1 pre-dose).
Data collected after early treatment discontinuation due to reasons other than lack of efficacy were included. Any data after the earliest of rescue medication start date or prohibited medications impacting efficacy or treatment discontinuation due to lack of efficacy were set to missing and imputed by WOCF.

Percentage Change in DLQI from Baseline to Week 16 and to Week 24

Mean (SD) DLQI scores were balanced in the treatment and placebo groups at baseline in the 250 mg Q4W with 500 mg loading dose, 250 mg Q4W, 125 mg Q4W, and 62.5 mg Q4W amlitelimab groups (14.78 (6.17), 15.41 (6.98), 15.60 (6.91), 15.95 (7.19), respectively) and 15.41 (7.23) in the placebo group.

### Week 16

A nominally statistically significant decrease in the percent change in DLQI score from baseline was observed at week 16 in all of the amlitelimab dose regimens as compared to placebo. The 62.5 mg Q4W arm dose showed the greatest numerical difference at week 16 compared to placebo and compared to other arms. (Table 30)

### Week 24

A nominally statistically significant decrease in the percent change in ADCT score from baseline was observed at week 24 in all of the amlitelimab dose regimens as compared to placebo. The 250 mg Q4W with 500 mg loading dose arm showed the greatest numerical difference at week 24 compared to placebo and compared to other arms. (Table 30)

TABLE 30
Other secondary endpoints, main analysis: DLQI Index, percentage change from
baseline using WOCF at week 16 and week 24 - full analysis set for part 1

| | | | | | |
|---|---|---|---|---|---|
| | Amlitelimab | Amlitelimab | | | |
| | 250 mg | 250 mg | Amlitelimab | Amlitelimab | |
| | (500 mg LD) | (no LD) | 125 mg | 62.5 mg | Placebo |
| Part 1 Treatment | (N = 77) | (N = 78) | (N = 77) | (N = 79) | (N = 79) |

Baseline

| | | | | | |
|---|---|---|---|---|---|
| N | 76 | 78 | 77 | 79 | 79 |
| Mean (SD) | 14.78 (6.168) | 15.41 (6.978) | 15.60 (6.908) | 15.95 (7.186) | 15.41 (7.233) |
| Median | 14.50 | 16.00 | 15.00 | 15.00 | 15.00 |
| Q1, Q3 | 11.00, 19.00 | 10.00, 21.00 | 10.00, 21.00 | 11.00, 22.00 | 9.00, 21.00 |
| Min, Max | 3.0, 29.0 | 2.0, 30.0 | 3.0, 30.0 | 0.0, 30.0 | 1.0, 30.0 |

Percent Change from Baseline to Week 16

| | | | | | |
|---|---|---|---|---|---|
| N | 67 | 67 | 73 | 75 | 68 |
| Mean (SD) | −46.69 (40.563) | −41.27 (43.616) | −34.02 (62.565) | −48.96 (29.801) | −11.35 (51.739) |
| Median | −54.55 | −50.00 | −46.15 | −53.85 | −9.81 |
| Q1, Q3 | −75.00, −17.65 | −75.00, −16.00 | −72.73, −7.14 | −75.00, −28.57 | −43.78, 7.14 |
| Min, Max | −100.0, 83.3 | −100.0, 150.0 | −100.0, 350.0 | −100.0, 38.5 | −100.0, 200.0 |
| LS Adjusted Mean (SE)^{a} | −47.3 (6.32) | −41.6 (6.17) | −33.7 (5.96) | −48.6 (5.91) | −11.9 (6.21) |

LS Mean Difference versus Placebo

| | | | | |
|---|---|---|---|---|
| (SE)^{a} | −35.3 (8.01) | −29.7 (8.00) | −21.8 (7.84) | −36.7 (7.78) |
| 95% CI^{a} | −51.1:−19.6 | −45.4:−13.9 | −37.2:−6.4 | −52.0:−21.4 |
| P-value versus Placebo^{a} | <0.0001 | 0.0002 | 0.0057 | <0.0001 |

Percent Change from Baseline to Week 24

| | | | | | |
|---|---|---|---|---|---|
| N | 70 | 68 | 70 | 70 | 67 |
| Mean (SD) | −51.84 (41.442) | −42.36 (45.550) | −33.28 (72.816) | −45.63 (33.860) | −11.91 (55.694) |
| Median | −62.00 | −44.60 | −52.86 | −50.00 | −10.53 |
| Q1, Q3 | −86.67, −17.24 | −79.51, −13.26 | −80.00, 0.00 | −71.43, −21.74 | −45.45, 7.14 |
| Min, Max | −100.0, 83.3 | −100.0, 150.0 | −100.0, 350.0 | −100.0, 38.5 | −100.0, 200.0 |
| LS Adjusted Mean (SE)^{a} | −46.7 (6.74) | −37.7 (6.75) | −28.0 (6.59) | −40.2 (6.67) | −7.1 (6.77) |

LS Mean Difference versus Placebo

| | | | | |
|---|---|---|---|---|
| (SE)^{a} | −39.6 (8.68) | −30.6 (8.73) | −20.9 (8.67) | −33.1 (8.67) |
| 95% CI^{a} | −56.7:−22.6 | −47.8:−13.4 | −37.9:−3.8 | −50.2:−16.1 |
| P-value versus Placebo^{a} | <0.0001 | 0.0005 | 0.0165 | 0.0002 |

CL: confidence interval; DLQI: dermatology quality of life index; SD: standard deviation; SE: standard error; LD: loading dose; LS: least square; WOCF: Worst observation carried forward.
^{a}ANCOVA model with the corresponding baseline value, treatment group, regions and disease severity (EASI ≤ 21 versus EASI > 21) at baseline as covariates at each visit.
Baseline is the last assessment prior to the first doze or in case of subjects not treated, randomization date (Day 1 pre-dose).
Note:
Data collected after early treatment discontinuation due to reasons other than lack of efficacy were included. Any data after the earliest of rescue medication start date or treatment discontinuation due to lack of efficacy were set to missing and imputed by WOCF.

### Safety Summary

Labs by visit: all assessments including early termination or safety follow-up visits are mapped to analysis visits and are included in the safety summary tables for part 1 while unscheduled assessments are excluded. Some week 24 assessments are mapped to week 28 analysis visit and are presented.

Vital signs, physical exam findings, and ECGs by visit: all assessments including unscheduled, early termination or safety follow-up visits are mapped to analysis visits and are included in the safety summary tables for part 1. Some week 24 assessments are mapped to week 28 analysis visit and are presented in this table.

PCSA tables (for labs, urine analysis, and vital signs): the abnormality (PCSA) at unscheduled, early termination or safety follow-up visits including those done at week 24 that are mapped to week 28 analysis visits are considered in PCSA summary tables for part 1.

For the treatment-emergent AEs, all AEs that started on or after the first IMP date up to week 24 visit (re-randomized IMP in Part 2 are included in the TEAE summary tables for part 1).

Amlitelimab was well tolerated and had an acceptable safety profile in this study of participants with AD.

See Table 31 for the overview of adverse event profile in the safety population.

TABLE 31
Summary of treatment-emergent adverse events up to week 24 - safety analysis set for part 1

| | Amlitelimab | | | | | | | | | | | |
|---|---|---|---|---|---|---|---|---|---|---|---|---|
| Number Of | 250 mg | | Amlitelimab | | Amlitelimab | | Amlitelimab | | Amlitelimab | | | |
| Participants | (500 mg LD) | | 250 mg | | 125 mg | | 62.5 mg | | Total | | Placebo | |
| With | (N = 77) | | (N = 78) | | (N = 77) | | (N = 78) | | (N = 310) | | (N = 78) | |
| Any adverse events | 52 | (67.5) | 52 | (66.7) | 52 | (67.5) | 53 | (67.9) | 209 | (67.4) | 47 | (60.3)) |
| Severe adverse | 4 | (5.2) | 1 | (1.3) | 2 | (2.6) | 6 | (7.7) | 13 | (4.2) | 3 | (3.8) |
| events | | | | | | | | | | | | |
| Drug-related | 3 | (3.9) | 8 | (10.3) | 7 | (9.1) | 6 | (7.7) | 24 | (7.7) | 6 | (7.7) |
| adverse events | | | | | | | | | | | | |
| Serious adverse | 2 | (2.6) | 0 | | 1 | (1.3) | 5 | (6.4) | 8 | (2.6) | 1 | (1.3) |
| events | | | | | | | | | | | | |
| Drug-related | 0 | | 0 | | 1 | (1.3) | 0 | | 1 | (0.3) | 0 | |
| Serious adverse | | | | | | | | | | | | |
| events | | | | | | | | | | | | |
| Adverse events | 3 | (3.9) | 5 | (6.4) | 1 | (1.3) | 5 | (6.4) | 14 | (4.5) | 5 | (6.4) |

| leading to treatment | | | | | | | | | | | |
|---|---|---|---|---|---|---|---|---|---|---|---|
| discontinuation | | | | | | | | | | | |
| Adverse events | 1 | (1.3) | 2 | (2.6) | 1 | (1.3) | 0 | 4 | (1.3) | 4 | (5.1) |

| leading to study | | | | | | | | | | | |
|---|---|---|---|---|---|---|---|---|---|---|---|
| discontinuation | | | | | | | | | | | |
| Adverse events of | 3 | (3.9) | 0 | 2 | (2.6) | 1 | (1.3) | 6 | (1.9) | 1 | (1.3) |

| | | | | | | |
|---|---|---|---|---|---|---|
| special interest | | | | | | |
| All deaths | 0 | 0 | 0 | 0 | 0 | 0 |

Adverse events occurring with a frequency ≥5% in any group

| | | | | | | | | | | | | |
|---|---|---|---|---|---|---|---|---|---|---|---|---|
| Dermatitis Atopic | 9 | (11.7) | 16 | (20.5) | 15 | (19.5) | 13 | (16.7) | 53 | (17.1) | 30 | (38.5) |
| Nasopharyngitis | 14 | (18.2) | 6 | (7.7) | 9 | (11.7) | 5 | (6.4) | 34 | (11.0) | 7 | (9.0) |
| COVID-19 infection | 6 | (7.8) | 7 | (9) | 7 | (9.1) | 4 | (5.1) | 24 | (7.7) | 5 | (6.4) |
| Headache | 4 | (5.2) | 4 | (5.1) | 4 | (5.2) | 7 | (9.0) | 19 | (6.1) | 2 | (2.6) |
| Upper respiratory | 2 | (2.6) | 2 | (2.6) | 4 | (5.2) | 6 | (7.7) | 14 | (4.5) | 5 | (6.4%) |

| tract infection | | | | | | | | | | | |
|---|---|---|---|---|---|---|---|---|---|---|---|
| Blood creatine | 4 | (5.2) | 2 | (2.6) | 0 | 2 | (2.6) | 8 | (2.6) | 2 | (2.6) |

| phosphokinase | | | | | | | | | | | |
|---|---|---|---|---|---|---|---|---|---|---|---|
| increased | | | | | | | | | | | |
| Hypertension | 2 | (2.6) | 1 | (1.3) | 1 | (1.3) | 4 | (5.1) | 8 | (2.6) | 0 |

COVID-19: coronavirus disease 2019;
LD: loading dose;
mg: milligrams
Percentages are based on the number of participants who take at least one dose of study treatment, including Placebo.
Adverse Events that are missing the relationship to treatment will be considered as Treatment-Related Adverse Events.
Adverse Events were coded using MedDRA, Version 26.0.
For Part 1, a treatment-emergent adverse event is an event with the start date observed after first administration of IMP up to Week 24 visit, or with a start date no more than 140 days after last administration of IMP in case of early treatment discontinuation in part 1. Source Data: Listing 16.3.2.1

The overall incidence (256/388, 66%) of TEAEs from baseline to week 24 was slightly higher in pooled amlitelimab total (209/310, 67.4%) in comparison to placebo (47/78, 60.3%). The 7.1% difference was primarily driven by nasopharyngitis, headache and hypertension TEAEs. The incidence of SAEs was slightly higher in pooled amlitelimab arms in comparison to placebo (2.6% versus 1.3% respectively), with the greatest incidence in the 62.5 mg Q4W arm (6.4%). The incidence of TEAEs leading to permanent intervention discontinuation was lower in pooled amlitelimab arms compared with the placebo arm (4.5% versus 6.4% respectively). The incidence of adverse events of special interest (AESI) was similar in pooled amlitelimab arms compared with the placebo arm (1.9% versus 1.3% respectively). There were no deaths during the study. Although there was no clear relationship between dose of amlitelimab and incidence of AEs, there was a trend showing a higher incidence in the 250 mg Q4W with 500 mg loading dose compared to other arms for two specific AEs: nasopharyngitis (14 cases [18.2%] in 250 mg with 500 mg loading dose arm) and blood CPK increase (4 cases [5.2%] in 250 mg Q4W with 500 mg loading dose arm). Both of these AEs are discussed in more detail below.

The most frequent TEAEs (>5% in any dose arm) in descending order of total incidence were atopic dermatitis (17.1% pooled total amlitelimab versus 38.5% placebo), nasopharyngitis (11.0% pooled total amlitelimab versus 9.0% placebo), COVID-19 (7.7% pooled total amlitelimab versus 6.4% placebo), headache (6.1% pooled total amlitelimab versus 2.6% placebo), upper respiratory tract infection (4.5% pooled total amlitelimab versus 6.4% placebo), blood CPK elevated (2.6% pooled total amlitelimab versus 2.6% placebo) and hypertension (2.6% pooled total amlitelimab versus 0% placebo).

When comparing any single amlitelimab dose arm versus placebo, three imbalances were noted in TEAEs (any single amlitelimab arm versus placebo with a ≥5% difference, see Table 38). The first was nasopharyngitis (11% pooled amlitelimab versus 9.0% placebo), with the 250 mg Q4W with 500 mg loading dose arm showing the greatest incidence (18.2%). The second imbalance was observed with headache (6.1% pooled amlitelimab versus 2.6% placebo), with the 62.5 mg Q4W arm showing the highest incidence (9.0%). The third imbalance observed was hypertension (2.6% pooled amlitelimab versus 0% placebo), with the 62.5 mg Q4W arm with highest incidence (5.1%). None of these events showed any obvious dose-dependence, although nasopharyngitis was most common in the highest dose arm (250 mg Q4W with 500 mg loading dose).

The majority of TEAEs were mild or moderate intensity (39.3% of mild, 56.6% of moderate and 4.1% of severe). TEAEs with severity grade of 3 or greater (“severe”) were reported in 13 (4.2%) participants in the amlitelimab total group and 3 (3.8%) in the placebo group. There did not appear to be a dose-dependent relationship between amlitelimab and number of participants with severe events; the 62.5 mg Q4W dose arm had the most participants with severe events (6 [7.7%]) participants (Table 32).

TABLE 32
TEAEs by severity up to week 24

| | Amlitelimab | | Amlitelimab | | | | | | | |
|---|---|---|---|---|---|---|---|---|---|---|
| System Organ Class | 250 mg | | 250 mg | | Amlitelimab | | Amlitelimab | | | |
| Severity | (500 mg LD) | | (no LD) | | 125 mg | | 62.5 mg | | Placebo | |
| Preferred Term | (N = 77) | | (N = 78) | | (N = 77) | | (N = 78) | | (N = 78) | |
| Severity | n (%) | | n (%) | | n (%) | | n (%) | | n (%) | |
| Total number of TEAEs | 122 | | 108 | | 111 | | 105 | | 122 | |
| Mild | 77 | | 61 | | 52 | | 52 | | 48 | |
| Moderate | 37 | | 46 | | 57 | | 47 | | 69 | |
| Severe | 8 | | 1 | | 2 | | 6 | | 5 | |
| Number of participants with at | 52 | (67.5) | 52 | (66.7) | 52 | (67.5) | 53 | (67.9) | 47 | (60.3) |
| least one TEAEs | | | | | | | | | | |
| Mild | 29 | (37.7) | 23 | (29.5) | 22 | (28.6) | 20 | (25.6) | 10 | (12.8) |
| Moderate | 19 | (24.7) | 28 | (35.9) | 28 | (36.4) | 27 | (34.6) | 34 | (43.6) |
| Severe | 4 | (5.2) | 1 | (1.3) | 2 | (2.6) | 6 | (7.7) | 3 | (3.8) |

LD: loading dose;
TEAE: Treatment-emergent adverse event
The total number of TEAEs counts all TEAEs for participants. Each participant is counted once for each applicable specific TEAE at the maximum severity and a participant with multiple TEAEs within an SOC/PT is counted once for that SOC/PT at the maximum severity.
Adverse Events were coded using MedDRA, Version 26.0.

There were a total of 9 participants with serious TEAEs (SAEs), with a total of 11 events (n=2 [4 events] in 250 mg Q4W with 500 mg loading dose arm, n=0 [0 event] in 250 mg Q4W arm, n=1 [1 event] in 125 mg Q4W arm, n=5 [5 events] in 62.5 mg Q4W arm and n=1 [1 event] in placebo arm). The SAEs reported were mostly reflective of underlying comorbidities of individual AD participants or had clear explanations independent of amlitelimab treatment (See Table 32, Table 33, and patient brief narratives below).

TABLE 33
Serious TEAEs up to week 24 - safety analysis set

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| System Organ Class | (N = 77) | (N = 78) | (N = 77) | (N = 78) | (N = 78) |
| Preferred Term | n (%) | n (%) | n (%) | n (%) | n (%) |
| Total number of | 4 | 0 | 1 | 5 | 1 |
| serious TEAES | | | | | |
| Number of | 2 (2.8) | 0 | 1 (1.3) | 5 (6.4) | 1 (1.3) |
| participants with at | | | | | |
| least one serious | | | | | |
| TEAEs | | | | | |
| Infections and | 0 | 0 | 0 | 2 (2.6) | 0 |
| infestations | | | | | |
| Appendicitis | 0 | 0 | 0 | 1 (1.3) | 0 |
| Pharyngitis | 0 | 0 | 0 | 1 (1.3) | 0 |
| Metabolism and | 1 (1.3) | 0 | 0 | 0 | 0 |
| nutrition disorders | | | | | |
| Metabolic acidosis | 1 (1.3) | 0 | 0 | 0 | 0 |
| Psychiatric | 1 (1.3) | 0 | 0 | 0 | 0 |
| disorders | | | | | |
| Alcohol withdrawal | 1 (1.3) | 0 | 0 | 0 | 0 |
| syndrome | | | | | |
| Nervous system | 1 (1.3) | 0 | 0 | 0 | 0 |
| disorders | | | | | |
| Tension headache | 1 (1.3) | 0 | 0 | 0 | 0 |
| Cardiac disorders | 1 (1.3) | 0 | 0 | 0 | 1 (1.3) |
| Atrial fibrillation | 0 | 0 | 0 | 0 | 1 (1.3) |
| Supraventricular | 1 (1.3) | 0 | 0 | 0 | 0 |
| tachycardia | | | | | |
| Gastrointestinal | 0 | 0 | 0 | 1 (1.3) | 0 |
| disorders | | | | | |
| Hemorrhoidal | 0 | 0 | 0 | 1 (1.3) | 0 |
| hemorrhage | | | | | |
| Skin and | 0 | 0 | 1 (1.3) | 0 | 0 |
| subcutaneous tissue | | | | | |
| disorders | | | | | |
| Dermatitis bullous | 0 | 0 | 1 (1.3) | 0 | 0 |
| Musculoskeletal and | 0 | 0 | 0 | 1 (1.3) | 0 |
| connective tissue | | | | | |
| disorders | | | | | |
| Osteoarthritis | 0 | 0 | 0 | 1 (1.3) | 0 |
| Injury, poisoning | 0 | 0 | 0 | 1 (1.3) | 0 |
| and procedural | | | | | |
| complications | | | | | |
| Forearm fracture | 0 | 0 | 0 | 1 (1.3) | 0 |

LD: loading dose;
TEAE: Treatment-emergent adverse event
The total number of serious TEAEs counts all serious TEAEs for participants. At each level of participant summarization, a subject is counted once for the most related event if the participant reported one or more events.
Adverse Events were coded using MedDRA, Version 28.0.
Source Data: Listing 16.3.2.4

There were a total of 19 participants with AEs leading to treatment discontinuation with a total of 21 events (n=3 [5 events] in 250 mg Q4W with 500 mg loading dose arm, n=5 [5 events] in 250 mg Q4W arm, n=1 [1 event] in 125 mg Q4W arm, n=5 [5 events] in 62.5 mg Q4W arm and n=5 [5 events] in placebo arm]). Out of the 21 TEAEs that led to permanent treatment intervention discontinuation overall, nine were serious TEAEs and the others were events of moderate intensity. The most common reason for treatment discontinuation was atopic dermatitis with 4 events across all arms (1 [1.3%] in 250 mg Q4W with 500 mg loading dose arm, 2 [2.6%] in 250 mg Q4W arm, 1 [1.3%] in placebo arm). There was no one reason/PT for treatment discontinuation that was notably more common (Table 31 and Table 34).

TABLE 34
TEAEs leading to permanent study intervention discontinuation up to week 24 - safety analysis set

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| System Organ Class | (N = 77) | (N = 78) | (N = 77) | (N = 78) | (N = 78) |
| Preferred Term | n (%) | n (%) | n (%) | n (%) | n (%) |
| Total number of TEAES leading to | 5 | 5 | 1 | 5 | 5 |
| treatment discontinuation | | | | | |
| Number of participants with at | 3 (3.9) | 5 (6.4) | 1 (1.3) | 5 (6.4) | 5 (6.4) |
| least one TEAES leading to | | | | | |
| treatment discontinuation | | | | | |
| Infections and infestations | 0 | 1 (1.3) | 0 | 1 (1.3) | 1 (1.3) |
| Appendicitis | 0 | 0 | 0 | 1 (1.3) | 0 |
| Chronic sinusitis | 0 | 0 | 0 | 0 | (1.3) |
| Sinusitis | 0 | 1 (1.3) | 0 | 0 | 0 |
| Blood and lymphatic system | 0 | 2 (2.6) | 0 | 1 (1.3) | |
| disorders | | | | | |
| Anemia | 0 | 1 (1.3) | 0 | 0 | 0 |
| Anemia of chronic disease | 0 | 0 | 0 | 1 (1.3) | 0 |
| Iron deficiency anemia | 0 | 1 (1.3) | 0 | 0 | |
| Immune system disorders | 0 | 0 | 0 | 0 | 1 (1.3) |
| Drug hypersensitivity | 0 | 0 | 0 | 0 | 1 (1.3) |
| Metabolism and nutrition | 1 (1.3) | 0 | 0 | 0 | 0 |
| disorders | | | | | |
| Metabolic acidosis | 1 (1.3) | 0 | 0 | 0 | 0 |
| Psychiatric disorders | 1 (1.3) | 0 | 0 | 0 | 0 |
| Alcohol withdrawal syndrome | 1 (1.3) | 0 | 0 | 0 | 0 |
| Cardiac disorders | 1 (1.3) | 0 | 0 | 0 | 1 (1.3) |
| Atrial fibrillation | 0 | 0 | 0 | 0 | 1 (1.3) |
| Supraventricular tachycardia | 1 (1.3) | 0 | 0 | 0 | 0 |
| Gastrointestinal disorders | 0 | 0 | 0 | 1 (1.3) | 0 |
| Hemorrhoidal hemorrhage | 0 | 0 | 0 | 1 (1.3) | 0 |
| Skin and subcutaneous tissue | 1 (1.3) | 2 (2.6) | 1 (1.3) | 0 | 2 (2.6) |
| disorders | | | | | |
| Dermatitis atopic | 1 (1.3) | 2 (2.6) | 0 | 0 | 1 (1.3) |
| Dermatitis bullous | 0 | 0 | 1 (1.3) | 0 | 0 |
| Hypersensitivity vasculitis | 0 | 0 | 0 | 0 | 1 (1.3) |
| Musculoskeletal and connective | 0 | 0 | 0 | 1 (1.3) | 0 |
| tissue disorders | | | | | |
| Osteoarthritis | 0 | 0 | 0 | 1 (1.3) | 0 |
| General disorders and | 1 (1.3) | 0 | 0 | 0 | 0 |
| administration site conditions | | | | | |
| Face oedema | 1 (1.3) | 0 | 0 | 0 | 0 |
| Injury, poisoning and | 0 | 0 | 0 | 1 (1.3) | 0 |
| procedural complications | | | | | |
| Forearm fracture | 0 | 0 | 0 | 1 (1.3) | 0 |

LD: leading dose;
PT: preferred term;
SOC: system organ class;
Note:
TEAE: Treatment-emergent adverse event
The total number of TEAEs counts all TEAEs for participants. Percentages are based on the number of participants who take at least one dose of study treatment, including Placebo.
At each level of participant summarisation, a participant is counted once if the participant reported one or more events.
Adverse Events were coded using MedDRA, Version 26.0.

There were a total of seven participants with AESIs with a total of nine events (n=3 [5 events] in 250 mg Q4W with 500 mg loading dose arm, n=0) [0 event] in 250 mg Q4W arm, n=2 [2 events] in 125 mg Q4W arm, n=1 [1 event] in 62.5 mg Q4W arm and n=1 [1 event] in placebo arm]) (see Table 31 and Table 35).

TABLE 35
Treatment emergent AESIs up to week 24 - safety analysis set

| | Amlitelimab | Amlitelimab | | | |
|---|---|---|---|---|---|
| | 250 mg | 250 mg | Amlitelimab | Amlitelimab | |
| | (500 mg LD) | (no LD) | 125 mg | 62.5 mg | Placebo |
| AESI Category | (N = 77) | (N = 78) | (N = 77) | (N = 78) | (N = 78) |
| Preferred Term | n (%) | n (%) | n (%) | n (%) | n (%) |
| Total number of treatment-emergent | 5 | 0 | 2 | 1 | 1 |
| AESIs | | | | | |
| Number of participants with at least | 3 (3.9) | 0 | 2 (2.6) | 1 (1.3) | 1 (1.3) |
| one treatment-emergent AESIs | | | | | |
| Increase in ALT | 2 (2.6) | 0 | 1 (1.3) | 1 (1.3) | 1 (1.3) |
| Alanine aminotransferase increased | 2 (2.6) | 0 | 1 (1.3) | 1 (1.3) | 1 (1.3) |
| Allergic reaction | 1 (1.3) | | 1 (1.3) | 0 | 0 |
| Conjunctivitis allergic | 1 (1.3) | 0 | 0 | 0 | 0 |
| Dermatitis bullous | 0 | 0 | 1 (1.3) | 0 | 0 |
| Face oedema | 1 (1.3) | 0 | 0 | 0 | 0 |

AESI: Adverse event of special interest; ALT: alanine aminotransferase; LD: leading dose.
The total number of AESIs counts all treatment-emergent AESIs for participants. At each level of participant summarization, a participant is counted once for the most related event if the participant reported one or more events.
Adverse Events were coded using MedDRA, Version 26.0.

Overall, no cases of symptomatic eosinophilic conditions, anaphylaxis reaction, or serum sickness have been reported. There were three potential cases of hypersensitivity reactions (with two cases that were reported as AESI). All cases were in participants with negative ADA. The third case was a 43-year-old female participant (250 mg Q4W amlitelimab arm) who developed a “lip swelling” one day after 1st IMP administration. This event was reported to be mild, not related, as a “slight swelling of the lips (allergic)/allergen unknown.” The participant fully recovered in two days, with no corrective treatment. Amlitelimab was continued and the participant received four more administrations with no recurrence of symptoms. The treatment was ultimately discontinued on day 110 due to the use of systemic steroids for a case of sinusitis. This event has not been reported as an AESI.

There were five participants with six cases of ALT elevation >3× ULN (4 [1.3%]) in pooled amlitelimab versus 1 [1.3%] in placebo). Regarding participants who had received amlitelimab, all events can be explained by either a known prior history of hepatic steatosis or recent alcohol intake reported by the participant. All events were reported as not related to amlitelimab as per investigator's judgement; mild or moderate in intensity and all cases were resolved. Two participants out of the 5 discontinued the study (withdrawal of consent).

There were three injection site concerns (discoloration, erythema and pruritis) reported as TEAEs in amlitelimab arms. Specifically, there was one case in each amlitelimab dose arm except for the 250 mg with 500 mg loading dose arm, compared to zero cases in the placebo arm. According to an analysis of all recorded local skin reactions to injections, rates of injection site reactions were higher in amlitelimab pooled groups (53 [17.1%]) compared to placebo (8 [10.3%]). Of these recorded reactions, all were mild or moderate with the exception of one participant (62.5 mg Q4W arm) who had severe injection site pain. The incidence of severe injection site pain was not reported as an AE by the Investigator-however, it was associated with a reported AE of presyncope during the first IMP injection. Per the Investigator, the participant was very afraid of the injection and suffered pain during the injection after which the participant's blood pressure “decreased for a moment” before normalizing with the participant lying down. The event resolved after one minute. It was reported as moderate intensity. The participant went on to complete the study with no further reports of injection-related AEs. Overall, there was no apparent relationship between dose and injection site reactions. No injection site reactions were serious, persistent, or resulted in premature intervention discontinuation. No cases of chills but one case of pyrexia was observed in amlitelimab arms (67 days after last dose and not related to injections). No cases of aphthous ulcers were reported.

No malignancies were reported, including no non-melanoma skin cancer.

No symptomatic overdoses were reported.

To be noted: overdose with IMP that was suspected by the Investigator was defined as at least twice the intended dose during an interval of less than 21 days. In the safety population, IMP overdose was reported for 7 [2.3%] participants in the amlitelimab group (1 participant in 250 mg Q4W with 500 mg loading dose arm, 3 participants in 250 mg Q4W dose arm, 1 participant in 125 mg Q4W dose arm and 2 participants in 62.5 mg Q4W dose arm) with 2 consecutive doses taken within 14 and 20 days; and 1 [1.3%] participant in the placebo group with 2 consecutive doses taken within 14 days. None of these participants experienced any symptoms. Therefore, no symptomatic overdoses were reported in this study. These dose interval irregularities were reported as protocol deviations, and primarily occurred as a result of visit scheduling logistics (i.e., participant having to delay or advance visits due to personal reasons).

No pregnancies were reported.

No parasitic infections or serious, opportunistic infections were reported. Overall, the safety data support the conclusion that amlitelimab use is not associated with an increased risk of serious and/or severe infections. For the participant that developed a bullous and pustular dermatitis (which was reported as serious and related to study drug by the Investigator, a biopsy suggested a pustular reaction. In the context of AD, this may have been an infection

Overall incidences of herpes infections were reassuring when compared to placebo. There were seven herpes viral infections (pooled PTs of “oral herpes,” “herpes simplex reactivation,” “herpes dermatitis” and “eczema herpeticum”) (2.3%) participants in pooled amlitelimab arms versus 2 (2.5%) participants in the placebo arm.

Overall incidences of conjunctivitis were reassuring when compared to placebo. For conjunctivitis (including PTs of “conjunctivitis allergic,” “conjunctivitis” and “conjunctivitis bacterial”), there were 5 (1.6%) participants in pooled amlitelimab arms versus 3 (3.8%) participants in the placebo arm with respectively 3 (1.0%) participants in pooled amlitelimab versus 1 (1.3%) participants in placebo group for the PT “conjunctivitis allergic,” 1 (0.3%) participants in pooled amlitelimab versus 1 (1.3%) participants in placebo group for the PT “conjunctivitis” and 1 (0.3%) participants in pooled amlitelimab versus 1 (1.3%) participants in placebo group for the PT “conjunctivitis bacterial.”

No participants died during this study.

### Pharmacokinetics

Pharmacokinetics of amlitelimab in participants with moderate to severe AD was documented using sparse PK sampling (primarily pre-dose samples) collected in Study KY1005-CT05/DRI17366 (STREAM-AD). In addition, a Population PK (PopPK) analysis was conducted using pooled data from phase 1 studies in healthy subjects and phase 2 studies in AD participants.

### Amlitelimab Trough Concentration in Serum

Concentrations of amlitelimab in serum were measured at baseline and pre-dose at week 4, week 8, week 12, week 16, week 20 and week 24. In addition, samples were collected post-dosing at week 1, week 2 and week 17. Descriptive statistics for amlitelimab concentrations are summarized in Table 36.

TABLE 36
Concentration (μg/mL) of amlitelimab in
serum up to week 24 (part 1) - PK population

| | Amlitelimab | Amlitelimab | | |
|---|---|---|---|---|
| Visit | 250 mg | 250 mg | Amlitelimab | Amlitelimab |
| Amlitelimab | (500 mg LD) | (no LD) | 125 mg | 62.5 mg |
| 5 (ug/ml) | (N = 77) | (N = 76) | (N = 77) | (N = 78) |
| Baseline (Week 0) | | | | |
| N | 77 | 74 | 76 | 73 |
| Mean (SD) | 0.00 (0.014) | 0.00 (0.014) | 0.01 (0.074) | 0.00 (0.000) |
| Median | 0.00 | 0.00 | 0.00 | 0.00 |
| Min, Max | 0.0, 0.1 | 0.0, 0.1 | 0.0, 0.6 | 0.0, 0.0 |
| CV % | 618.9 | 860.2 | 614.8 | |
| Week 1 | | | | |
| N | 72 | 73 | 74 | 73 |
| Mean (SD) | 58.29 (24.674) | 30.90 (18.530) | 14.18 (5.060) | 8.49 (3.331) |
| Median | 54.85 | 29.70 | 14.50 | 8.89 |
| Min, Max | 9.6, 161.0 | 0.0, 162.0 | 0.0, 27.8 | 0.0, 14.7 |
| CV % | 42.3 | 60.0 | 35.7 | 39.2 |
| Week 2 | | | | |
| N | 65 | 69 | 67 | 70 |
| Mean (SD) | 47.56 (17.274) | 25.09 (9.041) | 13.56 (3.886) | 7.57 (2.947) |
| Median | 48.50 | 24.90 | 13.60 | 7.29 |
| Min, Max | 0.0, 86.5 | 0.0, 47.7 | 7.8, 23.4 | 2.2, 16.9 |
| CV % | 36.3 | 36.0 | 28.7 | 38.9 |
| Week 4 | | | | |
| N | 62 | 65 | 69 | 68 |
| Mean (SD) | 38.49 (22.532) | 19.00 (7.869) | 9.67 (3.728) | 5.09 (2.176) |
| Median | 32.60 | 19.00 | 8.74 | 5.08 |
| Min, Max | 6.1, 176.0 | 0.0, 41.8 | 0.0, 20.5 | 0.8, 11.3 |
| CV % | 58.5 | 41.4 | 38.6 | 42.8 |
| Week 8 | | | | |
| N | 53 | 48 | 56 | 61 |
| Mean (SD) | 37.67 (15.157) | 32.14 (16.502) | 15.07 (6.383) | 7.92 (3.263) |
| Median | 33.90 | 29.55 | 13.55 | 8.09 |
| Min, Max | 14.8, 93.6 | 9.1, 104.0 | 4.3, 32.9 | 1.3, 19.3 |
| CV % | 40.2 | 51.4 | 42.4 | 41.2 |
| Week 12 | | | | |
| N | 50 | 45 | 54 | 60 |
| Mean (SD) | 39.26 (33.847) | 34.67 (15.191) | 18.12 (8.439) | 9.22 (4.116) |
| Median | 34.00 | 32.10 | 17.25 | 9.16 |
| Min, Max | 15.9, 260.0 | 8.7, 65.4 | 6.7, 48.0 | 1.7, 23.2 |
| CV % | 86.2 | 43.8 | 46.6 | 44.6 |
| Week 16 | | | | |
| N | 49 | 48 | 49 | 52 |
| Mean (SD) | 40.12 (28.610) | 38.09 (13.536) | 18.39 (7.245) | 10.13 (4.515) |
| Median | 33.60 | 38.55 | 17.30 | 10.08 |
| Min, Max | 0.0, 166.0 | 10.2, 60.3 | 3.5, 34.9 | 0.0, 20.8 |
| CV % | 71.3 | 35.5 | 39.4 | 44.6 |
| Week 17 | | | | |
| N | 49 | 54 | 54 | 51 |
| Mean (SD) | 62.34 (28.081) | 64.64 (24.169) | 29.26 (11.659) | 16.79 (6.677) |
| Median | 58.30 | 62.50 | 26.95 | 15.50 |
| Min, Max | 12.0, 184.0 | 11.5, 126.0 | 3.6, 59.4 | 3.9, 34.3 |
| CV % | 45.0 | 37.4 | 39.8 | 39.8 |
| Week 20 | | | | |
| N | 49 | 49 | 54 | 56 |
| Mean (SD) | 37.92 (14.171) | 48.26 (30.691) | 19.31 (7.203) | 10.68 (3.715) |
| Median | 34.80 | 45.60 | 19.00 | 10.30 |
| Min, Max | 13.5, 68.1 | 9.5, 183.0 | 3.7, 38.5 | 2.8, 19.8 |
| CV % | 37.4 | 63.6 | 37.3 | 34.8 |
| Week 24 | | | | |
| N | 41 | 43 | 47 | 43 |
| Mean (SD) | 41.98 (23.087) | 43.81 (17.403) | 20.25 (10.575) | 11.07 (3.918) |
| Median | 36.10 | 43.20 | 18.20 | 11.80 |
| Min, Max | 15.1, 151.0 | 10.8, 81.8 | 0.0, 53.5 | 2.2, 21.9 |
| CV % | 55.0 | 39.7 | 52.2 | 35.4 |

CV: coefficient of variation; LD: loading dose; NA: not applicable; SD: standard deviation; SE: standard error.
Notes:
The baseline value is defined as the last non-missing value before the first dose of study drug.
Only pre-injection measurements are included from visits with dosing (per schedule: every 4 weeks starting from Week 0 until Week 48 if participant is rerandomized or Week 20 if not).
Measurements below LLOQ are imputed as 0.

Pharmacokinetic steady-state appeared to be reached by week 4 in the 250 mg Q4W arm with 500 mg loading dose, and by week 12 in the dose arms without loading dose. At week 24, the mean trough concentration (standard deviation) of amlitelimab was 42.0 (23.1) μg/mL, 43.8 (17.4) μg/mL, 20.3 (10.6) μg/mL and 11.1 (3.92) μg/mL in the 250 mg Q4W with 500 mg loading dose, 250 mg Q4W, 125 mg Q4W and 62.5 mg Q4W arms, respectively.

### Immunogenicity

Table 38 provides a summary of ADA incidence in all randomized participants that have at least one ADA measurement post-baseline up to the primary analysis cut-off date. Three participants with pre-existing ADA were reported: one in the 250 mg arm with a loading dose; one in the 250 mg Q4W arm without 500 mg loading dose; and one in the 125 mg Q4W arm. A dose-dependent effect was observed regarding ADA response, with higher incidences of treatment-emergent ADA at 62.5 mg Q4W (28.2%) and 125 mg Q4W (13.2%) compared to 250 mg Q4W with or without 500 mg loading dose (3.8% and 2.6%, respectively). All cases were identified as drug-induced ADA.

TABLE 38
Summary of ADA incidence - ADA population

| | Amlitelimab | Amlitelimab | | |
|---|---|---|---|---|
| | 250 mg | 250 mg | Amlitellmab | Amlitelimab |
| | (500 mg LD) | (no LD) | 125 mg | 62.5 mg |
| ADA status | (N = 77) | (N = 78) | (N = 76) | (N = 78) |
| Pre-existing ADA | 1 (1.3%) | 1 (1.3%) | 1 (1.3%) | 0 |
| Without treatment-emergent ADA | 75 (97.4%) | 75 (96.2%) | 66 (86.8%) | 56 (71.8%) |
| Treatment emergent ADA | 2 (2.6%) | 3 (3.8%) | 10 (13.2%) | 22 (28.2%) |
| Treatment-induced ADA | 2 (2.6%) | 3 (3.8%) | 10 (13.2%) | 22 (28.2%) |
| Treatment-boosted ADA | 0 | 0 | 0 | 0 |
| Unclassified ADA | 0 | 0 | 0 | 0 |
| Inconclusive ADA | 0 | 0 | 0 | 0 |

ADA: antidrug antibody:
LD: loading dose
Immunogenicity population in this summary table includes all measurements from randomized participants with at least one post-baseline ADA result (positive, negative or inconclusive) up to the primary analysis cut-off date of 28 APR. 2023 (primary analysis)

Table 39 summarizes treatment-emergent ADA in participants. The median time for treatment-emergent ADA response onset ranged from 16 to 20 weeks. The median ADA peak titer remained low (=5) across all dose arms, with higher titers (≥20) observed in the lower dose groups (125 mg Q4W and 62.5 mg Q4W arms). Only one participant in the 62.5 mg Q4W arm had persistent ADA, while the response for other participants with treatment-emergent ADA was indeterminate.

TABLE 39
Summary of treatment-emergent ADA - ADA population

| | Amlitelimab | Amlitelimab | | |
|---|---|---|---|---|
| | 250 mg | 250 mg | Amlitelimab | Amlitelimab |
| | (500 mg LD) | (no LD) | 125 mg | 62.5 mg |
| Treatment-induced ADA | (N = 77) | (N = 78) | (N = 76) | (N = 78) |
| Time of onset (weeks) | | | | |
| N | 2 | 3 | 10 | 22 |
| Median | 19.86 | 18.14 | 15.79 | 16.29 |
| Q1, Q3 | 15.71, 24.00 | 16.00, 18.29 | 4.57, 16.43 | 16.14, 16.71 |
| Min, Max | 15.7, 24.0 | 16.0, 18.3 | 2.0, 36.3 | 4.1, 26.0 |
| ADA peak fiter | | | | |
| N | 2 | 3 | 10 | 22 |
| Median | 5.00 | 5.00 | 5.00 | 5.00 |
| Q1, Q3 | 5.00, 5.00 | 5.00, 5.00 | 5.00, 20.00 | 5.00, 5.00 |
| Min, Max | 5.0, 5.0 | 5.0, 5.0 | 5.0, 80.0 | 5.0, 20.0 |
| ADA response | | | | |
| Persistent ADA | 0 | 0 | 0 | 1 (1.3%) |
| Transient ADA | 0 | 0 | 0 | 0 |
| Indeterminate ADA | 2 (2.6%) | 3 (3.8%) | 10 (13.2%) | 21 (26.9%) |
| Duration (weeks) for | | | | |
| Persistent ADA | | | | |
| N | 0 | 0 | 0 | 1 |
| Median | | | | 19.57 |
| Q1, Q3 | | | | 19.57, 19.57 |
| Min, Max | | | | 19.6, 19.6 |

ADA: antidrug antibody;
LD: loading dose
Immunogenicity population in this summary table includes all measurements from randomized participants with at least one post-baseline ADA result (positive, negative or inconclusive) up to the primary analysis cut-off date of 26 APR. 2023 (primary analysis)

### Analyses of Potential Association Between ADA and Pharmacokinetics

In the population PK analysis that included the KY1005-CT02 and KY1005-CT05/DRI17366 (STREAM-AD) studies, ADA were not found as impacting amlitelimab PK.

### Analyses of Potential Association Between ADA and Treatment-Emergent Adverse Events

No relationship between TEAEs and ADA response status was observed. In addition, a more focused analysis was performed to evaluate any potential association between ADA response status and hypersensitivity reactions by using the SMQs of “anaphylaxis” and “hypersensitivity reactions” and comparing ADA status of participants with TEAEs within these categories (Table 40). No evidence of relationship between anaphylaxis and hypersensitivity reactions and ADA positive status was observed.

TABLE 40
Anaphylaxis and hypersensitivity reactions by ADA response status - ADA population part 1

| | Amlitelimab | | Amlitelimab | | | | | |
|---|---|---|---|---|---|---|---|---|
| | 250 mg | | 250 mg | | Amlitelimab | | Amlitelimab | |
| | (500 mg LD) | | (no LD) | | 125 mg | | 62.5 mg | |
| | (N = 77) | | (N = 78) | | (N = 76) | | (N = 78) | |
| | | ADA | | ADA | | ADA | | ADA |
| | ADA positive | negative | ADA positive | negative | ADA positive | negative | ADA positive | negative |
| n(%) | (N = 2) | (N = 75) | (N = 3) | (N = 75) | (N = 10) | (N = 66) | (N = 22) | (N = 56) |
| Anaphylaxis | 0 | 3 (4.0) | 0 | 4 (5.3) | 1 (10.0) | 6 (9.1) | 0 | 1 (1.8) |
| Erythema | 0 | 0 | 0 | 0 | 1 (10.0) | 0 | 0 | 0 |
| Asthma | 0 | 0 | 0 | 0 | 0 | 2 (3.0) | 0 | 0 |
| Cough | 0 | 2 (2.7) | 0 | 0 | 0 | 2 (3.0) | 0 | 1 (1.8) |
| Face oedema | 0 | 1 (1.3) | 0 | 0 | 0 | 0 | 0 | 0 |
| Hypotension | 0 | 0 | 0 | 1 (1.3) | 0 | 0 | 0 | 0 |
| Lip swelling | 0 | 0 | 0 | 1 (1.3) | 0 | 0 | 0 | 0 |
| Pruritus | 0 | 0 | 0 | 2 (2.7) | 0 | 0 | 0 | 0 |
| Pruritus allergic | 0 | 0 | 0 | 1 (1.3) | 0 | 0 | 0 | 0 |
| Swelling of eyelid | 0 | 0 | 0 | 0 | 0 | 1 (1.5) | 0 | 0 |
| Urticaria | 0 | 0 | 0 | 0 | 0 | 1 (1.5) | 0 | 0 |
| Hypersensitivity | 0 | 0 | 0 | 3 (4.0) | 1 (10.0) | 2 (3.0) | 0 | 0 |
| reaction | | | | | | | | |
| Erythema | 0 | 0 | 0 | 0 | 1 (10.0) | 0 | 0 | 0 |
| Asthma | 0 | 0 | 0 | 0 | 0 | 2 (3.0) | 0 | 0 |
| Conjunctivitis | 0 | 0 | 0 | 1 (1.3) | 0 | 0 | 0 | 0 |
| Pruritus | 0 | 0 | 0 | 2 (2.7) | 0 | 0 | 0 | 0 |

LD: loading dose
Search criteria: SMQ Hypersensitivity for Hypersensitivity reaction and SMQ Anaphylactic reaction for Anaphylaxis.
No imputation is used for missing ADA results.
The number of positive ADA participants are participants with treatment-induced ADAs or treatment-boosted ADAs at any time point during the treatment-emergent period.
For Part 1, a treatment-emergent adverse event is an event with the start date observed after first administration of IMP up to Week 24 visit, or with a start date no more than 140 days after last administration of IMP in case of early treatment discontinuation in part 1.
Note:
Table sorted by decreasing frequency of PT in the overall ADA positive group.

The results of this Phase 2b study (KY1005-CT05/DRI17366 [STREAM-AD]) indicate that amlitelimab is efficacious across multiple domains of the disease and provides treatment benefit on signs and symptoms of moderate-to-severe AD, as well as on QoL. Amlitelimab was generally well tolerated and had a favorable safety profile in the treatment of participants with moderate-to-severe AD who have a documented history, within 6 months prior to baseline, of either inadequate response to topical treatments or inadvisability of topical, with a positive benefit-risk profile. The safety profile of amlitelimab observed in this Phase 2b study (KY1005-CT05/DRI17366 [STREAM-AD]) is consistent with the findings of the Phase 2a study (KY1005-CT02) with no new safety concerns observed.

The study met its primary endpoint of percentage change in EASI score from baseline at 16 weeks, with continued improvement seen through 24 weeks. Improvements also seen in all key secondary endpoints at 16 and 24 weeks

Data support amlitelimab as a potential first-in-class anti-OX40-Ligand monoclonal antibody for adults with moderate-to-severe atopic dermatitis, addressing type 2 and non-type 2 inflammation.

The primary endpoint and all key secondary endpoints were met in a Phase 2b study (STREAM-AD) evaluating the efficacy and safety of amlitelimab, a novel investigational human monoclonal antibody targeting key immune system regulator OX40-Ligand, compared to placebo in adults with moderate-to-severe atopic dermatitis whose disease cannot be adequately controlled with topical medications or for whom topical medications are not a recommended treatment approach.

In the study, treatment with amlitelimab resulted in statistically significant improvements in average EASI score from baseline at 16 weeks compared to placebo for all four subcutaneous doses that were studied. There were also improvements in key secondary endpoints including achievement of at least 75% reduction in EASI from baseline, an Investigator Global Assessment (IGA) score of 0) (clear) or 1 (almost clear) with a reduction from baseline of at least 2 points, and proportion of patients with improvement (reduction) of weekly average of pruritus Numerical Rating Scale (NRS) of at least 4. Continued treatment improvements on these measures were observed through week 24. Biomarker results support an effect on both type 2 and non-type 2 pathways.

Amlitelimab was well-tolerated in the study across all dose arms with no new safety signals identified. The most commonly reported adverse events that occurred in at least 5% of patients were atopic dermatitis, COVID-19 infections, increased blood creatine phosphokinase, headache, hypertension, nasopharyngitis and upper respiratory tract infection. With the exception of atopic dermatitis, none of these events led to treatment discontinuation.

STREAM-AD, a Phase 2b study, was a randomized double-blind, placebo-controlled study, evaluating amlitelimab in adult patients with moderate-to-severe atopic dermatitis whose disease was inadequately controlled with topical therapies or where such therapies were not advisable. In the trial, participants were randomized (1:1:1:1:1) to either 250 mg every four weeks with a 500 mg loading dose, or either 250 mg, 125 mg, 62.5 mg, or placebo every four weeks with no loading dose.

The primary endpoint was percentage change in EASI from baseline at 16 weeks. Key secondary endpoints included change in EASI from baseline at 24 weeks, percentage of patients with at least a 75% reduction from baseline in EASI at 16 and 24 weeks, percentage of patients with a response of IGA 0 (clear) or 1 (almost clear) and a reduction from baseline ≥2 points at 16 and 24 weeks, and proportion of patients with improvement (reduction) of weekly average of pruritus NRS >4 with a baseline pruritus of ≥4 from baseline at 16 and 24 weeks.

The study enrolled 390 people.

### Study Design and Primary Analysis Specifics

24-week induction period (KRM focus)+28-week randomized withdrawal period examining 5 arms (250 mg Q4W with 500 mg LD, 250 mg Q4W, 125 mg Q4W, 62.5 mg Q4W, pbo) in mod-to-severe AD patients (n=390).

PA comprises all randomized pts who completed Wk24/early discontinuation (efficacy analysis) and all treated (safety analysis, n=388).

### Primary Endpoint Result (Week 16)

ΔEASI: All dose arms showed statistically significant mean % reductions in EASI score vs. pbo at week 16 and nominally significant at week 24 with 250 mg LD showing highest response (week 16 and week 24) vs. placebo and vs. other arms.

No dose-response proportionality observed.

Key secondary endpoints at week 16 and week 24:
EASI-75: met nominal statistical significance in all dose arms with 250 mg LD showing early separation (week 2) and highest response vs. pbo and other arms (week 24);
IGA 0/1: met nominal statistical significance in all dose arms (except 250 mg at wk16) with 250 mg LD showing early separation (week 4) and highest response vs. pbo and vs. other arms (week 24);
Reduction 4-pts in PP-NRS: met nominal statistical significance in all dose arms with 250 mg LD and 62.5 mg showing early separation (week 3) and 250 mg LD showing highest response vs. pbo and vs. other arms (week 16 and week 24).

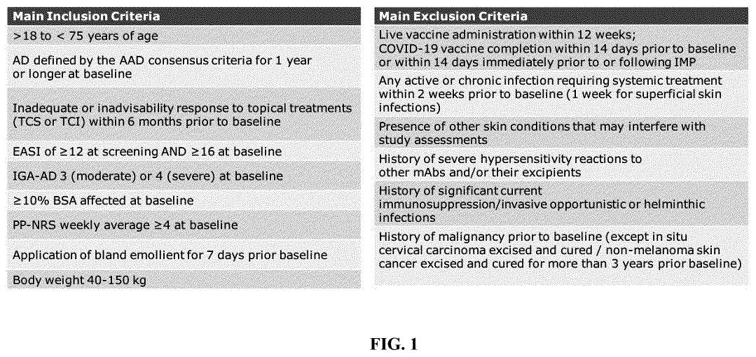

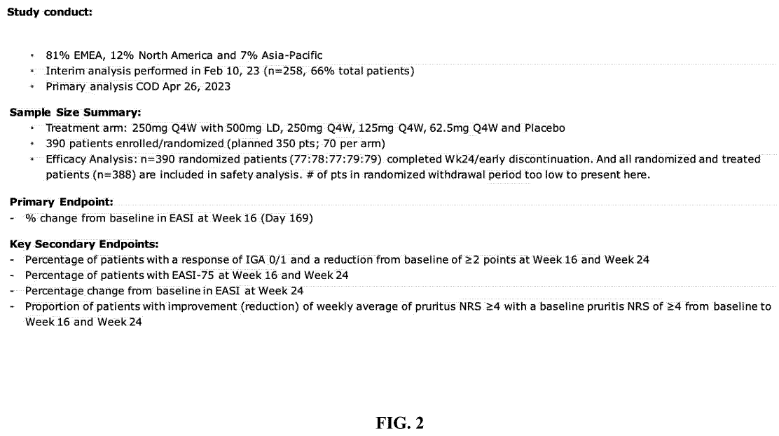

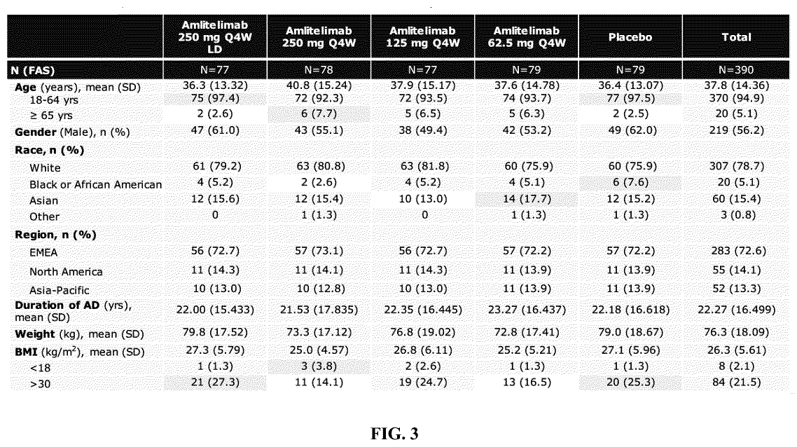

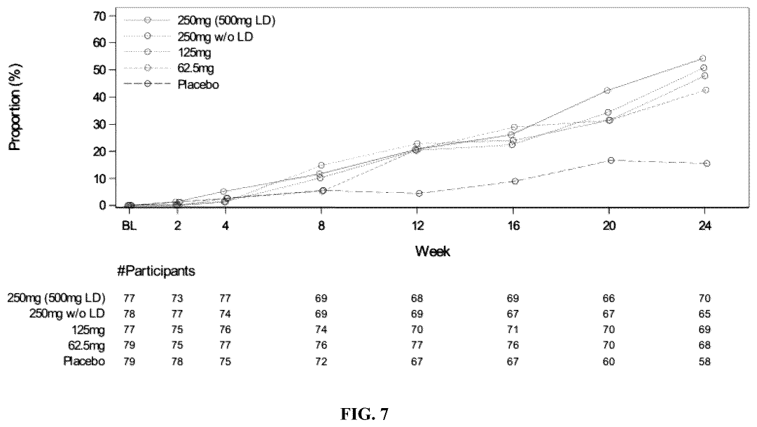

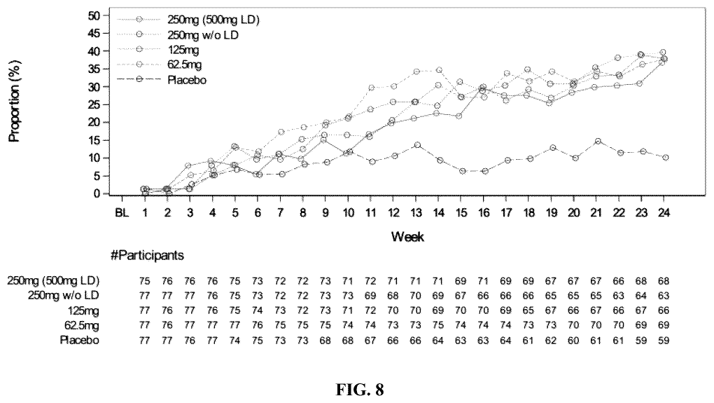

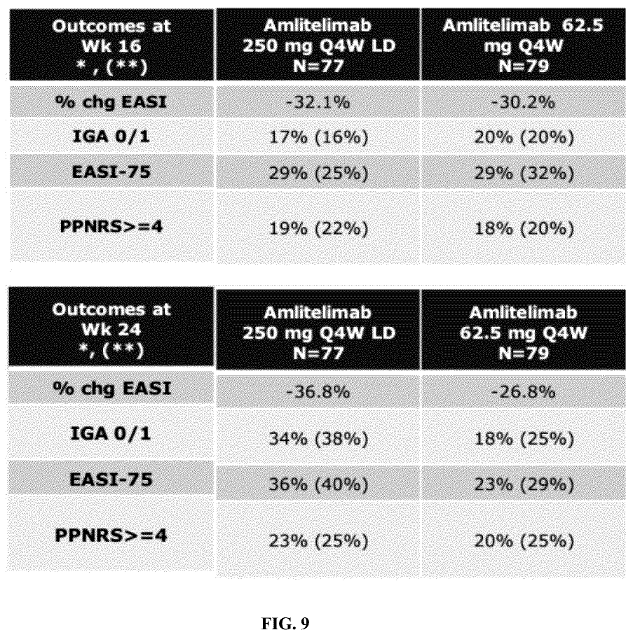

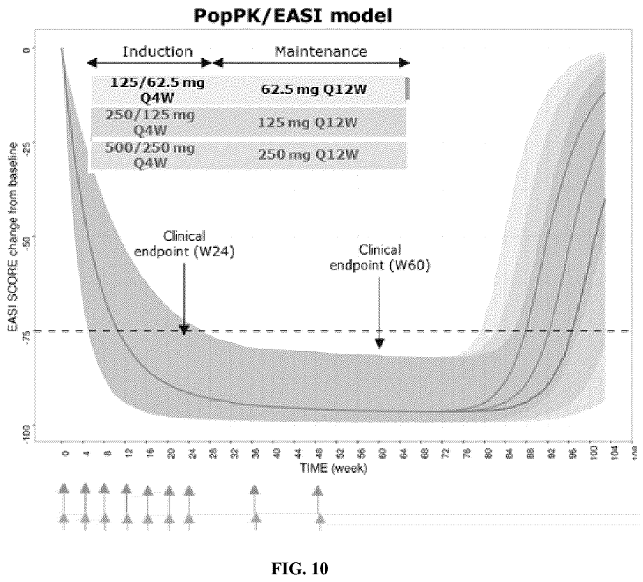

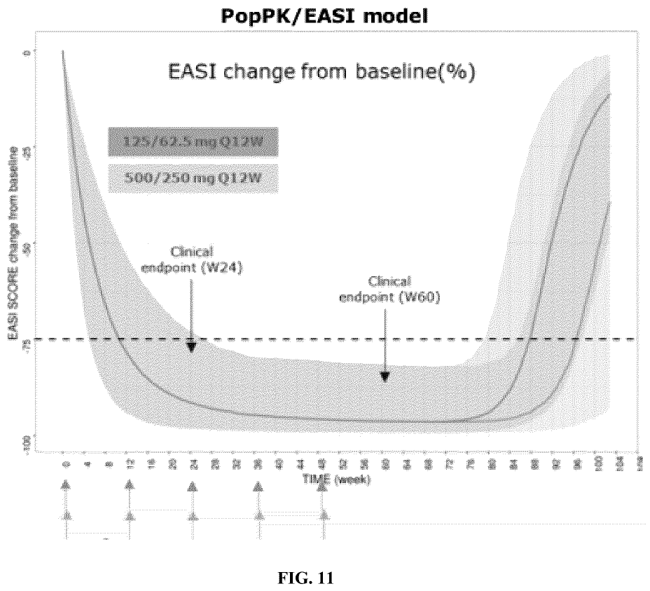

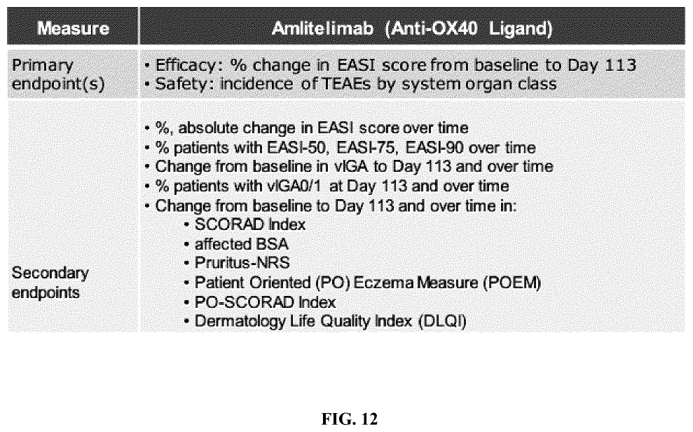

### See FIGS. 1-13.

### Safety Summary

Amlitelimab was well tolerated and demonstrated an acceptable safety profile across all 4 dose arms with no safety concerns identified in both the Phase 1 healthy volunteers' studies (KY1005-CT01 [NCT03161288], KY1005-CT04 [NCT04449939]) and Phase 2a study KY1005-CT02 [NCT03754309]. Results from the Phase 2a study in participants with AD also indicated that amlitelimab was effective and provided treatment benefit on signs and symptoms of moderate-to-severe AD.

Slightly increased incidence of overall TEAEs in pooled amlitelimab arms vs. placebo (67.1% vs. 60.3%) with comparable incidence in treated arms

No significant difference in profile across dose groups.

In the study, treatment with amlitelimab resulted in statistically significant improvements in average Eczema Area and Severity Index (EASI) score from baseline at 16 weeks compared to placebo for all four subcutaneous doses that were studied. There were also improvements in key secondary endpoints including achievement of at least 75% reduction in EASI from baseline, an Investigator Global Assessment (IGA) score of 0 (clear) or 1 (almost clear) with a reduction from baseline of at least 2 points, and proportion of patients with improvement (reduction) of weekly average of pruritus Numerical Rating Scale (NRS) of at least 4. Continued treatment improvements on these measures were observed through week 24.

A dose response effect was observed in ADA response with a higher incidence of treatment-emergent ADA at 62.5 mg Q4W (28.2%) and 125 mg Q4W (13.2%) compared to 250 mg Q4W with or without loading (2.6% and 3.8%, respectively).

### Change in Blood Biomarkers, Eosinophil Counts and LDH

Treatment effects on blood biomarkers were assessed through measurement of serum total IgE, IL-13, IL-17A, IL-22 and TARC at baseline and weeks 4, 16, 24, and measurement of serum IL-5, IL-31 and plasma eotaxin-3 at baseline and week 16. Treatment effects on blood eosinophil counts and lactate dehydrogenase (LDH) were additionally assessed at baseline and weeks 4, 16 and 24. Analyses of treatment effects on blood biomarkers were performed on the non-rescue medication population as it was determined to be more relevant to the mechanism of action of amlitelimab.

Blood levels of the evaluated biomarkers at baseline for the non-rescue medication population are presented as the median (Q1, Q3) in Table 41. All the evaluated biomarkers showed an asymmetric distribution with a higher mean versus median versus value at baseline. Overall, there appeared to be a potential mild imbalance of biomarker levels at baseline with generally higher median and mean levels observed for the 250 mg Q4W with 500 mg loading dose treatment arm and generally lower median and mean levels observed for placebo.

TABLE 41
Analysis value at baseline (Median, Q1-Q3)

| | | | | | | Eotaxin- | | | | Blood |
|---|---|---|---|---|---|---|---|---|---|---|
| Treatment | IgE | IL-13 | IL-17 | IL-22 | TARC | 3 | IL-5 | IL-31 | LDH | eosinophils |
| group | (kU/L) | (pg/mL) | (pg/mL) | (pg/mL) | (pg/mL) | (pg/mL) | (pg/mL) | (pg/mL) | (IU/L) | (10⁹/L) |
| 250 mg | 2731 | 17 | 0.38 | 19.5 | 1205 | 324 | 0.32 | 0.42 | 215 | 0.42 |
| (500 mg LD) | (254, | (6.1, 42) | (0.23, | (7.42, | (642.25, | (154, | (0.15, | (0.12, | (181, | (0.21, 0.71) |
| Amlitelimab | 8728) | | 0.66) | 54) | 2922.5) | 606) | 0.63) | 1.3) | 265) | |
| n = 77 | | | | | | | | | | |
| Amlitelimab | 603 | 12 | 0.37 | 14 (6.1, | 1150 | 226 | 0.18 | 0.28 | 211.5 | 0.33 |
| 250 mg | (187, | (4.85, | (0.2, | 62) | (441, | (136, 444) | (0.07, | (0.08, | (166.25, | (0.16, 0.59) |
| (no LD) | 5970) | 31) | 0.61) | | 2670) | | 0.53) | 1.2) | 261.75) | |
| n = 78 | | | | | | | | | | |
| Amlitelimab | 958 | 16 | 0.34 | 16.5 | 1020 | 300 | 0.32 | 0.33 | 223 | 0.34 |
| 125 mg | (96.4, | (5.35, | (0.25, | (4.98, | (552, | (169, 641) | (0.14, | (0.11, | (183, | (0.19. 0.65) |
| n = 77 | 7465) | 39) | 0.55) | 53.5) | 4030) | | 0.66) | 1.33) | 272) | |
| Amlitelimab | 1059 | 15 | 0.32 | 19 | 1080 | 255 | 0.26 | 0.31 | 214 | 0.32 |
| 62.5 mg | (206, | (6.4, | (0.21, | (7.5, | (507.75, | (144, | (0.14, | (0.14, | (181.25, | (0.2, 0.56) |
| n = 78 | 4065) | 29.5) | 0.46) | 48.5) | 2772.5) | 428.75) | 0.47) | 0.88) | 266.5) | |
| Placebo | 1336 | 11 | 0.31 | 14 | 981 | 225 | 0.21 | 0.26 | 210 | 0.26 |
| n = 78 | (148, | (5.5, 25) | (0.19, | (5.77, | (448, | (118.75, | (0.12, | (0.08, | (169.5, | (0.13, 0.42) |
| | 6750) | | 0.48) | 39.75) | 2840) | 400.75) | 0.4) | 0.99) | 249.75) | |
| Amlitelimab | 7840.19 | 27.51 | 0.46 | 79.38 | 3566.95 | 476.16 | 0.55 | 1.47 | 237.39 | 0.59 |
| 250 mg | (+/−1376.56) | (+/−3.43) | (+/−0.04) | (+/−18.36) | (+/−748.73) | (+/−61.85) | (+/−0.08) | (+/−0.28) | (+/−9.64) | (+/−0.07) |
| (500 mg LD) | | | | | | | | | | |
| n = 77 | | | | | | | | | | |
| Amlitelimab | 5752.56 | 28.34 | 0.53 | 61.99 | 3457.09 | 371.85 | 0.49 | 2.20 | 225.49 | 0.49 |
| 250 mg | (+/−1217.55) | (+/−4.61) | (+/−0.08) | (+/−11.38) | (+/−737.83) | (+/−46.16) | (+/−0.11) | (+/−1.02) | (+/−8.07) | (+/−0.07) |
| (no LD) | | | | | | | | | | |
| n = 78 | | | | | | | | | | |
| Amlitelimab | 7890.65 | 25.80 | 0.47 | 63.38 | 4601.74 | 433.90 | 0.53 | 1.96 | 238.62 | 0.51 |
| 125 mg | (+/−1596.93) | (+/−3.09) | (+/−0.04) | (+/−14.03) | (+/−1014.39) | (+/−45.82) | (+/−0.08) | (+/−0.54) | (+/−9.38) | (+/−0.06) |
| n = 77 | | | | | | | | | | |
| Amlitelimab | 3885.88 | 24.11 | 0.40 | 50.01 | 2077.91 | 326.85 | 0.41 | 1.17 | 235.62 | 0.44 |
| 62.5 mg | (+/−751.25) | (+/−3.67) | (+/−0.04) | (+/−11.04) | (+/−304.73) | (+/−29.28) | (+/−0.05) | (+/−0.29) | (+/−8.86) | (+/−0.05) |
| n = 78 | | | | | | | | | | |
| Placebo | 5750.56 | 19.11 | 0.43 | 48.54 | 2025.92 | 329.18 | 0.52 | 0.76 | 220.28 | 0.38 |
| n = 78 | (+/−1152.42) | (+/−2.53) | (+/−0.06) | (+/−10.74) | (+/−274.24) | (+/−37.22) | (+/−0.16) | (+/−0.14) | (+/−7.50) | (+/−0.05) |

Ig: immunoglobulin;
IL: interleukin;
LD: loading dose;
LDH: lactate dehydrogenase;
n: number of participants with non-missing value per treatment arm;
Q1: 1^{st} quartile,
Q3: 3^{rd} quartile;
TARC: thymus and activation-regulated chemokine.
Values are presented in original scale
Ig: immunoglobulin;
IL: interleukin;
LD: loading dose;
LDH: lactate dehydrogenase;
n: number of participants with non-missing value per treatment arm;
SEM: Standard Error of the Mean;
TARC: thymus and activation-regulated chemokine.

TABLE 42
Fold change from baseline to week 4, week 16 and week 24 - estimate (95% CI) p-value

| Visit | | | | | | | |
|---|---|---|---|---|---|---|---|
| Treatment | | | | | | | |
| arm | IgE | Eos | IL-13 | II-22 | IL-17A | TARC | LDH |
| Placebo | 1.15 | 1.13 | 0.96 | 0.89 | 1.06 | 0.96 | 1.00 |
| n = 75 | (1.04, 1.28) | (0.96, 1.32) | (0.80, 1.15) | (0.71, 1.12) | (0.90, 1.26) | (0.79, 1.17) | (0.95, 1.06) |
| | 0.007 | 0.136 | 0.671 | 0.328 | 0.481 | 0.702 | 0.869 |
| Week 16 | | | | | | | |
| Amlitelimab | 0.75 | 0.50 | 0.42 | 0.34 | 0.62 | 0.41 | 0.85 |
| 250 mg | (0.67, 0.84) | (0.42, 0.59) | (0.35, 0.51) | (0.27, 0.43) | (0.52, 0.74) | (0.34, 0.50) | (0.81, 0.90) |
| (500 mg LD) | <0.001 | <0.001 | <0.001 | <0.001 | <0.001 | <0.001 | <0.001 |
| n = 69 | | | | | | | |
| Amlitelimab | 0.79 | 0.64 | 0.46 | 0.42 | 0.67 | 0.52 | 0.87 |
| 250 mg | (0.71, 0.89) | (0.54, 0.76) | (0.38, 0.56) | (0.33, 0.54) | (0.56, 0.81) | (0.42, 0.64) | (0.82, 0.92) |
| (no LD) | <0.001 | <0.001 | <0.001 | <0.001 | <0.001 | <0.001 | <0.001 |
| n = 68 | | | | | | | |
| Amlitelimab | 0.79 | 0.62 | 0.42 | 0.40 | 0.71 | 0.48 | 0.86 |
| 125 mg | (0.71, 0.89) | (0.53, 0.74) | (0.35, 0.50) | (0.31, 0.51) | (0.60, 0.85) | (0.39, 0.58) | (0.82, 0.91) |
| n = 70 | <0.001 | <0.001 | <0.001 | <0.001 | <0.001 | <0.001 | <0.001 |
| Amlitelimab | 0.76 | 0.64 | 0.48 | 0.40 | 0.68 | 0.53 | 0.85 |
| 62.5 mg | (0.68, 0.84) | (0.54, 0.75) | (0.40, 0.58) | (0.31, 0.50) | (0.57, 0.82) | (0.44, 0.65) | (0.80, 0.89) |
| n = 73 | <0.001 | <0.001 | <0.001 | <0.001 | <0.001 | <0.001 | <0.001 |
| Placebo | 1.04 | 1.06 | 1.00 | 0.86 | 1.15 | 0.92 | 0.98 |
| n = 63 | (0.93, 1.16) | (0.89, 1.25) | (0.83, 1.20) | (0.68, 1.10) | (0.96, 1.38) | (0.75, 1.14) | (0.93, 1.04) |
| | 0.500 | 0.525 | 0.976 | 0.224 | 0.120 | 0.452 | 0.534 |
| Week 24 | | | | | | | |
| Amlitelimab | 0.63 | 0.48 | 0.34 | 0.28 | 0.55 | 0.36 | 0.81 |
| 250 mg | (0.56, 0.70) | (0.40, 0.56) | (0.29, 0.42) | (0.22, 0.36) | (0.46, 0.66) | (0.29, 0.44) | (0.77, 0.86) |
| (500 mg LD) | <0.001 | <0.001 | <0.001 | <0.001 | <0.001 | <0.001 | <0.001 |
| n = 67 | | | | | | | |
| Amlitelimab | 0.71 | 0.64 | 0.41 | 0.32 | 0.62 | 0.51 | 0.83 |
| 250 mg | (0.63, 0.80) | (0.54, 0.76) | (0.34, 0.49) | (0.25, 0.42) | (0.51, 0.75) | (0.41, 0.63) | (0.78, 0.88) |
| (no LD) | <0.001 | <0.001 | <0.001 | <0.001 | <0.001 | <0.001 | <0.001 |
| n = 64 | | | | | | | |
| Amlitelimab | 0.64 | 0.58 | 0.36 | 0.36 | 0.65 | 0.41 | 0.83 |
| 125 mg | (0.58, 0.72) | (0.49, 0.68) | (0.30, 0.43) | (0.26, 0.45) | (0.54, 0.78) | (0.34, 0.50) | (0.79, 0.88) |
| n = 68 | <0.001 | <0.001 | <0.001 | <0.001 | <0.001 | <0.001 | <0.001 |
| Amlitelimab | 0.69 | 0.57 | 0.45 | 0.36 | 0.69 | 0.55 | 0.86 |
| 62.5 mg | (0.62, 0.77) | (0.49, 0.67) | (0.37, 0.54) | (0.29, 0.46) | (0.58, 0.82) | (0.45, 0.67) | (0.81, 0.90) |
| n = 70 | <0.001 | <0.001 | <0.001 | <0.001 | <0.001 | <0.001 | <0.001 |
| Placebo | 1.03 | 1.23 | 1.05 | 0.90 | 1.06 | 1.01 | 1.02 |
| n = 57 | (0.92, 1.15) | (1.04, 1.47) | (0.86, 1.27) | (0.70, 1.15) | (0.88, 1.27) | (0.82, 1.26) | (0.97, 1.08) |
| | 0.639 | 0.018 | 0.635 | 0.383 | 0.564 | 0.899 | 0.418 |

CI: confidence interval;
Eos: eosinophil;
Ig: immunoglobulin;
IL: interleukin;
LD: loading dose;
LDH: lactate dehydrogenase;
n: number of participants with non-missing values per treatment arm;
TARC: thymus and activation-regulated chemokine.

TABLE 43
Fold change from baseline to week 16 - estimate (95% CI) p-value

| | Visit | | |
|---|---|---|---|
| | Treatment arm | | |
| Week 16 | Eotaxin-3 | IL-5 | IL-31 |
| Amlitelimab 250 mg | 0.65 | 0.38 | 0.26 |
| (500 mg LD) | (0.55, 0.77) | (0.29, 0.50) | (0.18, 0.40) |
| n = 69 | <0.001 | <0.001 | <0.001 |
| Amlitelimab 250 mg | 0.62 | 0.48 | 0.45 |
| (no LD) | (0.52, 0.74) | (0.35, 0.64) | (0.29, 0.68) |
| n = 66 | <0.001 | <0.001 | <0.001 |
| Amlitelimab 125 mg | 0.60 | 0.40 | 0.40 |
| n = 70 | (0.51, 0.71) | (0.30, 0.53) | (0.27, 0.59) |
| | <0.001 | <0.001 | <0.001 |
| Amlitelimab 62.5 mg | 0.64 | 0.53 | 0.41 |
| n = 73 | (0.55, 0.75) | (0.41, 0.70) | (0.28, 0.62) |
| | <0.001 | <0.001 | <0.001 |
| Placebo | 1.03 | 0.98 | 1.12 |
| n = 63 | (0.87, 1.22) | (0.74, 1.29) | (0.73, 1.70) |
| | 0.761 | 0.863 | 0.605 |

CI: confidence interval, IL: Interleukin; LD: loading dose; n: number of participants with non-missing value per treatment arm.

Fold changes from baseline at week 16 and week 24 in the amlitelimab group were significantly lower than both baseline and placebo for all evaluated biomarkers (Tables 42 and 43). A consistent trend was observed for a numerically greater fold reduction of most measured biomarkers evaluated at both week 16 and week 24 in the 250 mg Q4W with 500 mg loading dose treatment arm. In addition, a trend consistent with a dose-response was observed for select biomarkers including IL-13, TARC, IL-17A and IgE. Of the biomarkers evaluated, only eosinophil counts in the 250 mg Q4W with 500 mg loading dose arm showed significant fold change from baseline at week 4. A reduction in levels of LDH was observed over time, expected in AD participants who are improving.

Atopic dermatitis features hyperactive and dysregulated Th2 and Th22 inflammatory responses as a key contributor to disease pathophysiology with varying degrees of contribution of Th1/Th17 inflammatory pathways across disease endotypes. Consistent with targeting an upstream costimulatory pathway, amlitelimab treatment reduced levels of Th2 cytokines: IL-13, IL-5, IL-31, Th2-associated chemokines: TARC/CCL17 and Eotaxin-3/CCL26, as well as levels of Th22/Th17 associated cytokines: IL-22 and IL-17A with the largest effect observed at the highest dose with loading dose. In addition, amlitelimab treatment significantly reduced total IgE levels and blood eosinophil counts, both of which are known to be elevated in atopic diseases including AD, and of which only IgE is currently modulated by currently approved AD biologics.

### Example II—Summary of Population PK (PopPK) and Population PK/EASI Modeling

A population PK (PopPK) model was developed and validated to assess the PK of amlitelimab. A total of 439 participants were included in the PopPK model, i.e., 78 (18%) healthy subjects from phase 1 studies (KY1005-CT01, KY1005-CT04 and PKM17597), and 361 (82%) participants with moderate-to-severe AD (59 from study KY1005-CT02 and 302 from study KY1005-CT05/DRI17366 [STREAM-AD]).

The median age of the study population was 33 years (ranging from 18 to 72 years). The majority of participants were male (62%), 83% of participants were Caucasian and 11% were Asian. Overall, the median body weight was 74.5 kg with body weight ranging from 40.5 to 148 kg.

The selection of the proposed dose and regimens for phase 3 clinical trials is informed by the analysis of the 24-week safety, efficacy, and exposure-response data from the KY1005-CT05/DRI17366 (STREAM-AD), which evaluated 4 doses of amlitelimab administered SC (250 mg Q4W with or without a loading dose of 500 mg, 125 mg Q4W and 62.5 mg Q4W) versus placebo. While all amlitelimab dose regimens demonstrated statistical superiority compared to placebo on the primary and key secondary endpoints, the 250 mg Q4W regimen with a 500 mg loading dose consistently demonstrated the greatest effect size at week 24 and the largest separation from placebo at early timepoints in responder analyses suggesting more rapid disease control. These findings are also supported by observations made across multiple disease activity biomarkers.

Dose regimen to be studied in Phase 3 are:
Amlitelimab 250 mg Q4W with 500 mg loading dose,
Amlitelimab 250 mg Q12W with 500 mg loading dose,
Matching placebo Q4W with matching placebo loading dose

The less frequent dosing intervals (Amlitelimab 250 mg Q12W with 500 mg loading dose) are expected to result in drug exposure levels that are in the range of those studied in the Phase 2b (KY1005-CT05/DRI17366 [STREAM-AD]) (i.e., between 62.5 mg Q4W and 250 mg Q4W with 500 mg loading dose) and that demonstrated efficacy and an acceptable safety profile.

Additional rationale for studying an extended dosing interval includes bringing added value for participants by reducing the frequency of injections and increasing convenience and adherence to treatment. This can improve QoL for participants with chronic conditions by reducing burden of treatment. Over the longer term, extending subcutaneous regimen may reduce healthcare visits and associated procedures (i.e., health care resource utilization).

The lowest dose studied in Phase 2b (KY1005-CT05/DRI17366 [STREAM-AD]) study was 62.5 mg Q4W and considered to be as comparatively efficacious and safe as the 250 mg Q4W with 500 mg loading dose. The efficacy results observed on lesions and itch in this dose arm were clinically meaningful and nominally statistically significant at Week 16 and Week 24. Regarding biomarkers, the 62.5 mg Q4W dose arm also significantly reduced AD relevant biomarkers including IgE, eosinophil counts, TARC, and IL-22 at week 16 and week 24 and IL-31 at week 16 compared to placebo.

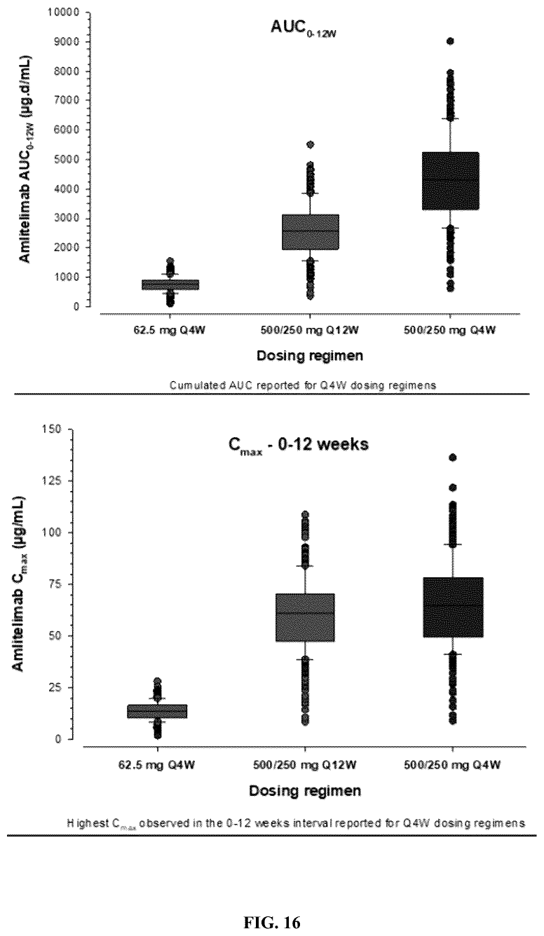

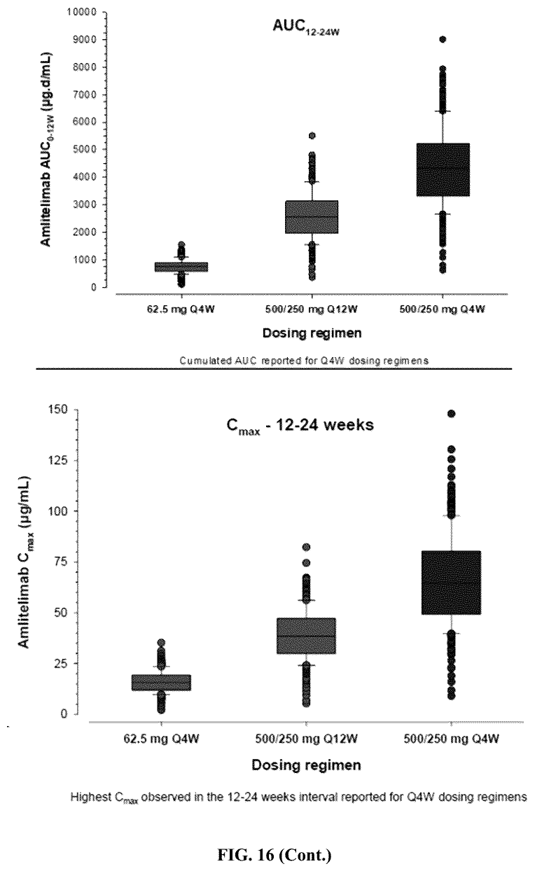

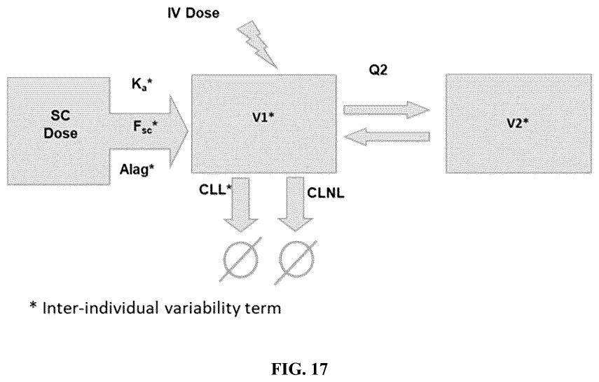

Simulations performed in participants from KY1005-CT05/DRI17366 (STREAM-AD) based on the Population PK model showed that exposures after 250 mg Q12W with a 500 mg loading dose are predicted to be in the range of the exposures documented in Study KY1005-CT05/DRI17366 (STREAM-AD), i.e., in between 62.5 mg Q4W and 250 mg Q4W with 500 mg loading dose (FIG. 16).

### Summary of Population PK (Pop-PK) Study

### Methodology:

Five clinical studies composed the data set used to develop the PopPK model. Details are given in Table 44.

TABLE 44
Summary of clinical studies included in the population PK analysis.

| | | | | Dosing | Database |
|---|---|---|---|---|---|
| Study | Phase | subjects | Route | regimen (mg) | Lock Date |
| CT-01 | 1 | 58 HV | IV | SD: 0.006 to 0.05 mg/kg | completed |
| | | | | 0.15 + 0.075 × 2 to | |
| | | | | 12.0 + 6.0 × 2 mg/kg | |
| | | | | Q4W (3 doses) | |
| CT-04 | 1 | 24 HV | 8 IV/16 | SD: 125 and 250 mg | completed |
| | | | SC | | |
| PKM17597 | 1 | 6 on 32 | SC | SD: 250 mg | Part 1 completed |
| | | HV (part1) | | | (Part 2 ongoing |
| | | | | | and not included) |
| CT-02 | 2 | 59 AD | IV | 1 × 200 mg + | completed |
| | | patients | | 3 × 100 mg; | |
| | | | | 1 × 500 mg + | |
| | | | | 250 × 3 | |
| | | | | Q4W (12 weeks) | |
| DRI17366 | 2 | 302 AD | SC | 62.5; 125; | On going data |
| | | patients | | 250; 1 × 500 | available |
| | | | | mg + 250 mg | as of |
| | | | | Q4W up to 52 weeks | 2 May 2023 |

HV: Healthy volunteer;
AD: Atopic dermatitis patient

### PopPK Analysis

The PopPK analysis was performed with the NONMEM computer program (version 7.5.1) running on a Linux cluster of multi-processor computers. All runs were performed using the First Order Conditional Estimate method with Interaction option during the development of the model.

Once outliers detected from a preliminary pharmacostatistical model, the Initial Dataset was renamed Total Dataset. Initially the population parameters (fixed and random effects) together with the individual estimates were computed assuming no dependency between

### PK Parameters and Covariates.

Based on a preliminary model, a 2-compartment structure with an absorption part characterized by a first order constant of absorption, a lag time and a bioavailability factor was used. One (linear or no linear) or two (linear+no linear) time-dependent or time-independent clearance(s) completed with proportional, additional or combined residual error model were evaluated in the current study.

The relationship between the individual estimates and the covariates was investigated.

The baseline demographic characteristics such as body weight (except for the previously allometric-scaled parameters), age, gender, race, renal function (CLCR in mL/min), alkaline phosphatase (ALP in IU/L), alanine amino transferase (ALT in IU/L), aspartate amino transferase (AST in IU/L), bilirubin (BILI in umol/L) and the albumin level (ALB in g/L) were considered.

The occurrence of anti-drug antibodies either coded as binary covariate (at least once/never over study follow-up coded as ADAMAX) or longitudinal binary covariate (ADA), the two clinical endpoints EASI and vIGA baseline scores as well as healthy versus AD patient status coded as binary covariate (IND) were also investigated.

Due to the specificity of the dataset, including adults with baseline bodyweight varying from 40.5 kg to 148 kg allometric scaling of the parameters was privileged and tested before the covariate selection process.

The selected covariates were added individually (forward selection method) to the model and tested for statistical significance. The covariates providing a significant change (p<0.05, log likelihood ratio test) in the objective function (OFV), when introduced in the model, were retained in the model. A backward deletion was performed to assess the covariate parameters relationships. Only the covariates associated with a significant change of the objective function with a p value <0.001 were retained in the final model. The population parameters were then re-estimated considering the relationship with the covariates. Before qualification, model verification was performed by examination of the goodness-of-fit plots and by estimation of several quality criteria such as bias, precision or Absolute Average Fold Error.

The validation of the PopPK model predictive ability was performed using different approaches (examination of individual concentration versus time curves, Prediction-Corrected Visual Predictive Check and Sampling Important Resampling).

Finally, the final PopPK model was used to calculate the individual PK parameters estimates and derived exposure variables (Cmax, Ctrough and AUC) for AD patients treated with Q4W SC doses corresponding to study DRI17366, evaluating alternative dosing regimens proposed for the future phase3 clinical studies. These simulations were completed by the generation of virtual patients to further evaluate the impact of the included covariates on the exposure parameters.

### Results

The characteristics of the subjects included in the PopPK analysis are presented in Table 45.

TABLE 45
Demographic characteristics (baseline values) of the patients included in the Initial Dataset.

| | CT01 | | CT04 | | PKM17597 | | CT02 | | DRI17366 | | Total | |
|---|---|---|---|---|---|---|---|---|---|---|---|---|
| Parameter | (n = 48) | | (n = 24) | | (n = 6) | | (n = 59) | | (n = 302) | | (n = 439) | |
| Age (yrs) | 26.1 | (5.78) | 32.9 | (6.4) | 36.7 | (13.7) | 34.4 | (12.4) | 37.9 | (14.5) | 35.9 | (13.7) |
| | 24 | [18-44] | 33 | [21-45] | 31.5 | [19-55] | 33 | [18-66] | 36 | [18-72] | 33 | [18-72] |
| 18-64 yrs | 48 | (100%) | 24 | (100%) | 6 | (100%) | 58 | (98.3%) | 285 | (94.4%) | 421 | (95.9%) |
| ≥65 yrs | 0 | (0%) | 0 | (0%) | 0 | (0%) | 1 | (1.7%) | 17 | (5.6%) | 18 | (4.4%) |
| Body weight (kg) | 77.4 | (9.77) | 76.3 | (8.89) | 75.3 | (15.5) | 73.3 | (14.4) | 75.5 | (17.7) | 75.5 | (16.2) |
| | 75.7 | [60.1-99.2] | 76.2 | [60.2-94.5] | 74.8 | [55-99] | 73 | [42-119] | 74.2 | [40.5-148] | 74.5 | [40.5-148] |
| <50 kg | (0%) | | (0%) | | (0%) | | 2 | (3.4%) | 14 | (4.64%) | 16 | (3.64%) |
| 50-99 kg | 48 | (100%) | 24 | (100%) | 6 | (100%) | 55 | (93.2%) | 260 | (86.1%) | 393 | (89.5%) |
| ≥100 kg | 0 | (0%) | 0 | (0%) | 0 | (0%) | 2 | (3.4%) | 28 | (9.27%) | 30 | (6.83%) |
| BMI (kg/m²) | 23.1 | (2.28) | 24.1 | (2.19) | 25.4 | (3.68) | 24.9 | (3.93) | 26.0 | (5.39) | 25.4 | (4.90) |
| | 22.6 | [18.6-28.8] | 24.8 | [19.4-27.3] | 25 | [20.4-30] | 24.7 | [17.4-36.7] | 25.1 | [16.4-51.4] | 24.7 | [16.4-51.4] |
| Albumin (g/L) | 50.0 | (2.02) | 40.9 | (1.54) | NA | | 45.4 | (3.06) | 46.5 | (3.16) | 46.4 | (3.45) |
| | 50 | [46-57] | 41 | [37-44] | | | 45 | [37-53] | 47 | [38-55] | 47 | [37-57] |
| total bilirubin | 13.6 | (7.58) | 17.4 | (5.55) | 9.69 | (2.55) | 7.16 | (3.87) | 7.89 | (4.99) | 8.95 | (5.89) |
| (μmol/L) | 10.5 | [6-38] | 16.0 | [9.20-32.9] | 9.41 | [6.84-13.7] | 5.13 | [1.71-22.2] | 6.50 | [2.60-31] | 7 | [1.71-38] |
| Aspartate Amino | 23.5 | (6.98) | 23.5 | (4.13) | 18.8 | (3.89) | 29.8 | (14.5) | 23.6 | (10.3) | 24.4 | (10.6) |
| Transferase | 22 | [14-43] | 23 | [18-31] | 18 | [14-26] | 25 | [15-80] | 21 | [10-120] | 22 | [10-120] |
| (IU/L) | | | | | | | | | | | | |
| Alanine amino | 22.4 | (13.8) | 21.9 | (9.43) | 22.5 | (6.75) | 29.0 | (20.9) | 23.0 | (15.6) | 23.7 | (16.0) |
| transferase (IU/L) | 19.5 | [7-74] | 19 | [12-49] | 21 | [15-33] | 24 | [5-143] | 19 | [5-141] | 20 | [5-143] |
| Alkaline | 79.5 | (19.3) | 53.0 | (12.8) | 65.7 | (17.9) | 69.9 | (21.2) | 75.9 | (21.9) | 74.1 | (21.9) |
| phosphatase | 75.5 | [46-131] | 51 | [30-82] | 58.5 | [46-94] | 68 | [24-139] | 73 | [35-162] | 71 | [24-162] |
| (IU/L) | | | | | | | | | | | | |
| creatinine | 133 | (23.5) | 132 | (17.6) | 125 | (49.6) | 125 | (33.0) | 140 | (43.9) | 137 | (40.2) |
| clearance | 132 | [86.0-201] | 130 | [93.8-164] | 116 | [68.4-222] | 124 | [52.3-256] | 135 | [56.1-310] | 132 | [52.3-310] |

| (mL/min) | | | | | | | | | |
|---|---|---|---|---|---|---|---|---|---|
| EASI | NA (0) | NA (0) | NA (0) | 31.3 | (12.4) | 29.3 | (11.0) | 29.7 | (11.3) |
| | | | | 29.1 | [16.1-63.6] | 27.3 | [14.6-69.6] | 27.4 | [14.6-69.6] |
| Severe (>21) | NA (0) | NA (0) | NA (0) | 46 | (78.0%) | 217 | (71.9%) | 263 | (72.9%) |
| Moderate (<=21) | NA (0) | NA (0) | NA (0) | 13 | (22.0%) | 85 | (28.1%) | 98 | (27.1%) |

| | | | | | | |
|---|---|---|---|---|---|---|
| vIGA-AD (3/4) | NA (0) | NA (0) | NA (0) | 38 (64.4%)/ | 217 (71.9%)/ | 255 (70.6%)/ |
| | | | | 21 (35.6%) | 85 (28.1%) | 106 (29.4%) |
| Male-Female | 100%-0% | 100%-0% | 66.7%-33.3% | 54.2%-45.8% | 54.3%-45.7% | 62%-38% |
| Caucasian/ | 41 (85.4)/ | 16 (66.7)/ | 6 (100)/0 | 59 | 241 (79.8)/ | 363 (82.7)/19 |
| Black/Asian/Others | 2 (4.2)/1 | 3 (12.5)/2 | (0)/0 (0)/0 | (100)/0 | 14 (4.64)/ | (4.33)/49 |
| | (2.1)/4 | (8.3)/3 | (0) | (0)/0 | 46 (15.2)/ | (11.2)/8(1.82) |
| | (8.3) | (12.5) | | (0)/0 (0) | 1 (0.33) | |
| ADAMAX (0/1) | 31 | 9 | 6 (100%)/ | 42 | 271 | 359 (81.8%)/ |
| | (64.6%)/17 | (37.5%)/15 | 0 (0%) | (71.2%)/ | (89.7%)/31 | 80 (18.2%) |
| | (35.4%) | (62.5%) | | 17 | (10.3%) | |
| | | | | (28.8%) | | |

Prior to model selection, additional data cleaning and outliers' detection led to the deletion of 82 (1.73%) samples and the exclusion of only one AD patient of the DRI17366 who had only one concentration time point available at the time of the analysis. A total of 438 subjects (including 301 AD patients from the study DRI17366) and 4660 concentration time points have been included in the Final Dataset of the PopPK analysis

The pharmacostatistical model was a two-compartment model with both a linear (first order process) clearance, (CL) and non-linear, Michaelis Menten, clearance (characterized by Vm and Km parameters) from the central compartment. The two compartments were represented by distribution volumes (V1, and V2 for the central and the peripheral compartments, respectively) linked by one inter-compartmental clearance (Q2). The SC absorption process was described by a first-order constant of absorption (Ka) from the SC compartment to the central compartment (V1), a lag time (ALAG) and a bioavailability factor (Fsc) as depicted in Error! Reference source not found.

After allometric scaling and covariate selection processes, the final PopPK model included individual estimated (no fixed values) baseline bodyweight dependent allometric scaling factors on CL, V1 and V2 parameters. In addition, a significant effect of the baseline EASI score on CL and baseline albumin on Fsc parameters was identified.

The CL parameter was related to the bodyweight (BWT) and the EASI score (BEASI) by the following equation:

$$CL = TVCL \times {( \frac{B W T}{75} )}^{AlloCL} + COV 1 * BEASI$$

The V1 parameter was related to the bodyweight (BWT) by the following equation:

$$V 1 = TVVI \times {( \frac{B W T}{75} )}^{AlloV 1}$$

The V2 parameter was related to bodyweight (BWT) and by the following equation:

$$V 2 = TVV2 \times {( \frac{B W T}{75} )}^{AlloV 2}$$

The Fsc parameter was related to albumin (BALB) by the following equation:

$$Fsc = TVFsc + COV 2 * ( ( B ALB / 47 ) - 1 )$$

As summarized in the supportive information (Section 1.2), no important systematic deviations or major bias in any of the goodness of fit plots were observed. The performance and quality criteria of the model stratified by route of administration (IV or SC) or subject status (AD patients or HV subjects) were comparable. The predictive performances using Prediction-Corrected Visual Predictive Check technique and the evaluation of the uncertainties of the estimate values using Sampling Importance Resampling approach qualified the model.

The final population PK parameters and their relationship to covariates are presented in Table 46.

TABLE 46
Final population PK parameters and their relationship to covariates

| Parameter | Estimate (CV) | RSE (%) | 95% CI (shrinkage) |
|---|---|---|---|
| TVV1 (L) | 3.46 | 2.30% | [3.3; 3.62] |
| TVV2 (L) | 2.48 | 3.12% | [2.33; 2.64] |
| TVCLL (L/day) | 0.115 | 2.77% | [0.109; 0.122] |
| TVQ2 (L/day) | 0.569 | 7.36% | [0.485; 0.653] |
| TVFsc | 0.888 | 1.88% | [0.855; 0.921] |
| TVVM (μg/day) | 0.0362 | 12.6% | [0.027; 0.0453] |
| TVKM (μg/mL) | 0.0783 | 21.6% | 0.0445; 0.112] |
| TVALAG (day) | 0.0351 | 8.61% | [0.029; 0.0411] |
| TVKa (day⁻¹) | 0.233 | 4.96% | [0.21; 0.257] |
| BWT effect on V1 | 0.901 | 10.7% | [0.708; 1.09] |
| BWT effect on V2 | 0.35 | 35.8% | [0.0994; 0.601] |
| BWT effect on CL | 1.2 | 6.69% | [1.04; 1.36] |
| BEASI effect on CL | 0.00111 | 9.84% | [0.000889; 0.00132] |
| BALB effect on Fsc | 0.598 | 21.8% | [0.338; 0.858] |
| Inter-individual variability | | | |
| ω² V1 | 0.0491 (22.4%) | 18.2% | [0.0316; 0.0667] |
| | | | (35.1%) |
| ω block(V1, CL) | 0.024 | 18.2% | [0.0155; 0.0325] |
| ω² CL | 0.0482 (22.2%) | 9.55% | [0.0391; 0.0572] |
| | | | (17.3%) |
| ω² V2 | 0.0693 (26.8%) | 28.70% | [0.0303; 0.108] |
| | | | (42.9%) |
| ω² Fsc | 1.18 | 18.10% | [0.761; 1.6] |
| | | | (42.8%) |
| ω² ALAG | 0.151 (40.3%) | 35.3% | [0.0464; 0.255] |
| | | | (78.5%) |
| ω² Ka | 0.135 (38%) | 26.5% | [0.0649; 0.206] |
| | | | (45.4%) |
| Residual variability | | | |
| σ² proportional | 0.0248 (15.8%) | 2.52% | [0.0236; 0.0261] |

RSE (%): Percentage of Relative Standard Error (100% * SE/Estimate).
95% CI: 95% confidence interval;
The coefficients of variation were calculated considering log-normal variability for parameters using CV (e^{η}) = \sqrt{(e^{ω}^{²} − 1)}

The final PopPK model was then used to estimate individual exposure parameters of AD patients from DRI13677 study receiving SC doses. Descriptive statistics on exposures are presented by dose regimen after first dose and last dose in Table 47.

TABLE 47
Descriptive statistics on amlitelimab exposures by dose arm in atopic dermatitis patients after
first dose (week 1) and last dose (week 20) following Q4W administration - Study DRI17366.

| | Cₘₐₓ (μg/mL) | | | | AUC_{4w} (μg · day/mL) | | | | C_{trough} (μg/mL) | | | |
|---|---|---|---|---|---|---|---|---|---|---|---|---|
| Dose | First dose | | Last dose | | First dose | | Last dose | | First dose | | Last dose | |
| 62.5 mg | 8.23 | (31.0%) | 17.7 | (31.9%) | 186 | (31.6%) | 412 | (32.3%) | 5.34 | (31.3%) | 11.1 | (33.8%) |
| (n = 77) | 8.38 | [2.92-13.0] | 17.2 | [5.46-31.1] | 186 | [65.4-293] | 399 | [120-731] | 5.23 | [1.83-8.51] | 10.8 | [2.72-20.7] |
| 125 mg | 14.8 | (25.9%) | 31.9 | (33.5%) | 337 | (28.0%) | 748 | (36.2%) | 9.76 | (32.3%) | 20.3 | (41.1%) |
| (n = 75) | 15.0 | [8.10-27.1] | 30.0 | [17.2-70.9] | 331 | [191-646] | 691 | [359-1718] | 9.26 | [4.76-19.9] | 18.4 | [7.83-50.0] |
| 250 mg | 29.3 | (28.6%) | 63.1 | (31.9%) | 659 | (29.7%) | 1471 | (33.7%) | 19.0 | (32.4%) | 40.0 | (37.0%) |
| (n = 73) | 29.9 | [4.19-49.8] | 64.3 | [8.77-110] | 658 | [95.5-981] | 1498 | [206-2568] | 20.0 | [2.77-33.2] | 41.5 | [5.54-72.3] |
| 500/250 | 56.0 | (30.9%) | 60.0 | (34.8%) | 1249 | (31.1%) | 1385 | (36.0%) | 34.9 | (33.5%) | 37.1 | (39.1%) |
| mg | 55.9 | [10.5-106] | 58.7 | [11.7-121] | 1244 | [242-2432] | 1309 | [278-2982] | 33.7 | [7.23-69.0] | 34.1 | [7.63-87.4] |

(n = 76)

Mean (CV %);
Median [Minimum-Maximum]

The final PopPK model was used to simulate exposures for an extended regimen of 250 mg Q12W with 500 mg loading dose for 24 weeks in all patients from Study DRI17366. Exposures for the lowest and highest dosing regimens tested in study DRI17366 (i.e., 62.5 mg Q4W and 250 mg Q4W with 500 mg loading dose for 24 weeks, respectively) were also simulated in all patients. Descriptive statistics on exposures are presented by dose regimen in Table 48.

TABLE 48
Descriptive statistics on amlitelimab predicted exposures in patients with atopic dermatitis
from DRI17366 study after 250 mg Q12W with a 500 mg loading dose compared to 62.5 mg Q4W
(without loading dose) and 250 mg Q4W with a 500 mg loading dose for 24 weeks (n = 301).

| | Cₘₐₓ (μg/mL) | | | | AUC_{12w} (μg · day/mL) | | | | C_{trough} (μg/mL) | | | |
|---|---|---|---|---|---|---|---|---|---|---|---|---|
| | 0-12 | | 12-24 | | 0-12 | | 12-24 | | 0-12 | | 12-24 | |
| Dose | weeks | | weeks | | weeks | | weeks | | weeks | | weeks | |
| 62.5 mg | 13.8 | (32.0%) | 15.9 | (33.8%) | 756 | (32.4%) | 1082 | (34.8%) | 8.67 | (36.2%) | 10.0 | (38.5%) |
| Q4W | 13.7 | [1.81-28.0] | 15.6 | [2.05-35.2] | 751 | [100-1546] | 1053 | [140-2442] | 8.40 | [1.10-19.9] | 9.82 | [1.24-24.8] |
| 500/250 | 65.6 | (31.6%) | 65.9 | (34.0%) | 4362 | (32.5%) | 4598 | (35.3%) | 40.9 | (37.2%) | 41.7 | (38.6%) |
| mg | 64.6 | [8.95-136] | 64.5 | [8.94-148] | 4321 | [615-9016] | 4468 | [632-10602] | 39.4 | [5.65-96.6] | 40.8 | [5.65-105] |
| Q4W | | | | | | | | | | | | |
| 500/250 | 60.1 | (30.0%) | 39.2 | (32.1%) | 2591 | (33.2%) | 1702 | (37.0%) | 10.8 | (46.3%) | 7.02 | (52.3%)] |
| mg | 60.9 | [8.42-109] | 38.4 | [5.27-82.2] | 2561 | [361-5506] | 1653 | [223-4145] | 10.3 | [1.14-31.1] | 6.69 | [0.54-23.0] |
| Q12W | | | | | | | | | | | | |

Mean (CV %);
Median [Minimum-Maximum]
For Q4W dosing regimens, cumulated AUC_{4w} was reported

Simulations were performed based on the final PopPK model with the objective to generate amlitelimab exposures in patients <40 kg (range: 25-40 kg) within the exposure range observed in AD patients ≥40 kg (range: 40-150 kg) at the selected dosing regimens for phase 3 program (i.e., 500 mg loading dose followed by 250 mg administered Q4W, and 500 mg loading dose followed by 250 mg administered Q12W for 24 weeks). Simulations in 1000 virtual AD patients with body weight ≥25 kg and <40 kg were performed for 24 weeks at 125 mg Q4W with a loading dose of 250 mg and 125 mg Q12W with a loading dose of 250 mg.

Descriptive statistics on predicted exposures in patients with atopic dermatitis after a loading dose followed by a Q4W dosing regimen are presented for patients <40 kg and patients ≥40 kg in Table 49. Descriptive statistics on predicted exposures after a loading dose followed by a Q12W dosing regimen are presented in Table 50.

TABLE 49
Descriptive statistics on amlitelimab predicted exposures by body weight category
in patients with atopic dermatitis after a loading dose followed by a Q4W dosing
regimen - 250/125 mg in patients <40 kg and 500/250 mg in patients ≥40 kg.

| | Body | | Cₘₐₓ (μg/mL) | | | | AUC_{4w} (μg · day/mL) | |
|---|---|---|---|---|---|---|---|---|
| | weight | | First | | Last | | First | |
| Category | (kg) | | dose | | dose | | dose | |
| <40 kg | 34.9 | (11%) | 46.5 | (26%) | 55.3 | (31%) | 1065 | (26%) |
| (n = 1000) | 35.8 | [25.1-39.99] | 45.9 | [26.6-68.9] | 53.6 | [30.9-86.0] | 1054 | [616-1565] |
| ≥40 kg | 70.6 | (20%) | 60.4 | (30%) | 65.8 | (33%) | 1340 | (29%) |
| (n = 1000) | 70.2 | [40-126] | 58.2 | [33.2-92.8] | 63.3 | [33.1-104] | 1293 | [747-2049] |

| | AUC_{4w} (μg · day/mL) | | C_{trough} (μg/mL) | | | |
|---|---|---|---|---|---|---|
| | Last | | First | | Last | |
| Category | dose | | dose | | dose | |
| <40 kg | 1314 | (33%) | 31.4 | (29%) | 36.4 | (36%) |
| (n = 1000) | 1260 | [697-2086] | 31.0 | [17.2-46.8] | 34.5 | [17.7-60.1] |
| ≥40 kg | 1528 | (35%) | 37.6 | (32%) | 41.2 | (39%) |
| (n = 1000) | 1468 | [749-2503] | 36.8 | [19.8-59.0] | 39.4 | [18.4-71.0] |

Mean (CV %);
Median [Minimum-Maximum] for body weight, Mean (CV %);
Median [5^{th}-95^{th} percentiles] for Cₘₐₓ, AUC_{4w} and C_{trough}

TABLE 50
Descriptive statistics on amlitelimab predicted exposures by body weight category
in patients with atopic dermatitis after a loading dose followed by a Q12W dosing
regimen - 250/125 mg in patients <40 kg and 500/250 mg in patients ≥40 kg.

| | Body | | Cₘₐₓ (μg/mL) | | | | AUC_{12w} (μg · day/mL) | |
|---|---|---|---|---|---|---|---|---|
| | weight | | First | | Last | | First | |
| Category | (kg) | | dose | | dose | | dose | |
| <40 kg | 34.9 | (12%) | 46.5 | (29%) | 31.9 | (31%) | 2117 | (31%) |
| (n = 1000) | 35.8 | [25.1-39.99] | 45.9 | [26.6-68.9] | 31.2 | [17.7-48.7] | 2085 | [1196-3183] |
| ≥40 kg | 70.8 | (23%) | 60.5 | (34%) | 39.4 | (36%) | 2564 | (36%) |
| (n = 1000) | 70.2 | [40-126] | 59.5 | [31.3-91.8] | 38.3 | [20.1-62.2] | 2501 | [1324-4088] |

| | AUC_{12w} (μg · day/mL) | | C_{trough} (μg/mL) | | | |
|---|---|---|---|---|---|---|
| | Last | | First | | Last | |
| Category | dose | | dose | | dose | |
| <40 kg | 1469 | (39%) | 10.0 | (52%) | 6.9 | (64%) |
| (n = 1000) | 1396 | [750-2404] | 9.28 | [3.58-18.7] | 6.08 | [1.96-14.9] |
| ≥40 kg | 1699 | (41%) | 10.8 | (55%) | 7.16 | (64%) |
| (n = 1000) | 1601 | [821-2865] | 9.98 | [3.74-21.4] | 6.23 | [2.00-15.6] |

Mean (CV %);
Median [Minimum-Maximum] for body weight, Mean (CV %);
Median [5^{th}-95^{th} percentiles] for Cₘₐₓ, AUC_{4w} and C_{trough}

### Population PK/EASI Model

Using the Population PK/EASI model developed based on Phase 2 clinical studies in AD

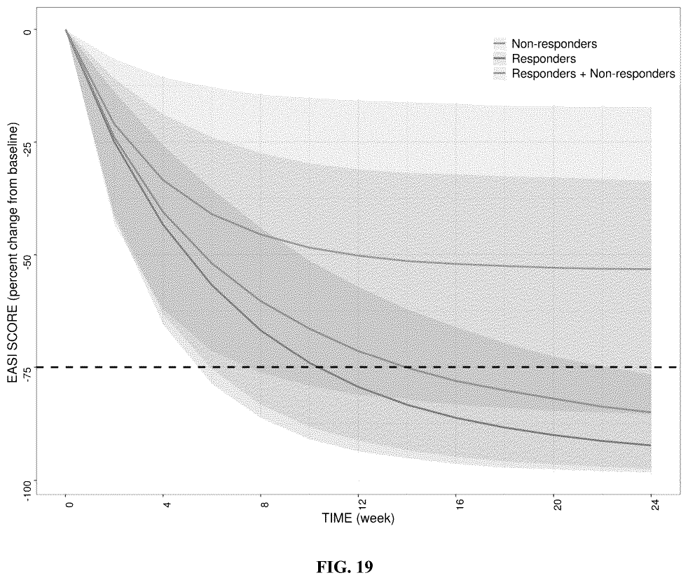

participants (KY1005-CT02 and KY1005-CT05/DRI17366 [STREAM-AD]), predicted percent change from baseline in EASI score was simulated in responder and non-responder participants at 250 mg Q12W with a 500 mg loading dose (FIG. 19). The results showed that all responders had a percent change from baseline in EASI score in the range of those observed in the Phase 2 (KY1005-CT05/DRI17366 [STREAM-AD]) clinical study. In addition, the predicted percent changes from baseline in EASI in the whole population are in accordance with Phase 2b (KY1005-CT05/DRI17366 [STREAM-AD]) (FIG. 19).

Methodology: Two clinical studies (KY1005-CT02 and KY1005-CT05/DRI17366 [STREAM-AD]) composed the data set used to develop the PopPK/EASI model. Details are given in Table 51.

TABLE 51
Summary of clinical studies included in the PopPK/EASI analysis.

| | Phase | | | Database Lock |
|---|---|---|---|---|
| Study | subjects | Route | Dosing regimen (mg) | Date |
| CT-02 | 2 | 59 AD IV | 1 × 200 mg + 3 × 100 mg; | completed |
| | | verum | 1 × 500 mg + 250 × 3 | |
| | | patients | Q4W (12 weeks) | |
| DRI17366 | 2 | 302 AD SC | 62.5; 125; 250; 1 × 500 | On going |
| | | verum | mg + 250 mg Q4W up to | data available as of |
| | | patients | 52 weeks | 2 May 2023 |

AD: Atopic dermatitis

### PopPK/EASI Analysis

The PopPK/EASI analysis was performed with the NONMEM computer program (version 7.5.1) running on a Linux cluster of multi-processor computers. All runs were performed using the First Order Conditional Estimate method with Interaction option during the development of the model.

Initially the population parameters (fixed and random effects) together with the individual estimates were computed assuming no dependency between parameters and covariates.

Direct and turnover response model (type I—loss of induction) in which amlitelimab concentrations exerted an inhibitory effect on EASI score production rate were explored. The relationship between concentration and EASI score was explored using Imax, power or linear equations. The exploration was completed by evaluating proportional, additional or combined residual error models.

The relationship between the individual estimates and the covariates was investigated. The baseline demographic characteristics such as body weight, age, gender, race, renal function (CLCR in mL/min), alkaline phosphatase (ALP in IU/L), alanine amino transferase (ALT in IU/L), aspartate amino transferase (AST in IU/L), bilirubin (BILI in μmol/L) and the albumin level (ALB in g/L) were considered.

The occurrence of anti-drug antibodies either coded as binary covariate (at least once/never over study follow-up coded as ADAMAX) or longitudinal binary covariate (ADA), were also investigated.

In order to better characterize the responder and non-responder patients, an additional covariate (RESP) was investigated. Responders were defined by achieving VIGA-AD 0 or 1 at week 16 in Study CT02 and by achieving vIGA-AD 0 or 1 and/or EASI-75 response at week 24 in study DRI17366

The selected covariates were added individually (forward selection method) to the model and tested for statistical significance. The covariates providing a significant change (p<0.05, log likelihood ratio test) in the objective function (OFV), when introduced in the model, were retained in the model. A backward deletion was performed to assess the covariate parameters relationships. Only the covariates associated with a significant change of the objective function with a p value <0.001 were retained in the final model. The population parameters were then re-estimated considering the relationship with the covariates. Before qualification, model verification was performed by examination of the goodness-of-fit plots and by estimation of several quality criteria such as bias, precision or Absolute Average Fold Error.

The validation of the PopPK/EASI model predictive ability was performed using different approaches (examination of individual EASI score versus time curves, Visual Predictive Check and Sampling Important Resampling).

Finally, the final PopPK/EASI model was used to evaluate alternative dosing regimens proposed for the future phase 3 clinical studies.

### Results

At the time of the present analysis, 289 AD patients on amlitelimab (53 from CT02 and 236 from DRI17366) were available for the efficacy analysis and corresponded to the Initial Dataset.

Prior to model selection, data cleaning was performed:
All EASI score data collected with the time after dose higher than 50% of the expected value were excluded from the analysis.
All EASI score data collected after a prohibited treatment were excluded from the analysis.
After these 2 steps of cleaning, all patients with data available only for the first 4 weeks of the treatment (number of available EASI score data ≤3) were excluded from the analysis.
The PopPK/EASI model was developed using 2477 EASI score measurements collected from 269 patients (48 from CT02 and 221 from DRI17366) including data from the induction phase and withdrawal phase (up to 32 weeks after last dose). At the time of the analysis, this was considered the Final Dataset of the analysis.
The characteristics of the subjects included in the PopPK/EASI analysis are presented in Table 52.

TABLE 52
Demographic characteristics (baseline values) of the
patients included in the Final PopPK/EASI Dataset.

| | Parameter | | |
|---|---|---|---|
| Mean (SD) Median [Min-Max] | CT02 (n = 48) | DRI17366 (n = 221) | Total (n = 269) |
| Age (yrs) | 34.6 (12.8) 34 | 38.4 (14.5) 36 | 37.7 (14.3) 35 |
| | [18-66] | [18-72] | [18-72] |
| 18-64 yrs | 47 (97.9%) | 207 (93.7%) | 254 (94.4%) |
| ≥65 yrs | 1 (2.1%) | 14 (6.3%) | 15 (5.6%) |
| Body weight (kg) | 72.9 (13.5) 73.5 | 76.7 (18.2) 75.8 | 76.0 (17.5) 75 |
| | [42-119] | [41.5-148] | [41.5-148] |
| <50 kg | 1 (2.1%) | 11 (5%) | 12 (4.4%) |
| 50-99 kg | 46 (95.8%) | 189 (85.5%) | 235 (87.4%) |
| ≥100 kg | 1 (2.1%) | 21 (9.5%) | 22 (8.2%) |
| BMI (kg/m²) | 24.9 (3.87) 24.7 | 26.4 (5.47) 25.6 | 26.2 (5.25) 25.5 |
| | [17.4-36.7] | [16.6-51.4] | [16.6-51.4] |
| Albumin (g/L) | 45.4 (3.11) 45 | 46.3 (2.98) 46 | 46.2 (3.02) 46 |
| | [37-53] | [39-55] | [37-55] |
| total bilirubin | 7.52 (3.98) 5.98 | 7.86 (5.11) 6.50 | 7.80 (4.93) 6.50 |
| (μmol/L) | [3.42-22.2] | [2.60-31] | [2.60-31] |
| Aspartate Amino | 29.9 (14.1) 25 | 22.9 (8.65) 21 | 24.2 (10.2) 21 |
| Transferase (IU/L) | [15-80] | [11-70] | [11-80] |
| Alanine amino | 29.7 (21.4) 24 | 22.6 (15.1) 18 | 23.8 (16.7) 20 |
| transferase (IU/L) | [5-143] | [5-141] | [5-143] |
| Alkaline | 70.6 (21.7) 67.5 | 74.2 (20.4) 72 | 73.6 (20.7) 71 |
| phosphatase (IU/L) | [24-139] | [35-128] | [24-139] |
| creatinine | 124 (34.1) 124 | 141 (43.9) 138 | 138 (42.8) 134 |
| clearance (mL/min) | [52.3-256] | [60.4-310] | [52.3-310] |
| EASI | 31.5 (12.2) 27.4 | 29.2 (10.6) 27.6 | 29.6 (10.9) 27.5 |
| | [16.2-63.6] | [16-69.3] | [16-69.3] |
| Severe (>21) | 39 (81.3%) | 158 (71.5%) | 197 (73.2%) |
| Moderate (<=21) | 9 (18.8%) | 63 (28.5%) | 72 (26.8%) |
| vIGA-AD | 3.40 (0.489) 3 | 3.29 (0.451) 3 | 3.30 (0.460) 3 |
| | [3-4] | [3-4] | [3-4] |
| Male-Female | 27 (56.3%)/ | 112 (50.7%)/ | 139 (51.7%)/ |
| | 21 (43.8%) | 109 (49.3%) | 130 (48.3%) |
| Caucasian/Black/ | 48 (100)/0 (0)/0 | 184 (83.3%)/ | 232 (86.2%)/ |
| Asian/Others | (0)/0 (0) | 8 (3.62%)/ | 8 (2.97%)/ |
| | | 28 (12.7%)/ | 28 (10.4%)/ |
| | | 1 (0.452%) | 1 (0.372%) |
| ADAMAX (0/1) | 32 (66.7%)/ | 194 (87.8%)/ | 226 (84.0%)/ |
| | 16 (33.3%) | 27 (12.2%) | 43 (16.0%) |
| RESP (0/1) | 26 (45.8%)/ | 80 (36.2%)/ | 106 (39.4%)/ |
| | 22 (54.2%) | 141 (63.8%) | 163 (60.6%) |

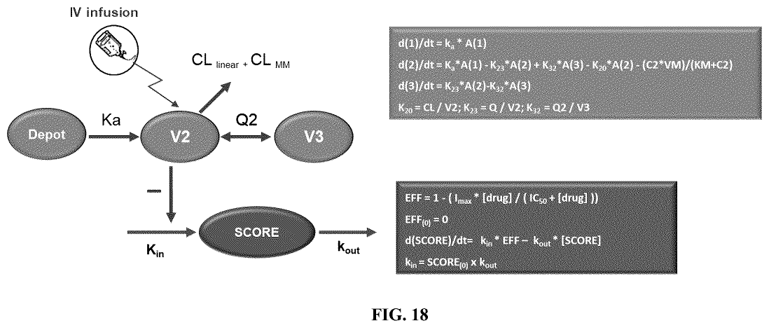

The relationship between EASI score and amlitelimab concentrations was best characterized by a turnover response model (type I—loss of induction) in which amlitelimab concentrations exerted an inhibitory effect on EASI score production rate. The model was parametrized with Kout, the first order rate constant of EASI score decrease. Imax, the maximum drug induced inhibitory effect and IC50, the amlitelimab concentration at 50% of maximum drug inhibitory effect. The inter-individual variability was estimated for Imax and Kout parameters through exponential error models. A combined (proportional+additive) error model was used to model the residual variability. The PopPK and PopPK/EASI are schematically depicted in FIG. 18.

After covariate selection processes, the final PopPK/EASI model included a significant effect of the responder status (RESP) on both Imax and Kout parameters.

The Imax parameter was related to the responder status (RESP) by the following equation:

$$Imax = COV 1 * ( 1 - RESP ) + TVIMAX * RESP$$

The Kout parameter was related to the responder status (RESP) by the following equation:

$$Kout = COV 2 * ( 1 - RESP ) + TVKOUT * RESP$$

No important systematic deviations or major bias in any of the goodness of fit plots were observed. The predictive performances using Visual Predictive Check technique and the evaluation of the uncertainties of the estimate values using Sampling Importance Resampling approach qualified the model.
The performances and quality of the model were in the same range for responder and non-responder patients.
Final population PK/EASI parameters and their relationship to covariates are presented in Table 53.

TABLE 53
Population PK/EASI parameters of the final model.

| Parameter | Estimate (CV) | RSE (%) | 95% CI (shrinkage) |
|---|---|---|---|
| Kout (day⁻¹) | 0.0224 | 4.07% | [0.0206; 0.0242] |
| Imax | 0.968 | 0.39% | [0.961; 0.976] |
| IC50 (*10⁻⁴ μg/mL) | 0.166 | 21.5% | [0.0944; 0.237] |
| Effect of No RESP on Imax | 0.537 | 6.45% | [0.468; 0.607] |
| Effect of No RESP on Kout | 0.0396 | 11.30% | [0.0306; 0.0485] |
| Inter-individual variability | | | |
| w² Kout | 0.222 (49.8%) | 13.60% | [0.163; 0.281] (23.9%) |
| w² Imax | 1.65 | 12.60% | [1.24; 2.05] (16%) |
| Residual variability | | | |
| σ² proportional | 0.201 (44.8%) | 4.25% | [0.184; 0.218] |
| σ² additive | 0.218 (46.7%) | 19.70% | [0.134; 0.302] |

RSE (%): Percentage of Relative Standard Error (100% * SE/Estimate).
95% CI: 95% confidence interval;
The coefficients of variation were calculated considering log-normal variability for parameters using CV (e^{η}) = \sqrt{(e^{ω}^{²} − 1)}

The final PopPK/EASI model was used to predict percent change from baseline in EASI score in 2000 virtual patients (1200 responders and 800 non-responders) at 250 mg Q12W with a 500 mg loading dose. Results are presented in FIG. 19.

Overall, exposure data and exposure-efficacy relationship support the opportunity to translate the 62.5 mg Q4W dose (lowest efficacious dose observed in the Phase 2b clinical study (KY1005-CT05/DRI17366 [STREAM-AD]) into an extended regimen of 250 mg Q12W with 500 mg loading dose with reassuring predictability and consistency in the expected PK profile and treatment responses. Immunogenicity and safety results will be evaluated in the Phase 3 program but based on predicted exposures from this regimen, safety is expected to be consistent with safety profile observed thus far. Immunogenicity in the Phase 2b clinical study (KY1005-CT05/DRI17366 [STREAM-AD]) using a 250 mg Q4W regimen with 500 mg loading dose was significantly attenuated compared to a 62.5 mg Q4W regimen, likely due to the suppressive mode of action of amlitelimab on a humoral immune response. Also, no increase of ADA responses up to 4 weeks after the last amlitelimab was observed in this study, indicating a sustained suppression of immunogenicity. Therefore, the proposed regimen of 250 mg Q12W with 500 mg loading dose is not expected to present a significant risk for immunogenicity of amlitelimab.

Descriptive statistics on amlitelimab exposures by dose after first dose (week 1) and last dose (week 20) following Q4W administration are summarized in Table 54 and illustrated in FIG. 14.

TABLE 54
Population PK model - predicted post-hoc exposures of amlitelimab after the first dose (week 1) and the last dose (week 20) in participants

| | Cₘₐₓ (μg/mL) | | | | AUC_{4w} (μg · day/mL) | | | | C_{trough} (μg/mL) | | | |
|---|---|---|---|---|---|---|---|---|---|---|---|---|
| Dose | First dose | | Last dose | | First dose | | Last dose | | First dose | | Last dose | |
| 62.5 mg | 8.23 | (31.0%) | 17.7 | (31.9%) | 186 | (31.6%) | 412 | (32.3%) | 5.34 | (31.3%) | 11.1 | (33.8%) |
| (N = 77) | 8.38 | [2.92-13.0] | 17.2 | [5.46-31.1] | 186 | [65.4-293] | 399 | [120-731] | 5.23 | [1.83-8.51] | 10.8 | [2.72-20.7] |
| 125 mg | 14.8 | (25.9%) | 31.9 | (33.5%) | 337 | (28.0%) | 748 | (36.2%) | 9.76 | (32.3%) | 20.3 | (41.1%) |
| (N = 75) | 15.0 | [8.10-27.1] | 30.0 | [17.2-70.9] | 331 | [191-646] | 691 | [359-1718] | 9.26 | [4.76-19.9] | 18.4 | [7.83-50.0] |
| 250 mg | 29.3 | (28.6%) | 63.1 | (31.9%) | 659 | (29.7%) | 1471 | (33.7%) | 19.0 | (32.4%) | 40.0 | (37.0%) |
| (N = 73) | 29.9 | [4.19-49.8] | 64.3 | [8.77-110] | 658 | [95.5-981] | 1498 | [206-2568] | 20.0 | [2.77-33.2] | 41.5 | [5.54-72.3] |
| 500/250 mg | 56.0 | (30.9%) | 60.0 | (34.8%) | 1249 | (31.1%) | 1385 | (36.0%) | 34.9 | (33.5%) | 37.1 | (39.1%) |
| (N = 76) | 55.9 | [10.5-106] | 58.7 | [11.7-121] | 1244 | [242-2432] | 1309 | [278-2982] | 33.7 | [7.23-69.0] | 34.1 | [7.63-87.4] |

Mean (CV %);
Median [Minimum-Maximum]

Amlitelimab exposures in AD participants increased with no major deviation in dose proportionality between 62.5 mg Q4W to 250 mg Q4W, with a 500 mg loading dose consistent with saturation of the target from the lowest dose. Steady state conditions after Q4W administration were reached from the 1st administration with the loading dose regimen and 3rd administration (with an accumulation ratio of two) without the loading dose. Absolute bioavailability of amlitelimab SC doses was 86%.

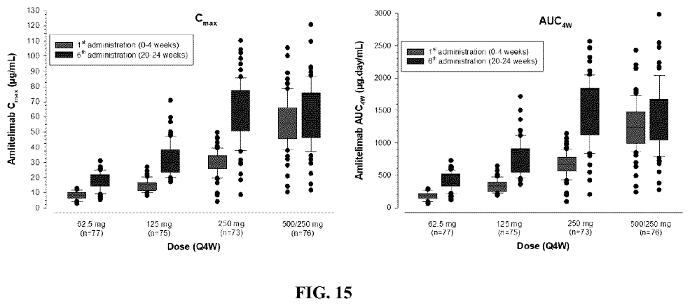

Amlitelimab steady state exposures (AUC4W) in AD participants are illustrated by baseline body weight category (<median body weight versus ≥median body weight) and in moderate versus severe AD participant for each dose arm in FIG. 15.

A 42% to 58% higher median amlitelimab steady state AUC4w was observed in participants with body weight <74 kg (median body weight in the data set) compared to participants ≥74 kg. In moderate AD participants, median steady state AUC4w was at the maximum 32% higher compared severe AD participants. A negligible increase in exposures was documented with increasing albumin.

### Example III—Simulation of Amlitelimab Dosing in a Mechanistic Systems Pharmacology Model for Atopic Dermatitis

### Overview Over the Mechanistic Systems Pharmacology Model

A mechanistic systems pharmacology model was developed to identify the optimal dosing intervals for amlitelimab in the treatment of atopic dermatitis. The model describes the relevant physiological and pharmacological processes in the skin lesion and the blood/lymph node compartment of a moderate to severe atopic dermatitis patient.

The model represented relevant pathways for the effects of OX40L (TNFSF4), IL-4, IL-5, IL-13, IL-22, mast cell mediators, chemokines, and pro- and anti-inflammatory cytokines as well as the related immune and skin cells (type 2 and type 22 T-helper cells, regulatory T-cells, dendritic cells, mast cells, neurons, and keratinocytes) in the pathology of atopic dermatitis. The model includes the pharmacokinetics and the treatment effects of drugs targeting OX40L, IL-13, and the IL-4 receptor. Treatment effects can be simulated as effects on clinical disease scores (EASI, SCORAD, IGA) and relevant biomarkers (IgE, TARC/CCL17, eotaxin-3 and eosinophil count in plasma).

The model was optimized to reproduce the clinical trial data of monoclonal antibody X (Simpson et al. 2016), monoclonal antibody Y (Guttman-Yassky et al. 2020) and the phase 2a data of amlitelimab (Weidinger et al. 2023). The model was validated by “blind prediction” of the follow-up (beyond week 16) amlitelimab phase 2a data and tralokinumab clinical trial data of ECZTRA1/2 (Wollenberg et al. 2021).

The model was fitted to the individual patient data of the amlitelimab phase 2a trial (Weidinger et al. 2023) to generate virtual twins. This virtual twin population was used to predict the variability in the simulation of a virtual clinical trial.

The model was implemented and simulations were performed in the ‘Matlab’ software package (version 2020b, The MathWorks, Inc. USA) using the ‘Simbiology’ toolbox.

### Simulation Result

Amlitelimab treatment with a dosing interval of 12 weeks (Q12W dosing) was simulated in the mechanistic systems pharmacology model for atopic dermatitis. A subcutaneous application was replicated with two different dosings:
125 mg loading dose at week 0 and 62.5 mg every 12 weeks starting at week 12.
500 mg loading dose at week 0 and 250 mg every 12 weeks starting at week 12.
   The last dose was applied at week 48 and the end of treatment was one dosing interval after the last dose (week 60). A follow-up of 24 weeks was included in the simulation.

Simulations were done with a cohort of 200 virtual patients that represented amlitelimab responders (virtual patients had been filtered for those achieving an EASI75 at week 24). Virtual patients had been generated by sampling parameters from the parameter distribution of the virtual twin population (virtual twins generated from the phase 2a results as described above).

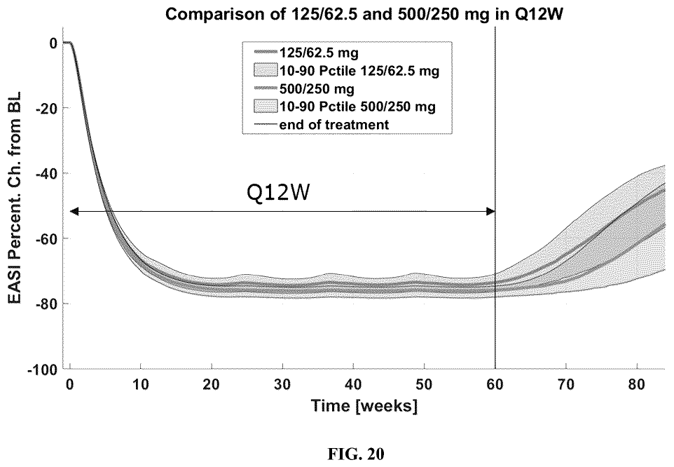

FIG. 20 shows the simulation result of the percent change in EASI from baseline over the time course of 84 weeks. As can be seen Q12W results in a very similar reduction of the EASI score for both doses simulated. Only in the follow-up (after week 60) a faster relapse can be seen for the lower dose.

### Detailed Description of the Mechanistic Systems Pharmacology Model for Atopic Dermatitis

The model consists of two compartments: the skin compartment and the blood/lymph node compartment. Each compartment contains the cell-types and mediators (cytokines, chemokines, mast cell mediators) relevant for the disease processes. The cell-types exist in two or three different states of development and/or activation. Production, state transition, migration to a different compartment and elimination of model components are controlled by the concentrations/amounts of other model components. The reactions are implemented as ordinary differential equations (ODEs).

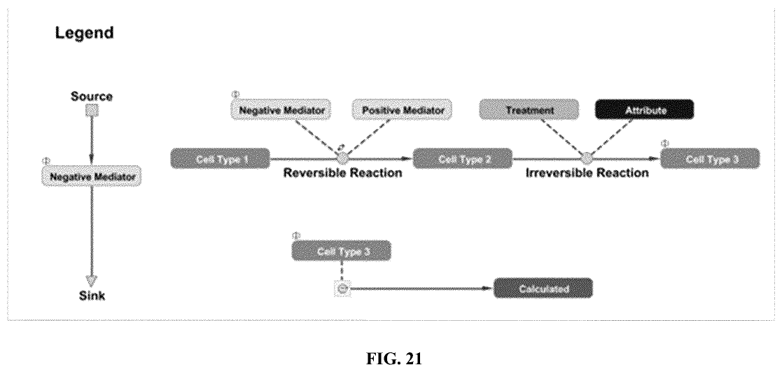

The individual components are described in the following sections. A general legend for reading the pathway figures is shown in FIG. 21.

### Blood/Lymph Node Compartment

### Pharmacokinetics of Amlitelimab

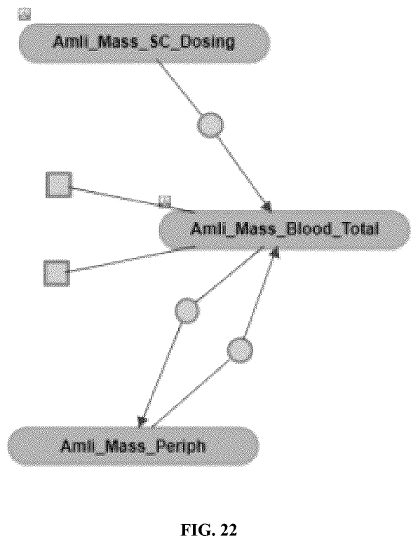

The PK model of amlitelimab consists of a central and a peripheral compartment as well as a subcutaneous depot (FIG. 22). Drug dosing on the subcutaneous and the intravenous route can be simulated. Dosing in this model is done in drug mass (unit milligram).

### Pharmacokinetics of an Anti-IL-13 Antibody and an Anti-IL-4 Receptor Antibody

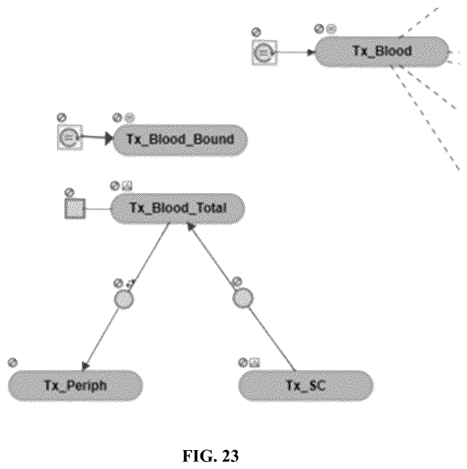

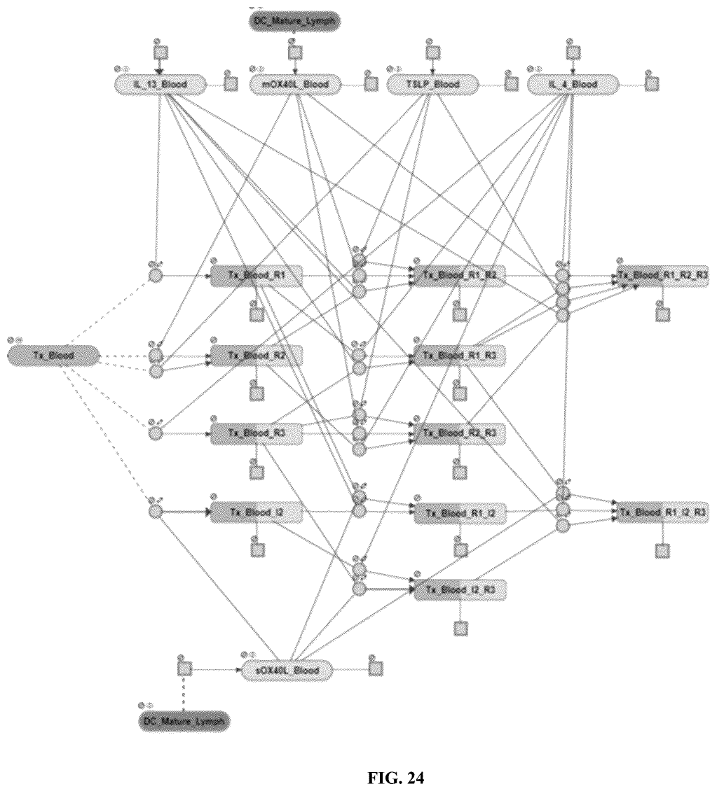

A second pharmacokinetics model for dosing drugs in units of concentration (picomole/liter). This model (FIG. 23) allows subcutaneous and intravenous dosing and was used for an anti-IL-13 antibody (antibody Y) and an anti-IL-4 receptor antibody (antibody X).

### Binding to Drug Targets

Two sub-models of binding of antibody drugs to their respective target have been implemented. OX40L exists in a membrane bound form (mOX40L) and a soluble form (sOX40L), the first sub-model is shown in FIG. 24. The second sub-model implements the binding of IL-4, IL-13 and monoclonal antibody X to the IL-4 receptor/IL-13receptor complexes. The model implements an IL-13 receptor that can be activated by IL-4 and IL-13, and an IL-4 receptor that can only be activated by IL-4. Since both receptors are dimers that contain the monoclonal antibody X target (the IL-4R alpha subunit, not explicitly implemented in the model) both receptors can be blocked by monoclonal antibody X.

### Biomarker Production

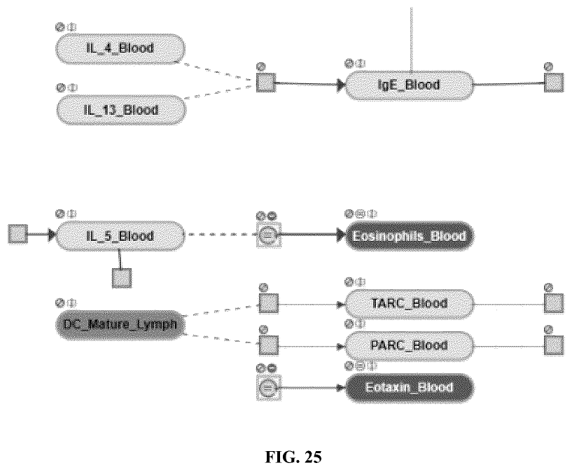

IgE levels are controlled by IL-4 and IL-13. The IgE can transit into the skin compartment. Eosinophil production is mainly dependent on the IL-5 levels. Production of TARC (CCL17) and PARC (CCL18) is controlled by dendritic cells in the lymph compartment at different individual rates. Eotaxin levels are calculated from the disease severity (see skin compartment). (See FIG. 25)

### Cell Migration and T-Cell Priming

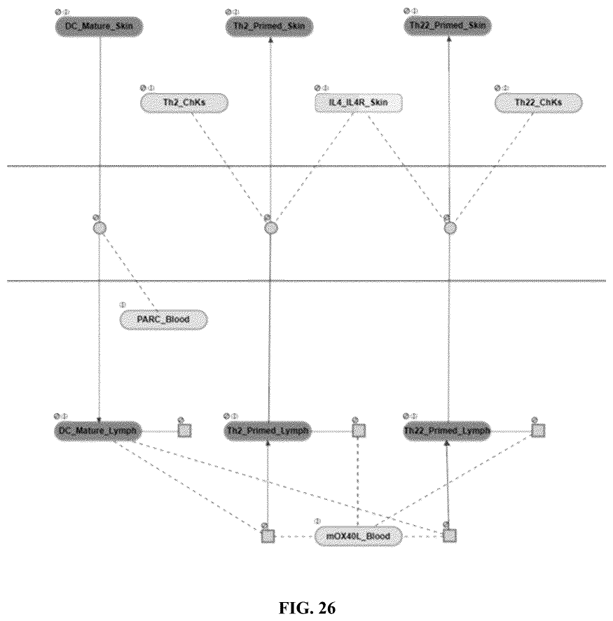

Mature dendritic cells migrate from skin to the lymph compartment. This migration is accelerated by PARC. The dendritic cells then prime the T helper cells (Th2 and Th22) in cooperation with OX40L that is expressed on the dendritic cells. Primed T helper cells migrate to the skin compartment. This process is stimulated by chemokines and IL-4 (represented by the activated IL4 receptor in the model) (See FIG. 26).

### Skin Compartment

### Binding to Drug Targets

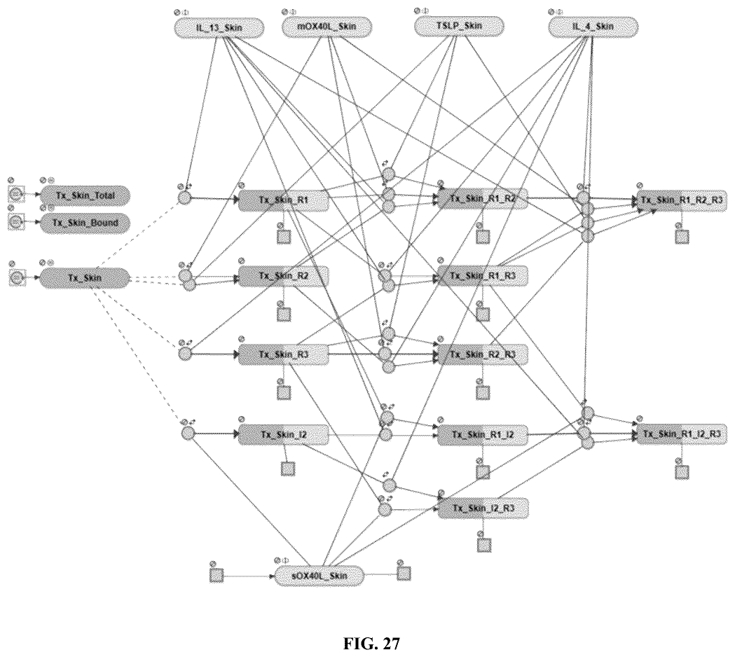

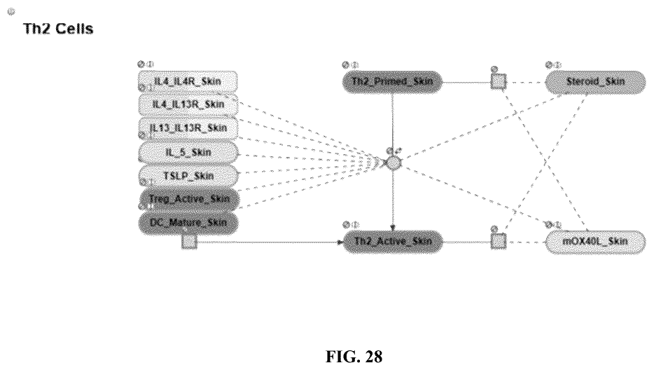

The skin compartment contains the same two sub-models of binding of antibody drugs to their respective target as the blood/lymph node compartment (See FIG. 27). The distribution of antibody drugs to skin is set according to the antibody biodistribution coefficients from Shah and Betts 2013.

### Type 2 T Helper Cells

Type 2 T-helper cells (Th2 cells) are primed in the lymph compartment and migrate into the skin compartment (see section on “Cell migration and T-cell priming” above). In skin, primed Th2 cells are activated to become active Th2 cells. The activation and the elimination of Th2 cells in skin is controlled by other model components as shown in FIG.

### Type 22 T Helper Cells

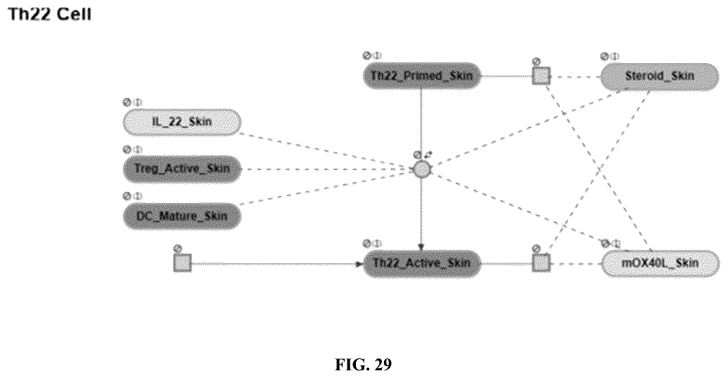

Type 22 T-helper cells (Th22 cells) are primed in the lymph compartment and migrate into the skin compartment (see section on “Cell migration and T-cell priming” above). In skin, primed Th22 cells are activated to become active Th22 cells. The activation and the elimination of Th22 cells in skin is controlled by other model components as shown in FIG. 29.

### Regulatory T-Cells

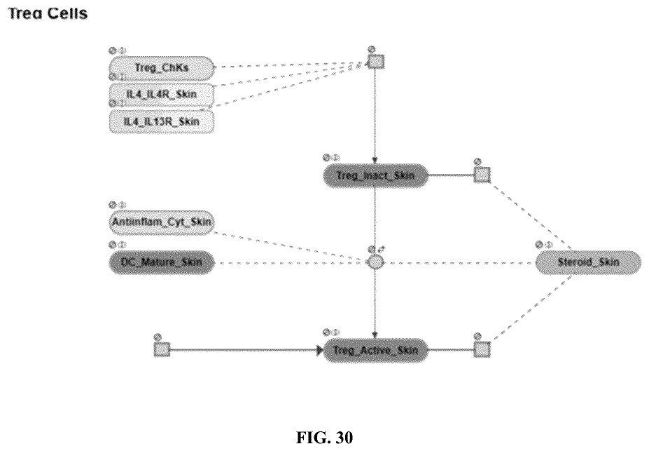

Regulatory T-cells (Treg cells) exert an immunosuppressive role in the model. They are produced as an inactive Treg species and can be activated. Factors controlling production, activation, and elimination in the life cycle of regulatory T-cells are shown in FIG. 30.

### Mast Cells

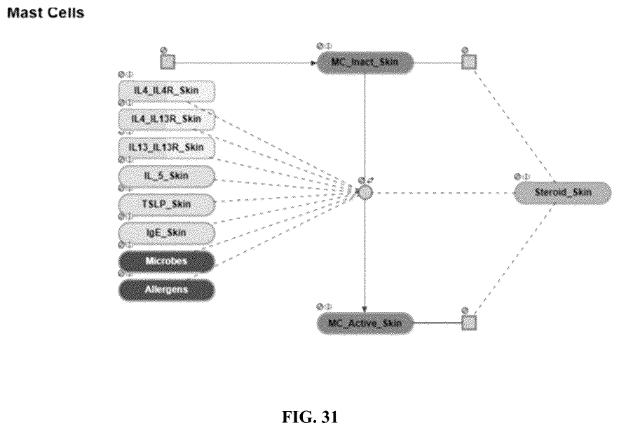

Mast cells have an important role in atopic immune responses. In response to e.g. allergens or antigens bound to IgE they can release a variety of mediators that have a role in the inflammatory response. In the model mast cells are produced in an inactive form and can be activated through a number of stimuli. The factors controlling mast cell activation and life cycle are shown in FIG. 31.

### Dendritic Cells

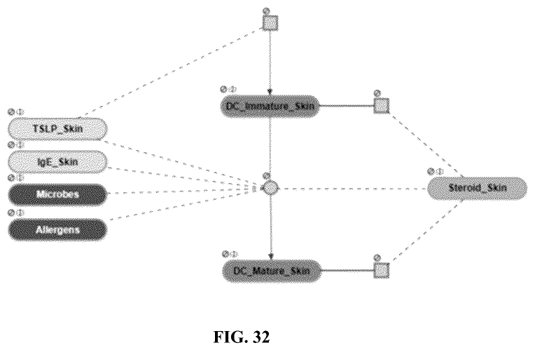

Dendritic cells are antigen presenting cells. In the skin compartment they encounter allergens and microbial antigens that have crossed the skin barrier. Together with alarmins (mediators of the organism that signal damage to a tissue) they move from an immature to a mature state. Mature dendritic cells then migrate to the lymphatic tissue to prime lymphocytes (see also section on “Cell migration and T-cell priming” above). The factors controlling dendritic call maturation and life cycle are shown in FIG. 32.

### Keratinocyte Life Cycle

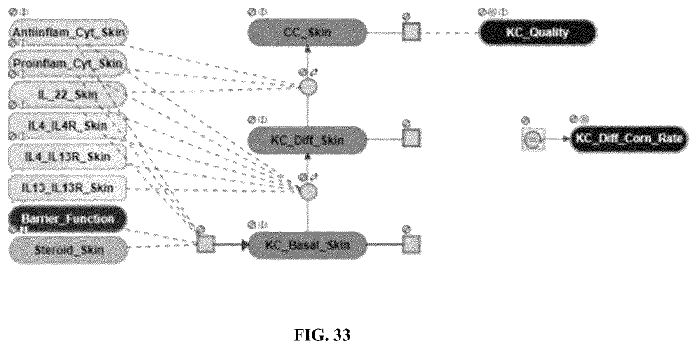

The epidermis is modeled as three layers of keratinocytes. Basal keratinocytes (KC Basal Skin) develop into differentiated keratinocytes (KC Diff Skin) which then become corneocytes (CC skin) in a process called cornification. The factors controlling the life cycle of keratinocytes are shown in FIG. 33.

### Mediator Production

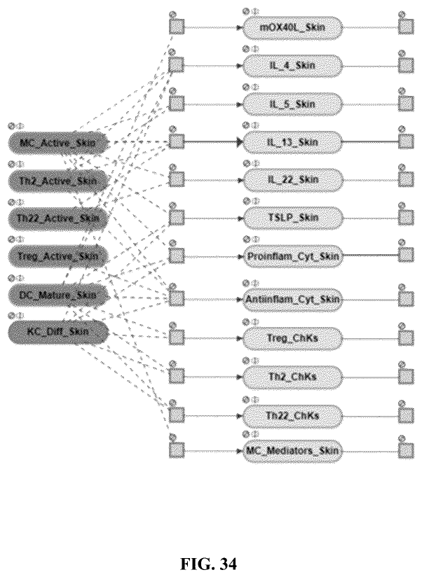

The cell types mentioned above produce the relevant mediators (small proteins, peptides or small molecules) that play a role in atopic dermatitis. These mediators encompass Th2 and Th22 cytokines (IL-4, IL-5, IL-13 and IL-22), the alarmin TSLP, other pro-inflammatory cytokines, anti-inflammatory cytokines, mast cell mediators, the membrane bound form of OX40L and three cell-type specific chemokine groups (Treg ChKs, Th2 ChKs and Th22 ChKs). The model structure for production and elimination of these mediators is shown in FIG. 34.

### Pruritus

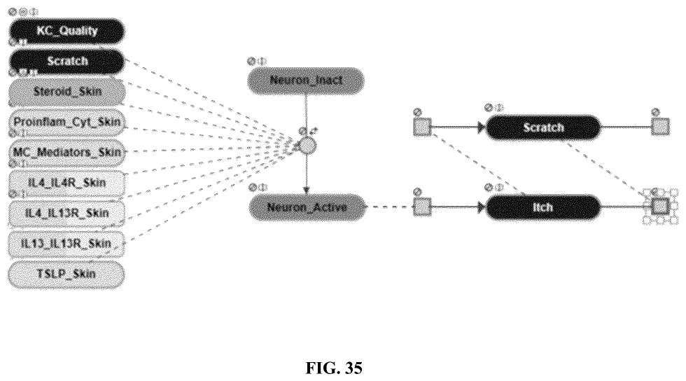

In this model, pruritus is caused by an activation of sensory neurons in skin. This activation results in the sensation of itch. Itch then causes the reaction of scratching the skin region. This leads to a temporal relief of the itch sensation but also results in a damage of the skin barrier. The processes that generate and control pruritus are shown in FIG. 35.

### Keratinocyte and Skin Quality

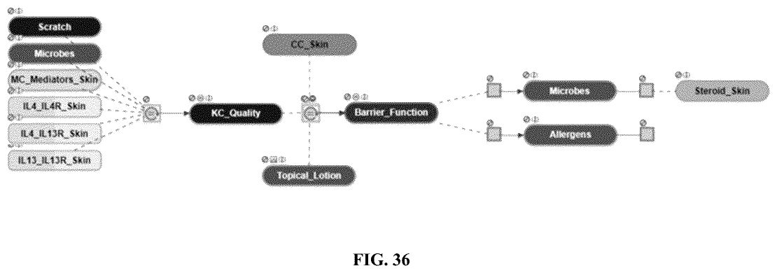

An important factor in the pathology of atopic dermatitis is the integrity of the skin barrier function. In this model the barrier function has values on an arbitrary scale. It is calculated from a calculated value of keratinocyte quality and the number of corneocytes. Improvements of barrier function reduce the intrusion of allergens and microbes. The sequence of calculations for and the control of barrier function and keratinocyte quality is shown in FIG. 36.

### Calculation of Clinical Outcome Scores

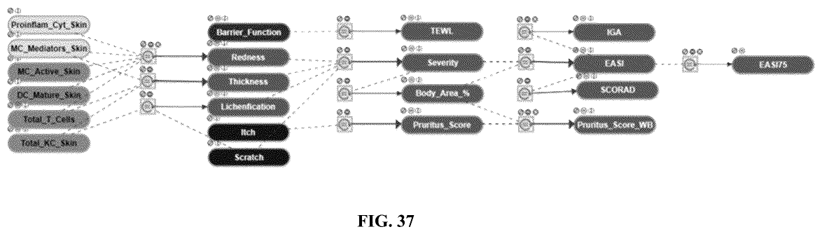

Clinical outcome scores are calculated by a sequence of algebraic equations (repeated assignment rules in MATLAB Simbiology). In general, first subcomponents of the various scores are calculated and then combined to build the clinical score. The sequence of calculations is shown in FIG. 37.

### REFERENCES

Guttman-Yassky, E., A. Blauvelt et al. (2020) “Efficacy and Safety of Lebrikizumab, a High-Affinity Interleukin 13 Inhibitor, in Adults With Moderate to Severe Atopic Dermatitis. A Phase 2b Randomized Clinical Trial.” JAMA Dermatol. 156 (4): 411-420.
Shah, D. K., A. M. Betts (2013). “Antibody biodistribution coefficients: Inferring tissue concentrations of monoclonal antibodies based on the plasma concentrations in several preclinical species and human.” mAbs 5 (2): 297-305.
Simpson, E. L., T. Bieber, et al. (2016). “Two Phase 3 Trials of Dupilumab versus Placebo in Atopic Dermatitis.” N Engl J Med 375:2335-48.
Weidinger, S., T. Bieber, et al. (2023). “Safety and efficacy of amlitelimab, a fully human nondepleting, noncytotoxic anti-OX40 ligand monoclonal antibody, in atopic dermatitis: results of a phase IIa randomized placebo-controlled trial.” Br J Dermatol advance publication.
Wollenberg, A., A. Blauvelt, et al. (2021). “Tralokinumab for moderate-to-severe atopic dermatitis: results from two 52-week, randomized, double-blind, multicentre, placebo-controlled phase III trials (ECZTRA 1 and ECZTRA 2).” Br J Dermatol 184:437-449.

### Example IV

A Phase 3, Randomized, Double-Blind, Placebo-Controlled, Parallel-Group, 3-Arm, Multinational, Multicenter Study to Evaluate the Efficacy and Safety of Amlitelimab Monotherapy by Subcutaneous Injection in Participants Aged 18 Years and Older with Moderate-to-Severe Atopic Dermatitis (U1111-1275-9715)

This is a parallel group, Phase 3, multinational, multicenter, randomized, double blind, placebo-controlled, 3-arm monotherapy study for treatment of participants diagnosed with moderate to severe atopic dermatitis (AD), whose disease is not adequately controlled with topical prescription therapies or when those therapies are not advisable. The purpose of this study is to measure the efficacy and safety of treatment with amlitelimab solution for subcutaneous (SC) injection compared with placebo in participants with moderate to severe AD aged 18 years and older.

### Study Details

At the end of the treatment period, participants will have an option to enter a separate study: the blinded extension study EFC17600 (ESTUARY). For participants not entering the blinded extension Study EFC17600 (ESTUARY), the study duration will be up to 44 weeks including a 2 to 4-week screening, a 24-week randomized double-blind period, and a 16-week safety follow-up.

For participants entering the blinded extension Study EFC17600 (ESTUARY), the study duration will be up to 28 weeks including a 2 to 4-week screening and a 24-week randomized double-blind period. The total treatment duration will be up to 24 weeks. The total number of visits will be up to 10 visits (or 9 visits for those entering the blinded extension study EFC17600] (ESTUARY).

### Study Objectives

### Primary Endpoints

To demonstrate the efficacy of amlitelimab monotherapy administered by SC injection in comparison to placebo in participants aged 18 years and older with moderate-to-severe AD. (See Table 55)

### Secondary Endpoints

To assess the efficacy of amlitelimab monotherapy administered by SC injection in comparison to placebo in participants aged 18 years and older with moderate-to-severe AD.
To assess the safety profile of amlitelimab monotherapy administered by SC injection in participants aged 18 years and older with moderate-to-severe AD.
To characterize the pharmacokinetic profile of amlitelimab monotherapy administered by SC injection in participants aged 18 years and older with moderate-to-severe AD.
To characterize immunogenicity of amlitelimab monotherapy administered by SC injection in
   participants aged 18 years and older with moderate-to-severe AD.

### (See Table 56)

### Inclusion Criteria

Participants must be 18 years of age (when signing informed consent form).
Diagnosis of AD for at least 1 year (defined by the American Academy of Dermatology Consensus Criteria).
Documented history (within 6 months before screening) of either inadequate response or inadvisability to topical treatments, and/or inadequate response to systemic therapies (within 12 months before screening).
v-IGA-AD of 3 or 4 at baseline visit.
EASI score of 16 or higher at baseline.
AD involvement of 10% or more of BSA at baseline.
Weekly average of daily PP-NRS of ≥4 at baseline visit.
Able and willing to comply with requested study visits and procedures.
Body weight ≥40 kg.

### Exclusion Criteria

Participants are excluded from the study if any of the following criteria apply:
Skin co-morbidity that would adversely affect the ability to undertake AD assessments.
Known history of or suspected significant current immunosuppression.
Any malignancies or history of malignancies prior to baseline (excluding for nonmelanoma skin cancer excised and cured >5 years prior to baseline).
History of solid organ or stem cell transplant.
Any active or chronic infection including helminthic infection requiring systemic treatment within 4 weeks prior baseline (1 week in the event of superficial skin infections).
Positive for human immunodeficiency virus (HIV), Hepatitis B or hepatitis C at screening visit.
Having active tuberculosis (TB), latent TB, a history of incompletely treated TB, suspected extrapulmonary TB infection, or who are at high risk of contracting TB.
Having received any of the specified therapy within the specified timeframe(s) prior to the baseline visit.
In the Investigator's opinion, any clinically significant laboratory results or protocol specified laboratory abnormalities at screening.
History of hypersensitivity or allergy to any of the excipients or investigational medicinal product (IMP).

TABLE 55
Primary Endpoints.

| | | Time |
|---|---|---|
| | | frame for |
| Primary Endpoint title | Endpoint Description | evaluation |
| EU, EU reference countries, and | The vIGA-AD is an Investigator-completed | Week 24 |
| Japan: Proportion of participants | assessment scale used to determine severity | |
| with Validated Investigator Global | of AD and clinical response to treatment. It | |
| Assessment scale for Atopic | is based on a 5-point scale, ranging from 0 | |
| Dermatitis (vIGA-AD) of 0 (clear) | (clear) to 4 (severe). | |
| or 1 (almost clear) and a reduction | | |
| from baseline of ≥2 points at | | |
| Week 24 | | |
| EU, EU reference countries, and | The EASI is an Investigator-assessed | Week 24 |
| Japan: Proportion of participants | validated tool used to measure the | |
| reaching 75% reduction from | extent (area) and severity of AD. Total | |
| baseline in Eczema Area and | score ranges from 0 to 72 with a | |
| Severity Index (EASI) score (EASI- | higher score indicating increased | |
| 75) at Week 24 | extent and severity of AD. | |
| US and US reference countries: | The vIGA-AD is an Investigator-completed | Week 24 |
| Proportion of participants with | assessment scale used to | |
| vIGA-AD of 0 (clear) or 1 (almost | determine severity of AD and clinical | |
| clear) and a reduction from baseline | response to treatment. It is based on a | |
| of ≥2 points at Week 24 | 5-point scale, ranging from 0 (clear) to 4 | |
| | (severe). | |

TABLE 56
Secondary Endpoints.

| | | Time |
|---|---|---|
| | | frame for |
| Secondary Endpoint title | Endpoint Description | evaluation |
| Proportion of participants reaching | The EASI is an Investigator-assessed | Week 24 |
| EASI-75 at Week 24 (for US and | validated tool used to measure the extent | |
| US reference countries only) | (area) and severity of AD. Total score | |
| | ranges from 0 to 72 with a higher score | |
| | indicating increased extent and severity of | |
| | AD. EASI-75 is 75% reduction from | |
| | baseline in EASI score. | |
| Proportion of participants with | The vIGA-AD is an Investigator-completed | Baseline to |
| vIGA-AD 0 (clear) or 1 (almost | assessment scale used to determine severity | Week 24 |
| clear) with presence of only barely | of AD and clinical response to treatment. It | |
| perceptible erythema (no | is based on a 5-point scale, ranging from 0 | |
| induration/papulation, no | (clear) to 4 (severe). | |
| lichenification, no oozing or | | |
| crusting) | | |
| Proportion of participants with ≥4- | The PP-NRS is a validated single item 0-10 | Baseline to |
| point reduction in weekly average of | numeric rating scale assessing | Week 24 |
| daily Peak Pruritus-Numerical | peak pruritus (itch) associated with AD with | |
| Rating Scale (PP-NRS) from | 0 = no itch and 10 = worst itch | |
| baseline in participants with | imaginable. | |
| baseline weekly average of daily | | |
| PP-NRS ≥4 | | |
| Proportion of participants | The EASI is an Investigator-assessed | Baseline to |
| reaching EASI-75 | validated tool used to measure the extent | Week 20 |
| | (area) and severity of AD. Total score | |
| | ranges from 0 to 72 with a higher score | |
| | indicating increased extent and severity of | |
| | AD. EASI-75 is 75% reduction from | |
| | baseline in EASI score. | |
| Proportion of participants with | The vIGA-AD is an Investigator-completed | Baseline to |
| vIGA-AD of 0 (clear) or 1 (almost | assessment scale used to determine severity | Week 20 |
| clear) and a reduction from | of AD and clinical response to treatment. It | |
| baseline of ≥2 points | is based on a 5-point scale, ranging from 0 | |
| | (clear) to 4 (severe). | |
| Proportion of participants | The EASI is an Investigator-assessed | Baseline to |
| reaching EASI-90 | validated tool used to measure the | Week 24 |
| | extent (area) and severity of AD. Total score | |
| | ranges from 0 to 72 with a | |
| | higher score indicating increased extent and | |
| | severity of AD. EASI-90 is 90% reduction | |
| | from baseline in EASI score. | |
| Proportion of participants | The EASI is an Investigator-assessed | Baseline to |
| reaching EASI-100 | validated tool used to measure the | Week 24 |
| | extent (area) and severity of AD. Total score | |
| | ranges from 0 to 72 with a | |
| | higher score indicating increased extent and | |
| | severity of AD. EASI-100 is 100% | |
| | reduction from baseline in EASI score. | |
| Change in Dermatology Quality of | The DLQI is a validated 10-item | Baseline to |
| Life Index (DLQI) from baseline | questionnaire to measure dermatology- | Week 24 |
| | specific quality of life (QoL) in adult | |
| | patients. Overall scoring ranges from 0 to | |
| | 30, with a higher score indicating a poorer | |
| | QoL. | |
| Proportion of participants with a | The DLQI is a validated 10-item | Baseline to |
| reduction in DLQI ≥4 from baseline | questionnaire to measure dermatology- | Week 24 |
| in participants with DLQI | specific quality of life (QoL) in adult | |
| baseline ≥4 | patients. Overall scoring ranges from 0 | |
| | to 30, with a higher score indicating | |
| | a poorer QoL. | |
| Change in Hospital Anxiety | The HADS is 14-item questionnaire with | Baseline to |
| Depression Scale (HADS) from | two subscales: anxiety & depression. Each | Week 24 |
| baseline | subscale (anxiety & depression) ranges 0- | |
| | 21. The total HADS score ranges 0-42 with | |
| | higher score indicating a poorer state. | |
| Proportion of participants with | HADS-A score ranges 0-21 with higher | Baseline to |
| HADS subscale Anxiety (HADS-A) | score indicating a poorer state. | Week 24 |
| <8 in participants with baseline | | |
| HADS-A ≥8 | | |
| Proportion of participants with | HADS-D score ranges 0-21 with higher | Baseline to |
| HADS subscale Depression | score indicating a poorer state. | Week 24 |
| (HADS-D) <8 in participants with | | |
| HADS-D baseline ≥8 | | |
| Change in weekly average of daily | The SP-NRS is a single item 0-10 numeric | Baseline to |
| Skin Pain-Numerical Rating Scale | rating scale assessing skin pain associated | Week 24 |
| (SP-NRS) from baseline | with AD with 0 = no pain and 10 = worst | |
| | possible pain imaginable. | |
| Proportion of participants with a | The SP-NRS is a single item 0-10 numeric | Baseline to |
| reduction in weekly average of daily | rating scale assessing skin pain associated | Week 24 |
| SP-NRS ≥4 from baseline in | with AD with 0 = no pain and 10 = worst | |
| participants with baseline weekly | possible pain imaginable. | |
| average of daily SP-NRS ≥4 | | |
| Change in weekly average of daily | The SD-NRS is a single item 0-10 numeric | Baseline to |
| Sleep Disturbance-Numerical | rating scale assessing sleep disturbance | Week 24 |
| Rating Scale (SD-NRS) from | associated with AD with 0 = no sleep loss | |
| baseline | and 10 = did not sleep at all. | |
| Proportion of participants with a | The SD-NRS is a single item 0-10 numeric | Baseline to |
| reduction in weekly average of daily | rating scale assessing sleep disturbance | Week 24 |
| SD-NRS ≥3 from baseline in | associated with AD with 0 = no sleep loss | |
| participants with Baseline weekly | and 10 = did not sleep at all. | |
| average of daily SD-NRS ≥3 | | |
| Proportion of participants with a | The SD-NRS is a single item 0-10 numeric | Baseline to |
| reduction in weekly average of daily | rating scale assessing sleep disturbance | Week 24 |
| SD-NRS ≥5 from baseline in | associated with AD with 0 = no sleep loss | |
| participants with baseline weekly | and 10 = did not sleep at all. | |
| average of daily SD-NRS ≥5 | | |
| Percent change in EASI score from | The EASI is an Investigator-assessed | Baseline to |
| baseline | validated tool used to measure the extent | Week 24 |
| | (area) and severity of AD. Total score | |
| | ranges from 0 to 72 with a higher score | |
| | indicating increased extent and severity of | |
| | AD. | |
| Percent change in weekly average of | The PP-NRS is a validated single item 0-10 | Baseline to |
| daily PP-NRS from baseline | numeric rating scale assessing peak pruritus | Week 24 |
| | (itch) associated with AD with 0 = no itch | |
| | and 10 = worst itch imaginable. | |
| Proportion of participants reaching | The EASI is an Investigator-assessed | Baseline to |
| EASI-50 | validated tool used to measure the extent | Week 24 |
| | (area) and severity of AD. Total score | |
| | ranges from 0 to 72 with a higher score | |
| | indicating increased extent and severity of | |
| | AD. EASI-50 is 50% | |
| | reduction from baseline in EASI score. | |
| Proportion of participants with | The EASI is an Investigator-assessed | Baseline to |
| EASI ≤7 | validated tool used to measure the extent | Week 24 |
| | (area) and severity of AD. Total score | |
| | ranges from 0 to 72 with a higher score | |
| | indicating increased extent and severity of | |
| | AD. | |
| Change in percent Body Surface | | Baseline to |
| Area (BSA) affected by AD from | | Week 24 |
| baseline | | |
| Percent change in Scoring Atopic | The SCORAD index is a clinical tool to | Baseline to |
| Dermatitis (SCORAD) index from | evaluate the extent and severity of AD. | Week 24 |
| baseline | Total score ranges from 0 (absent disease) | |
| | to 103 (severe disease). | |
| Proportion of participants with a | The SCORAD index is a clinical tool to | Baseline to |
| reduction in SCORAD ≥8.7 points | evaluate the extent and severity of AD. | Week 24 |
| from baseline in participants with | Total score ranges from 0 (absent disease) | |
| baseline SCORAD score ≥8.7 | to 103 (severe disease). | |
| Proportion of participants with a | The POEM is a 7-item self-assessment | Baseline to |
| reduction in Patient Oriented | questionnaire that assesses disease | Week 24 |
| Eczema Measure (POEM) ≥4 from | symptoms on a 5-point scale; 0 (no days) to | |
| baseline in participants with POEM | 4 (every day in the last week). The sum of | |
| Baseline ≥4 | the 7 items gives the total POEM score of 0 | |
| | (absent disease) to 28 (very severe). Higher | |
| | scores indicated more severe disease and | |
| | poor quality of life. | |
| Change in POEM from baseline | The POEM is a 7-item self-assessment | Baseline to |
| | questionnaire that assesses disease | Week 24 |
| | symptoms on a 5-point scale; 0 (no days) to | |
| | 4 (every day in the last week). The sum of | |
| | the 7 items gives the total POEM score of 0 | |
| | (absent disease) to 28 (very severe). Higher | |
| | scores indicated more severe disease and | |
| | poor quality of life. | |
| Proportion of participants with | | Baseline to |
| rescue medication use [from | | Week 24 |
| baseline to Week 24] | | |
| Percentage of participants who | | Baseline to |
| experienced Treatment-Emergent | | Week 40 |
| Adverse Events (TEAEs), | | |
| experienced Treatment-Emergent | | |
| Serious Adverse Events (TESAEs) | | |
| and/or Treatment-Emergent | | |
| Adverse Events of Special Interest | | |
| (AESI) | | |
| Serum amlitelimab concentrations | | Baseline to |
| | | Week 40 |
| Incidence of antidrug antibodies | | Baseline to |
| (ADAs) of amlitelimab | | Week 40 |

### Example V

A Phase 3, Randomized, Double-Blind, Placebo-Controlled, Parallel-Group, 3-Arm, Multinational, Multicenter Study to Evaluate the Efficacy and Safety of Amlitelimab Monotherapy by Subcutaneous Injection in Participants Aged 18 Years and Older with Moderate-to-Severe Atopic Dermatitis (U1111-1275-9665)

This is a parallel group, Phase 3, multinational, multicenter, randomized, double blind, placebo-controlled, 3-arm monotherapy study for treatment of participants diagnosed with moderate to severe atopic dermatitis (AD), whose disease is not adequately controlled with topical prescription therapies or when those therapies are not advisable. The purpose of this study is to measure the efficacy and safety of treatment with amlitelimab solution for subcutaneous (SC) injection compared with placebo in participants with moderate to severe AD aged 18 years and older.

### Study Details

At the end of the treatment period, participants will have an option to enter a separate study: the blinded extension study EFC17600 (ESTUARY). For participants not entering the blinded extension Study EFC17600 (ESTUARY), the study duration will be up to 44 weeks including a 2 to 4-week screening, a 24-week randomized double-blind period, and a 16-week safety follow-up. For participants entering the blinded extension Study EFC17600 (ESTUARY), the study duration will be up to 28 weeks including a 2 to 4-week screening and a 24-week randomized double-blind period. The total treatment duration will be up to 24 weeks. The total number of visits will be up to 10 visits (or 9 visits for those entering the blinded extension study EFC17600 (ESTUARY).

### Study Objectives

### Primary Endpoints

To demonstrate the efficacy of amlitelimab monotherapy administered by subcutaneous (SC) injection in comparison to placebo in participants aged 18 years and older with moderate-to-severe atopic dermatitis (AD) (See Table 57)

### Secondary Endpoints

To assess the efficacy of amlitelimab monotherapy administered by SC injection in comparison to placebo in participants aged 18 years and older with moderate-to-severe AD.
To assess the safety profile of amlitelimab monotherapy administered by SC injection in participants aged 18 years and older with moderate-to-severe AD.
To characterize the pharmacokinetic profile of amlitelimab monotherapy administered by SC injection in participants aged 18 years and older with moderate-to-severe AD.
To characterize immunogenicity of amlitelimab monotherapy administered by SC injection in participants aged 18 years and older with moderate-to-severe AD.

### (See Table 58)

### Inclusion Criteria

Participants must be 18 years of age (when signing informed consent form).
Diagnosis of AD for at least 1 year (defined by the American Academy of Dermatology Consensus Criteria).
Documented history (within 6 months before screening) of either inadequate response or inadvisability to topical treatments, and/or inadequate response to systemic therapies (within 12 months before screening).
v-IGA-AD of 3 or 4 at baseline visit.
EASI score of 16 or higher at baseline.
AD involvement of 10% or more of BSA at baseline.
Weekly average of daily PP-NRS of ≥4 at baseline visit.
Able and willing to comply with requested study visits and procedures.
Body weight ≥40 kg.

### Exclusion Criteria

Participants are excluded from the study if any of the following criteria apply:
Skin co-morbidity that would adversely affect the ability to undertake AD assessments.
Known history of or suspected significant current immunosuppression.
Any malignancies or history of malignancies prior to baseline (excluding for nonmelanoma skin cancer excised and cured >5 years prior to baseline).
History of solid organ or stem cell transplant.
Any active or chronic infection including helminthic infection requiring systemic treatment within 4 weeks prior baseline.
Positive for human immunodeficiency virus (HIV), Hepatitis B or hepatitis C at screening visit.
Having active tuberculosis (TB), latent TB, a history of incompletely treated TB, suspected extrapulmonary TB infection, or who are at high risk of contracting TB.
Having received any of the specified therapy within the specified timeframe(s) prior to the baseline visit.
In the Investigator's opinion, any clinically significant laboratory results or protocol specified laboratory abnormalities at screening.
History of hypersensitivity or allergy to any of the excipients or investigational medicinal product (IMP).

TABLE 57
Primary endpoints.

| | | Time |
|---|---|---|
| | | frame for |
| Primary Endpoint title | Endpoint Description | evaluation |
| EU, EU reference countries, and | The vIGA-AD is an Investigator-completed | Week 24 |
| Japan: Proportion of participants | assessment scale used to determine severity of | |
| with Validated Investigator | AD and clinical response to treatment. It is | |
| Global Assessment scale for | based on a 5-point scale, ranging from 0 (clear) | |
| Atopic Dermatitis (vIGA-AD) | to 4 (severe). | |
| of 0 (clear) or 1 (almost clear) | | |
| and a reduction from baseline | | |
| of ≥2 points at Week 24 | | |
| EU, EU reference countries, and | The EASI is an Investigator-assessed validated | Week 24 |
| Japan: Proportion of participants | tool used to measure the | |
| reaching 75% reduction from | extent (area) and severity of AD. Total score | |
| baseline in Eczema Area and | ranges from 0 to 72 with a | |
| Severity Index (EASI) score | higher score indicating increased extent and | |
| (EASI-75) at Week 24 | severity of AD. | |
| US and US reference countries: | The vIGA-AD is an Investigator-completed | Week 24 |
| Proportion of participants with | assessment scale used to | |
| vIGA-AD of 0 (clear) or 1 | determine severity of AD and clinical response | |
| (almost clear) and a reduction | to treatment. It is based on a | |
| from baseline of ≥2 points | 5-point scale, ranging from 0 (clear) to 4 | |
| at Week 24 | (severe). | |

TABLE 58
Secondary Endpoints.

| | | Time |
|---|---|---|
| | | frame for |
| Secondary Endpoint title | Endpoint Description | evaluation |
| Proportion of participants | The EASI is an Investigator-assessed validated | Week 24 |
| reaching EASI-75 at Week 24 | tool used to measure the extent (area) and | |
| (for US and US reference | severity of AD. Total score ranges from 0 to 72 | |
| countries only) | with a higher score indicating increased extent | |
| | and severity of AD. EASI-75 is 75% reduction | |
| | from baseline in EASI score. | |
| Proportion of participants with | The vIGA-AD is an Investigator-completed | Baseline to |
| vIGA-AD 0 (clear) or 1 (almost | assessment scale used to determine severity of | Week 24 |
| clear) with presence of only | AD and clinical response to treatment. It is | |
| barely perceptible erythema (no | based on a 5-point scale, ranging from 0 (clear) | |
| induration/papulation, no | to 4 (severe). | |
| lichenification, no oozing or | | |
| crusting) | | |
| Proportion of participants | The PP-NRS is a validated single item 0-10 | Baseline to |
| with ≥4-point reduction in weekly | numeric rating scale assessing | Week 24 |
| average of daily Peak Pruritus- | peak pruritus (itch) associated with AD | |
| Numerical Rating Scale (PP- | with 0 = no itch and 10 = worst itch | |
| NRS) from baseline in | imaginable. | |
| participants with baseline weekly | | |
| average of daily PP-NRS ≥4 | | |
| Proportion of participants | The EASI is an Investigator-assessed validated | Baseline to |
| reaching EASI-75 | tool used to measure the extent (area) and | Week 20 |
| | severity of AD. Total score ranges from 0 to 72 | |
| | with a higher score indicating increased extent | |
| | and severity of AD. EASI-75 is 75% reduction | |
| | from baseline in EASI score. | |
| Proportion of participants with | The vIGA-AD is an Investigator-completed | Baseline to |
| vIGA-AD of 0 (clear) or 1 | assessment scale used to determine severity of | Week 20 |
| (almost clear) and a reduction | AD and clinical response to treatment. It is | |
| from baseline of ≥2 points | based on a 5-point scale, ranging from 0 (clear) | |
| | to 4 (severe). | |
| Proportion of participants | The EASI is an Investigator-assessed validated | Baseline to |
| reaching EASI-90 | tool used to measure the | Week 24 |
| | extent (area) and severity of AD. Total score | |
| | ranges from 0 to 72 with a | |
| | higher score indicating increased extent and | |
| | severity of AD. EASI-90 is 90% reduction | |
| | from baseline in EASI score. | |
| Proportion of participants | The EASI is an Investigator-assessed validated | Baseline to |
| reaching EASI-100 | tool used to measure the | Week 24 |
| | extent (area) and severity of AD. Total score | |
| | ranges from 0 to 72 with a | |
| | higher score indicating increased extent and | |
| | severity of AD. EASI-100 is 100% reduction | |
| | from baseline in EASI score. | |
| Change in Dermatology Quality | The DLQI is a validated 10-item questionnaire | Baseline to |
| of Life Index (DLQI) from | to measure dermatology-specific quality of life | Week 24 |
| baseline | (QoL) in adult patients. Overall scoring ranges | |
| | from 0 to 30, with a higher score indicating a | |
| | poorer QoL. | |
| Proportion of participants with a | The DLQI is a validated 10-item questionnaire | Baseline to |
| reduction in DLQI ≥4 from | to measure dermatology-specific quality of life | Week 24 |
| baseline in participants with | (QoL) in adult patients. Overall scoring ranges | |
| DLQI baseline ≥4 | from 0 to 30, with a higher score indicating a | |
| | poorer QoL. | |
| Change in Hospital Anxiety | The HADS is 14-item questionnaire with two | Baseline to |
| Depression Scale (HADS) from | subscales: anxiety & depression. Each subscale | Week 24 |
| baseline | (anxiety & depression) ranges 0-21. The total | |
| | HADS score ranges 0-42 with higher score | |
| | indicating a poorer state. | |
| Proportion of participants with | HADS-A score ranges 0-21 with higher score | Baseline to |
| HADS subscale Anxiety | indicating a poorer state. | Week 24 |
| (HADS-A) <8 in participants | | |
| with baseline HADS-A ≥8 | | |
| Proportion of participants with | HADS-D score ranges 0-21 with higher score | Baseline to |
| HADS subscale Depression | indicating a poorer state. | Week 24 |
| (HADS-D) <8 in participants | | |
| with HADS-D baseline ≥8 | | |
| Change in weekly average of | The SP-NRS is a single item 0-10 numeric | Baseline to |
| daily Skin Pain-Numerical | rating scale assessing skin pain associated | Week 24 |
| Rating Scale (SP-NRS) from | with AD with 0 = no pain and 10 = worst | |
| baseline | possible pain imaginable. | |
| Proportion of participants with a | The SP-NRS is a single item 0-10 numeric | Baseline to |
| reduction in weekly average of | rating scale assessing skin pain associated | Week 24 |
| daily SP-NRS ≥4 from baseline | with AD with 0 = no pain and 10 = worst | |
| in participants with baseline | possible pain imaginable. | |
| weekly average of daily | | |
| SP-NRS ≥4 | | |
| Change in weekly average of | The SD-NRS is a single item 0-10 numeric | Baseline to |
| daily Sleep Disturbance- | rating scale assessing sleep disturbance | Week 24 |
| Numerical Rating Scale | associated with AD with 0 = no sleep loss | |
| (SD-NRS) from baseline | and 10 = did not sleep at all. | |
| Proportion of participants with a | The SD-NRS is a single item 0-10 numeric | Baseline to |
| reduction in weekly average of | rating scale assessing sleep disturbance | Week 24 |
| daily SD-NRS ≥3 from baseline | associated with AD with 0 = no sleep loss | |
| in participants with Baseline | and 10 = did not sleep at all. | |
| weekly average of daily | | |
| SD-NRS ≥3 | | |
| Proportion of participants with a | The SD-NRS is a single item 0-10 numeric | Baseline to |
| reduction in weekly average of | rating scale assessing sleep disturbance | Week 24 |
| daily SD-NRS ≥5 from baseline | associated with AD with 0 = no sleep loss | |
| in participants with baseline | and 10 = did not sleep at all. | |
| weekly average of daily | | |
| SD-NRS ≥5 | | |
| Percent change in EASI score | The EASI is an Investigator-assessed validated | Baseline to |
| from baseline | tool used to measure the extent (area) and | Week 24 |
| | severity of AD. Total score ranges from 0 to 72 | |
| | with a higher score indicating increased extent | |
| | and severity of AD. | |
| Percent change in weekly | The PP-NRS is a validated single item 0-10 | Baseline to |
| average of daily PP-NRS | numeric rating scale assessing peak pruritus | Week 24 |
| from baseline | (itch) associated with AD with 0 = no itch | |
| | and 10 = worst itch imaginable. | |
| Proportion of participants | The EASI is an Investigator-assessed validated | Baseline to |
| reaching EASI-50 | tool used to measure the extent (area) and | Week 24 |
| | severity of AD. Total score ranges from 0 to 72 | |
| | with a higher score indicating increased extent | |
| | and severity of AD. EASI-50 is 50% | |
| | reduction from baseline in EASI score. | |
| Proportion of participants with | The EASI is an Investigator-assessed validated | Baseline to |
| EASI ≤7 | tool used to measure the extent (area) and | Week 24 |
| | severity of AD. Total score ranges from 0 to 72 | |
| | with a higher score indicating increased extent | |
| | and severity of AD. | |
| Change in percent Body Surface | | Baseline to |
| Area (BSA) affected by AD from | | Week 24 |
| baseline | | |
| Percent change in Scoring | The SCORAD index is a clinical tool to | Baseline to |
| Atopic Dermatitis (SCORAD) | evaluate the extent and severity of AD. Total | Week 24 |
| index from baseline | score ranges from 0 (absent disease) to 103 | |
| | (severe disease). | |
| Proportion of participants with a | The SCORAD index is a clinical tool to | Baseline to |
| reduction in SCORAD ≥8.7 | evaluate the extent and severity of AD. Total | Week 24 |
| points from baseline in | score ranges from 0 (absent disease) to 103 | |
| participants with baseline | (severe disease). | |
| SCORAD score ≥8.7 | | |
| Proportion of participants with a | The POEM is a 7-item self-assessment | Baseline to |
| reduction in Patient Oriented | questionnaire that assesses disease symptoms | Week 24 |
| Eczema Measure (POEM) ≥4 | on a 5-point scale; 0 (no days) to 4 (every day | |
| from baseline in participants | in the last week). The sum of the 7 items gives | |
| with POEM Baseline ≥4 | the total POEM score of 0 (absent disease) to | |
| | 28 (very severe). Higher scores indicated more | |
| | severe disease and poor quality of life. | |
| Change in POEM from baseline | The POEM is a 7-item self-assessment | Baseline to |
| | questionnaire that assesses disease symptoms | Week 24 |
| | on a 5-point scale; 0 (no days) to 4 (every day | |
| | in the last week). The sum of the 7 items gives | |
| | the total POEM score of 0 (absent disease) to | |
| | 28 (very severe). Higher scores indicated more | |
| | severe disease and poor quality of life. | |
| Proportion of participants with | | Baseline to |
| rescue medication use [from | | Week 24 |
| baseline to Week 24] | | |
| Percentage of participants who | | Baseline to |
| experienced Treatment- | | Week 40 |
| Emergent Adverse Events | | |
| (TEAEs), experienced | | |
| Treatment-Emergent Serious | | |
| Adverse Events (TESAEs) | | |
| and/or Treatment-Emergent | | |
| Adverse Events of Special | | |
| Interest (AESI) | | |
| Serum amlitelimab | | Baseline to |
| concentrations | | Week 40 |
| Incidence of antidrug antibodies | | Baseline to |
| (ADAs) of amlitelimab | | Week 40 |

### Example VI

### Adolescent Dosing

### Rationale and Objectives:

Patients <40 kg are anticipated to have a 1.7-fold (35-40 kg) to 2.0-fold (25-30 kg) higher median exposure with up 25% to 50% over-exposed patients as compared to AD adult patients ≥40 kg. To select a dose/dosing regimen for patients <40 kg (range 25-40 kg) to generate amlitelimab exposure within the exposure range observed in AD adult patients ≥40 kg.

The proposed dosing regimen and study duration for the adolescents ≥40 kg is the same as for adults. At the time of the protocol amendment to include adolescents in the Phase 3 program, a dose of 125 mg with a 250 mg loading dose is proposed for adolescents between 25 and 40 kg. This dose was selected based on simulations performed with the population PK model to generate amlitelimab exposure within the exposure range observed with the proposed dose regimen in AD adult participants ≥40 kg.

After 250 mg Q4W with a 500 mg loading dose, adolescent participants <40 kg are anticipated to have a 1.7-fold (35-40 kg) to 2.0-fold (25-30 kg) higher median exposure with up 25% to 50% overexposed participants as compared to AD participants >40 kg. In order to include adolescent participants <40 kg (range 25-40 kg) in the Phase 3 program, PK simulations were performed based on a PopPK model with body weight-based allometric scaling with the objective to generate amlitelimab exposures in participants <40 kg within the exposure range observed in AD adult participants ≥40 kg (range: 40-150 kg) at the selected dosing regimens. To select an appropriate Q4W dosing regimen, predicted exposures (AUC4W and Cmax) following 125 mg Q4W with a 250 mg loading dose in participants <40 kg were compared to predicted exposures following 250 mg Q4W with a 500 mg loading dose in participants >40 kg across different body weight bands through an exposure matching approach. A similar approach was applied to compare the exposures following 125 mg Q12W with a 250 mg loading dose in participants <40 kg and 250 mg Q12W with a 500 mg loading dose in participants ≥40 kg.

### Method:

Perform simulations in virtual AD patients using PopPK model with body weight allometric scaling with the following conditions:
1000 virtual patients by BW (body weight) band: 40-150 kg (reference) and 25-40 kg.
Dosing regimen:
Loading dose then Q4W dose up to week 20.
Other dosing regimens were tested (Q6W and Q8W)

### Results:

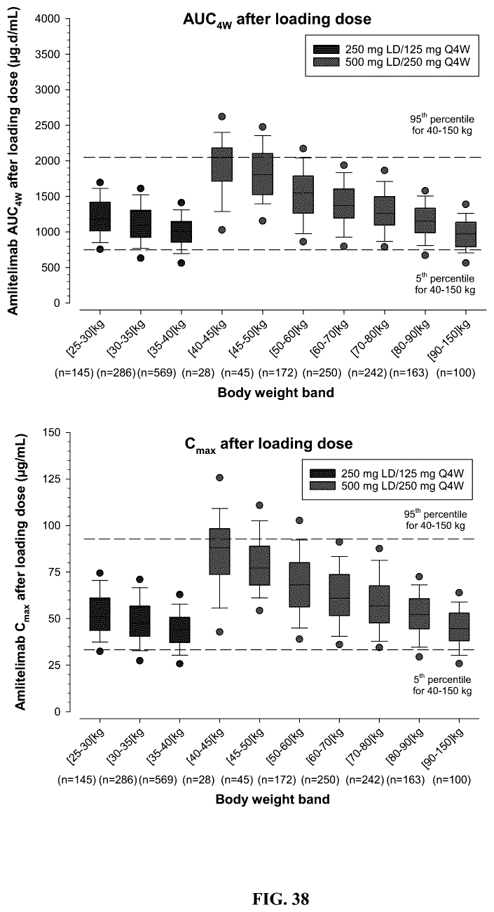

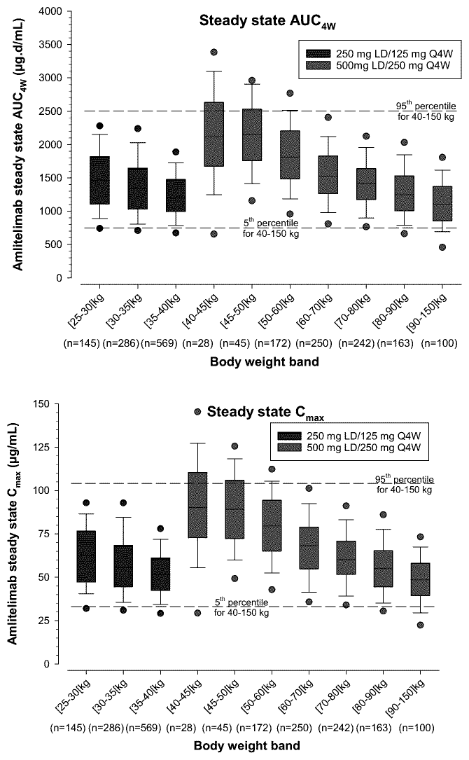

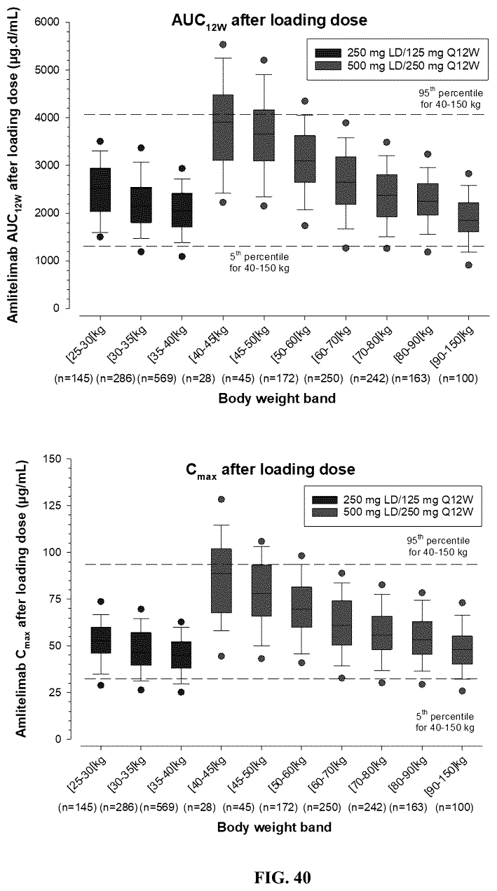

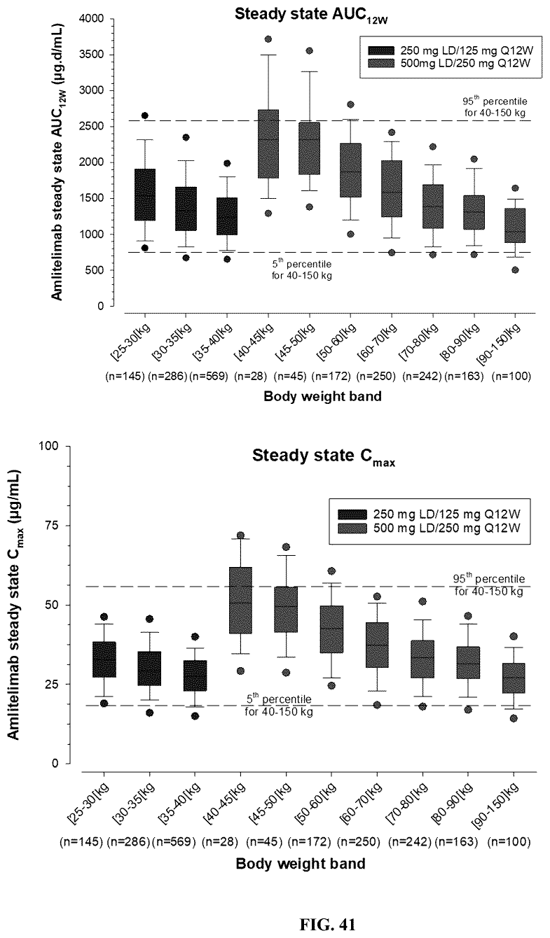

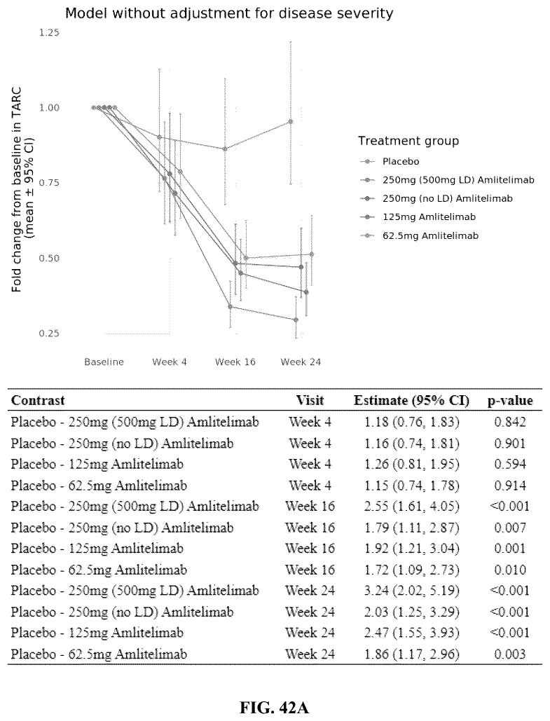

Simulations were performed by generating 1000 virtual participants by body weight band, i.e., 40-150 kg and 25-40 kg. The simulation results showed that for participants with a body weight between 25 and 40 kg, 125 mg Q4W with a 250 mg loading dose would have exposures in the range of that in participants with body weight between 40 and 150 kg following 250 mg Q4W with a 500 mg loading dose (FIG. 38 and FIG. 39). In addition, Cmax in participants <40 kg remained below the highest Cmax documented in participants ≥40 kg. Similarly, the simulation results showed that 125 mg Q12W with a 250 mg loading dose for participants with body weight between 25 and 40 kg would have exposures in the range to that in participants with body weight between 40 and 150 kg following 250 mg Q12W with a 500 mg loading dose (FIG. 40 and FIG. 41).

Based on the above simulations and an exposure matching approach, two dosing regimens for adolescents with body weight between 25 kg and 40 kg who will be included in the Phase 3 studies.

Proposed dose regimens are:
Amlitelimab 250 mg loading dose followed by 125 mg Q4W;
Amlitelimab 250 mg loading dose followed by 125 mg Q12W.

### Example VII

### Amlitelimab Significantly Reduces the Levels of TARC, LDH, IL-5 and Eosinophil Counts

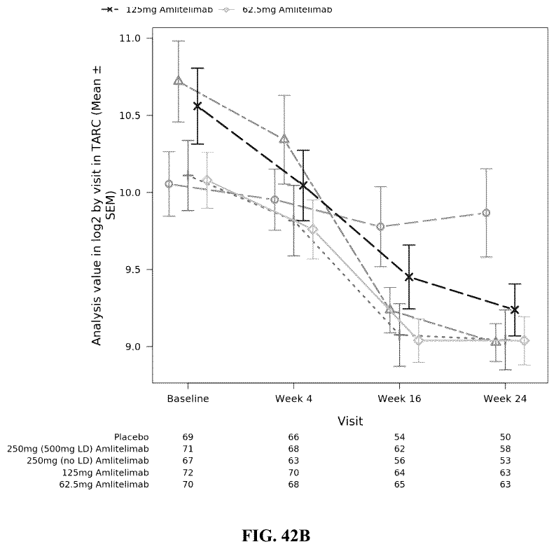

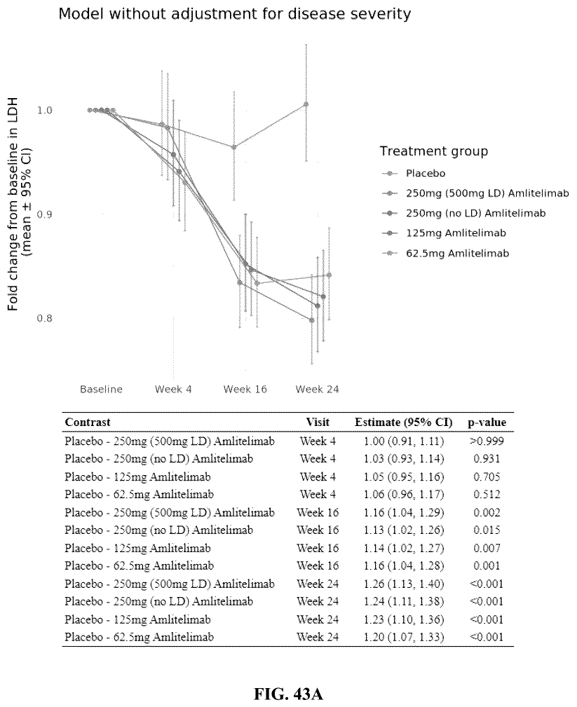

FIGS. 42A and 42B depict treatment effect assessment bases on biomarker thymus and activation-regulated chemokine (TARC) levels (+/−10%).

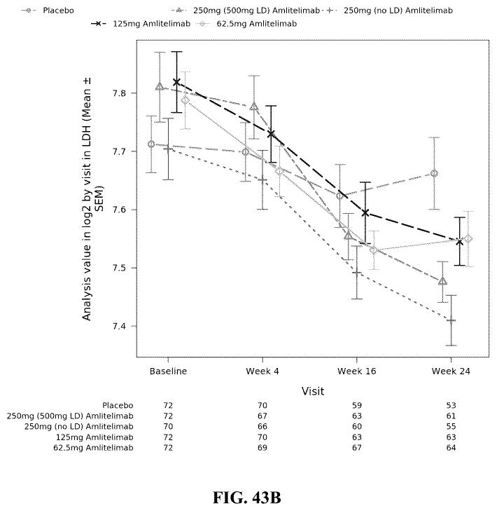

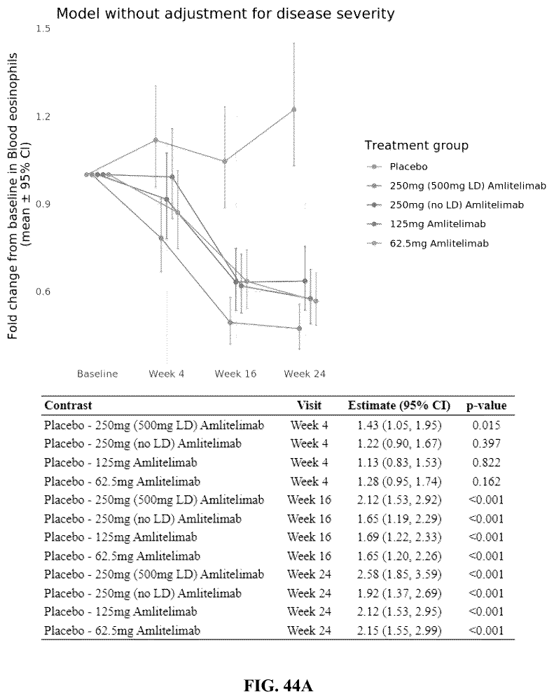

FIGS. 43A and 43B depict treatment effect assessment bases on biomarker lactate dehydrogenase (LDH) levels (+/−10%).

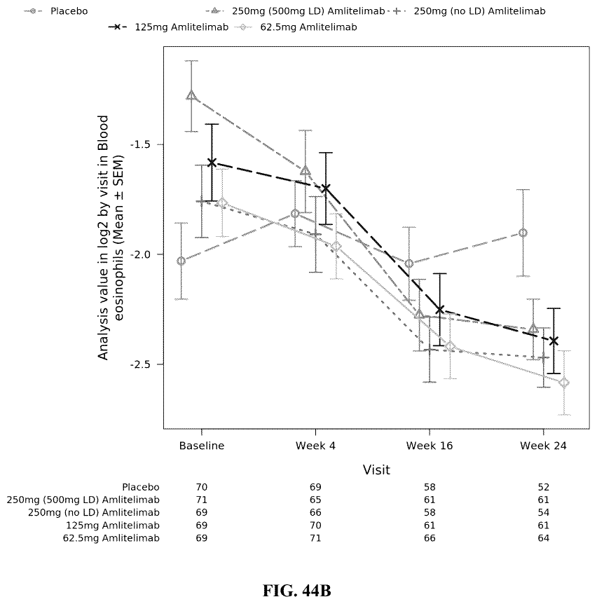

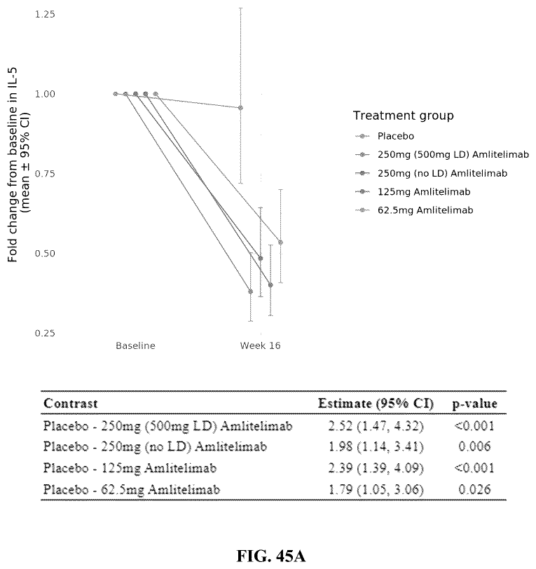

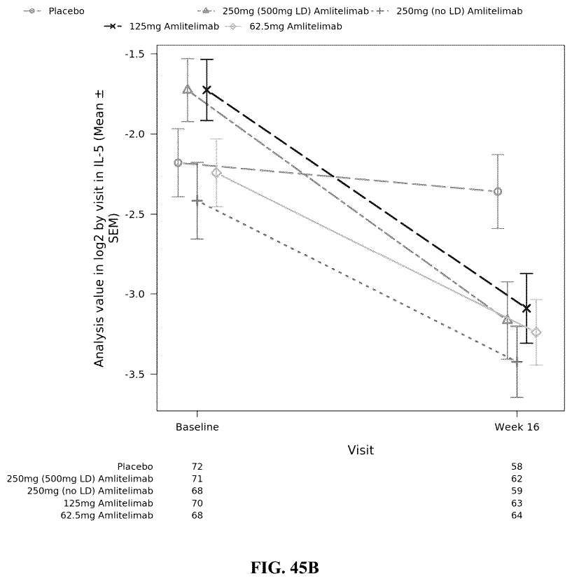

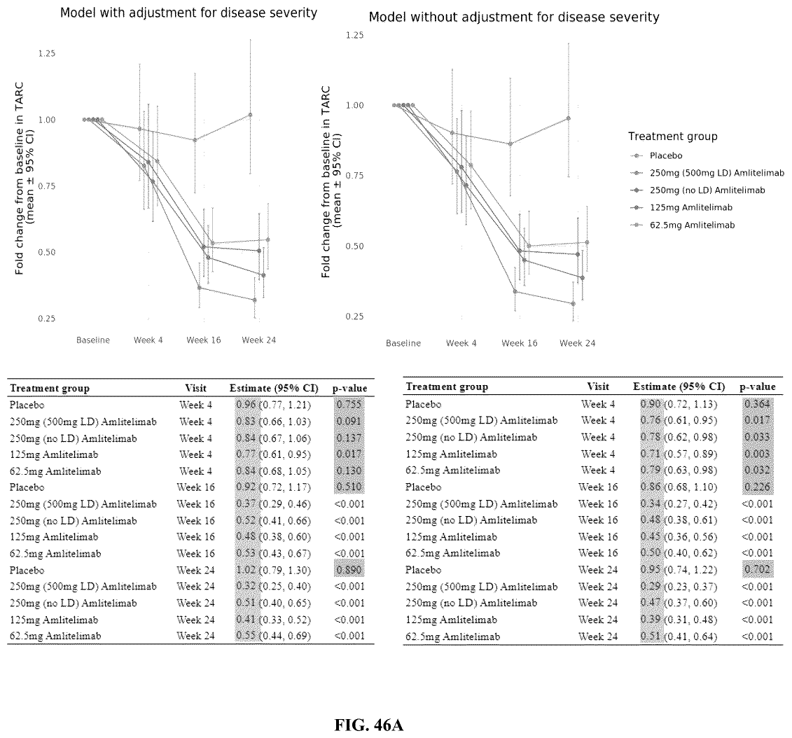

FIGS. 44A and 44B depict treatment effect assessment bases on eosinophil count levels (+/−10%).

FIGS. 45A and 45B depict treatment effect assessment bases on biomarker interleukin-5 (IL-5) levels (+/−10%).

### Example VIII

### Comparison Among the Models+/−Adjustment for Severity of Treatment

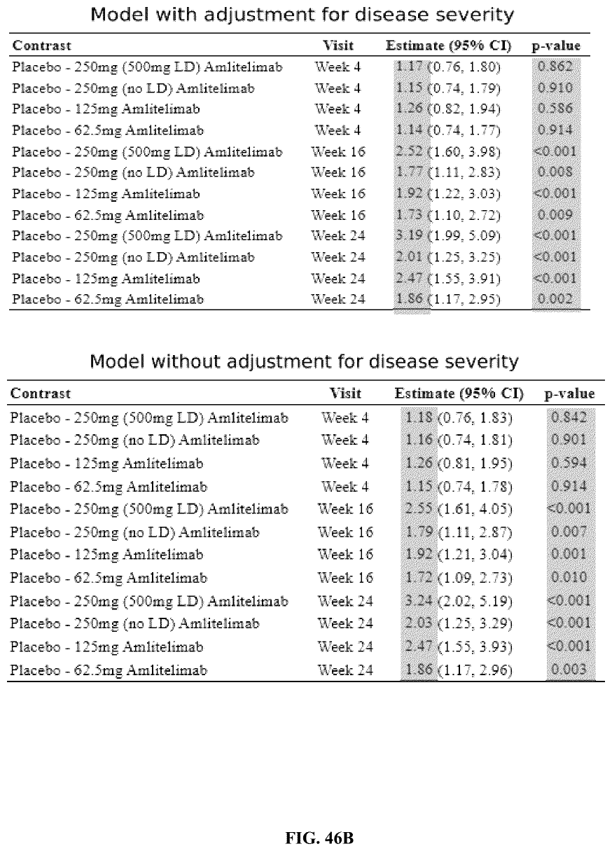

FIGS. 46A and 46B depict a treatment effect assessment model of TARC. A comparison between the models with or without adjustment for disease severity showed that the overall results were the same. Overall, the p-value of the estimates of the ratio to baseline was slightly lower in the model without adjustment than in the model with adjustment for disease severity.

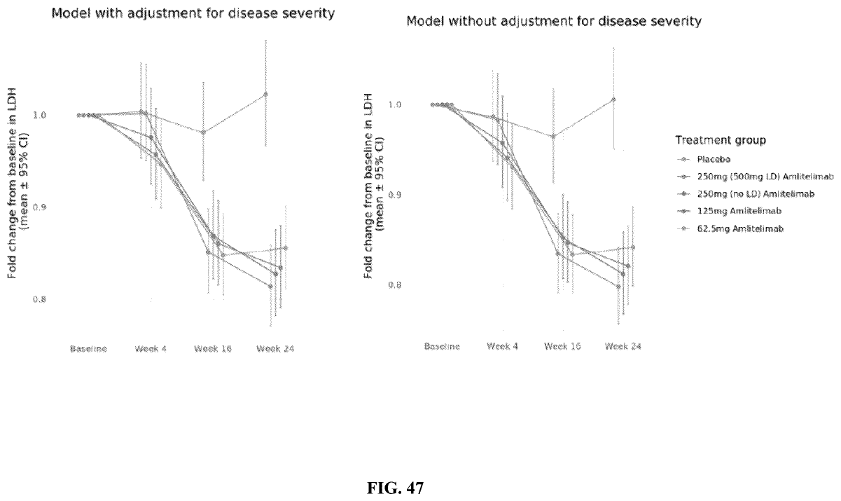

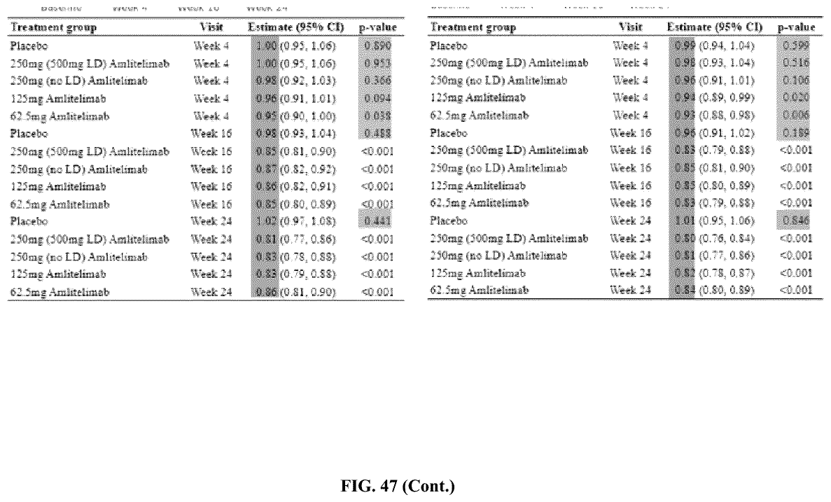

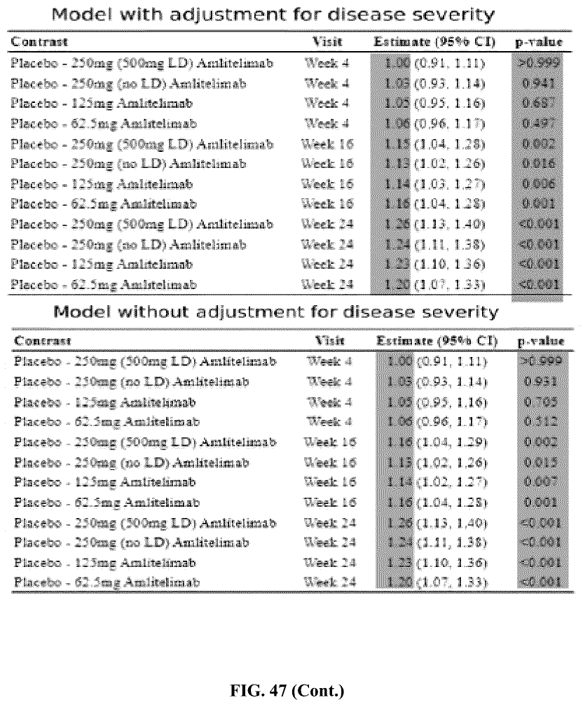

FIG. 47 depicts a treatment effect assessment model of LDH (+/−10%).

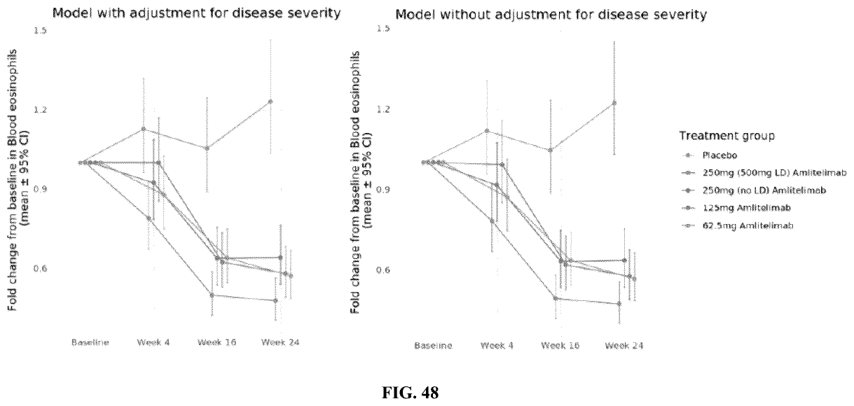

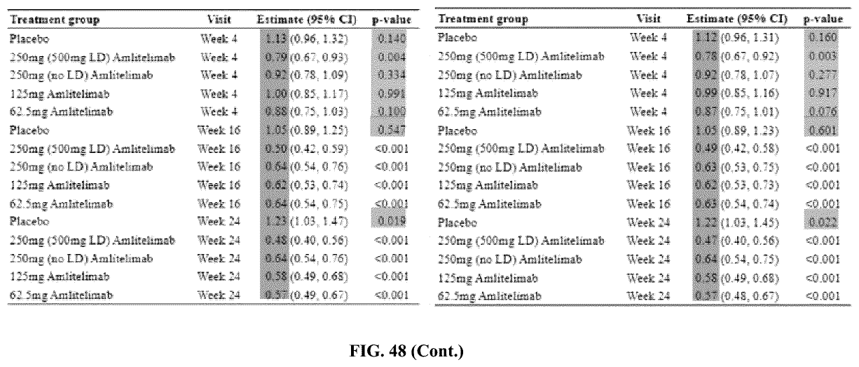

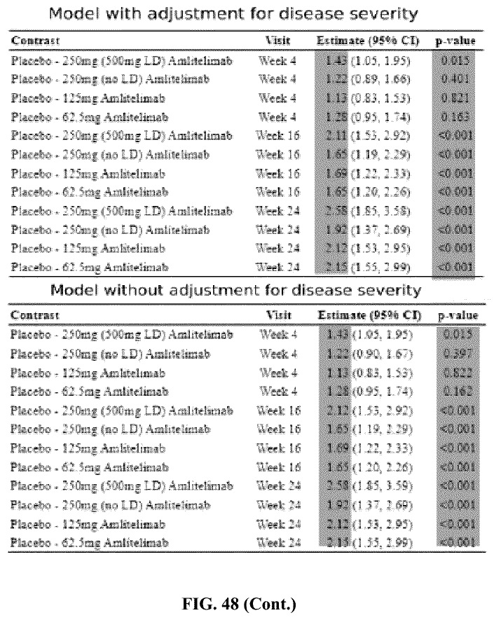

FIG. 48 depicts a treatment effect assessment model of blood eosinophils (+/−10%).

### Example IX

### Additional Biomarker Analysis

### Objectives:

PD: Assess dose-dependent effect of amlitelimab on biomarkers
Test hypothesis that amlitelimab decreases biomarkers associated with different immune pathways (e.g., Th2/17/22)
Th2-associated: IL-13, TARC, Eotaxin-3, IL-5, IL-31
Th17/22-associated: IL-17A and IL-22
Total IgE, LDH and eosinophil count

### Statistical Analysis Considerations:

PD: Treatment effect on biomarkers with repeated measures (baseline, W4, W16, W24)
MMRM approach: the log-transformed fold change from for each biomarker is modeled using a mixed model with repeated measures that includes treatment, visit, and disease severity as fixed effects and a treatment-by visit interaction term. This model is fitted with an unstructured covariance to capture the within-subject correlation between visits, i.e., to take account of the dependence between repeated measurements.
PD: Treatment effect on biomarkers with one post-baseline value (W16)
ANCOVA approach: the log-transformed fold change from baseline for each biomarker is modeled and assumed to depend on disease severity and treatment arm.

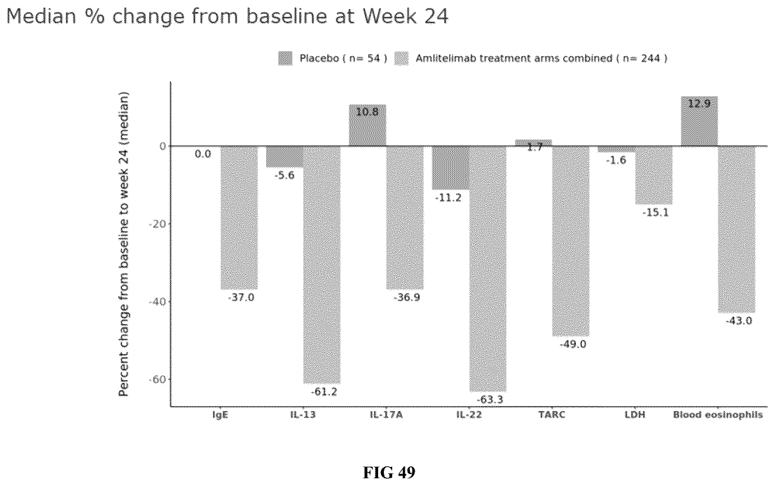

FIG. 49 depicts that amlitelimab modulated all measured biomarkers with the highest observed effect in Th2/Th22 biomarkers at week 24 (+/−10%). Median % change from baseline was measured at week 24 for IgE, IL-13, IL-17A, IL-22, TARC, LDH and eosinophils.

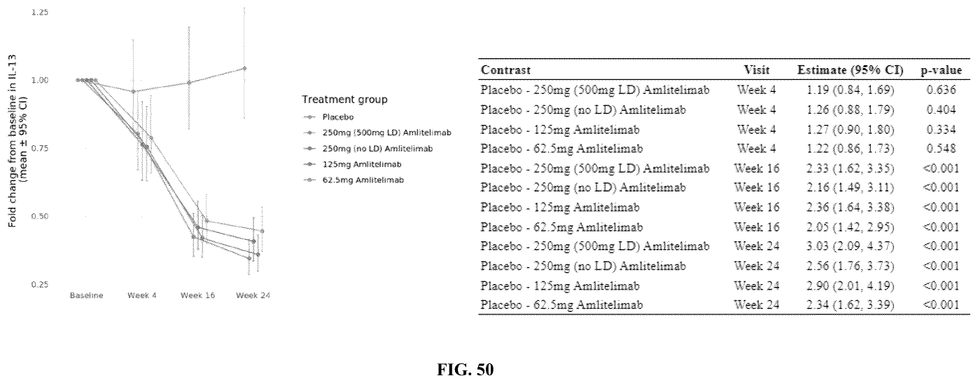

FIG. 50 depicts a treatment effect assessment model: Th2-associated biomarker IL-13 (+/−10%).

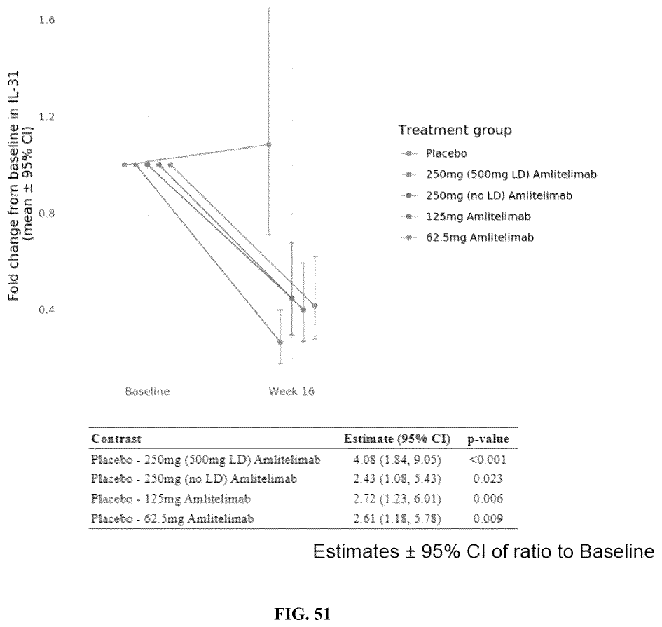

FIG. 51 depicts a treatment effect assessment model: Th2-associated biomarker IL-31 (+/−10%).

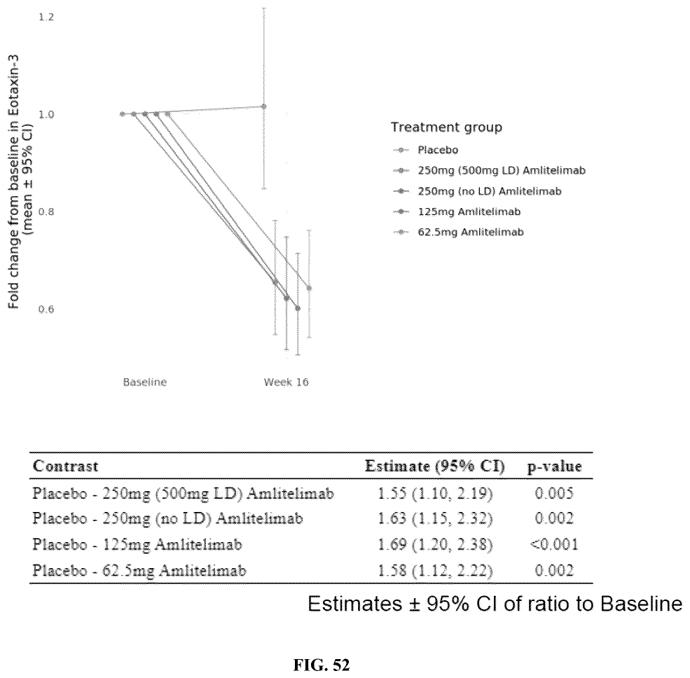

FIG. 52 depicts a treatment effect assessment model: Th2-associated biomarker eotaxin-3 (+/−10%).

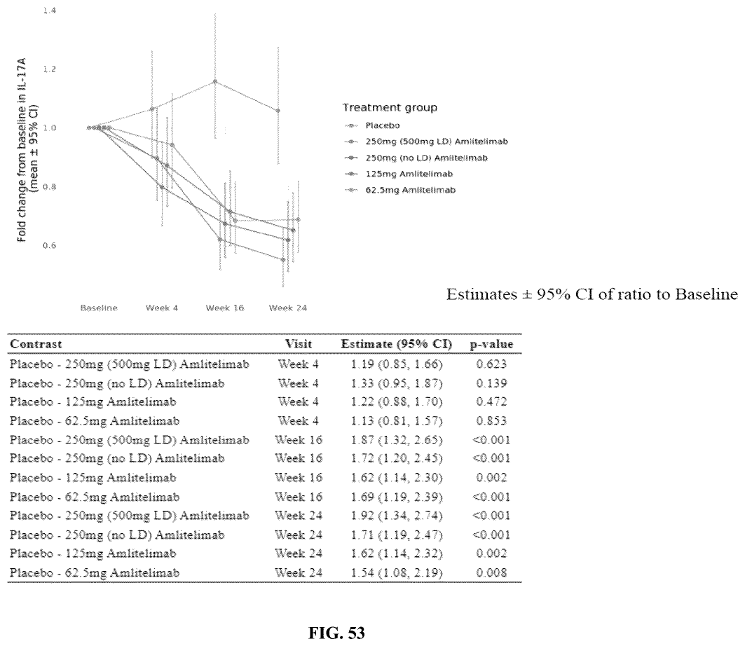

FIG. 53 depicts a treatment effect assessment model: IL-17A (+/−10%).

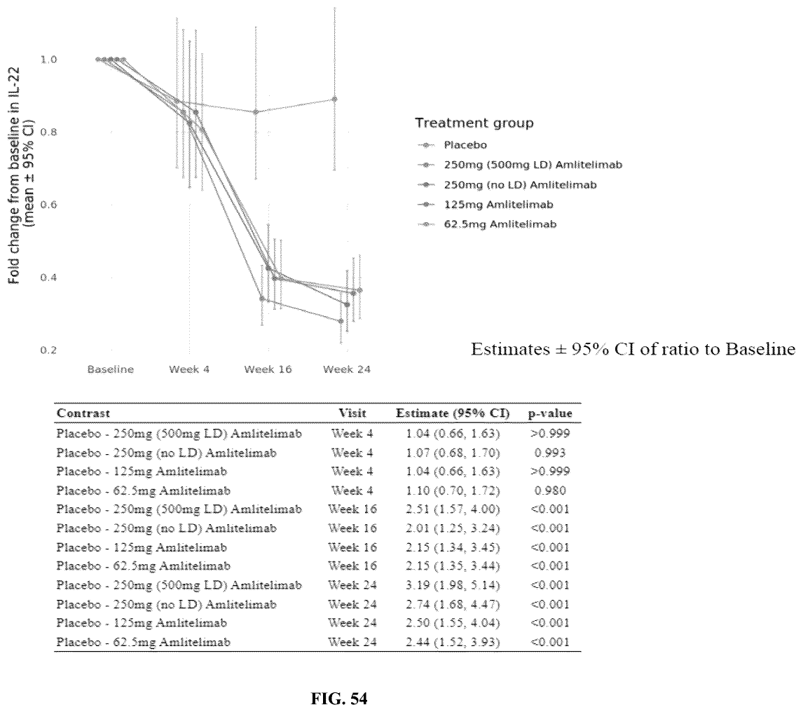

FIG. 54 depicts a treatment effect assessment model: IL-22 (+/−10%).

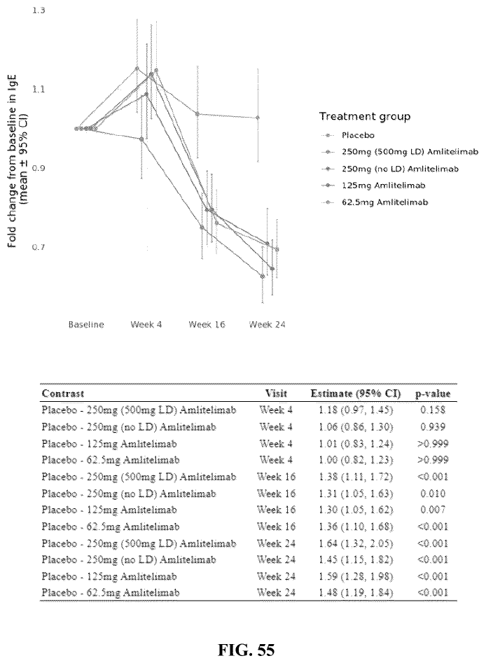

FIG. 55 depicts a treatment effect assessment model: IgE (+/−10%).

The results show:
Amlitelimab significantly reduced all presented biomarkers across all doses at weeks 16 and 24, demonstrating modulation of Th2/Th17/Th22 pathways.

### Treatment Effect Assessment

Fold-change estimates and descriptive raw values (log 2 transformed) were evaluated.

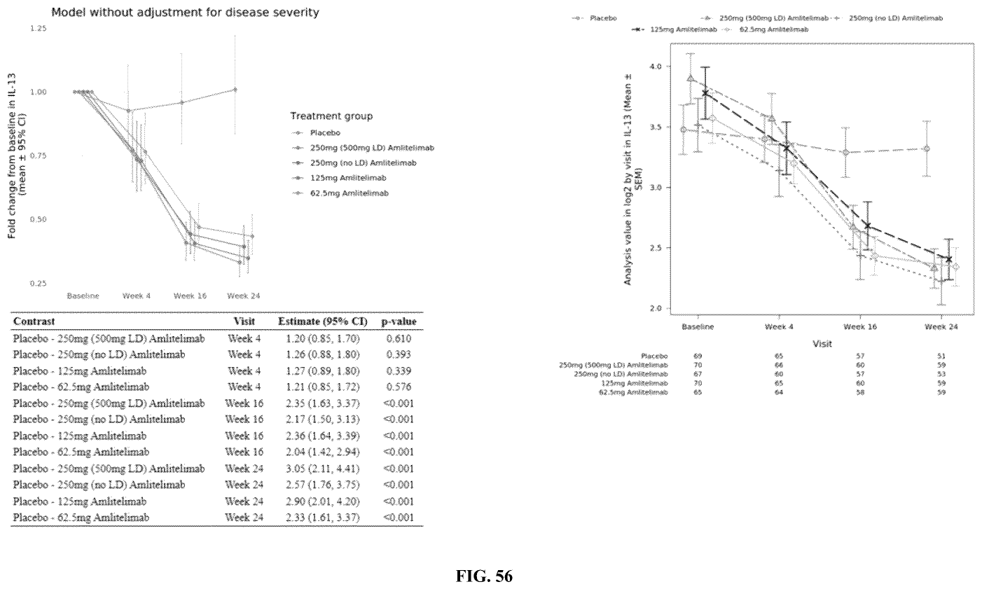

FIG. 56 depicts a treatment effect assessment model: Th2-associated biomarker IL-13 (+/−10%).

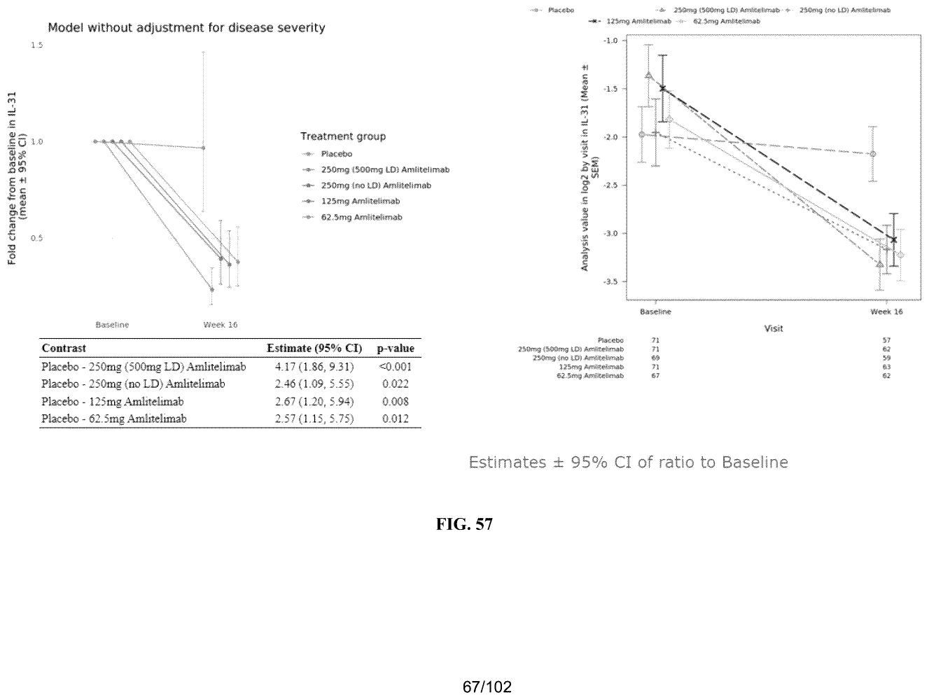

FIG. 57 depicts a treatment effect assessment model: Th2-associated biomarker IL-31 (+/−10%).

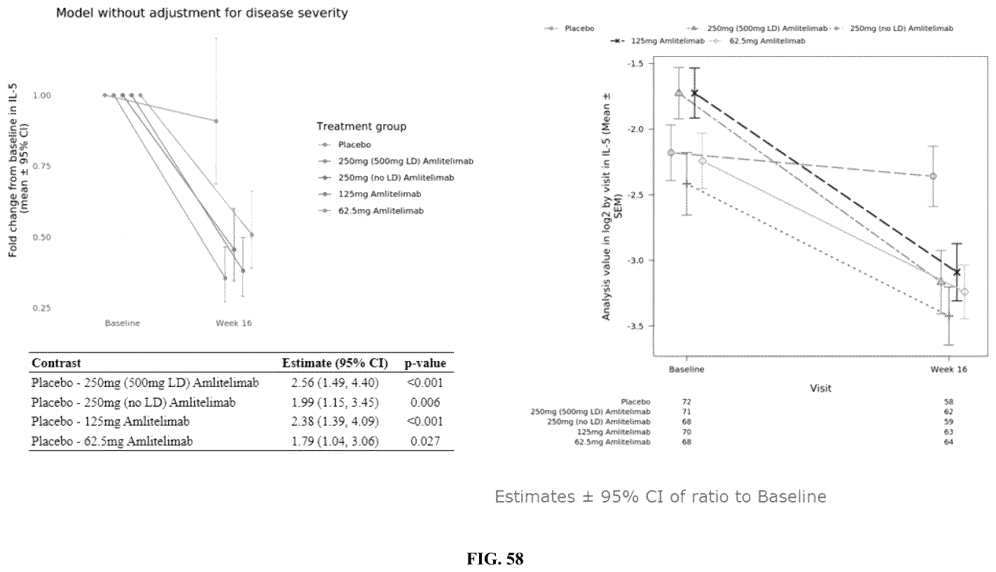

FIG. 58 depicts a treatment effect assessment model: Th2-associated biomarker IL-5 (+/−10%).

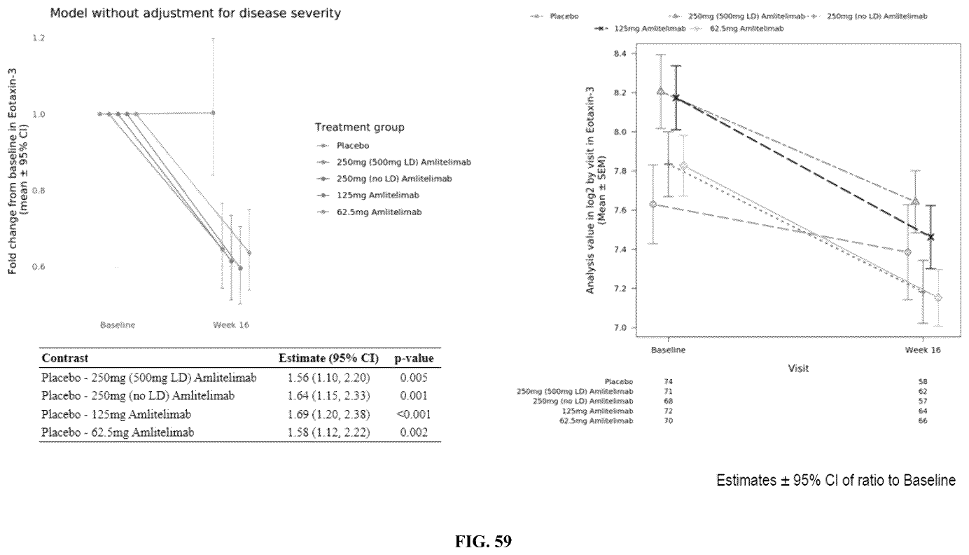

FIG. 59 depicts a treatment effect assessment model: Th2-associated biomarker eotaxin-3 (+/−10%).

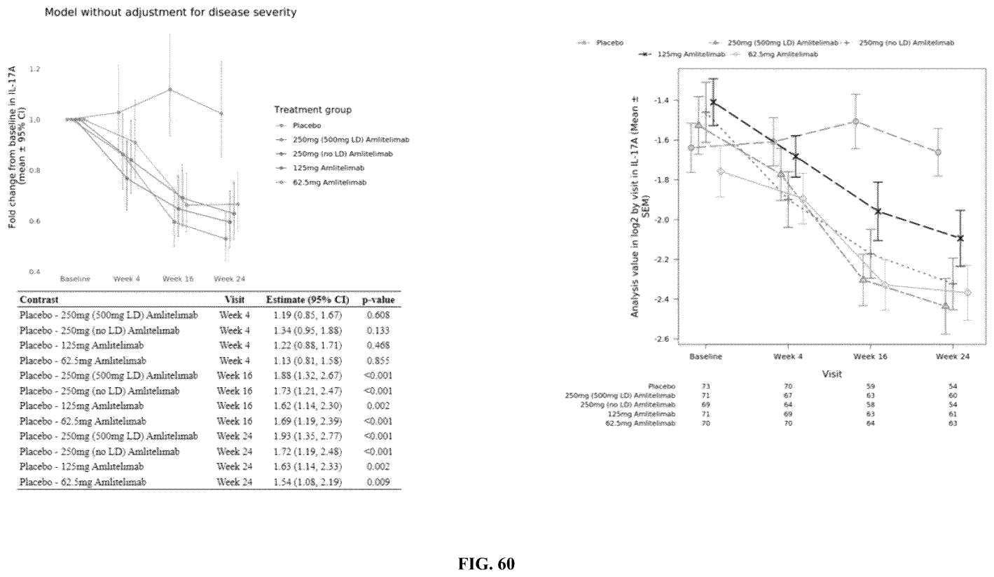

FIG. 60 depicts a treatment effect assessment model: Th17/22-associated biomarkers: IL-17A (+/−10%).

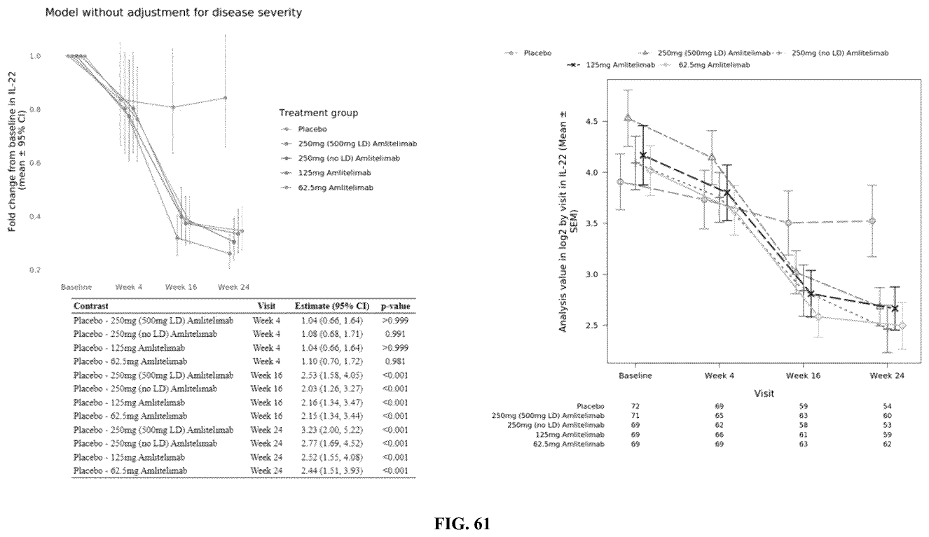

FIG. 61 depicts a treatment effect assessment model: Th17/22-associated biomarkers: IL-22 (+/−10%).

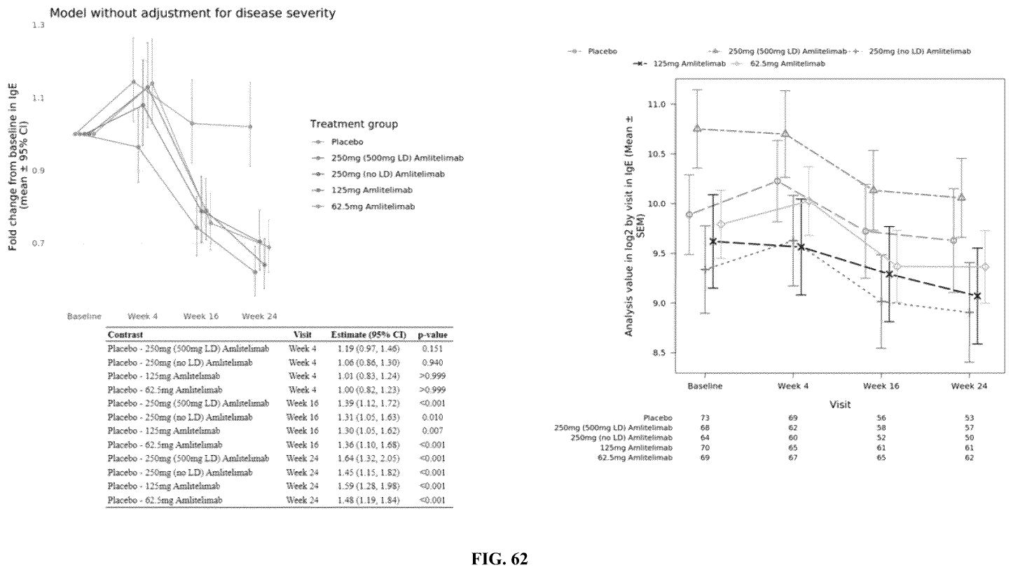

FIG. 62 depicts treatment atopic dermatitis disease related biomarkers IgE (+/−10%).

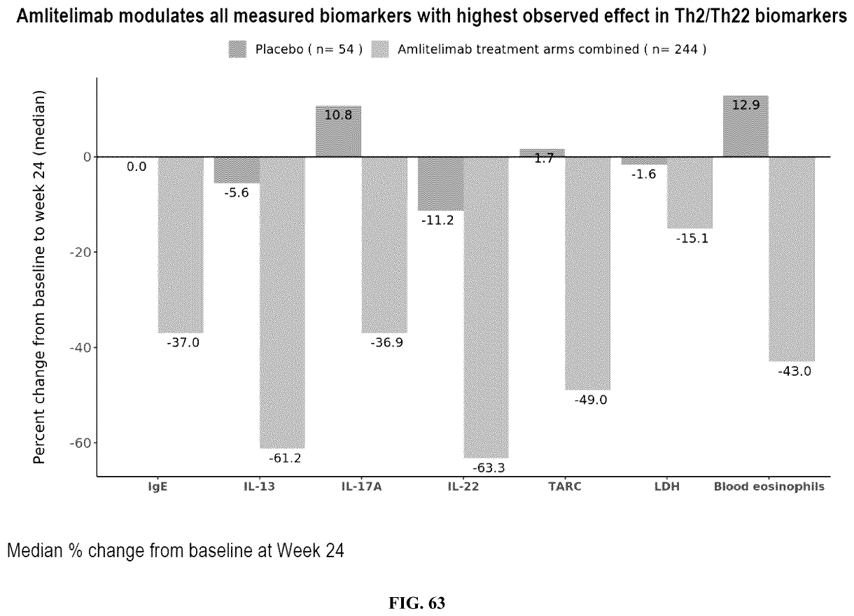

FIG. 63 depicts that amlitelimab modulated all measured biomarkers with the highest observed effect in Th2/Th22 biomarkers (+/−10%).

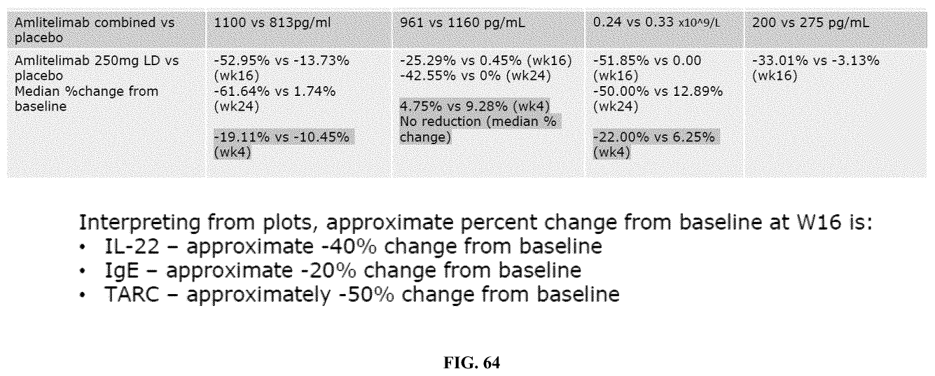

FIG. 64 is a table showing amlitelimab modulates biomarkers TARC, IgE, eosinophils, and eotaxin-3 (+/−10%).

Interpreting from plots, approximate percent change from baseline at week 16 was:
IL-22-approximate −40% change from baseline (+/−10%).
IgE-approximate −20% change from baseline (+/−10%).
TARC-approximately −50% change from baseline (+/−10%)

### Treatment Effect Assessment

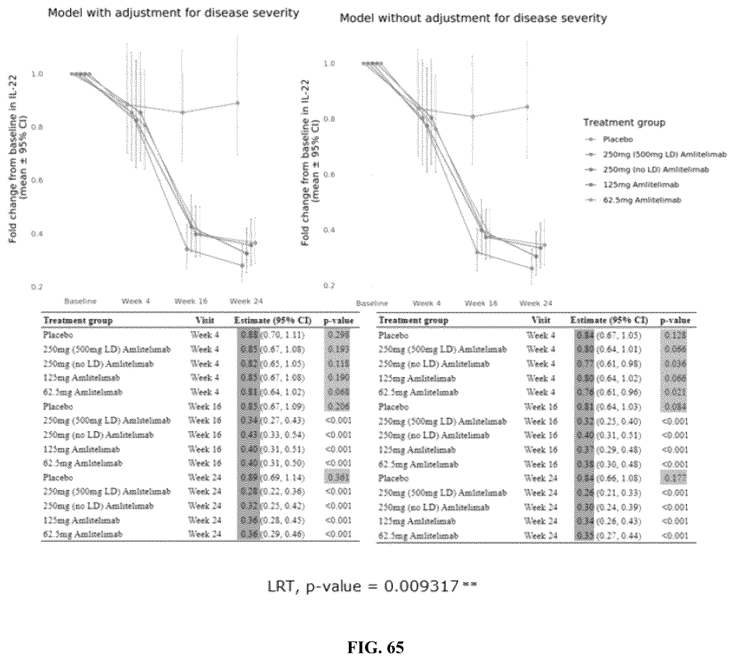

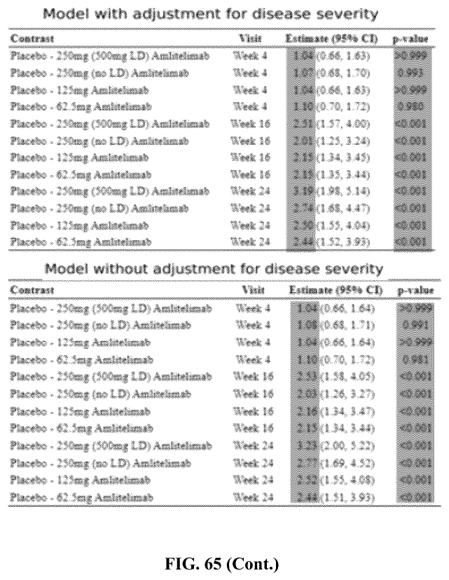

FIG. 65 depicts a treatment effect assessment model: IL-22 (+/−10%).

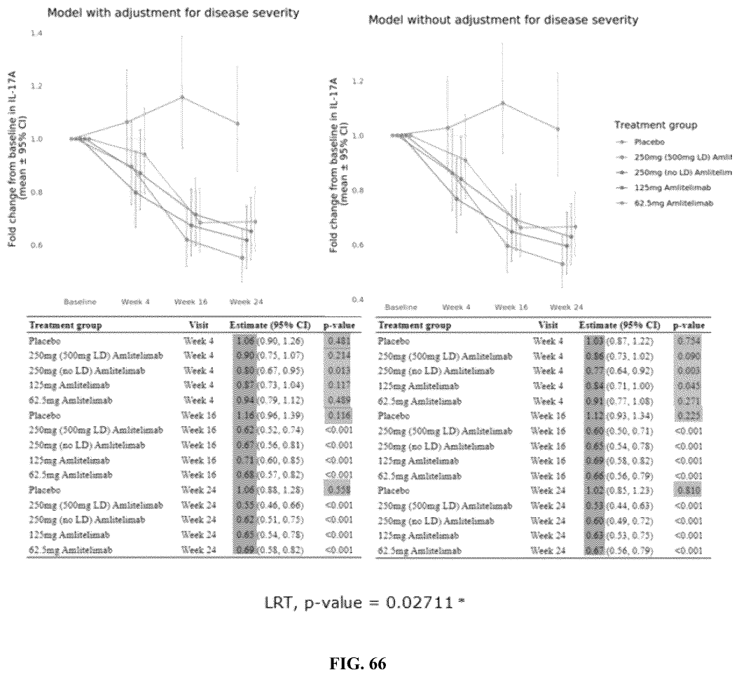

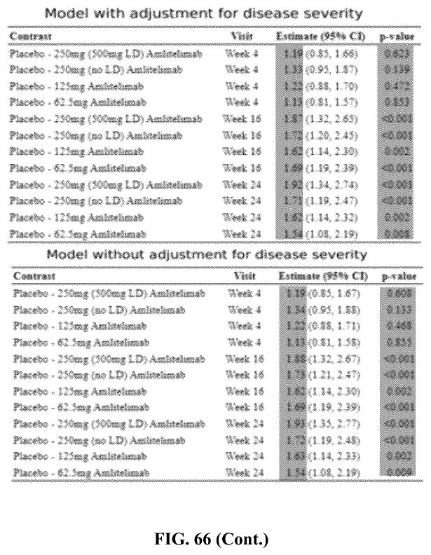

FIG. 66 depicts a treatment effect assessment model: IL-17A (+/−10%).

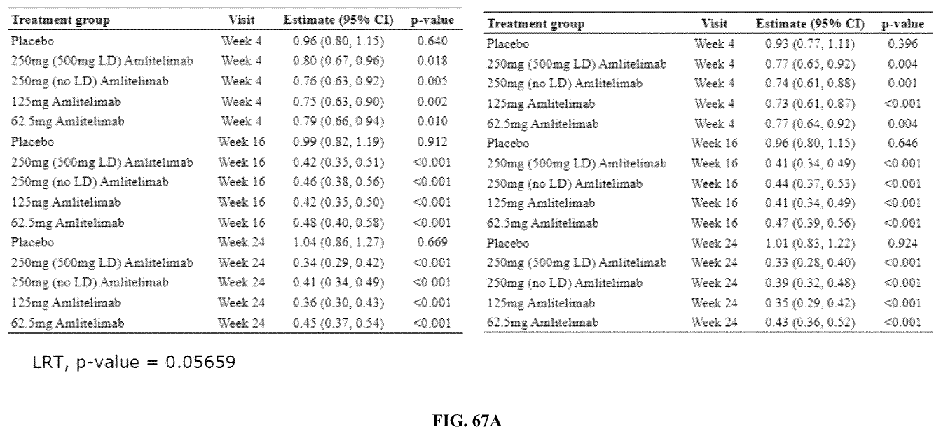

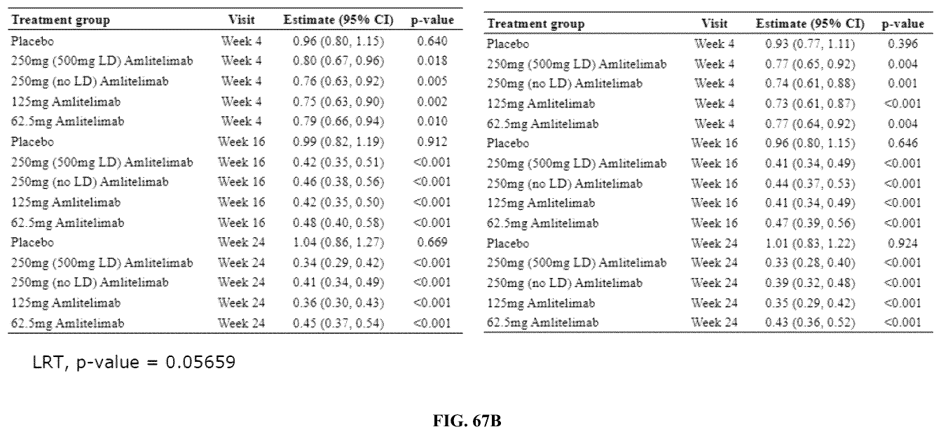

FIGS. 67A and 67B depict a treatment effect assessment model: IL-13 (+/−10%).

### Biomarker Levels at Week 52

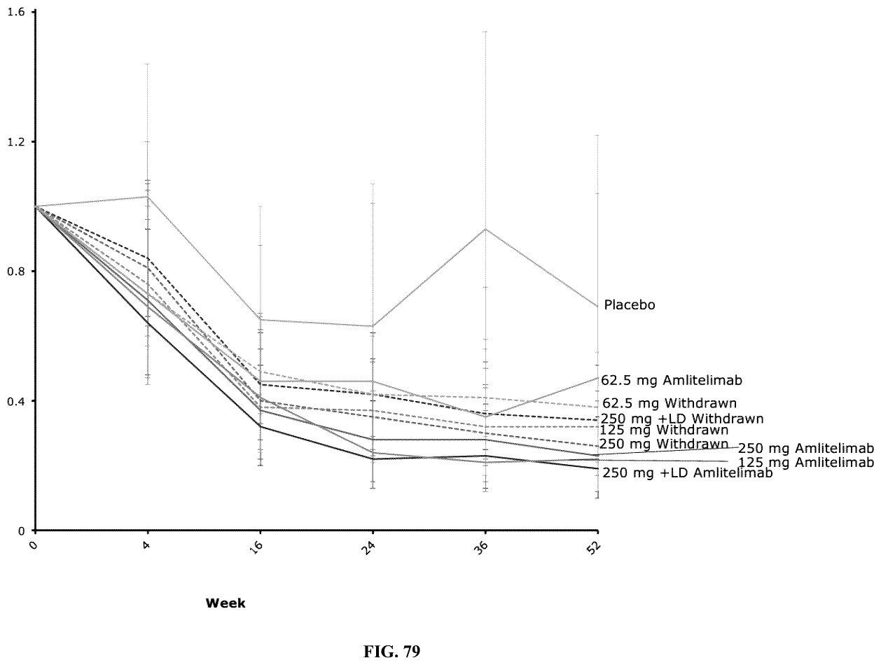

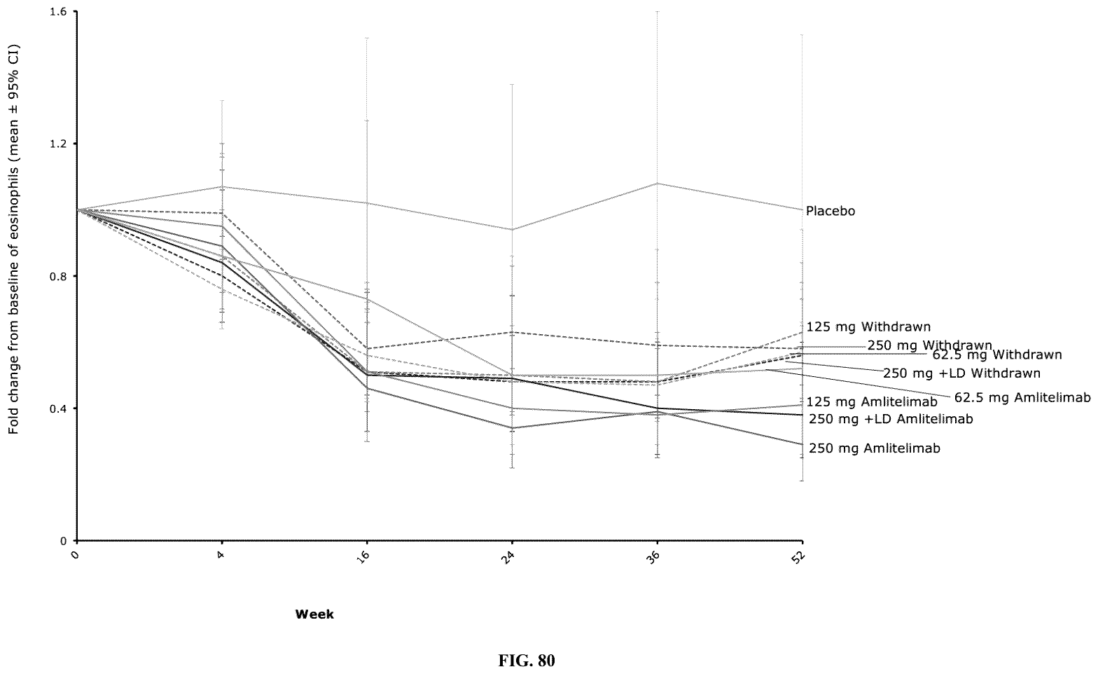

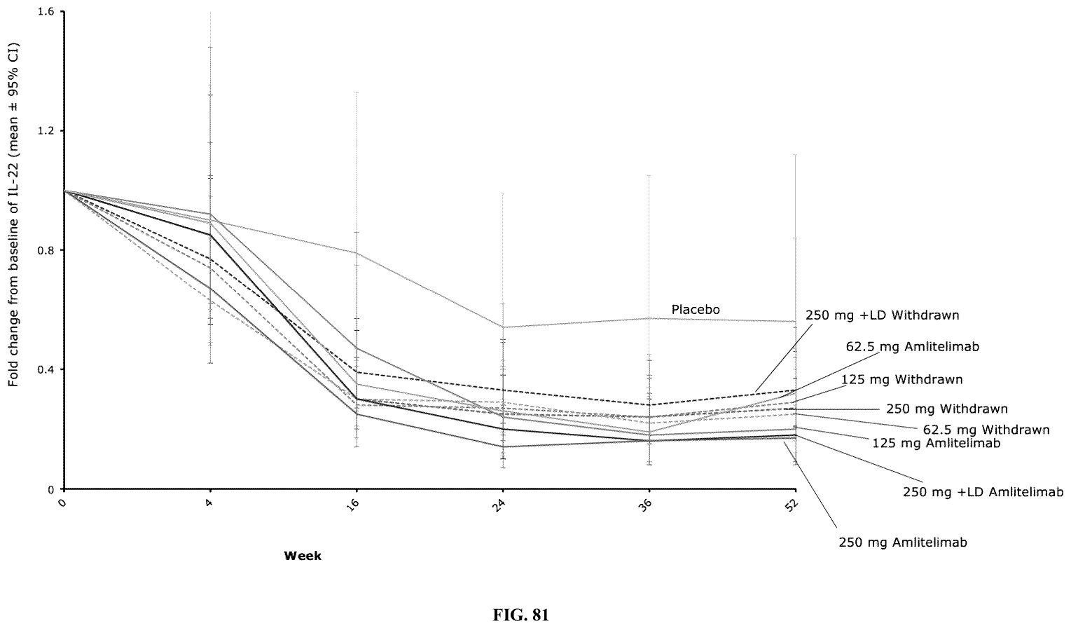

The AD-related biomarkers TARC (FIG. 79), eosinophils (FIG. 80) and IL-22 (FIG. 81) remained suppressed after amlitelimab was cleared from the serum up to week 52, further supporting durable disease control via normalization of inflammatory T-cells. Additionally, continued suppression of IL-13 and IL-17 was observed up to week 52.

Analyses of treatment effects on blood biomarkers were performed both on the modified biomarker evaluable set (full analysis set (FAS) population with a baseline and at least 1 post-baseline result) and on the biomarker evaluable set (non-rescue medication (NRM) population, defined as those not taking rescue and/or prohibited medication impacting efficacy while on investigational treatment, with a baseline and at least 1 postbaseline result) as it was determined to be relevant to the mechanism of action of amlitelimab.

Fold-changes from baseline of log-transformed biomarker concentrations over time were modeled with an MMRM approach and included treatment visit and disease severity as fixed factors, and a treatment by visit interaction term. For biomarkers with only baseline and week 16 measurements, fold-changes from baseline of log-transformed biomarker concentrations were modeled with an ANCOVA approach and assumed to depend on disease severity and treatment group.

Following a comprehensive review of the derivation rule of the rescue medication population flag in Part 1, it was noted that the rule inadvertently excluded participants who were ongoing in Part 1 but then left the study during this phase, without progressing to Part 2. The revised approach stipulates that the upper threshold of the derivation rules for defining a participant's rescue status should be based on the latest available data points up to Week 24.

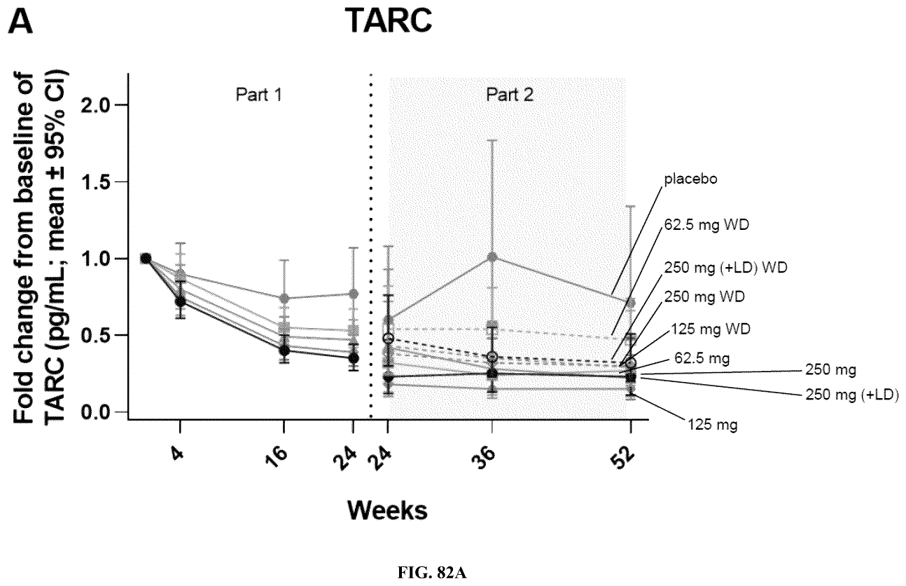

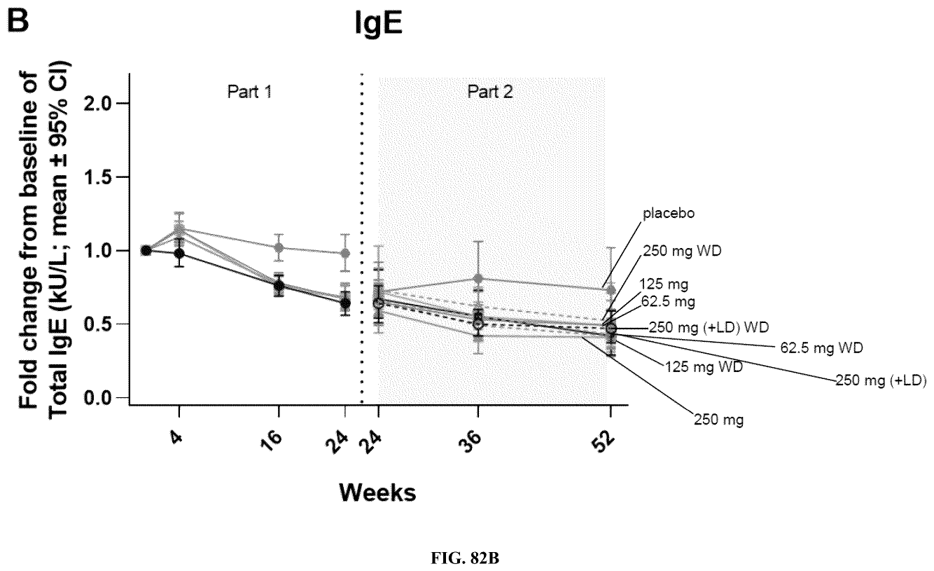

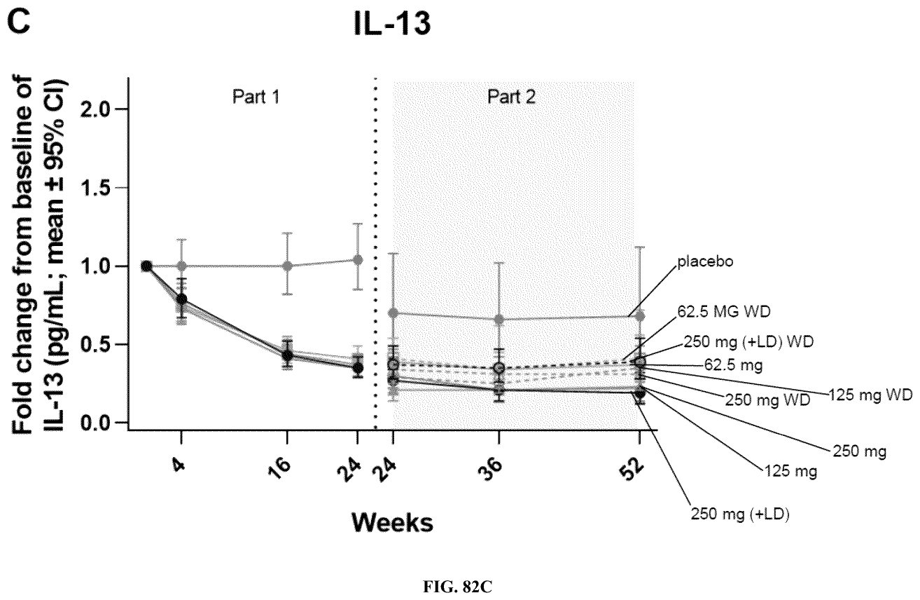

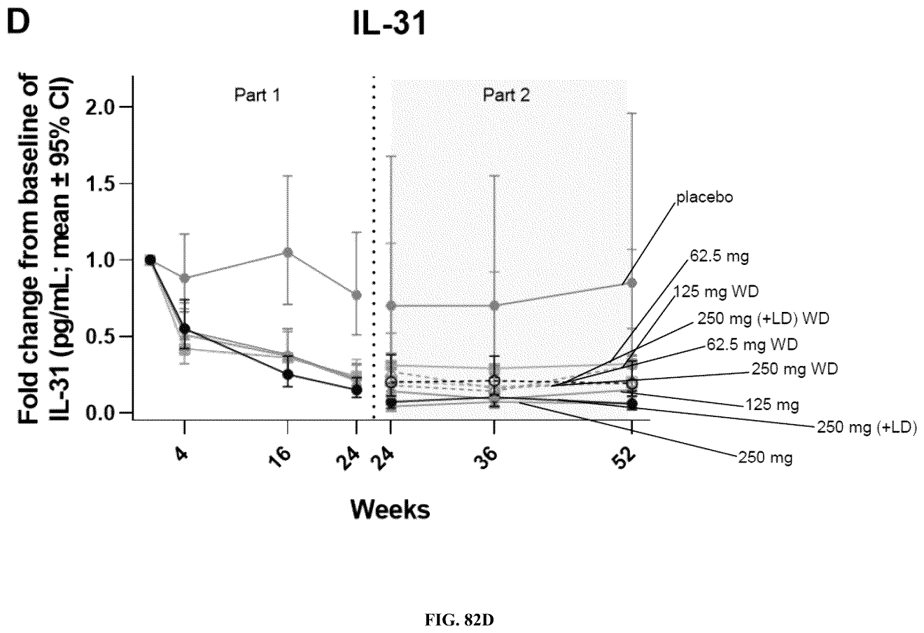

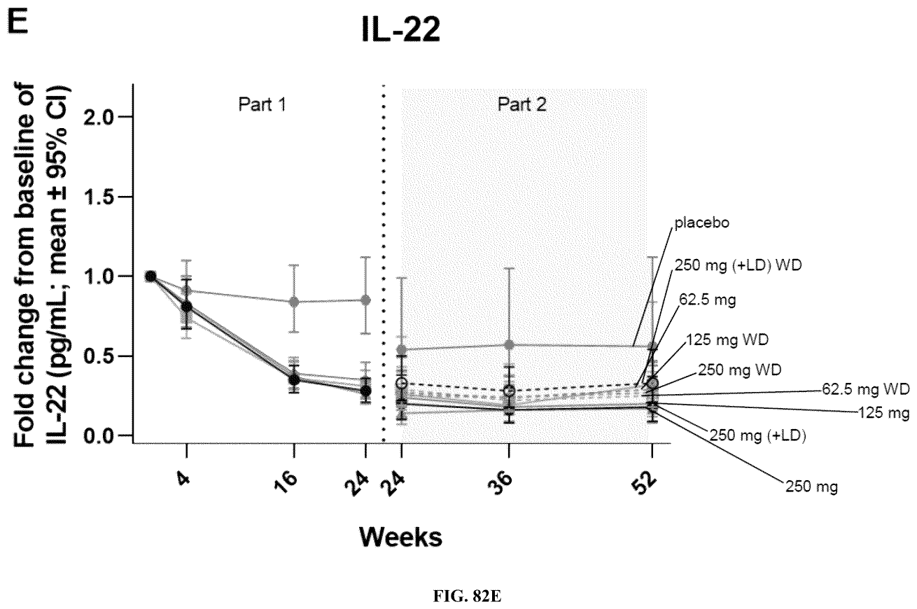

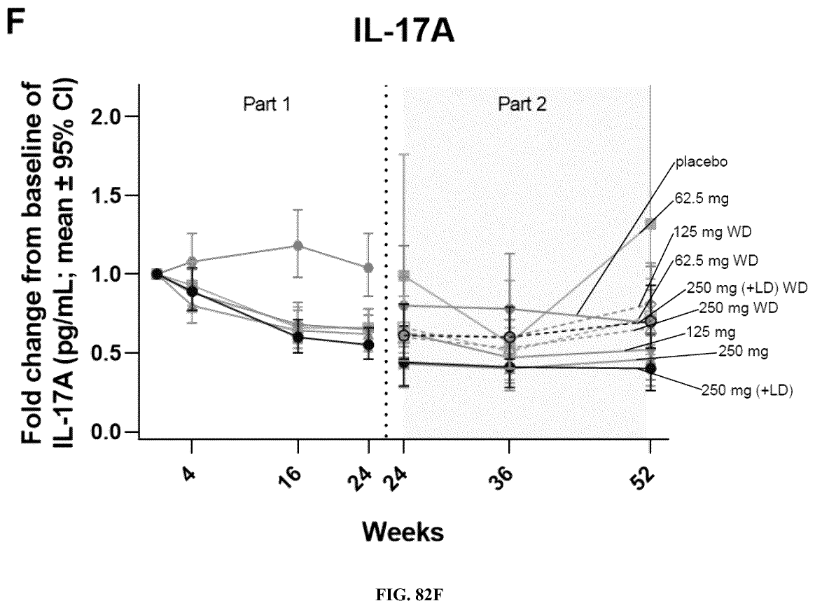

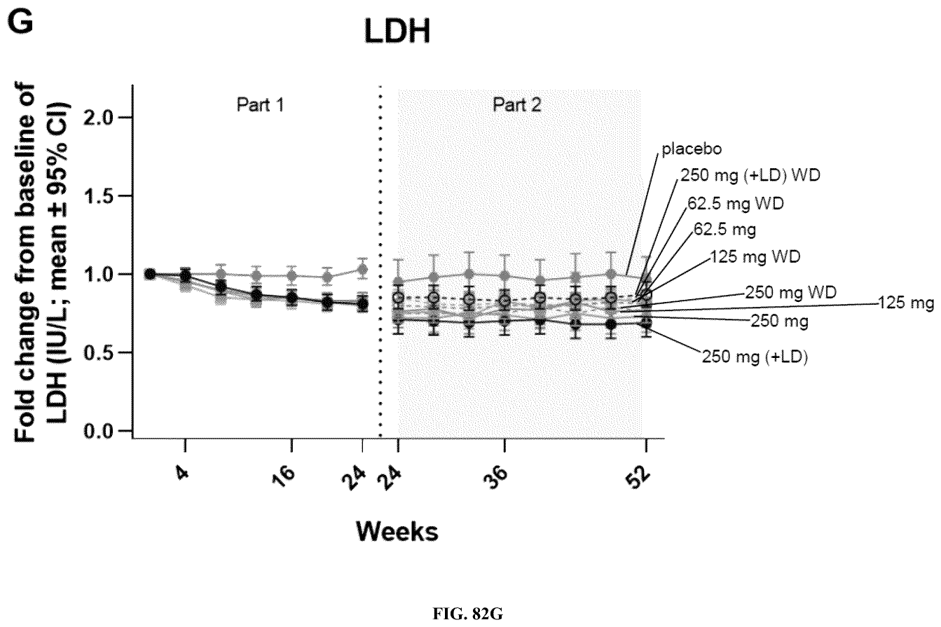

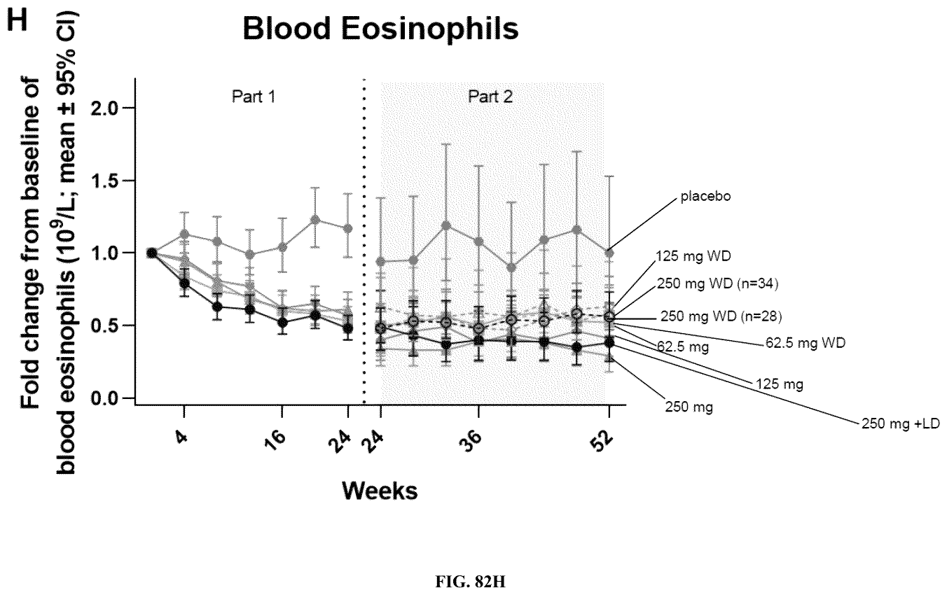

Consequently, this adjustment resulted in a refinement of the number of non-rescue participants for part 1, which was updated from 363 to 305 compared to the previous results. Amlitelimab treatment rapidly reduced serum biomarkers typically elevated in AD, including serum levels of Th2-related IL-13, IL-31, and TARC and serum levels of Th17/Th22-related IL-17A and IL-22. Amlitelimab treatment decreased serum LDH, total IgE, and blood eosinophil levels in the first 24 weeks of the study. Trends for the greatest fold-changes from baseline in TARC, blood eosinophils, and IL-17A were observed in the 250 mg+LD group. However, no clear dose-response relationship was observed. In part 2, serum IL-13, IL-31, TARC, IL-17A, IL-22, LDH, total IgE, and blood eosinophil levels remained reduced with both treatment continuation and withdrawal groups through week 52 (FIG. 82A-FIG. 82H).

### Example X

52-Week Results from a Phase 2b Trial (STREAM-AD)

### Background

Data from the 28-week amlitelimab maintenance/withdrawal period (Part 2) of the Phase 2b (STREAM-AD, NCT05131477) dose-ranging trial in adults with moderate-to-severe AD are presented in this example.

### Study Overview

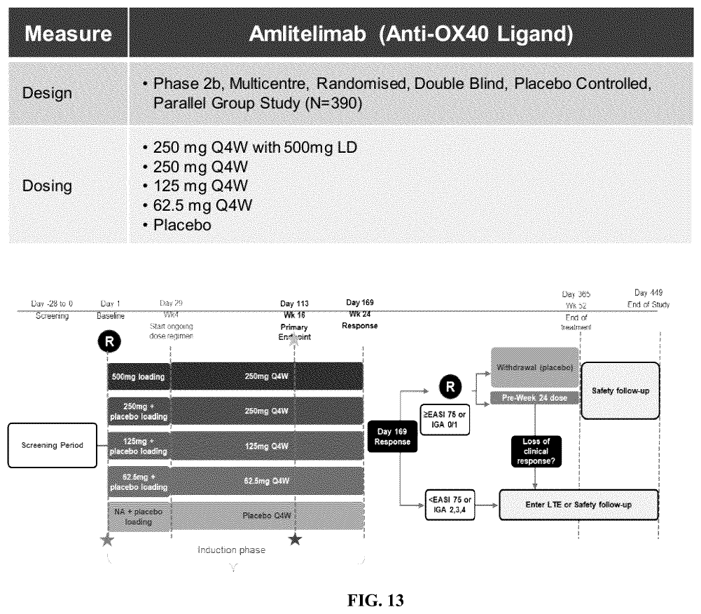

STREAM-AD Part 2 included clinical responders from Part 1, defined as participants achieving EASI-75 and/or IGA 0/1 at week 24 (FIG. 13). Of 390 participants enrolled in Part 1, 190 clinical responders (reaching EASI-75 and/or IGA 0/1) entered Part 2 withdrawal/maintenance period. Participants were re-randomized 3:1 to withdraw treatment or continue pre-week 24 subcutaneous Q4W dose (250 mg with 500 mg loading dose (LD), n=34 [treatment withdrawal]/n=13 [continuing]; 250 mg, n=28/n=12:125 mg, n=33/n=12; 62.5 mg, n=35/n=7; placebo responders continuing placebo, n=16), and were followed to week 52 for efficacy. Statistical analysis was conducted using two approaches: imputing endpoint as non-responder after rescue medication use (NRI) or including all measurements regardless of rescue use (treatment policy).

### Results

Maintenance of EASI-75 and/or IGA 0/1 response at week 52 was observed in 59%, 63%, 55%, and 66% of clinical responders withdrawn from Q4W dose of 250 mg with LD, 250 mg, 125 mg, and 62.5 mg, respectively (NRI). Using treatment policy, 77%, 82%, 67%, and 74% maintained response off-drug, respectively. Those continuing treatment had numerically higher maintenance response rates. AD-related biomarkers remained suppressed over 28 weeks, with >95% of the drug eliminated from serum for the last 8 weeks. The safety profile remained generally consistent with Part 1 without new concerns identified in Part 2.

### Conclusions

Maintenance of clinical responses was demonstrated for 28 weeks in the majority of patients, both on- and off-amlitelimab. Continued treatment for 28 weeks maintained high responder rates across all dose arms. 28 week withdrawal also demonstrated high responder rates with approximately 10-15 percentage points lower responder rates compared with continued treatment across all dose arms. IGA 0/1 showed a larger treatment difference compared to EASI-75 response rates.

Treatment withdrawal also demonstrated high responder rates with approximately 10 to 15 percentage points lower responder rates compared with continued treatment across all dose arms regardless of how rescue use was statistically handled.

No clear dose response was observed, providing further confidence in 250 mg Q12W dosing from initiation and/or as maintenance.

### Part 2 Study Specifics

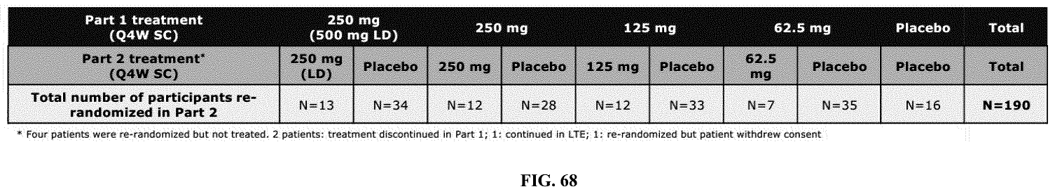

Part 1 enrolled/randomized 390 patients at study baseline in 5 arms: 250 mg Q4W with 500 mg LD (n=77), 250 mg Q4W (n=78), 125 mg Q4W (n=77), 62.5 mg Q4W (n=79) and placebo (n=70). At week 24, only responders (those reaching EASI-75 and/or IGA 0 or 1) entered Part 2 (n=190, 49%) (FIG. 68). Re-randomization 3:1 (withdrawal to placebo or pre-week 24 Q4W dose). Stratification was by IGA response (IGA 0/1 or IGA 2/3/4).

Participants that used rescue medications in Part 1 were eligible for re-randomized (n=28, 15%) which is different than other competitor studies which excluded them. However, results were similar whether or not these patients were included in the analysis. 167 participants (88%) completed the study to week 52. Of those who did not complete 52 weeks, n=7 (3.7%) transferred to long term extension (LTE) study, n=1 (0.5%) dropped out due to adverse events, n=1 (0.5%) dropped out due to lack of efficacy, and n=9 (4.7%) withdrew consent.

### Part 2 Objective

The objective of Part 2 was to explore the maintenance of clinical response on- and off-amlitelimab from week 24 through week 52 in those patients who achieved EASI-75 and/or who attained IGA 0/1 at the re-randomization visit.

### Topline Efficacy Endpoints

Topline efficacy endpoints were:
percentage of patients with IGA 0/1 at week 52 among patients with IGA 0/1 response at re-randomization visit;
percentage of patients with EASI-75 at Week 52 among patients with EASI-75 response at re-randomization visit; and
percentage of patients with EASI-75 and/or IGA 0/1 at Week 52 among patients with EASI-75 and/or IGA 0/1 response at re-randomization visit.

Statistical analysis was conducted using two approaches: 1) imputing endpoint as non-responder after rescue/prohibited medication use (NRI); or including all efficacy measurements regardless of rescue/prohibited use (treatment policy; missing data imputed as NR). Time-to-loss of EASI-75 up to week 52 in participants was randomized to withdrawal post-week 24

Other Endpoints (safety, PK/PD)

Other endpoints were:
summary of TEAEs from week 0 or from week 24 up to week 52 COD;
summary of incidence of ADAs;
serum concentrations of amlitelimab over time (from week 0 to week 52); and
summary of blood biomarkers (weeks 24, 36, 52).

### Baseline Demographics

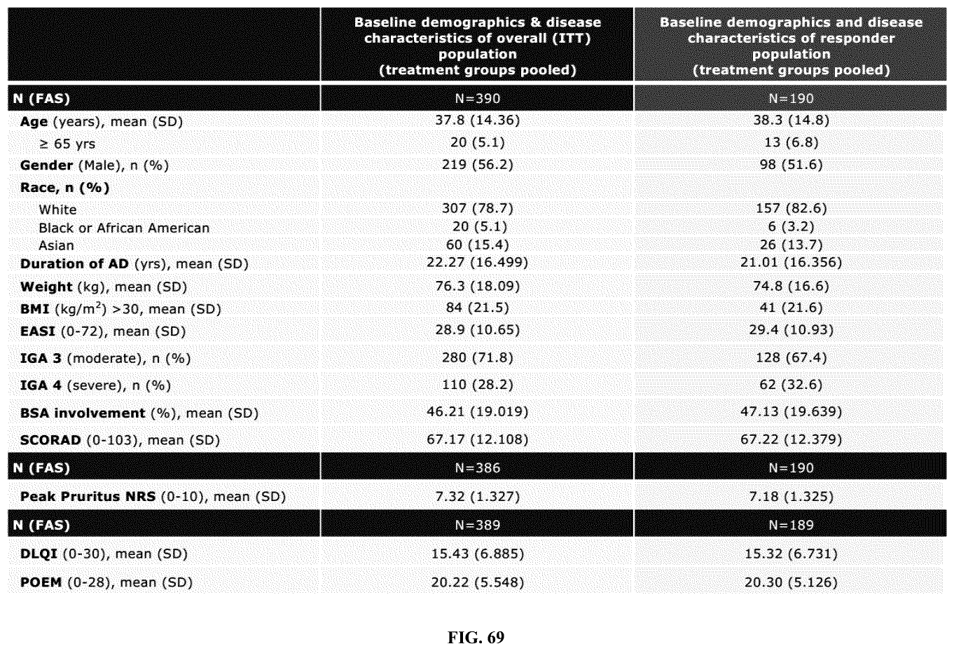

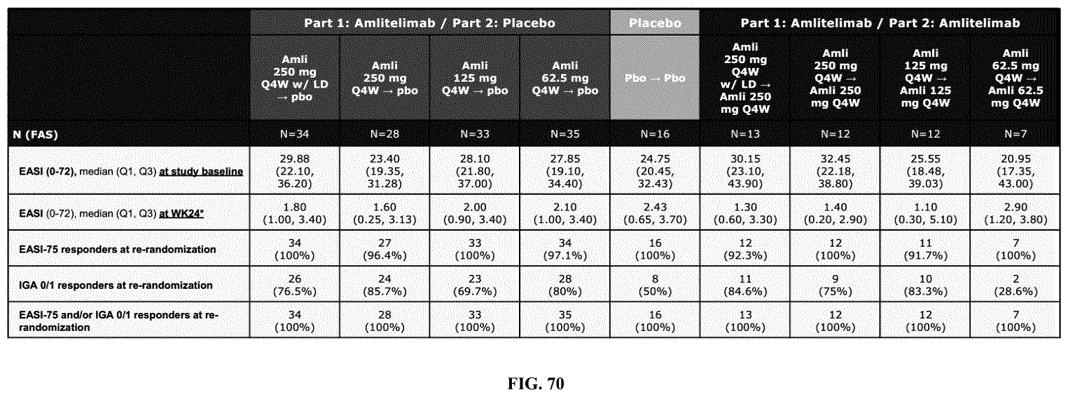

The baseline demographics and baseline disease characteristics were similar between the overall population and the responder population (FIG. 69). The baseline disease characteristics were well-balanced across dose arms and between those who continued amlitelimab treatment and those who underwent treatment withdrawal (FIG. 70).

### Topline Endpoint Data

### Maintenance of IGA 0/1 Response at Week 52

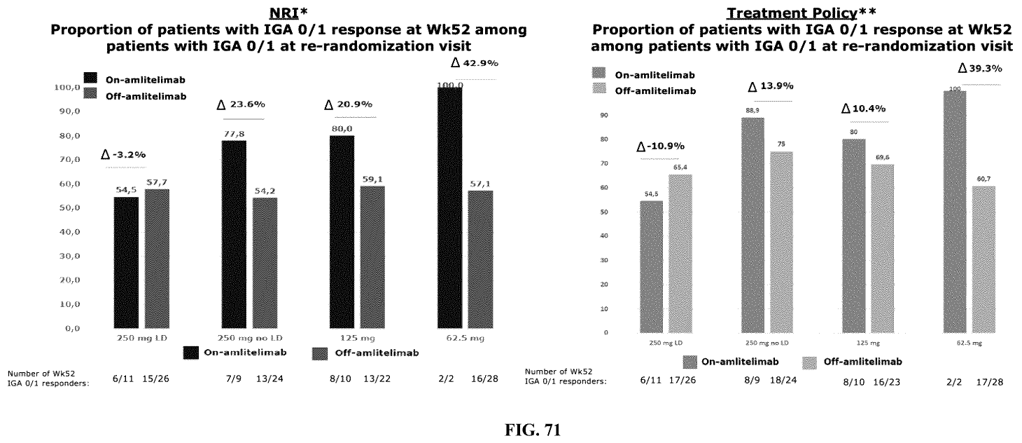

Continued treatment (on-amlitelimab) maintained high responder rates across all dose arms (FIG. 71). Treatment withdrawal (off-amlitelimab) also demonstrated high responder rates, with approximately 15 percentage points lower responder rates compared with continued treatment, with no clear dose-dependency. NRI analysis generally showed a larger delta between on- and off-amlitelimab responses relative to treatment policy analysis. Response rates in off-amlitelimab arms was similar to that observed in the amlitelimab Ph 2a study.

Pooled arms analysis: 72% on-amlitelimab and 57% off-amlitelimab with delta of 15 percentage points using NRI analysis: 75% on-amlitelimab and 67% off-amlitelimab with delta of 8 percentage points using treatment policy.

### Maintenance of EASI-75 Response at Week 24

Continued treatment (on-amlitelimab) maintained high responder rates across all dose arms (FIG. 72). Treatment withdrawal (off-amlitelimab) also demonstrated high responder rates with approximately 7 percentage points lower responder rates compared with continued treatment, with no clear dose-dependency. NRI analysis generally showed a larger delta between on-nd off-amlitelimab responses relative to treatment policy analysis.

Pooled arms analysis: 69% on-amlitelimab and 62% off-amlitelimab with delta of 7 percentage points using NRI analysis: 81% on-amlitelimab and 75% off-amlitelimab with delta of 6 percentage points using treatment policy.

Maintenance of IGA 0/1 and/or EASI-75 Response at Week 24 and Week 52

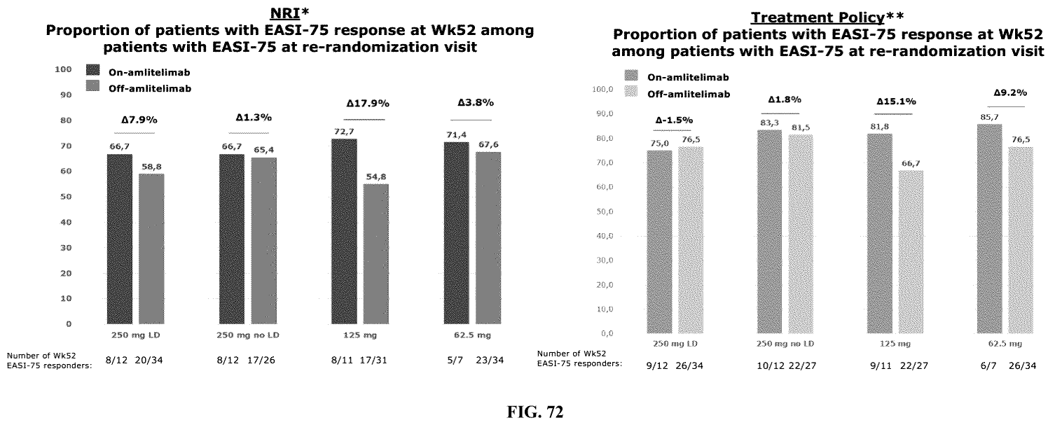

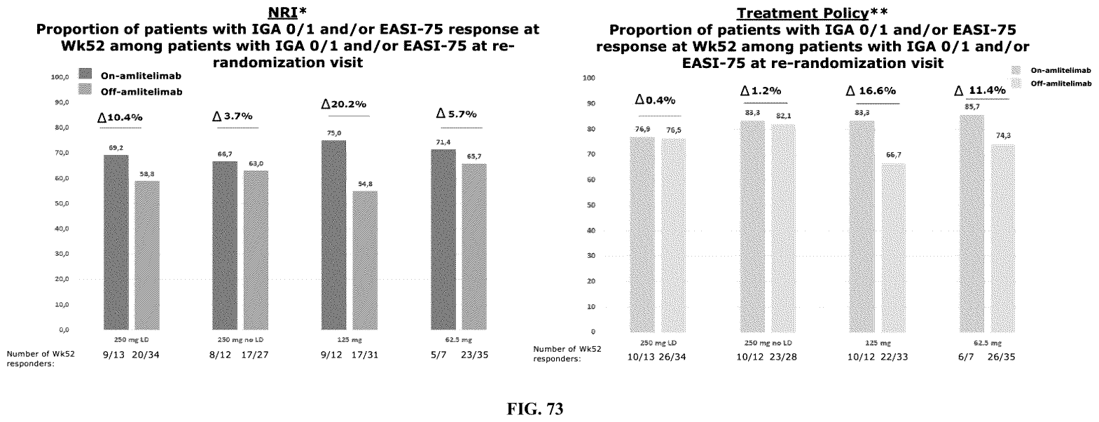

Continued treatment (on-amlitelimab) maintained high responder rates across all dose arms at week 24 (FIG. 73). Treatment withdrawal (off-amlitelimab) also demonstrated high responder rates, with approximately 9 percentage points lower responder rates compared with continued treatment, with no clear dose-dependency. NRI analysis generally showed a larger delta between on- and off-amlitelimab responses relative to treatment policy analysis.

Pooled arms analysis: 70% on-amlitelimab and 61% off-amlitelimab with delta of 9 percentage points using NRI analysis: 82% on-amlitelimab and 75% off-amlitelimab with delta of 7 percentage points using treatment policy.

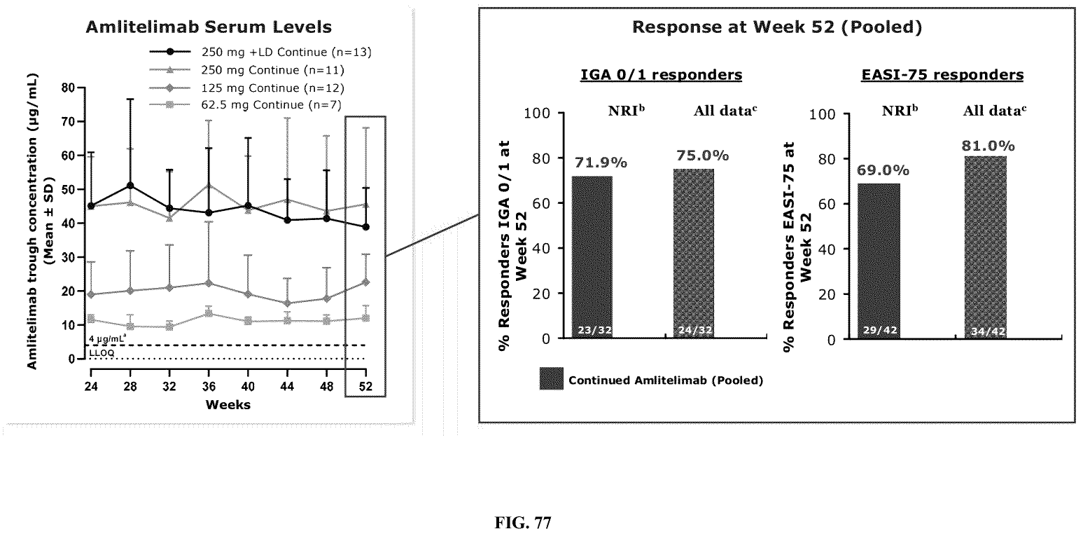

Durable clinical responses (IGA 0/1 and EASI-75) were maintained with treatment (pooled dose arms) at week 52 (FIG. 77).

Durability of EASI-75 and IGA 0/1 Responses after Treatment Withdrawal

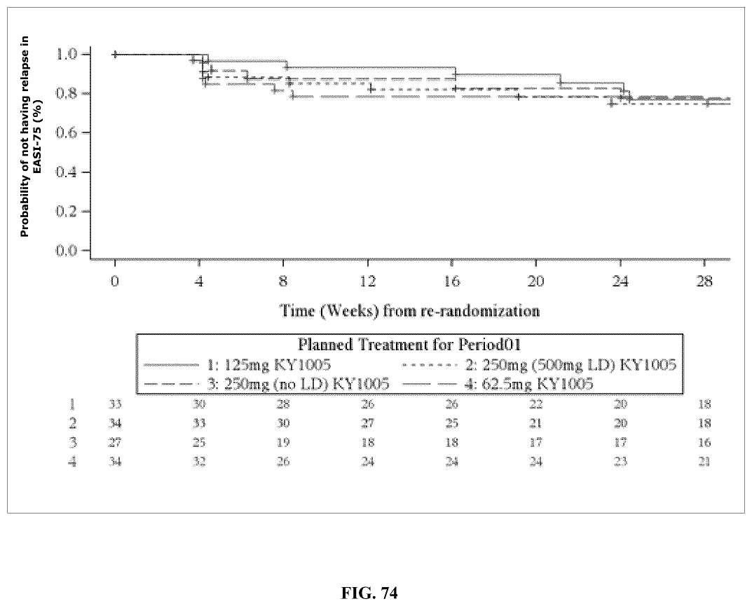

Time to loss of EASI-75 in patients reaching EASI-75 at the re-randomization week 24 visit (off-drug population) is depicted at FIG. 74.

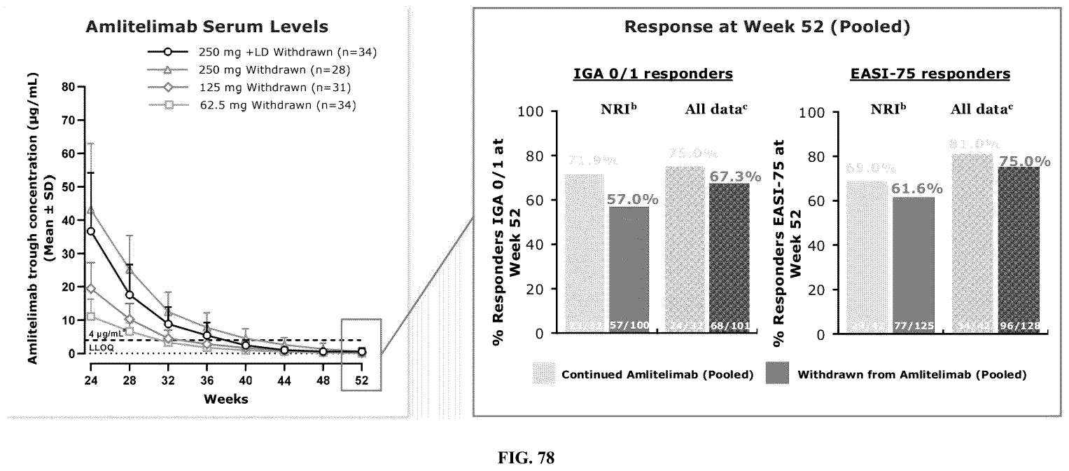

A durable clinical response (IGA 0/1 responders and EASI-75 responders) was maintained following drug withdrawal despite serum amlitelimab reaching negligible levels at week 52 (FIG. 78). Persistence of a response after withdrawal indicated potential normalization of inflammatory T-cells.

### Other Endpoint Data

### Safety

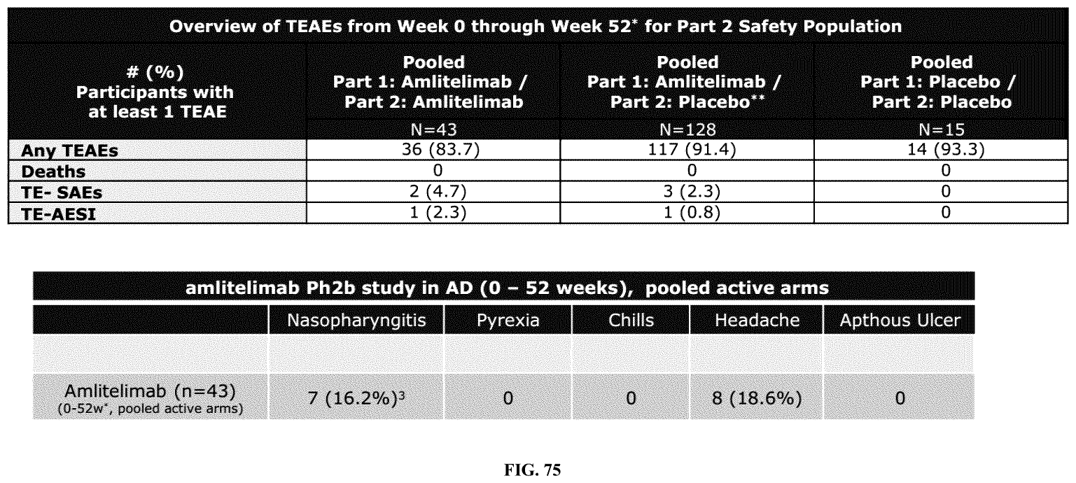

The overall safety profile remained generally consistent with the primary analysis without any new concerns identified. Amlitelimab was generally well-tolerated and demonstrated an acceptable safety profile across pooled dose arms for Part 2 safety population (week 0 to week 52) (FIG. 75). All SAEs and AESIs were reported as “not related” to amlitelimab by the investigator.

The most common TEAEs (≥5%/3 participants) in participants who received at least one dose of amlitelimab in both Part 1 and Part 2 (0-52 weeks) were: atopic dermatitis; upper respiratory tract Infection (includes viral URTI); headache; nasopharyngitis (including pharyngitis); COVID-19; or dizziness.

Part 1 pooled active arms (N=310, 0-24 weeks) vs. Part 2 (responders) pooled active arms (N=43, 24-52 weeks) (Adverse Events that occurred on re-randomization visit were counted in both Part 1 and Part 2).

Incidences of were TEAEs similar: Part 1=208 [67.4%], Part 2=30 [69.8%]. Incidences of SAEs were lower in Part 1: Part 1=8 [2.6%], Part 2=2 [4.7%]. Incidences of AESIs were similar: Part 1=6 [1.9%], Part 2=1 [2.3%].

There were no new concerns identified related to AESIs, selected TEAEs of interest, and most common AEs (FIG. 75). There were no reports of: allergic reactions that required immediate treatment/serious allergic reactions; malignancy; severe injection site reactions; severe/opportunistic/persistent infections; symptomatic overdoses; or conjunctivitis.

There were overall low incidences of: ALT elevation; COVID-19 (including patients with “COVID-19” and “suspected COVID-19”) (pooled amlitelimab/amlitelimab arms=1/43, 2.3%); and herpes (including patients with “herpes simplex” and “oral herpes”) (pooled amlitelimab/amlitelimab arms=2/43, 4.7%).

### Anti-Drug Antibody (ADA) Summary

ADA data were consistent with the primary analysis, with a low incidence of patients with treatment-emergent ADA. Maintained suppression of AD-related biomarkers (TARC, IL-13, IL-22, IgE, Eos) was observed in Part 2 in both withdrawal and original treatment groups through week 52. These biomarker findings were observed from week 44 to week 52, a time interval in which ≥95% of drug is cleared from serum.

The overall safety profile remained generally consistent with primary analysis without new concerns identified. ADA data were consistent with the primary analysis, with a low incidence of patients observed with treatment-emergent ADA.

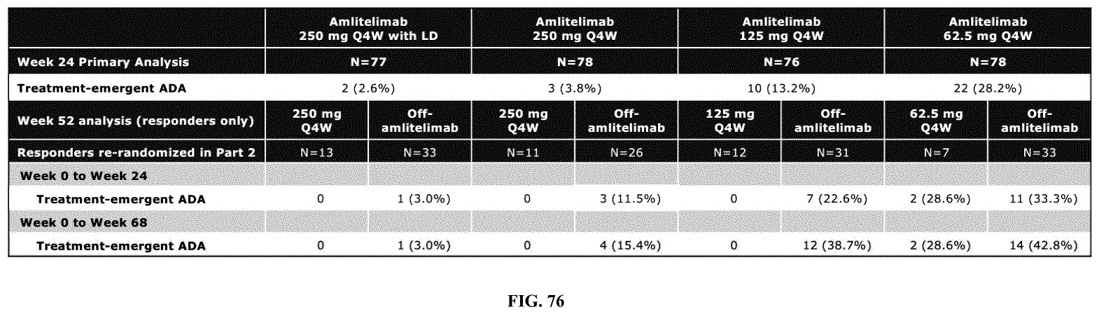

The ADA summary is shown at FIG. 76. Overall, the data were consistent with the week 24 primary analysis. A dose-dependent response in ADA with higher incidences of patients with treatment-emergent ADA was observed at 62.5 mg and 125 mg compared to 250 mg Q4W with or without LD. A low response level was observed across all dose arms with titer≤20 in all patients with the exception of 1 patient (125 mg) with a transient titer at 80.

In patients on-amlitelimab in Part 2, no additional patients became ADA positive after week 24.

In patients off-amlitelimab in Part 2, two patients (3.4%) at 250 mg (with or without LD), seven (22.6%) at 125 mg, and six (18.2%) at 62.5 mg became ADA positive while off-amlitelimab, and remained negative for 16 to 28 weeks after last administration with exception of one patient (125 mg) who became positive earlier

### Example XI

### A 26-Week SC Juvenile Toxicity Study of Amlitelimab Followed by a 13-Week Recovery Period in Cynomolgus Monkeys

A good laboratory practice (GLP)-compliant repeat dose toxicity study was conducted in juvenile (11 to 15 months old) cynomolgus monkeys to assess potential local and systemic toxicity of Amlitelimab, including bone morphologic and biochemical measurements, after repeated subcutaneous injection for 6 months (27 doses). Secondary objectives were to evaluate reversibility of any potential Amlitelimab-mediated effects during a 13 week treatment-free period and to evaluate toxicokinetics after repeated dosing of Amlitelimab.

Three groups of four male and four female juvenile cynomolgus monkeys each received once weekly subcutaneous injections of Amlitelimab at dose levels of 0 (vehicle control; 10 mM histidine, 220 mM sucrose, 0.06% polysorbate 80, 10 μM EDTA, pH 6.0), 10, and 50 mg/kg/week for 6 months (27 doses). Scheduled necropsies were conducted three days after the final dose (day 186). Two additional male and two additional female cynomolgus monkeys were dosed at 0 (vehicle only) or 50 mg/kg/week at the same time as the main study animals but were retained for an additional 13 week recovery period prior to scheduled necropsies on day 279.

The following parameters and endpoints were evaluated in this study; mortality, clinical signs, body weights, body weight gains, blood pressure, respiratory rates, ophthalmology and electrocardiogramhic examinations, clinical pathology parameters (hematology, coagulation, clinical chemistry, and urinalysis), bone turnover biomarkers (C-terminal Telopeptides of Type I Collagen (CTX-1) and Osteocalcin (OC)), bioanalytical and toxicokinetic parameters, organ weights, and macroscopic and microscopic examinations.

Immune function was assessed by evaluation of the T-cell dependent antibody response (TDAR) on generation of antibody (IgG and IgM) response to a keyhole limpet hemocyanin (KLH) challenge. KLH was administered as intramuscular injection at a dose of 3 mg/animal during study days 120 and 148. The weights of selected organs were recorded, and representative tissue samples (including bone growth plates) were examined microscopically from all monkeys euthanized at the end of the dosing period. Ex vivo assessments included bone scanning (femur and tibia) by micro-CT for bone densitometry parameters. Blood samples for toxicokinetic determinations were obtained on days 85 and 176 at pre-dose, and at 24, 48, 72, 96, and 168 hours post-dose.

There was no mortality, and there were no Amlitelimab-related effects on clinical observations, body weight, blood pressure, heart rate, respiratory rate, ophthalmic examinations, and electrocardiogramarameters. In TDAR, a slight but biologically non-relevant lowering of the primary antigen-specific IgG antibody responses in males and females and the primary antigen-specific IgM antibody responses in males to immunization with KLH was observed at 50 mg/kg/week. Amlitelimab had no effect on secondary IgG and IgM antibody responses to KLH.

There were no Amlitelimab-related effects on clinical pathology parameters (including bone turnover biomarkers CTX-1 and OC), hematology, coagulation, and urinalysis. There were no Amlitelimab-related organ weight changes, and no macroscopic or microscopic findings (including bone growth plate histopathology). There were no Amlitelimab-related effects on bone density observations measured by micro CT.

In conclusion, the administration of Amlitelimab at dose levels of 10 and 50 mg/kg/week to juvenile (11 to 15 months old) cynomolgus monkeys once weekly for 6 months (27 doses) by subcutaneous injection resulted in no local or systemic toxicity. The Non-Observable Effect Level (NOEL) for KY1005 was 50 mg/kg/week, the highest dose level tested.

### Example XII: Week 68 Safety Data

### Materials & Methods

STREAM-AD (NCT05131477) is a 2-part, Phase 2b, randomized, double-blind, placebo-controlled trial of amlitelimab in adults with moderate-to-severe AD. Part 1 involved a 24-week treatment period (last dose at week 20) with 388 participants treated with subcutaneous amlitelimab or placebo every 4 weeks (Q4W: 250 mg with 500 mg loading dose [250 mg+LD], n=77; 250 mg, n=78; 125 mg, n=77:62.5 mg, n=78; placebo, n=78). Among the participants who completed Part 1, 190 clinical responders (defined as achieving Eczema Area and Severity Index-75 (EASI-75) and/or Investigator Global Assessment (IGA) 0/1 at Week 24) entered Part 2 and were re-randomized 3:1 to placebo (withdrawal group) or to continue the pre-week 24 Q4W amlitelimab dose. Of those re-randomized, 186 participants were treated in Part 2 (250 mg+LD, n=34 [withdrawal]/n=13 [continuing]; 250 mg, n=28/n=11; 125 mg, n=32/n=12:62.5 mg, n=34/n=7; placebo responders continuing placebo, n=15). In Part 2, participants received their last dose (of amlitelimab or placebo) at week 48, with final efficacy analysis at week 52, and an additional 16-week safety follow-up to week 68 if they did not enroll in the long-term extension.

### Results

Weeks 0-68 safety data are presented from the 186 participants who completed Part 1, were re-randomized and received ≥1 dose of amlitelimab or placebo in Part 2. No dose-dependent relationship was observed in the total incidence of treatment-emergent adverse events (TEAEs), serious adverse events (SAEs), or adverse events of special interest; therefore, pooled data are presented (continued amlitelimab, n=43; withdrawn, n=128; continued placebo, n=15). From weeks 0-68, incidence of TEAEs were 83.7%, 92.2%, and 93.3% for continuing amlitelimab, withdrawn, and continuing placebo, respectively. The majority of TEAEs were mild or moderate in severity.

From weeks 0-68, the incidence of SAEs were 4.7%, 2.3%, and 0 for continuing amlitelimab, withdrawn, and continuing placebo, respectively, with 1 (0.8%) considered related to treatment in the withdrawn group by the investigator. One participant in the continued-treatment 125 mg arm had 4 TEAEs leading to treatment discontinuation (all related to laboratory abnormalities). These were considered not related to amlitelimab/placebo by the investigator.

No other TEAEs leading to treatment discontinuation were reported. There were no deaths in the study.

### Conclusions

In the STREAM-AD Phase 2b trial, amlitelimab was well-tolerated and demonstrated an acceptable safety profile in the Part 2 (responder) population up to 68 weeks.

### EMBODIMENTS OF THE INVENTION

The invention will now be described with reference to the following numbered embodiments.

1. A method of treating an immune-mediated disease in a subject in need thereof, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject receives directly doses every 12 weeks (Q12W), without prior dose administration every 4 weeks (Q4W).

2. A method of treating atopic dermatitis in a subject in need thereof, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject receives directly doses every 12 weeks (Q12W), without prior dose administration every 4 weeks (Q4W).

3. The method of embodiment 1 or 2, wherein the antibody or antigen binding fragment thereof is amlitelimab or a variant thereof.

4. The method of embodiment 1, 2 or 3, wherein the antibody is amlitelimab.

5. The method of any one of embodiments 1-4, wherein the antibody or antigen binding fragment thereof is administered by prefilled syringe, pen delivery device or autoinjector.

6. The method of any one of embodiments 1-4, wherein the subject receives an initial dose of about 500 mg of the antibody or antigen binding fragment thereof followed by one or more secondary doses of about 250 mg of the antibody or antigen binding fragment thereof.

7. The method of any of the preceding embodiments, wherein the atopic dermatitis is moderate-to-severe atopic dermatitis.

8. The method of embodiment 7, wherein the moderate-to-severe atopic dermatitis is not adequately controlled with topical prescription therapies or with systemic therapies or when those therapies are not advisable.

9. The method of any one of the preceding embodiments, wherein EASI score is reduced in the subject.

10. The method of embodiment 9, wherein the EASI score is selected from the group consisting of EASI-75, EASI-90 and EASI-100.

11. The method of any one of the preceding embodiments, wherein Investigator Global Assessment (IGA) score is reduced in the subject.

12. The method of any one of the preceding embodiments, wherein Peak Pruritis Numeric Rating Scale (PP-NRS) score is reduced in the subject.

13. The method of any one of the preceding embodiments, wherein Skin Pain Numeric Rating Scale (SP-NRS) score is reduced in the subject.

14. The method of any one of the preceding embodiments, wherein Sleep Disturbance Numeric Rating Scale (SD-NRS) score is reduced in the subject.

15. The method of any one of the preceding embodiments, wherein Dermatology Quality of Life Index (DLQI) score is reduced in the subject.

16. The method of any one of the preceding embodiments, wherein Hospital Anxiety and Depression Scale (HADS) score is improved in the subject.

17. A method of treating an immune mediated disease in an adolescent subject in need thereof, comprising administering to the adolescent subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60.

18. A method of treating atopic dermatitis in an adolescent subject in need thereof, comprising administering to the adolescent subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60.

19. The method of embodiment 17 or 18, wherein the antibody is amlitelimab or a variant thereof.

20. The method of embodiment 17, 18 or 19, wherein the antibody is amlitelimab.

21. The method of any one of embodiments 17-20, wherein the antibody or antigen binding fragment thereof is administered by prefilled syringe, pen delivery device or autoinjector.

22. The method of any one of embodiments 17-21, wherein the adolescent subject has a body weight ranging from about 25 kg to about 40 kg.

23. The method of any one of embodiments 17-22 wherein the adolescent subject receives an initial dose of about 250 mg of the antibody or antigen binding fragment thereof followed by one or more secondary doses of about 125 mg of the antibody or antigen binding fragment thereof.

24. The method of any one of embodiments 17-23, wherein each dose is administered Q12W.

25. The method of any one of embodiments 17-23, wherein the subject receives doses Q12W from the start of treatment.

26. The method of any one of embodiments 17-23, wherein the subject receives directly doses Q12W, without prior dose administration Q4W.

27. The method according to any one of embodiments 17-26, wherein the subject is aged 12 years or older (e.g. the subject is aged 12-17 years old).

28. The method of any one of embodiments 17-27, wherein the atopic dermatitis is moderate-to-severe atopic dermatitis.

29. The method of embodiment 28, wherein the moderate-to-severe atopic dermatitis is not adequately controlled with topical prescription therapies or with systemic therapies or when those therapies are not advisable.

30. The method of any one of embodiments 17-29, wherein EASI score is reduced in the subject.

31. The method of embodiment 30, wherein the EASI score is selected from the group consisting of EASI-75, EASI-90 and EASI-100.

32. The method of any one of embodiments 17-29, wherein IGA score is reduced in the subject.

33. The method of any one of embodiments 17-29, wherein PP-NRS score is reduced in the subject.

34. The method of any one of embodiments 17-29, wherein SP-NRS score is reduced in the subject.

35. The method of any one of embodiments 17-29, wherein SD-NRS score is reduced in the subject.

36. The method of any one of embodiments 17-29, wherein DLQI score is reduced in the subject.

37 The method of any one of embodiments 17-29, wherein HADS score is improved in the subject.

38 The method of any one of embodiments 17-29, wherein (a) the subject weighs equal to or greater than 25 kg and less than 40 kg, and wherein the subject is administered a formulation comprising a 62.5 mg/mL amlitelimab solution supplied as a prefilled syringe that delivers 125 mg of amlitelimab in a 2 mL injection, or (b) the subject weighs equal to or greater than 25 kg and less than 40 kg, and wherein the subject is administered a formulation comprising a 125 mg/mL amlitelimab solution supplied as a prefilled syringe that delivers 125 mg of amlitelimab in a 1 mL injection.

39. A method of treating an immune mediated disease in a subject in need thereof, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject has a body weight ranging from about 25 kg to about 40 kg.

40. A method of treating atopic dermatitis in a subject in need thereof, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject has a body weight ranging from about 25 kg to about 40 kg.

41. The method of embodiment 39 or 40, wherein the antibody is amlitelimab or a variant thereof.

42. The method of embodiment 39, 40 or 41, wherein the antibody is amlitelimab.

43. The method of any one of embodiments 39-42, wherein the antibody or antigen binding fragment thereof is administered by prefilled syringe, pen delivery device or autoinjector.

44. The method of any one of embodiments 39-43, wherein the subject receives an initial dose of about 250 mg of the antibody or antigen binding fragment thereof followed by one or more secondary doses of about 125 mg of the antibody or antigen binding fragment thereof.

45. The method of any one of embodiments 39-44, where each dose is administered Q12W.

46. The method of any one of embodiments 39-44, wherein the subject receives doses Q12W from the start of treatment.

47. The method of any one of embodiments 39-44, wherein the subject receives directly doses Q12W, without prior dose administration Q4W.

48. The method of embodiment 44, wherein each secondary dose is administered Q12W during 24 weeks or up to when the patient has achieved vIGA O/1, or has clear or substantially clear skin, or has achieved EASI 75 or has achieved EASI 90.

49. The method according to any one of embodiments 39-48, wherein the subject is aged 12 years or older (e.g. the subject is aged 12-17 years old).

50. The method of any one of embodiments 39-48, wherein the atopic dermatitis is moderate-to-severe atopic dermatitis.

51. The method of embodiment 50, wherein the moderate-to-severe atopic dermatitis is not adequately controlled with topical prescription therapies or with systemic therapies or when those therapies are not advisable.

52. The method of any one of embodiments 39-48, wherein EASI score is reduced in the subject.

53. The method of embodiment 52, wherein the EASI score is selected from the group consisting of EASI-75, EASI-90 and EASI-100.

54. The method of any one of embodiments 39-53, wherein IGA score is reduced in the subject.

55. A method of treating atopic dermatitis (AD) in a subject in need thereof, comprising: administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, and wherein the method results in a decrease of the level of at least one biomarker in the subject relative to a control, for example relative to the level of the at least one biomarker prior to administering an anti-OX40 ligand (OX40L) antibody or antigen binding fragment thereof.

56. A method of treating atopic dermatitis (AD) in a subject in need thereof, comprising:
administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60; and
reducing the level of at least one biomarker selecting from thymus and activation-regulated chemokine (TARC), interleukin-5 (IL-5), eosinophil count and lactate dehydrogenase (LDH), or combinations thereof.

57. A method of reducing a biomarker in a subject having atopic dermatitis (AD), comprising:
administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the biomarker is selected from thymus and activation-regulated chemokine (TARC), interleukin-5 (IL-5), eosinophil count and lactate dehydrogenase (LDH).

58. A method of treating atopic dermatitis (AD) in a subject in need thereof, comprising:
selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control; and
administering to the subject an effective amount of an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60

59. The method of any one of embodiments 55-70, wherein the antibody is amlitelimab or a variant thereof.

60. The method of any one of embodiments 55-70, wherein the antibody is amlitelimab.

61. The method of any one of embodiments 55 and 57-60, wherein the method results in a decrease in the level of at least one biomarker selecting from thymus and activation-regulated chemokine (TARC), interleukin-5 (IL-5), eosinophil count and lactate dehydrogenase (LDH), or combinations thereof, relative to the baseline level of the corresponding biomarker in the subject before administering the effective amount of an anti-OX40 ligand (OX40L) antibody or antigen binding fragment thereof.

62. The method of any one of embodiments 55-61, wherein the subject receives an initial dose of about 250 mg of the antibody or antigen binding fragment thereof followed by one or more secondary doses of about 125 mg of the antibody or antigen binding fragment thereof.

63. The method of any one of embodiments 55-61, wherein the subject receives an initial dose of about 500 mg of the antibody or antigen binding fragment thereof followed by one or more secondary doses of about 250 mg of the antibody or antigen binding fragment thereof.

64. The method of any one of embodiments 55-63, wherein the antibody or antigen binding fragment thereof is administered by prefilled syringe, pen delivery device or autoinjector.

65. The method of any one of one of embodiments 55-64, wherein the atopic dermatitis is moderate-to-severe atopic dermatitis.

66 The method of embodiment 65, wherein the moderate-to-severe atopic dermatitis is not adequately controlled with topical prescription therapies or with systemic therapies or wherein topical prescription therapies or systemic therapies are not advisable.

67. The method of any one of one of embodiments 55-66, wherein Eczema Area Surface Index (EASI) score is reduced in the subject.

68. The method of embodiment 67, wherein the EASI score is selected from the group consisting of EASI-75, EASI-90 and EASI-100.

69. The method of any one of embodiments 55-68, wherein Investigator Global Assessment (IGA) score is reduced in the subject.

70. The method of any one of embodiments 55-68, wherein Peak Pruritis Numeric Rating Scale (PP-NRS) score is reduced in the subject.

71. The method of any one of embodiments 55-68, wherein Skin Pain Numeric Rating Scale (SP-NRS) score is reduced in the subject.

72. The method of any one of embodiments 55-68, wherein Sleep Disturbance Numeric Rating Scale (SD-NRS) score is reduced in the subject.

73. The method of any one of embodiments 55-68, wherein Dermatology Quality of

Life Index (DLQI) score is reduced in the subject.

74. The method of any one of embodiments 55-68, wherein Hospital Anxiety and Depression Scale (HADS) score is improved in the subject.

75. A method of treating an immune-mediated disease in a subject in need thereof, comprising:
administering to the subject an anti-OX40 ligand (OX40L) antibody or antigen binding fragment thereof,
wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, and
wherein the immune-mediated disease is selected from the group consisting of atopic dermatitis (AD), asthma, interstitial lung disease-systemic sclerosis (ILD-SSc), systemic sclerosis (SSc), hidradenitis suppurativa (HS), alopecia areata (AA) and celiac disease.

76. A method of treating atopic dermatitis in a subject in need thereof, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein efficacy is maintained at week 28 off of treatment or at week 32 after final administration of the antibody or antigen binding fragment thereof to the subject.

77. The method of embodiment 76, wherein each dose is administered Q12W.

78. The method of embodiment 76 or 77, wherein the subject receives doses Q12W from the start of treatment.

79. The method of any one of embodiments 76-78, wherein the subject receives directly doses Q12W, without prior dose administration Q4W.

80. The method of any one of embodiments 76-79, wherein the subject is a vIGA 0/1 responder and vIGA O/1 response efficacy is maintained in the subject, or wherein the subject is an AESI 75 responder and EASI 75 response efficacy is maintained in the subject.

81. The method of embodiment 80, wherein the subject is a vIGA 0/1 responder and vIGA O/1 response efficacy is maintained in the subject.

82. The method of embodiment 80, wherein the subject is an AESI 75 responder and EASI 75 efficacy response is maintained in the subject.

83. The method of embodiment 80, wherein the subject does not experience a treatment-emergent adverse event (TEAE).

84. The method of embodiment 75, wherein the immune-mediated disease is AD.

85. The method of embodiment 75, wherein the immune-mediated disease is asthma.

86. The method of embodiment 75, wherein the immune-mediated disease is ILD-SSc.

87. The method of embodiment 75, wherein the immune-mediated disease is SSc.

88 The method of embodiment 75, wherein the immune-mediated disease is HS.

89. The method of embodiment 75, wherein the immune-mediated disease is AA.

90. The method of embodiment 75, wherein the immune-mediated disease is celiac disease.

91. The method of embodiment 75, wherein systemic therapies are not advisable.

92. A method of treating atopic dermatitis in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

93. A method of treating atopic dermatitis in an adolescent subject in need thereof, comprising administering to the adolescent subject amlitelimab, wherein the adolescent subject receives an initial dose of about 250 mg of amlitelimab followed by one or more secondary doses of about 125 mg of amlitelimab, and wherein the subject receives doses Q12W from the start of treatment.

94. A method of treating atopic dermatitis in a subject in need thereof, comprising administering to the subject amlitelimab, wherein the subject has a body weight ranging from about 25 kg to about 40 kg, wherein the subject receives an initial dose of about 250 mg of amlitelimab followed by one or more secondary doses of about 125 mg of amlitelimab, and wherein the subject receives doses Q12W from the start of treatment.

95. A method of treating atopic dermatitis (AD) in a subject in need thereof, comprising administering to the subject amlitelimab, wherein the subject receives directly doses every 12 weeks (Q12W), without prior dose administration every 4 weeks (Q4W), wherein the subject receives an initial dose of about 500 mg of amlitelimab followed by one or more secondary doses of about 250 mg of amlitelimab, and wherein the method results in a decrease of the level of at least one biomarker in the subject relative to a control.

96. A method of treating atopic dermatitis (AD) in a subject in need thereof, comprising administering to the subject amlitelimab, wherein the subject receives directly doses every 12 weeks (Q12W), without prior dose administration every 4 weeks (Q4W), wherein the subject receives an initial dose of about 500 mg of amlitelimab followed by one or more secondary doses of about 250 mg of amlitelimab, and reducing the level of at least one biomarker selecting from thymus and activation-regulated chemokine (TARC), interleukin-5 (IL-5), eosinophil count and lactate dehydrogenase (LDH), or combinations thereof.

97. A method of reducing a biomarker in a subject having atopic dermatitis (AD), comprising administering to the subject amlitelimab, wherein the subject receives directly doses every 12 weeks (Q12W), without prior dose administration every 4 weeks (Q4W), wherein the subject receives an initial dose of about 500 mg of amlitelimab followed by one or more secondary doses of about 250 mg of amlitelimab, and wherein the biomarker is selected from thymus and activation-regulated chemokine (TARC), interleukin-5 (IL-5), eosinophil count and lactate dehydrogenase (LDH).

98. A method of treating atopic dermatitis (AD) in a subject in need thereof, comprising selecting a subject having AD and an elevated level of at least one biomarker selected from the group consisting of thymus and activation-regulated chemokine (TARC), interleukin-5 (IL-5), eosinophil count, and lactate dehydrogenase (LDH) relative to a control, and administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

99. A method of treating atopic dermatitis (AD) in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg amlitelimab.

100. A method of treating atopic dermatitis (AD) in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

101. A method of treating asthma in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

102. A method of treating interstitial lung disease-systemic sclerosis in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

103. A method of treating systemic sclerosis in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

104. A method of treating hidradenitis suppurativa in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

105. A method of treating alopecia areata in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

106. A method of treating celiac disease in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab.

107. A method of treating atopic dermatitis in a subject in need thereof, comprising administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab, and wherein efficacy is maintained at week 28 off of treatment or at week 32 after final administration of amlitelimab.

108. The method of any one of embodiments 1, 2, 17, 18, 39, 40, 55-58, 75 and 76, wherein the antibody or antigen binding fragment thereof is an OX40L antagonist.

1. A method of treating an immune-mediated disease or atopic dermatitis in a subject in need thereof, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject receives directly doses every 12 weeks (Q12W), without prior dose administration every 4 weeks (Q4W).

2. (canceled)

3. The method of claim 1, wherein;
the antibody or antigen binding fragment thereof is amlitelimab or a variant thereof;
the antibody is amlitelimab;
the antibody or antigen binding fragment thereof is administered by prefilled syringe, pen delivery device or autoinjector; or
the subject receives an initial dose of about 500 mg of the antibody or antigen binding fragment thereof followed by one or more secondary doses of about 250 mg of the antibody or antigen binding fragment thereof.

4-6. (canceled)

7. The method of claim 1, wherein:
the atopic dermatitis is moderate-to-severe atopic dermatitis, optionally wherein the moderate-to-severe atopic dermatitis is not adequately controlled with topical prescription therapies or with systemic therapies or when those therapies are not advisable;
Eczema Area and Severity Index (EASI) score is reduced in the subject, optionally wherein the EASI score is selected from the group consisting of EASI-75, EASI-90 and EASI-100; and/or
Investigator Global Assessment (IGA) score is reduced in the subject.

8-11. (canceled)

12. A method of treating an immune mediated disease or atopic dermatitis in an adolescent subject in need thereof, comprising administering to the adolescent subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60.

13. (canceled)

14. The method of claim 12, wherein;
the antibody is amlitelimab or a variant thereof;
the antibody is amlitelimab; and/or
the antibody or antigen binding fragment thereof is administered by prefilled syringe, pen delivery device or autoinjector.

15-16. (canceled)

17. The method of claim 12, wherein;
the adolescent subject has a body weight ranging from about 25 kg to about 40 kg; and/or
the adolescent subject is aged 12 years or older and is optionally between 12 and 17 years old.

18. The method of claim 12, wherein:
the adolescent subject receives an initial dose of about 250 mg of the antibody or antigen binding fragment thereof followed by one or more secondary doses of about 125 mg of the antibody or antigen binding fragment thereof;
each dose is administered Q12W;
the adolescent subject receives doses Q12W from the start of treatment; or
the subject receives directly doses Q12W, without prior dose administration Q4W.

19-22. (canceled)

23. The method of claim 12, wherein;
the atopic dermatitis is moderate-to-severe atopic dermatitis, optionally wherein the moderate-to-severe atopic dermatitis is not adequately controlled with topical prescription therapies or with systemic therapies or when those therapies are not advisable;
wherein EASI score is reduced in the adolescent subject, optionally wherein the EASI score is selected from the group consisting of EASI-75, EASI-90 and EASI-100; or
wherein IGA score is reduced in the adolescent subject.

24-27. (canceled)

28. The method of claim 12, wherein;
(a) the subject weighs equal to or greater than 25 kg and less than 40 kg, and wherein the subject is administered a formulation comprising a 62.5 mg/mL amlitelimab solution supplied as a prefilled syringe that delivers 125 mg of amlitelimab in a 2 mL injection; or
(b) the subject weighs equal to or greater than 25 kg and less than 40 kg, and wherein the subject is administered a formulation comprising a 125 mg/mL amlitelimab solution supplied as a prefilled syringe that delivers 125 mg of amlitelimab in a 1 mL injection.

29. A method of treating an immune mediated disease or atopic dermatitis in a subject in need thereof, comprising administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein the subject has a body weight ranging from about 25 kg to about 40 kg.

30. (canceled)

31. The method of claim 29, wherein:
the antibody is amlitelimab or a variant thereof;
the antibody is amlitelimab; and/or
the antibody or antigen binding fragment thereof is administered by prefilled syringe, pen delivery device or autoinjector.

32-33. (canceled)

34. The method of claim 29, wherein;
the subject receives an initial dose of about 250 mg of the antibody or antigen binding fragment thereof followed by one or more secondary doses of about 125 mg of the antibody or antigen binding fragment thereof;
each dose is administered Q12W;
the subject receives doses Q12W from the start of treatment;
the subject receives directly doses Q12W, without prior dose administration Q4W; or
each secondary dose is administered Q12W during 24 weeks or up to when the patient has achieved vIGA O/1, or has clear or substantially clear skin, or has achieved EASI 75 or has achieved EASI 90.

35-38. (canceled)

39. The method according to claim 29, wherein the subject is aged 12 years or older, optionally wherein the subject is aged 12-17 years old.

40. The method of claim 29, wherein the atopic dermatitis is moderate-to-severe atopic dermatitis, optionally wherein the moderate-to-severe atopic dermatitis is not adequately controlled with topical prescription therapies or with systemic therapies or when those therapies are not advisable.

41. (canceled)

42. The method of claim 29, wherein;
EASI score is reduced in the subject, optionally wherein the EASI score is selected from the group consisting of EASI-75, EASI-90 and EASI-100; and/or
IGA score is reduced in the subject.

43-44. (canceled)

45. A method of treating atopic dermatitis in a subject in need thereof, comprising:
administering to the subject an anti-OX40 ligand (OX40L) antibody or antigen binding fragment thereof, wherein the antibody or antigen binding fragment thereof comprises a heavy chain complementarity determining region (HCDR) 1 of SEQ ID NO: 42, an HCDR2 of SEQ ID NO: 44, and an HCDR3 of SEQ ID NO: 46, and a light chain complementarity determining region (LCDR) 1 of SEQ ID NO: 56, an LCDR2 of SEQ ID NO: 58, and an LCDR3 of SEQ ID NO: 60, wherein efficacy is maintained at week 28 off of treatment or at week 32 after final administration of the antibody or antigen binding fragment thereof to the subject;
administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab;
administering to the subject amlitelimab, wherein the adolescent subject receives an initial dose of about 250 mg of amlitelimab followed by one or more secondary doses of about 125 mg of amlitelimab, and wherein the subject receives doses Q12W from the start of treatment, wherein the subject is an adolescent;
administering to the subject amlitelimab, wherein the subject has a body weight ranging from about 25 kg to about 40 kg, wherein the subject receives an initial dose of about 250 mg of amlitelimab followed by one or more secondary doses of about 125 mg of amlitelimab, and wherein the subject receives doses Q12W from the start of treatment;
administering to the subject amlitelimab, wherein the subject receives directly doses every 12 weeks (Q12W), without prior dose administration every 4 weeks (Q4W), wherein the subject receives an initial dose of about 500 mg of amlitelimab followed by one or more secondary doses of about 250 mg of amlitelimab, and wherein the method results in a decrease of the level of at least one biomarker in the subject relative to a control;
administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg amlitelimab;
administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab; or
administering to the subject amlitelimab, wherein the subject receives directly doses every 12 weeks (Q12W), without prior dose administration every 4 weeks (Q4W), and wherein the subject receives an initial dose of about 500 mg of amlitelimab followed by one or more secondary doses of about 250 mg of amlitelimab, and wherein efficacy is maintained at week 28 off of treatment or at week 32 after final administration of amlitelimab.

46. The method of claim 45, wherein:
each dose is administered Q12W;
the subject receives doses Q12W from the start of treatment; and/or
the subject receives directly doses Q12W, without prior dose administration Q4W.

47-48. (canceled)

49. The method of claim 45, wherein:
the subject is a vIGA 0/1 responder and vIGA O/1 response efficacy is maintained in the subject, or wherein the subject is an AESI 75 responder and EASI 75 response efficacy is maintained in the subject;
the subject is a vIGA 0/1 responder and vIGA 0/1 response efficacy is maintained in the subject; or
the subject is an AESI 75 responder and EASI 75 efficacy response is maintained in the subject.

50-58. (canceled)

59. The method of claim 1, wherein the antibody or antigen binding fragment thereof is an OX40L antagonist.

60. The method of claim 45, wherein the antibody or antigen binding fragment thereof is an OX40L antagonist.
